(19) United States
(12) Patent Application Publication
(10) Pub. No.: US 2026/0123278 A1
(43) Pub. Date: Apr. 30, 2026

(54) COMPOUND FOR ORGANIC ELECTRONIC DEVICE, ORGANIC ELECTRONIC DEVICE USING THE SAME, AND ELECTRONIC DEVICE THEREOF

(71) Applicant: DUK SAN NEOLUX CO., LTD., Cheonan-si, Chungcheongnam-do (KR)

(72) Inventors: Hyung Dong LEE, Cheonan-si, Chungcheongnam-do (KR); Sun Hee LEE, Cheonan-si, Chungcheongnam-do (KR); Zhao Yang ZHONG, Cheonan-si, Chungcheongnam-do (KR)

(73) Assignee: DUK SAN NEOLUX CO., LTD., Cheonan-si, Chungcheongnam-do (KR)

(21) Appl. No.: 19/120,459

(22) PCT Filed: Oct. 4, 2023
(86) PCT No.: PCT/KR2023/015207

§ 371 Date: Apr. 11, 2025

(30) Foreign Application Priority Data
Oct. 11, 2022 (KR) 10-2022-0129524

(51) Int. Cl. H10K 85/60; C07B 59/00; C07D 251/24; C07D 413/12; C07D 417/12; C09K 11/02; H10K 50/12

(52) U.S. Cl. CPC H10K 85/636; C07B 59/002; C07D 251/24; C07D 413/12; C07D 417/12; C09K 11/02; H10K 85/615; H10K 85/626; H10K 85/654; H10K 50/12; H10K 85/657; H10K 85/6574; H10K 85/6576

(57) ABSTRACT

Provided are a compound represented by Formula 1, an organic electronic element comprising a first electrode, a second electrode, and an organic layer disposed between the first and second electrodes, and an electronic device comprising the organic electronic element, wherein the inclusion of the compound represented by Formula 1 in the organic layer can lower the driving voltage of the organic electronic element and improves the luminous efficiency and lifetime thereof.

### TECHNICAL FIELD

The present invention relates to a compound for an organic electronic device, an organic electronic device comprising the same, and an electronic apparatus comprising the device.

### BACKGROUND ART

In general, organic electroluminescence refers to a phenomenon in which electrical energy is converted into light energy by an organic material. An organic electronic device utilizing organic electroluminescence typically includes an anode, a cathode, and an organic layer interposed therebetween. In many cases, the organic layer has a multi-layered structure comprising different materials, respectively, in order to improve the efficiency and stability of an organic electronic device. For example, the organic layer may comprise a hole-injecting layer, a hole-transporting layer, a light-emitting layer, an electron-transporting layer, and an electron-injecting layer.

The materials used in the organic layer of an organic electronic device may be classified, according to their functions, into a light-emitting material and charge-transporting materials such as a hole-injecting material, a hole-transporting material, an electron-transporting material, and an electron-injecting material. Further, the light-emitting material may be classified into a high molecular weight type and a low molecular weight type according to molecular weight, and may also be classified into a fluorescent material, which emits light from an excited singlet state, and a phosphorescent material, which emits light from an excited triplet state, according to its light-emission mechanism. Further, the light-emitting material may be classified, according to its emission color, into blue, green, and red light-emitting materials, and yellow and orange light-emitting materials, which are required for improved natural color reproduction.

Meanwhile, when a single material is used as a light-emitting material, problems may occur, such as a shift in the maximum emission wavelength toward a longer wavelength due to intermolecular interactions, a deterioration in color purity, or a reduction in luminous efficiency, thereby resulting in decreased efficiency of the corresponding device. Accordingly, a host/dopant system may be employed as the light-emitting material in order to enhance color purity and improve luminous efficiency through energy transfer. This is based on the principle that, when a small amount of a dopant having a smaller energy band gap than that of a host forming the light-emitting layer is mixed into the light-emitting layer, excitons generated in the light-emitting layer are transferred to the dopant, thereby enabling light emission with high efficiency. Here, since the emission wavelength of the host is shifted to the wavelength region of the dopant, light having a desired wavelength can be obtained depending on the type of the dopant.

Currently, the portable display market is expanding with the adoption of large-area displays, which require greater power consumption than conventional portable displays. Accordingly, power consumption has become a critical factor for portable displays that operate with limited battery power, and issues related to efficiency and lifespan must also be addressed.

Efficiency, lifespan, driving voltage, and the like are interrelated. An increase in efficiency may lead to a decrease in driving voltage, which in turn may reduce the crystallization of organic materials caused by Joule heating generated during device operation. As a result, the lifespan of the device may be extended. However, efficiency cannot be maximized solely by improving the organic layer. This is because both long lifespan and high efficiency can be simultaneously achieved only when an optimal combination is established among the energy levels, T₁ values, and intrinsic material properties (e.g., charge mobility, interfacial characteristics, etc.) of the respective layers constituting the organic layer.

Therefore, it is necessary to develop an emitting material that exhibits high thermal stability and can efficiently achieve charge balance in the light-emitting layer. That is, in order to fully realize the excellent characteristics of an organic electronic device, the materials constituting the organic layer—such as hole-injecting materials, hole-transporting materials, light-emitting materials, electron-transporting materials, and electron-injecting materials—must be based on materials that are both stable and efficient. In particular, it is crucial to develop a material for the light-emitting layer.

### DETAILED DESCRIPTION OF THE INVENTION

### Technical Challenge

An object of the present invention is to provide a compound capable of lowering the driving voltage of a device and improving the luminous efficiency and lifespan of the device, an organic electronic device comprising the same, and an electronic apparatus including the organic electronic device.

### Means of Solving Problems

In one aspect, the present invention provides a compound represented by the following formula:

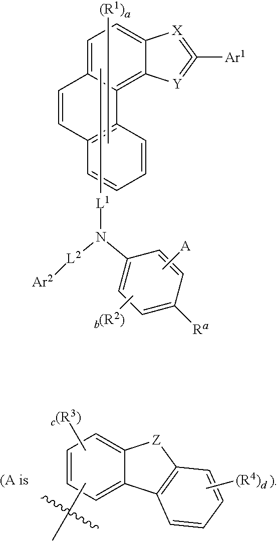

In another aspect, the present invention provides a method for recovering the compound represented by the above formula, wherein the compound can be reused after being recovered following deposition.

In another aspect, the present invention provides an organic electronic device comprising a compound represented by the above formula and an electronic apparatus comprising the organic electronic device.

In another aspect, the present invention provides an organic electronic device comprising both a compound represented by the above formula and a compound represented by the following formula, and an electronic apparatus comprising the organic electronic device.

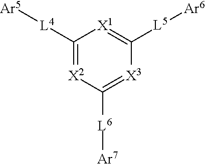

### Effect of Invention

According to an embodiment of the present invention, by using the compound, the driving voltage of a device can be reduced, and the luminous efficiency and lifetime of the device can be improved. In addition, the compound used in the deposition process can be recovered and reused.

### BRIEF DESCRIPTION OF THE DRAWINGS

FIGS. 1 to 3 illustrate an example of organic electroluminescent device according to the present invention.

[Description of Reference Numerals]

| | |
|---|---|
| 100, 200, 300: organic | 110: first electrode |
| electronic device | |
| 120: hole-injecting layer | 130: hole-transporting layer |
| 140: light-emitting layer | 150: electron-transporting layer |
| 160: electron-injecting layer | 170: second electrode |
| 180: layer for improving light | 210: buffer layer |
| efficiency | |
| 220: light-emitting auxiliary layer | 320: first hole-injecting layer |
| 330: first hole-transporting layer | 340: first light-emitting layer |
| 350: first electron-transporting layer | 360: first charge-generation layer |
| 361: second charge-generation layer | 420: second hole-injecting layer |
| 430: second hole-transporting layer | 440: second light-emitting layer |
| 450: second electron-transporting | CGL: charge generation layer |
| layer | |
| ST1: first stack | ST2: second stack |

### DETAILED DESCRIPTION

Unless otherwise stated, the term “aryl group” or “arylene group” as used herein refers to a group having 6 to 60 carbon atoms, but is not limited thereto. The aryl group or arylene group in the present invention may comprise a monocyclic ring, ring assemblies, a fused polycyclic system, a spiro compound, and the like.

As used herein, the term “fluorenyl group” refers to a fluorenyl moiety that may be substituted or unsubstituted, and the term “fluorenylene group” refers to a fluorenylene moiety that may be substituted or unsubstituted. The fluorenyl group or fluorenylene group employed in the present invention may comprise a spiro compound in which R and R′ are bonded to each other in the structure shown below, and may also comprise compounds in which adjacent R″ groups are linked together. The terms “substituted fluorenyl group” and “substituted fluorenylene group” mean that at least one of R, R′, or R″ in the following structure is a substituent other than hydrogen. In the following structure, the number of R″ groups may range from 1 to 8. Throughout this specification, the fluorenyl group and fluorenylene group may collectively be referred to as a “fluorene group” or “fluorene,” regardless of their valence.

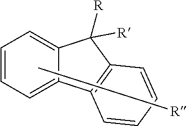

As used herein, the term “spiro compound” refers to a compound having a spiro linkage, which is a structure in which two rings are connected through a single common atom. The atom shared by the two rings is referred to as a “spiro atom,” and the compound may be classified as a monospiro, dispiro, or trispiro compound depending on the number of spiro atoms present in the molecule.

As used herein, the term “heterocyclic group” comprises both aromatic rings, such as a “heteroaryl group” or a “heteroarylene group,” and non-aromatic rings. Unless otherwise specified, the “heterocyclic group” refers to a ring structure containing one or more heteroatoms and having from 2 to 60 carbon atoms, but is not limited thereto. The term “heteroatom,” as used herein, refers to atoms such as nitrogen (N), oxygen (O), sulfur(S), phosphorus (P), or silicon (Si), and may also include heteroatomic groups such as SO₂, P═O, and the like, which can replace a carbon atom in the ring structure as shown in the following compound. The “heterocyclic group” may comprise monocyclic ring, ring assemblies, fused polycyclic system, a spiro compound, and the like.

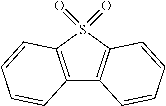 [SMILES: O=S1(=O)c2ccccc2-c2ccccc21]

As used herein, the term “aliphatic ring group” refers to a cyclic hydrocarbon excluding aromatic hydrocarbons, and comprises, but is not limited to, monocyclic rings, ring assemblies, fused polycyclic systems, spiro compounds, and the like. Unless otherwise specified, the aliphatic ring group may comprise a ring having from 3 to 60 carbon atoms. For example, a fused ring system composed of benzene, which is an aromatic ring, and cyclohexane, which is a non-aromatic ring, corresponds to an aliphatic ring group.

In this specification, the “group name” corresponding to an aryl group, an arylene group, a heterocyclic group, and the like, exemplified for each symbol and its substituent, may be expressed either as a functional group name reflecting the valence or as the name of the parent compound. For example, in the case of phenanthrene, which is a type of aryl group, it may be described as “phenanthryl (group)” when referring to a monovalent group, and as “phenanthrylene (group)” when referring to a divalent group. Alternatively, it may also be described by its parent compound name “phenanthrene,” regardless of valence. Similarly, in the case of pyrimidine, it may be referred to as “pyrimidine” regardless of its valence. Alternatively, it may be described by the name of the corresponding functional group, such as “pyrimidinyl (group)” for a monovalent group and “pyrimidylene (group)” for a divalent group.

In addition, in the present specification, numerical and alphabetical indicators of positions may be omitted when describing the name of a compound or a substituent. For example, compounds such as pyrido[4,3-d]pyrimidine, benzofuro[2,3-d]pyrimidine, and 9,9-dimethyl-9H-fluorene may be described in a simplified manner as pyridopyrimidine, benzofurropyrimidine, and dimethylfluorene, respectively. Accordingly, both benzo[g]quinoxaline and benzo[f]quinoxaline may be generally referred to as benzoquinoxaline.

In addition, unless otherwise specified, when any compound according to the present invention is represented by the following formula, each substituent corresponding to the respective index is defined as described below.

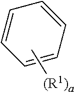

In the above formula, when a is zero, the substituent R¹ is absent, meaning that hydrogen atoms are bonded to all carbon atoms constituting the benzene ring. Here, chemical structures or compounds may be represented without explicitly indicating hydrogen atoms bonded to carbon atoms. In addition, when a is an integer of 1, one substituent R¹ is bonded to one of the carbon atoms constituting the benzene ring. When a is 2 or 3, the substituents may be bonded as exemplified below. In the case where a is an integer from 4 to 6, the substituents are similarly bonded to the carbon atoms of the benzene ring. Further, when a is greater than or equal to 2, the substituents R¹ may be identical or different from each other.

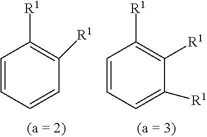

In addition, unless otherwise specified in the specification, the term ‘ring’ refers to an aryl ring, heteroaryl ring, fluorene ring, aliphatic ring, etc., and a number-membered (atom) ring may refer to the shape of a ring. For example, naphthalene corresponds to a two-fused (condensed) ring, anthracene to a three-fused (condensed) ring, thiophene or furan corresponds to a five-membered ring, and benzene or pyridine corresponds to a six-membered ring.

In addition, unless otherwise specified in the present specification, when adjacent groups are linked to each other to form a ring, the ring may be selected from the group consisting of a C₆-C₆₀ aromatic ring group, a fluorenyl group, a C₂-C₆₀ heterocyclic group containing at least one heteroatom selected from O, N, S, Si, and P, and a C₃-C₆₀ aliphatic ring group. Here, the aromatic ring group may comprise an aryl ring, and the heterocyclic group may comprise a heteroaryl ring.

Unless otherwise specified, the term “(between) adjacent groups,” as used herein, comprises not only the relationships such as “(between) R₁ and R₂,” “(between) R₂ and R₃” “(between) R₃ and R₄,” and “(between) R₅ and R₆” but also “(between) R₇ and R₈” sharing a common carbon atom. It may further comprise cases “(between) substituents” attached to different ring-forming atoms (e.g., carbon or nitrogen), such as “(between) R₁ and R₇,” “(between) R₁ and R₈,” or “(between) R₄ and R₅.” That is, even when substituents are not directly adjacent on the same atom, one substituent may be considered adjacent to another substituent attached to a neighboring ring-forming atom. Additionally, substituents bonded to the same carbon atom forming the ring may also be regarded as adjacent groups. In the following Formula, when substituents such as R₇ and R₈, which are bonded to the same carbon atom, are connected to form a ring, a compound containing a spiro moiety may be generated.

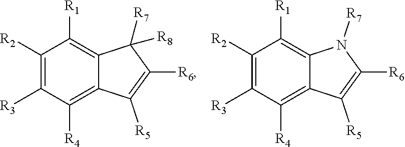

In addition, in the present specification, the expression “adjacent groups may be linked to each other to form a ring” is used in the same sense as “adjacent groups are selectively linked to each other to form a ring,” and refers to a case where at least one pair of adjacent groups may be bonded to form a ring structure.

In addition, unless otherwise specified in the present specification, substituents such as an aryl group, an arylene group, a fluorenyl group, a fluorenylene group, a heterocyclic group, an aliphatic ring group, an alkyl group, an alkenyl group, an alkynyl group, an alkoxyl group, an aryloxyl group, alkylthio group, arylthio group, etc., and a ring formed by adjacent groups may be each optionally substituted with one or more substituents selected from the group consisting of deuterium, halogen, a cyano group, a nitro group, siloxane group, a C₆-C₃₀ aryl group, a fluorenyl group, a C₂-C₃₀ heterocyclic group containing at least one heteroatom of O, N, S, Si and P, a C₃-C₃₀ aliphatic ring group, a C₁-C₂₀ alkyl group, a C₂-C₂₀ alkenyl group, a C₂-C₂₀ alkynyl group, a C₁-C₂₀ alkoxyl group, a C₆-C₂₀ aryloxy group, a C₁-C₂₀ alkylthio group, a C₆-C₂₀ arylthio group, a silane group unsubstituted or substituted with a C₁-C₂₀ alkyl group or a C₆-C₂₀ aryl group, and a phosphine oxide group unsubstituted or substituted with a C₁-C₂₀ alkyl group or a C₆-C₂₀ aryl group.

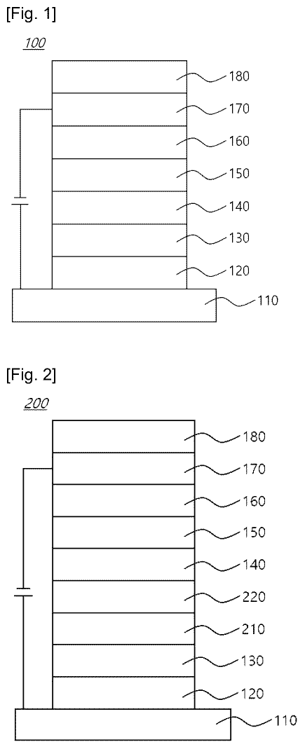

Hereinafter, with reference to FIGS. 1 to 3, the laminated structure of an organic electronic device comprising the compound according to the present invention will be described.

In the designation of reference numerals for components in the respective drawings, it should be understood that the same elements are denoted by the same reference numerals, even if they appear in different drawings. Furthermore, in the following description of the present invention, detailed explanations of well-known functions and configurations will be omitted where they may unnecessarily obscure the essence of the invention.

Terms such as “first,” “second,” “A,” “B,” “(a),” “(b),” and the like may be used to describe various components of the present invention. These terms are merely intended to distinguish one component from another and do not imply any particular order, importance, or essential characteristics. Furthermore, it should be understood that when a component is described as being “connected,” “coupled,” or “joined” to another component, this may include both direct connections as well as indirect connections through one or more intervening components.

Additionally, it is to be understood that when an element such as a layer, film, region, or substrate is described as being “on” or “over” another element, it may be positioned directly on the other element or with one or more intervening layers therebetween. In contrast, the expression “directly on” indicates that no intervening elements are present between the two elements.

FIGS. 1 to 3 respectively illustrate examples of an organic electronic device according to embodiments of the present invention.

Referring to FIG. 1, an organic electronic device 100 according to an embodiment of the present invention comprises a first electrode 110, a second electrode 170, and an organic layer disposed between the first electrode 110 and the second electrode 170 on a substrate (not shown). In some embodiments, an inorganic layer may also be interposed between the first electrode 110 and the second electrode 170.

For example, the first electrode 110 may function as an anode (positive electrode), and the second electrode 170 may function as a cathode (negative electrode). In an inverted organic electronic device, however, the first electrode may serve as a cathode, while the second electrode may serve as an anode.

The organic layer refers to a layer comprising at least one organic material. For example, the organic layer may include a hole-injecting layer 120, a hole-transporting layer 130, a light-emitting layer 140, an electron-transporting layer 150, and an electron-injecting layer 160. In certain embodiments, the electron-injecting layer 160 may be an inorganic layer that does not contain any organic material.

Specifically, a hole-injecting layer 120, a hole-transporting layer 130, a light-emitting layer 140, an electron-transporting layer 150, and an electron-injecting layer 160 may be sequentially formed on the first electrode 110.

Preferably, a layer for improving the luminous efficiency 180 may be formed on one side of the first electrode 110 or the second electrode 170, wherein the one side does not face the organic layer or inorganic layer. When the layer for improving the luminous efficiency 180 is formed, the luminous efficiency of the organic electronic device can be enhanced.

For example, the layer for improving the luminous efficiency 180 may be formed on the second electrode 170. As a result, in the case of a top-emission organic light emitting device, optical energy loss due to surface plasmon polaritons (SPPs) at the second electrode 170 may be reduced. In the case of a bottom-emission organic light emitting device, the layer for improving the luminous efficiency 180 may function as a buffer layer for the second electrode 170.

A buffer layer 210 or a light-emitting auxiliary layer 220 may additionally be formed between the hole-transporting layer 130 and the light-emitting layer 140, as will be described with reference to FIG. 2.

Referring to FIG. 2, an organic electronic device 200 according to another embodiment of the present invention may sequentially include a hole-injecting layer 120, a hole-transporting layer 130, a buffer layer 210, a light-emitting auxiliary layer 220, a light-emitting layer 140, an electron-transporting layer 150, an electron-injecting layer 160, and a second electrode 170 on a first electrode 110, and a layer for improving the luminous efficiency 180 may be formed on the second electrode 170.

Although not illustrated in FIG. 2, an electron transport auxiliary layer may additionally be formed between the light-emitting layer 140 and the electron-transporting layer 150.

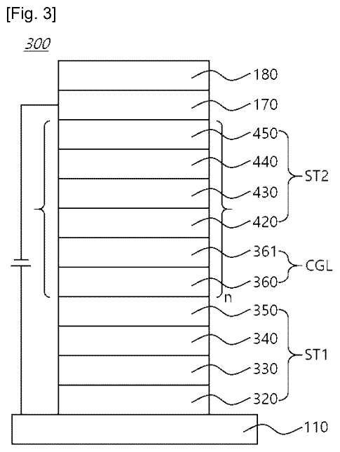

In addition, according to another embodiment of the present invention, the organic layer may be in the form of multiple stacks, each of which includes a hole-transporting layer, a light-emitting layer, and an electron-transporting layer. This will be described with reference to FIG. 3.

Referring to FIG. 3, in an organic electronic device 300 according to another embodiment of the present invention, two or more stacks of organic layers, ST1 and ST2, may be formed between the first electrode 110 and the second electrode 170. Each stack may include multiple organic layers, and a charge generation layer (CGL) may be formed between the stacks.

Specifically, the organic electronic device according to the embodiment of the present invention may comprise a first electrode 110, a first stack ST1, a charge generation layer CGL, a second stack ST2, and a second electrode 170 and a layer for improving light efficiency 180.

The first stack ST1 is an organic layer formed on the first electrode 110, and may comprise a first hole-injecting layer 320, a first hole-transporting layer 330, a first light-emitting layer 340, and a first electron-transporting layer 350. The second stack ST2 may comprise a second hole-injecting layer 420, a second hole-transporting layer 430, a second light-emitting layer 440, and a second electron-transporting layer 450. As such, the first stack and the second stack may have the same or different stacked structures of organic layers.

The charge generation layer CGL may be formed between the first stack ST1 and the second stack ST2. The charge generation layer CGL may comprise a first charge generation layer 360 and a second charge generation layer 361. It is positioned between the first light-emitting layer 340 and the second light-emitting layer 440 to enhance the current efficiency of each light-emitting layer and facilitate charge distribution.

The first light-emitting layer 340 may comprise a light-emitting material that comprises a blue host doped with a blue fluorescent dopant, and the second light-emitting layer 440 may comprise a light-emitting material that comprises a green host doped with both a greenish-yellow dopant and a red dopant. However, the materials of the first light-emitting layer 340 and the second light-emitting layer 440 according to an embodiment of the present invention are not limited thereto.

In FIG. 3, n may be an integer from 1 to 5, and when n is 2, a charge generation layer (CGL) and a third stack may be additionally formed on the second stack ST2.

When a plurality of light-emitting layers are formed in a multi-layer stack structure as shown in FIG. 3, it is possible to manufacture an organic electroluminescent element that emits not only white light but also various colors, where the white light is produced by the mixing of light emitted from each light-emitting layer.

Compound(s) represented by Formula 1 of the present invention may be included in an organic layer. For example, the compounds represented by Formula 1 of the present invention can be used as a material for a hole-injecting layer 120, 320, 420, a hole-transporting layer 130, 330, 430, a buffer layer 210, a light-emitting auxiliary layer 220, an electron-transporting layer 150, 350, 450, a light-emitting layer 140, 340, 440, and/or a light efficiency improving layer 180. More preferably, they may be used as a host material in the light-emitting layers 140, 340, or 440.

Even if the cores of the compounds are identical or similar, their band gaps, electronic properties, and interfacial characteristics may vary depending on which substituents are bonded and at which positions. Therefore, it is necessary to study the selection of the core structure and the combination with sub-substituents attached to the core. In particular, a long lifespan and high efficiency can be achieved simultaneously when the optimal combination of energy levels, T1 values, and intrinsic material properties (such as mobility and interfacial characteristics) is realized among the layers of the organic structure.

Therefore, by using the compound represented by Formula 1 as a material for the light-emitting layers 140, 340, and 440, it is possible to optimize the energy levels and T1 values between the respective layers of the organic layer, as well as the intrinsic material properties such as mobility and interfacial characteristics.

The organic electronic device according to an embodiment of the present invention may be fabricated using various deposition methods, comprising physical vapor deposition (PVD) or chemical vapor deposition (CVD). For example, the organic electronic device may be manufactured by forming the anode 110 on the substrate by depositing a metal, a conductive metal oxide, or a mixture thereof, then forming an organic layer comprising the hole-injecting layer 120, the hole-transporting layer 130, the light-emitting layer 140, the electron-transporting layer 150, and the electron-injecting layer 160 thereon, and finally depositing a material that can be used as the cathode 170. In addition, a light-emitting auxiliary layer 220 may be formed between the hole-transporting layer 130 and the light-emitting layer 140, and an electron transport auxiliary layer (not shown) may additionally be formed between the light-emitting layer 140 and the electron-transporting layer 150. As described above, these layers may constitute a stacked structure.

In addition, the organic layer may be manufactured with fewer layers by using various polymer materials through a solution process or solvent-based process, such as spin coating, nozzle printing, inkjet printing, slot coating, dip coating, roll-to-roll, doctor blading, screen printing, or thermal transfer, instead of deposition. Since the organic layer according to the present invention may be formed in various ways, the scope of protection of the present invention is not limited by the method of forming the organic layer.

The organic electronic device according to an embodiment of the present invention may be a top-emission type, a bottom-emission type, or a dual-emission type, depending on the materials used.

In addition, the organic electronic device according to an embodiment of the present invention may be selected from the group consisting of an organic electroluminescent device, an organic solar cell, an organic photoconductor, an organic transistor, a monochromatic illumination device, and a quantum dot display device.

Another embodiment of the present invention provides an electronic apparatus comprising a display device including the above-described organic electronic device and a control unit for controlling the display device. The electronic apparatus may be a wired or wireless communication terminal currently in use or to be developed in the future, and comprises all types of electronic devices, such as mobile communication terminals (e.g., cellular phones), navigation units, game players, various types of TVs, and computers.

Hereinafter, a compound according to one aspect of the present invention will be described.

A compound according to one aspect of the present invention is represented by Formula 1 below.

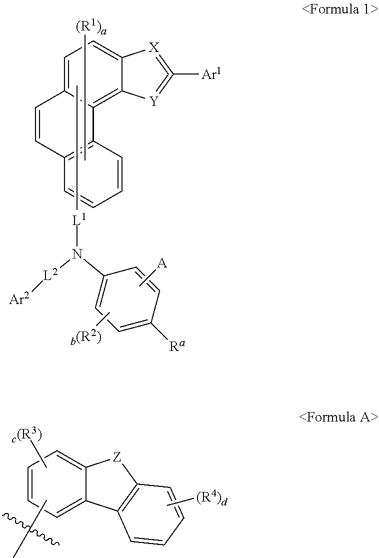

In Formula 1, each symbol may be defined as follows.

X and Y are each independently N, O or S, and one of X and Y is N and the other is O or S. In a pentagonal ring containing X and Y, the bond designation between X—C—Y means that when X is N and Y is O or S, the carbon to which Ar¹ is bonded and X are bonded by a double bond, and the carbon to which Ar¹ is bonded and Y are bonded by a single bond, and when X is O or S and Y is N, the carbon to which Ar¹ is bonded and X are bonded by a single bond, and the carbon to which Ar¹ is bonded and Y are bonded by a double bond.

Ar¹ and Ar² are each independently selected from the group consisting of a C₆-C₆₀ aryl group, a fluorenyl group, a C₃-C₆₀ aliphatic ring group, and a C₂-C₆₀ heterocyclic group containing at least one heteroatom of O, N, S, Si and P.

L¹ and L² are each independently selected from the group consisting of a single bond, a C₆-C₆₀ arylene group, a fluorenylene group, a C₃-C₆₀ aliphatic ring group, and a C₂-C₆₀ heterocyclic group containing at least one heteroatom of O, N, S, Si and P.

A is Formula A.

Z is O or S.

R¹ to R⁴, R^{a} are each independently selected from the group consisting of hydrogen, deuterium, halogen, a cyano group, a nitro group, a C₆-C₆₀ aryl group, a fluorenyl group, a C₂-C₆₀ heterocyclic group containing at least one heteroatom of O, N, S, Si and P, a C₃-C₆₀ aliphatic ring group, a C₁-C₂₀ alkyl group, a C₂-C₂₀ alkenyl group, a C₂-C₂₀ alkynyl group, a C₁-C₂₀ alkoxyl group, and a C₆-C₆₀ aryloxy group, and adjacent R³ or adjacent R⁴ may be bonded to each other to form a ring.

a is an integer of 0 to 7, b to c are each an integer of 0 to 3, d is an integer of 0 to 4.

A ring formed by adjacent groups, for example, adjacent R³s or adjacent R⁴s may be selected from the group consisting of a C₆-C₆₀ aromatic ring group, a fluorenylene group, a C₃-C₆₀ heterocyclic group containing at least one heteroatom of O, N, S, Si and P, and a C₆-C₆₀ aliphatic ring.

When an aromatic ring is formed by adjacent groups, the aromatic ring may be, for example, a C₆-C₂₀, a C₆-C₁₈, a C₆-C₁₆, a C₆-C₁₄, a C₆-C₁₃, a C₆-C₁₂, a C₆-C₁₀, a C₆, a C₁₀, a C₁₂, a C₁₄, a C₁₅, a C₁₆, or a C₁₈ aromatic ring, specifically, an aryl ring such as benzene, naphthalene, anthracene, phenanthrene, pyrene, etc.

When at least one of Ar¹, Ar², R¹ to R⁴, R^{a} is an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, or a C₁₈ aryl group, specifically, phenyl, biphenyl, naphthyl, terphenyl, phenanthrene, triphenylene, or the like.

When at least one of L¹ and L² is an arylene group, the arylene group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, or a C₁₈ arylene group, specifically, phenylene, biphenyl, naphthylene, terphenyl, phenanthrene, triphenylene, or the like.

When at least one of Ar¹, Ar², R¹ to R⁴, R^{a}, L¹ and L² is a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, or a C₂₉ heterocyclic group, specifically, pyridine, pyrimidine, pyrazine, pyridazine, triazine, furan, pyrrole, indene, indole, phenyl-indole, benzoindole, phenyl-benzoindole, pyrazinoindol, quinoline, isoquinoline, benzoquinoline, pyridoquinoline, quinazoline, benzoquinazoline, dibenzoquinazoline, phenanthroquinazoline, quinoxaline, benzoquinoxaline, dibenzoquinoxaline, benzofuran, naphthobenzofuran, dibenzofuran, dinaphthofuran, thiophene, benzothiophene, dibenzothiophene, naphthobenzothiophene, dinaphthothiophene, carbazole, phenyl-carbazole, benzocarbazole, phenyl-benzocarbazole, naphthyl-benzocarbazole, dibenzocarbazole, indolocarbazole, benzofuropyridine, benzothienopyridine, benzofuropyridine, benzothienopyrimidine, benzofuropyrimidine, benzothienopyrazine, benzofuropyrazine, benzoimidazole, benzothiazole, benzooxazole, benzosiloe, phenanthroline, dihydro-phenylphenazine, 10-phenyl-10H-phenoxazine, phenoxazine, phenothiazine, dibenzodioxin, benzodibenzodioxin, thianthrene, 9,9-dimethyl-9H-xanthene, 9,9-dimethyl-9H-thioxanthene, dihydrodimethylphenylacridine, spiro[fluorene-9,9′-xanthene] and the like.

When at least one of Ar¹, Ar², R¹ to R⁴, R^{a}, L¹ and L² is an aliphatic ring group, the aliphatic ring group, may be, for example, a C₃-C₃₀, a C₃-C₂₉, a C₃-C₂₈, a C₃-C₂₇, a C₃-C₂₆, a C₃-C₂₅, a C₃-C₂₄, a C₃-C₂₃, a C₃-C₂₂, a C₃-C₂₁, a C₃-C₂₀, a C₃-C₁₉, a C₃-C₁₈, a C₃-C₁₇, a C₃-C₁₆, a C₃-C₁₅, a C₃-C₁₄, a C₃-C₁₃, a C₃-C₁₂, a C₃-C₁₁, a C₃-C₁₀, a C₃-C₈, a C₃-C₆, a C₆, a C₁₀, a C₁₁, a C₁₂, a C₁₃, a C₁₄, a C₁₅, a C₁₆, a C₁₇ or a C₁₈ aliphatic ring group, specifically, a cyclopentanyl group, a cyclohexanyl group, a norbornyl group, an adamantyl group, etc.

When at least one of R¹ to R⁴, R^{a} is an alkyl group, the alkyl group may be, for example, a C₁-C₂₀, a C₁-C₁₀, a C₁-C₄, a C₁, a C₂, a C₃, or a C₄ alkyl group, for example, methyl, ethyl, t-butyl, etc.

The aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent R³ or adjacent R⁴ may be each substituted with one or more substituents selected from the group consisting of deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group.

When at least one of the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent R³ or adjacent R⁴ is substituted with an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, or a C₁₈ aryl group.

When at least one of the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent R³ or adjacent R⁴ is substituted with an aliphatic ring group, the aliphatic ring group may be, for example, a C₃-C₃₀, a C₃-C₂₉, a C₃-C₂₈, a C₃-C₂₇, a C₃-C₂₆, a C₃-C₂₅, a C₃-C₂₄, a C₃-C₂₃, a C₃-C₂₂, a C₃-C₂₁, a C₃-C₂₀, a C₃-C₁₉, a C₃-C₁₈, a C₃-C₁₇, a C₃-C₁₆, a C₃-C₁₅, a C₃-C₁₄, a C₃-C₁₃, a C₃-C₁₂, a C₃-C₁₁, a C₃-C₁₀, a C₃-C₈, a C₃-C₆, a C₆, a C₁₀, a C₁₁, a C₁₂, a C₁₃, a C₁₄, a C₁₅, a C₁₆, a C₁₇ or a C₁₈ aliphatic ring group, specifically, a cyclopentanyl group, a cyclohexanyl group, a norbornyl group, an adamantyl group, etc.

When at least one of the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent R³ or adjacent R⁴ is substituted with an alkyl group, the alkyl group may be, for example, a C₁-C₂₀, a C₁-C₁₀, a C₁-C₄, a C₁, a C₂, a C₃, or a C₄ alkyl group, for example, methyl, ethyl, t-butyl, etc.

Formula 1 may be represented by one of Formula 1-1 to Formula 1-6.

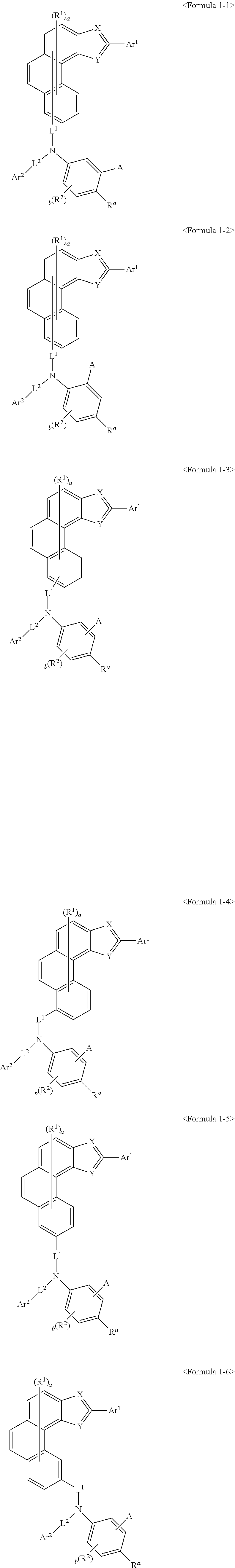

In Formula 1-1 to Formula 1-6, X, Y, R¹, R², R^{a}, L¹, L², Ar¹, Ar², A, a, b are the same as defined for Formula 1.

Formula A may be represented by one of Formula A-1 to Formula A-4, but there is no limitation thereto.

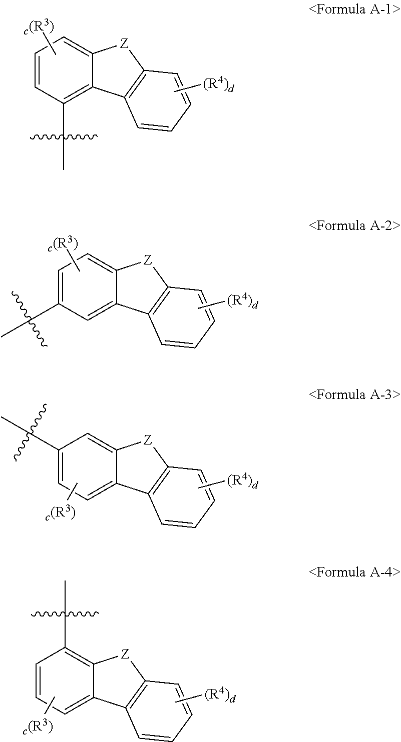

In Formula A-1 to Formula A-4, Z, R³, R⁴, c, d are the same as defined for Formula 1.

L¹ or L² may be selected from the group consisting of Formula L-1 to Formula L-3.

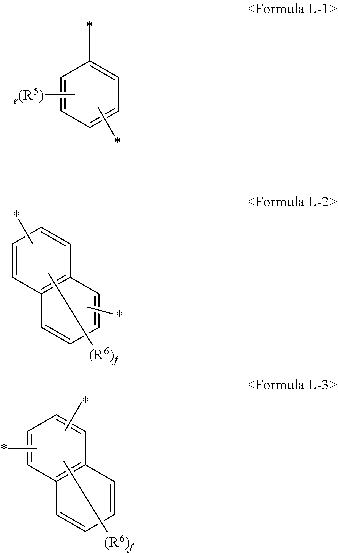

In Formula L-1 to Formula L-3, * indicates the binding position, R⁵ and R⁶ are selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, adjacent groups may be bonded to each other to form a ring, e is an integer of 0 to 4, and f is an integer of 0 to 6.

Formula L1 may be represented by one of the following Formula L1-1 to Formula L1-3.

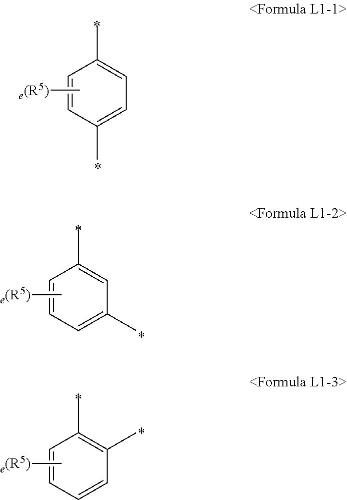

In Formula L1-1 to Formula L1-3, R⁵, e, * are the same as defined for Formula L1.

Formula L2 may be represented by one of the following Formula L2-1 to Formula L2-6.

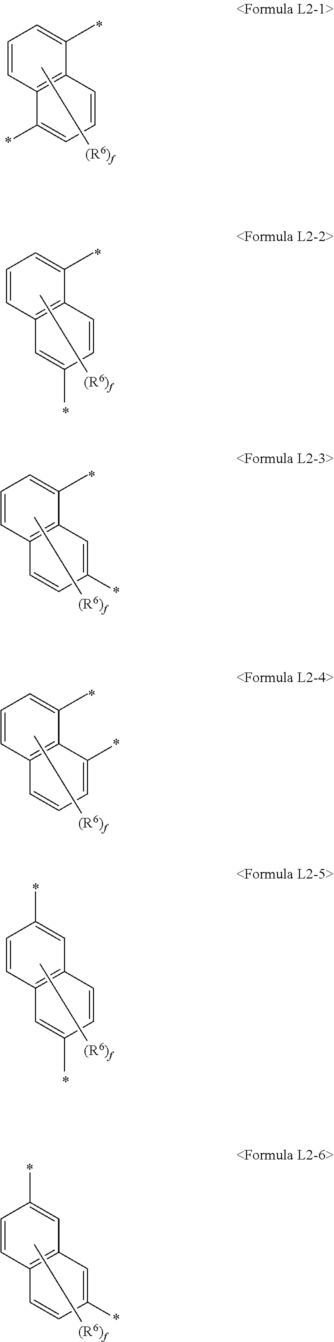

In Formula L2-1 to Formula L2-6, R⁶, f, * are the same as defined for Formula L2.

Formula L3 may be represented by one of the following Formula L3-1 to Formula L3-4.

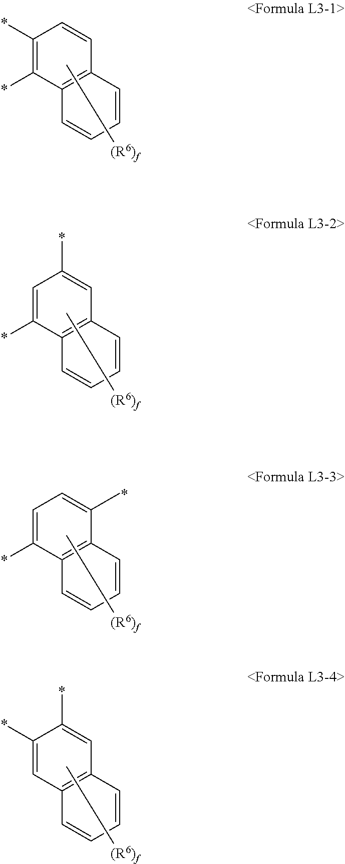

In Formula L3-1 to Formula L3-4, R⁶, f, * are the same as defined for Formula L3.

Specifically, the compound represented by Formula 1 may be one of the following compounds, but there is no limitation thereto.

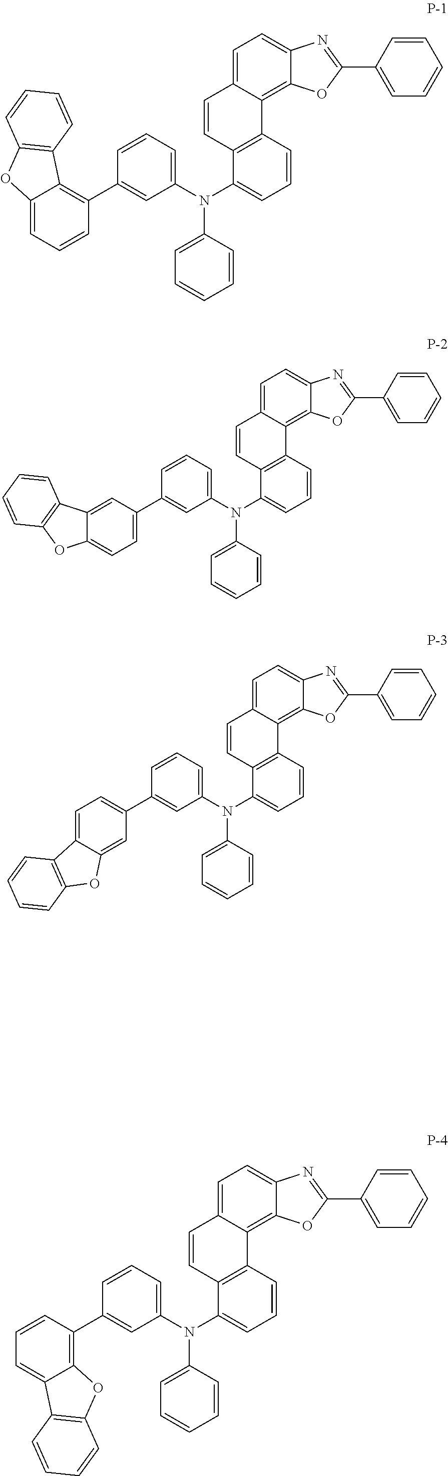 [SMILES: c1ccc(-c2nc3ccc4ccc5c(N(c6ccccc6)c6cccc(-c7cccc8oc9ccccc9c78)c6)cccc5c4c3o2)cc1 ; c1ccc(-c2nc3ccc4ccc5c(N(c6ccccc6)c6cccc(-c7ccc8oc9ccccc9c8c7)c6)cccc5c4c3o2)cc1 ; c1ccc(-c2nc3ccc4ccc5c(N(c6ccccc6)c6cccc(-c7ccc8c(c7)oc7ccccc78)c6)cccc5c4c3o2)cc1 ; c1ccc(-c2nc3ccc4ccc5c(N(c6ccccc6)c6cccc(-c7cccc8c7oc7ccccc78)c6)cccc5c4c3o2)cc1]

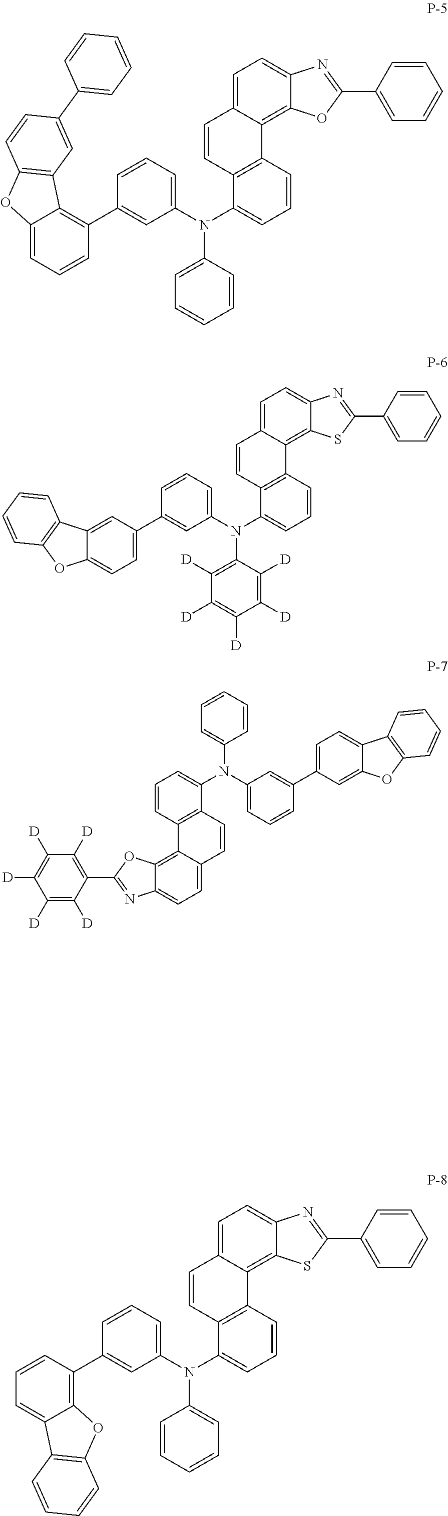 [SMILES: c1ccc(-c2ccc3oc4cccc(-c5cccc(N(c6ccccc6)c6cccc7c6ccc6ccc8nc(-c9ccccc9)oc8c67)c5)c4c3c2)cc1 ; [2H]c1c([2H])c([2H])c(N(c2cccc(-c3ccc4oc5ccccc5c4c3)c2)c2cccc3c2ccc2ccc4nc(-c5ccccc5)sc4c23)c([2H])c1[2H] ; [2H]c1c([2H])c([2H])c(-c2nc3ccc4ccc5c(N(c6ccccc6)c6cccc(-c7ccc8c(c7)oc7ccccc78)c6)cccc5c4c3o2)c([2H])c1[2H] ; c1ccc(-c2nc3ccc4ccc5c(N(c6ccccc6)c6cccc(-c7cccc8c7oc7ccccc78)c6)cccc5c4c3s2)cc1]

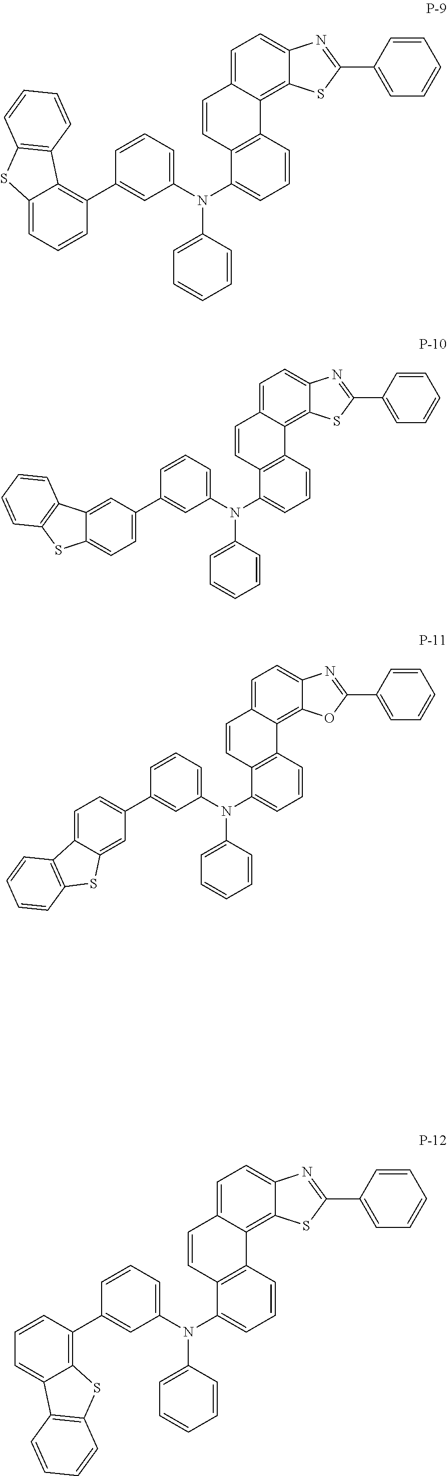 [SMILES: c1ccc(-c2nc3ccc4ccc5c(N(c6ccccc6)c6cccc(-c7cccc8sc9ccccc9c78)c6)cccc5c4c3s2)cc1 ; c1ccc(-c2nc3ccc4ccc5c(N(c6ccccc6)c6cccc(-c7ccc8sc9ccccc9c8c7)c6)cccc5c4c3s2)cc1 ; c1ccc(-c2nc3ccc4ccc5c(N(c6ccccc6)c6cccc(-c7ccc8c(c7)sc7ccccc78)c6)cccc5c4c3o2)cc1 ; c1ccc(-c2nc3ccc4ccc5c(N(c6ccccc6)c6cccc(-c7cccc8c7sc7ccccc78)c6)cccc5c4c3s2)cc1]

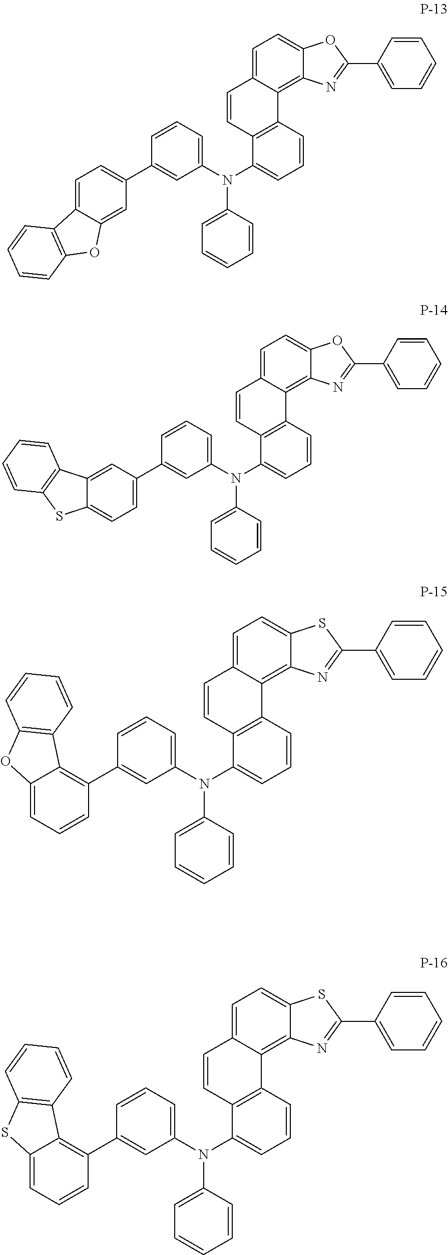 [SMILES: c1ccc(-c2nc3c(ccc4ccc5c(N(c6ccccc6)c6cccc(-c7ccc8c(c7)oc7ccccc78)c6)cccc5c43)o2)cc1 ; c1ccc(-c2nc3c(ccc4ccc5c(N(c6ccccc6)c6cccc(-c7ccc8sc9ccccc9c8c7)c6)cccc5c43)o2)cc1 ; c1ccc(-c2nc3c(ccc4ccc5c(N(c6ccccc6)c6cccc(-c7cccc8oc9ccccc9c78)c6)cccc5c43)s2)cc1 ; c1ccc(-c2nc3c(ccc4ccc5c(N(c6ccccc6)c6cccc(-c7cccc8sc9ccccc9c78)c6)cccc5c43)s2)cc1]

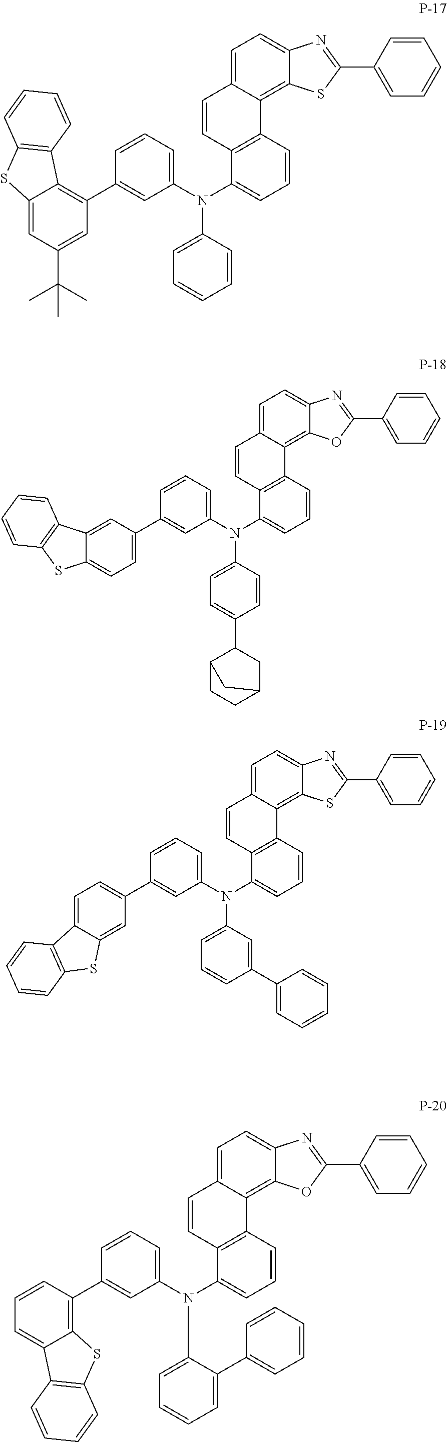 [SMILES: CC(C)(C)c1cc(-c2cccc(N(c3ccccc3)c3cccc4c3ccc3ccc5nc(-c6ccccc6)sc5c34)c2)c2c(c1)sc1ccccc12 ; c1ccc(-c2nc3ccc4ccc5c(N(c6ccc(C7CC8CCC7C8)cc6)c6cccc(-c7ccc8sc9ccccc9c8c7)c6)cccc5c4c3o2)cc1 ; c1ccc(-c2cccc(N(c3cccc(-c4ccc5c(c4)sc4ccccc45)c3)c3cccc4c3ccc3ccc5nc(-c6ccccc6)sc5c34)c2)cc1 ; c1ccc(-c2nc3ccc4ccc5c(N(c6cccc(-c7cccc8c7sc7ccccc78)c6)c6ccccc6-c6ccccc6)cccc5c4c3o2)cc1]

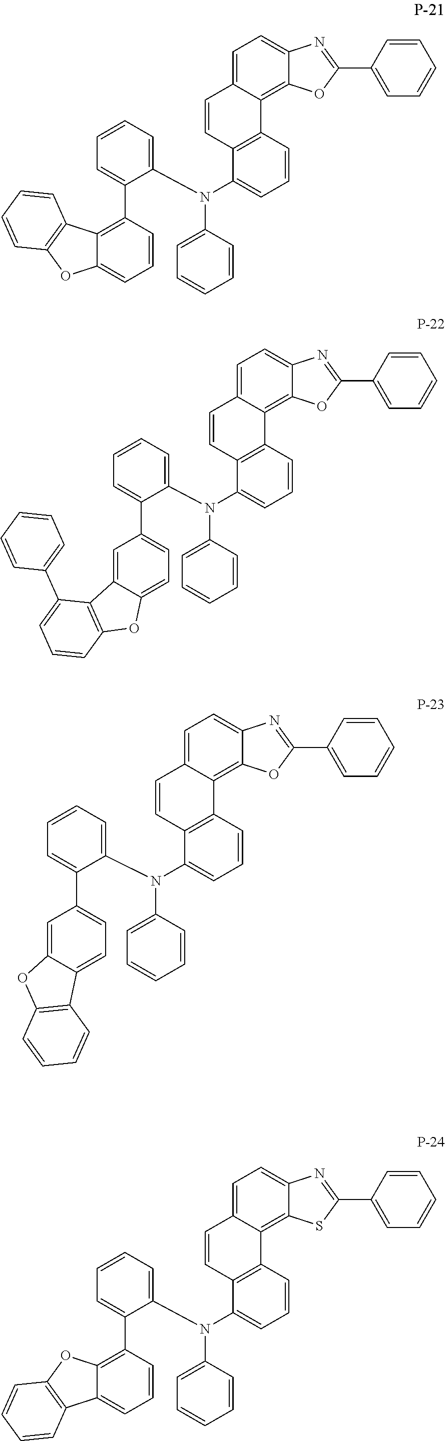 [SMILES: c1ccc(-c2nc3ccc4ccc5c(N(c6ccccc6)c6ccccc6-c6cccc7oc8ccccc8c67)cccc5c4c3o2)cc1 ; c1ccc(-c2nc3ccc4ccc5c(N(c6ccccc6)c6ccccc6-c6ccc7oc8cccc(-c9ccccc9)c8c7c6)cccc5c4c3o2)cc1 ; c1ccc(-c2nc3ccc4ccc5c(N(c6ccccc6)c6ccccc6-c6ccc7c(c6)oc6ccccc67)cccc5c4c3o2)cc1 ; c1ccc(-c2nc3ccc4ccc5c(N(c6ccccc6)c6ccccc6-c6cccc7c6oc6ccccc67)cccc5c4c3s2)cc1]

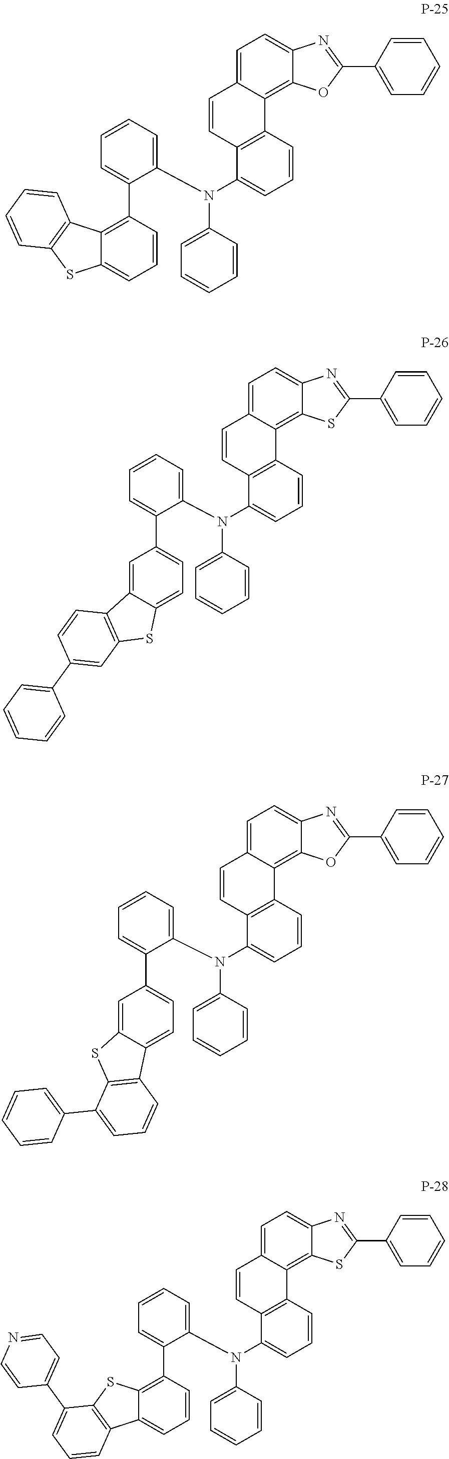 [SMILES: c1ccc(-c2nc3ccc4ccc5c(N(c6ccccc6)c6ccccc6-c6cccc7sc8ccccc8c67)cccc5c4c3o2)cc1 ; c1ccc(-c2ccc3c(c2)sc2ccc(-c4ccccc4N(c4ccccc4)c4cccc5c4ccc4ccc6nc(-c7ccccc7)sc6c45)cc23)cc1 ; c1ccc(-c2nc3ccc4ccc5c(N(c6ccccc6)c6ccccc6-c6ccc7c(c6)sc6c(-c8ccccc8)cccc67)cccc5c4c3o2)cc1 ; c1ccc(-c2nc3ccc4ccc5c(N(c6ccccc6)c6ccccc6-c6cccc7c6sc6c(-c8ccncc8)cccc67)cccc5c4c3s2)cc1]

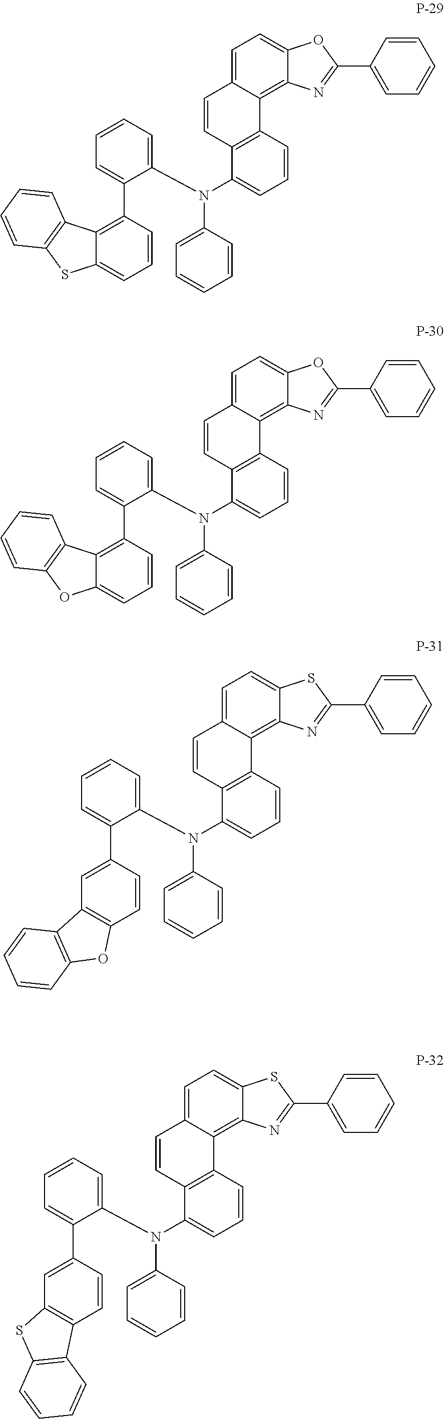 [SMILES: c1ccc(-c2nc3c(ccc4ccc5c(N(c6ccccc6)c6ccccc6-c6cccc7sc8ccccc8c67)cccc5c43)o2)cc1 ; c1ccc(-c2nc3c(ccc4ccc5c(N(c6ccccc6)c6ccccc6-c6cccc7oc8ccccc8c67)cccc5c43)o2)cc1 ; c1ccc(-c2nc3c(ccc4ccc5c(N(c6ccccc6)c6ccccc6-c6ccc7oc8ccccc8c7c6)cccc5c43)s2)cc1 ; c1ccc(-c2nc3c(ccc4ccc5c(N(c6ccccc6)c6ccccc6-c6ccc7c(c6)sc6ccccc67)cccc5c43)s2)cc1]

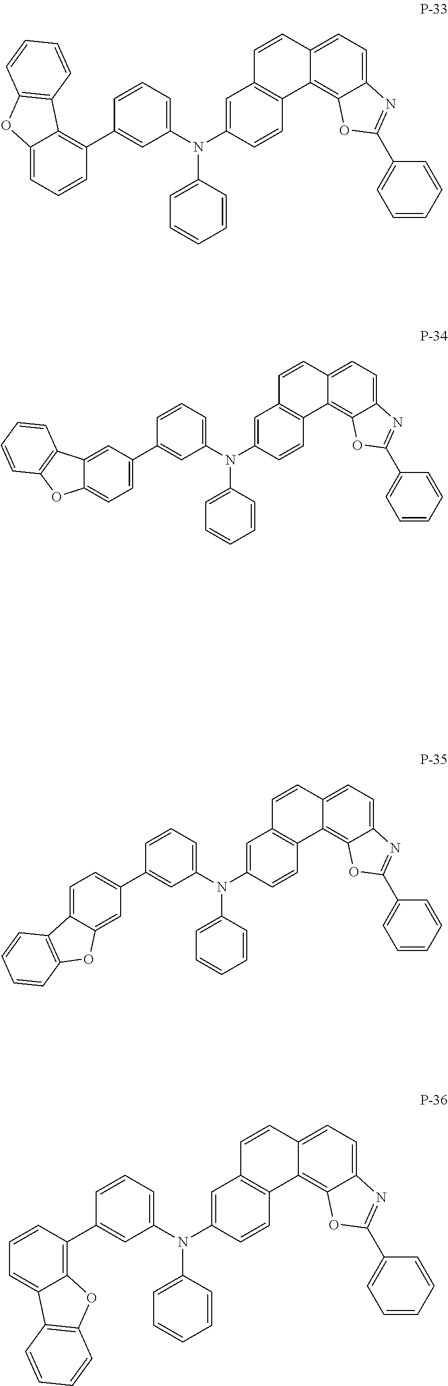 [SMILES: c1ccc(-c2nc3ccc4ccc5cc(N(c6ccccc6)c6cccc(-c7cccc8oc9ccccc9c78)c6)ccc5c4c3o2)cc1 ; c1ccc(-c2nc3ccc4ccc5cc(N(c6ccccc6)c6cccc(-c7ccc8oc9ccccc9c8c7)c6)ccc5c4c3o2)cc1 ; c1ccc(-c2nc3ccc4ccc5cc(N(c6ccccc6)c6cccc(-c7ccc8c(c7)oc7ccccc78)c6)ccc5c4c3o2)cc1 ; c1ccc(-c2nc3ccc4ccc5cc(N(c6ccccc6)c6cccc(-c7cccc8c7oc7ccccc78)c6)ccc5c4c3o2)cc1]

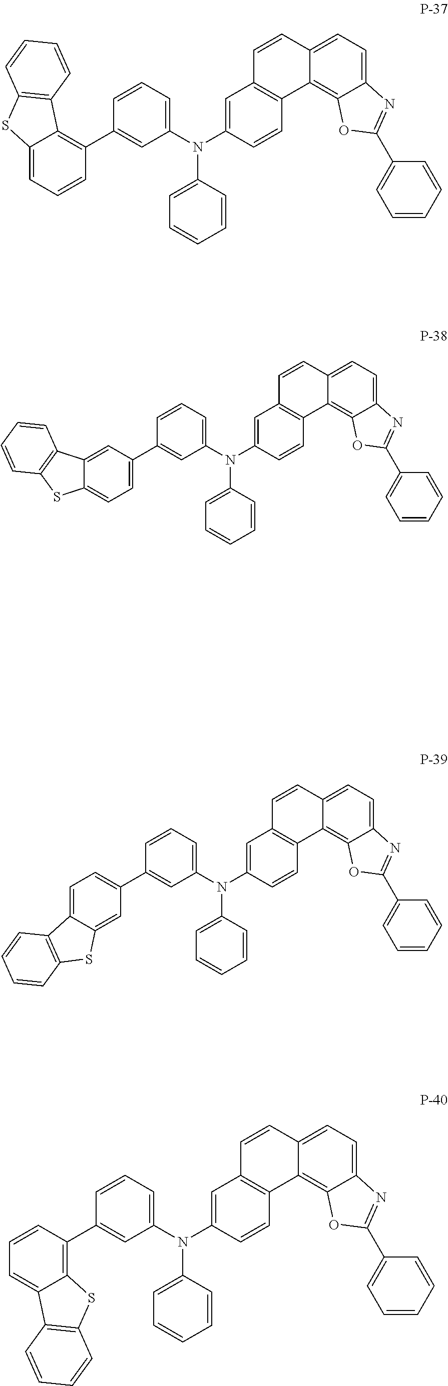 [SMILES: c1ccc(-c2nc3ccc4ccc5cc(N(c6ccccc6)c6cccc(-c7cccc8sc9ccccc9c78)c6)ccc5c4c3o2)cc1 ; c1ccc(-c2nc3ccc4ccc5cc(N(c6ccccc6)c6cccc(-c7ccc8sc9ccccc9c8c7)c6)ccc5c4c3o2)cc1 ; c1ccc(-c2nc3ccc4ccc5cc(N(c6ccccc6)c6cccc(-c7ccc8c(c7)sc7ccccc78)c6)ccc5c4c3o2)cc1 ; c1ccc(-c2nc3ccc4ccc5cc(N(c6ccccc6)c6cccc(-c7cccc8c7sc7ccccc78)c6)ccc5c4c3o2)cc1]

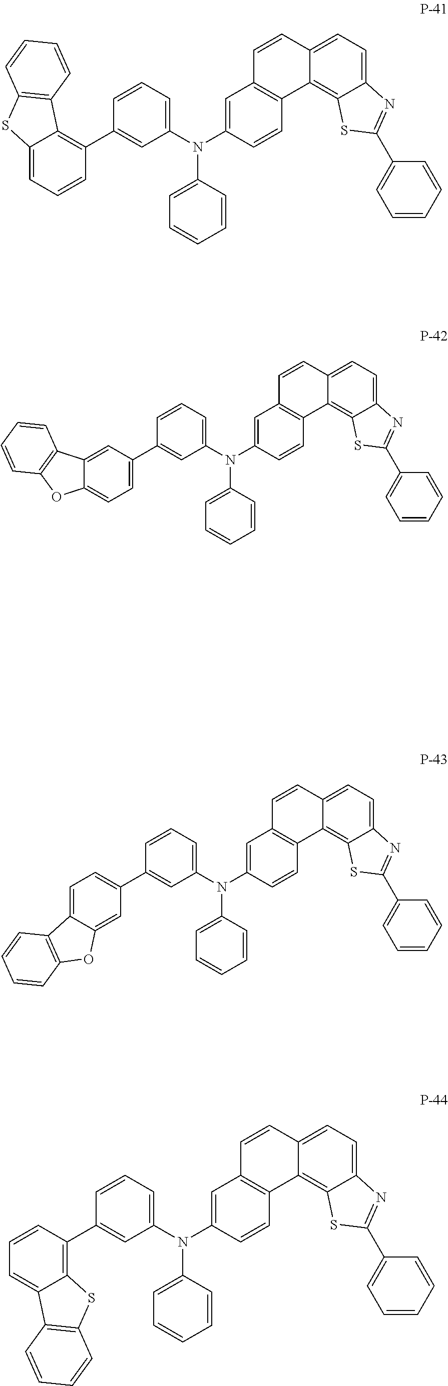 [SMILES: c1ccc(-c2nc3ccc4ccc5cc(N(c6ccccc6)c6cccc(-c7cccc8sc9ccccc9c78)c6)ccc5c4c3s2)cc1 ; c1ccc(-c2nc3ccc4ccc5cc(N(c6ccccc6)c6cccc(-c7ccc8oc9ccccc9c8c7)c6)ccc5c4c3s2)cc1 ; c1ccc(-c2nc3ccc4ccc5cc(N(c6ccccc6)c6cccc(-c7ccc8c(c7)oc7ccccc78)c6)ccc5c4c3s2)cc1 ; c1ccc(-c2nc3ccc4ccc5cc(N(c6ccccc6)c6cccc(-c7cccc8c7sc7ccccc78)c6)ccc5c4c3s2)cc1]

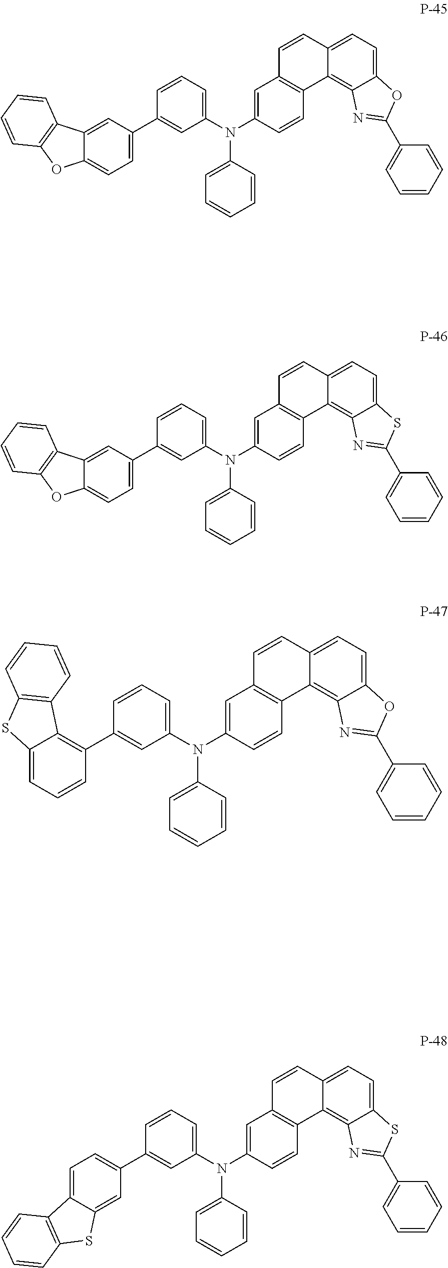 [SMILES: c1ccc(-c2nc3c(ccc4ccc5cc(N(c6ccccc6)c6cccc(-c7ccc8oc9ccccc9c8c7)c6)ccc5c43)o2)cc1 ; c1ccc(-c2nc3c(ccc4ccc5cc(N(c6ccccc6)c6cccc(-c7ccc8oc9ccccc9c8c7)c6)ccc5c43)s2)cc1 ; c1ccc(-c2nc3c(ccc4ccc5cc(N(c6ccccc6)c6cccc(-c7cccc8sc9ccccc9c78)c6)ccc5c43)o2)cc1 ; c1ccc(-c2nc3c(ccc4ccc5cc(N(c6ccccc6)c6cccc(-c7ccc8c(c7)sc7ccccc78)c6)ccc5c43)s2)cc1]

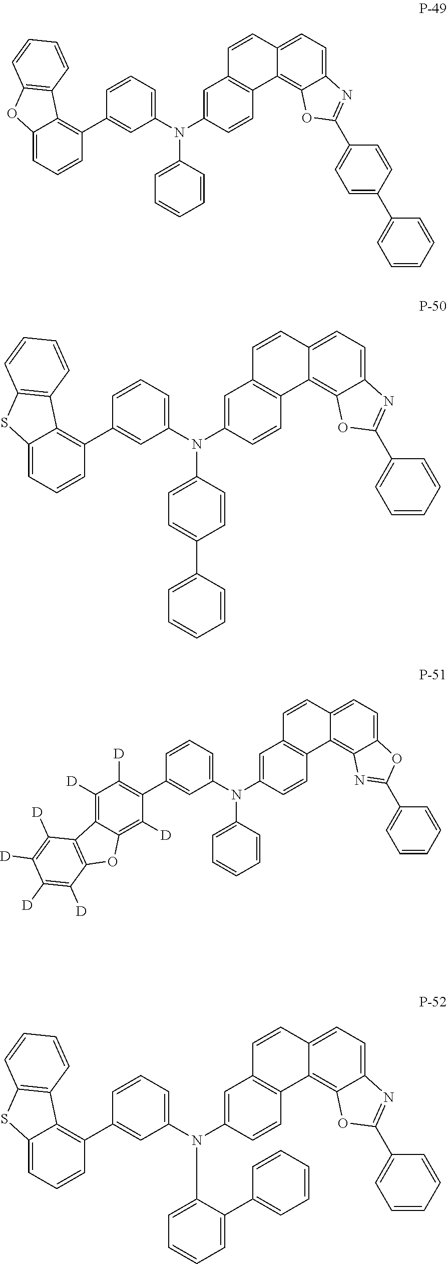 [SMILES: c1ccc(-c2ccc(-c3nc4ccc5ccc6cc(N(c7ccccc7)c7cccc(-c8cccc9oc%10ccccc%10c89)c7)ccc6c5c4o3)cc2)cc1 ; c1ccc(-c2ccc(N(c3cccc(-c4cccc5sc6ccccc6c45)c3)c3ccc4c(ccc5ccc6nc(-c7ccccc7)oc6c54)c3)cc2)cc1 ; [2H]c1c([2H])c([2H])c2c(oc3c([2H])c(-c4cccc(N(c5ccccc5)c5ccc6c(ccc7ccc8oc(-c9ccccc9)nc8c76)c5)c4)c([2H])c([2H])c32)c1[2H] ; c1ccc(-c2nc3ccc4ccc5cc(N(c6cccc(-c7cccc8sc9ccccc9c78)c6)c6ccccc6-c6ccccc6)ccc5c4c3o2)cc1]

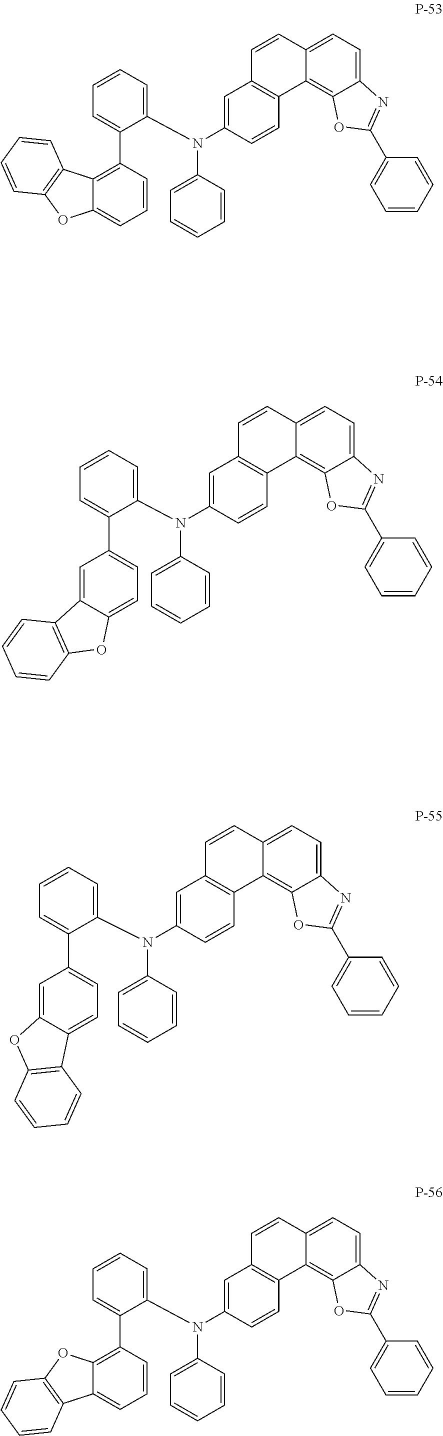 [SMILES: c1ccc(-c2nc3ccc4ccc5cc(N(c6ccccc6)c6ccccc6-c6cccc7oc8ccccc8c67)ccc5c4c3o2)cc1 ; c1ccc(-c2nc3ccc4ccc5cc(N(c6ccccc6)c6ccccc6-c6ccc7oc8ccccc8c7c6)ccc5c4c3o2)cc1 ; c1ccc(-c2nc3ccc4ccc5cc(N(c6ccccc6)c6ccccc6-c6ccc7c(c6)oc6ccccc67)ccc5c4c3o2)cc1 ; c1ccc(-c2nc3ccc4ccc5cc(N(c6ccccc6)c6ccccc6-c6cccc7c6oc6ccccc67)ccc5c4c3o2)cc1]

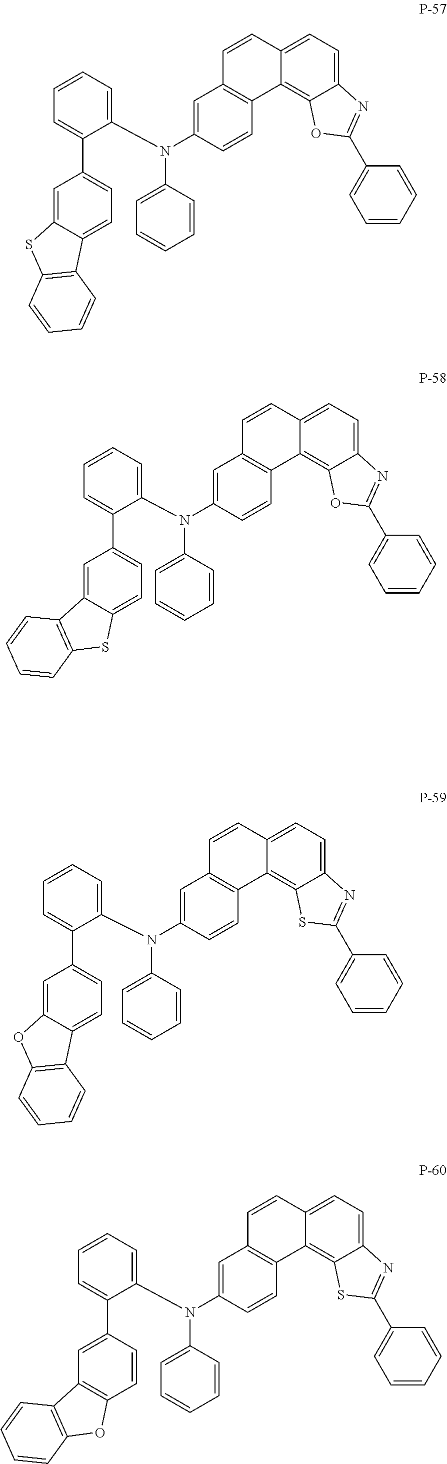 [SMILES: c1ccc(-c2nc3ccc4ccc5cc(N(c6ccccc6)c6ccccc6-c6ccc7c(c6)sc6ccccc67)ccc5c4c3o2)cc1 ; c1ccc(-c2nc3ccc4ccc5cc(N(c6ccccc6)c6ccccc6-c6ccc7sc8ccccc8c7c6)ccc5c4c3o2)cc1 ; c1ccc(-c2nc3ccc4ccc5cc(N(c6ccccc6)c6ccccc6-c6ccc7c(c6)oc6ccccc67)ccc5c4c3s2)cc1 ; c1ccc(-c2nc3ccc4ccc5cc(N(c6ccccc6)c6ccccc6-c6ccc7oc8ccccc8c7c6)ccc5c4c3s2)cc1]

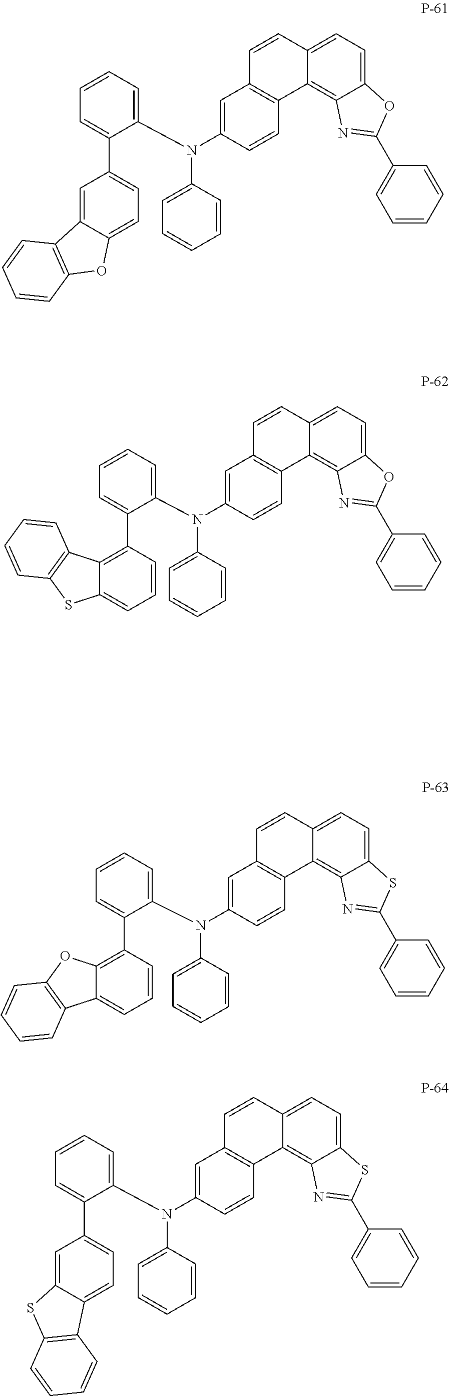 [SMILES: c1ccc(-c2nc3c(ccc4ccc5cc(N(c6ccccc6)c6ccccc6-c6ccc7oc8ccccc8c7c6)ccc5c43)o2)cc1 ; c1ccc(-c2nc3c(ccc4ccc5cc(N(c6ccccc6)c6ccccc6-c6cccc7sc8ccccc8c67)ccc5c43)o2)cc1 ; c1ccc(-c2nc3c(ccc4ccc5cc(N(c6ccccc6)c6ccccc6-c6cccc7c6oc6ccccc67)ccc5c43)s2)cc1 ; c1ccc(-c2nc3c(ccc4ccc5cc(N(c6ccccc6)c6ccccc6-c6ccc7c(c6)sc6ccccc67)ccc5c43)s2)cc1]

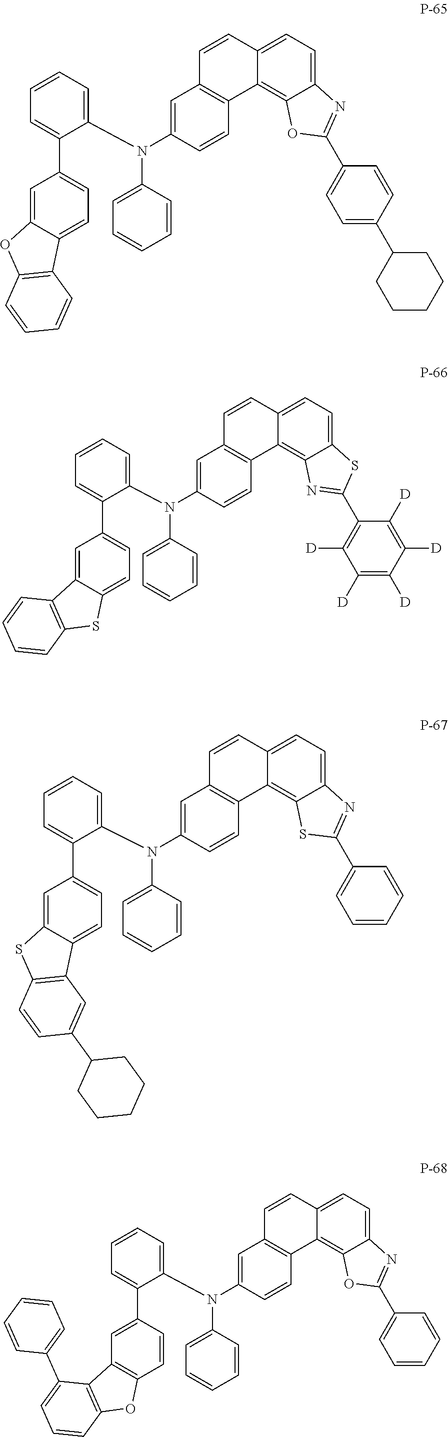 [SMILES: c1ccc(N(c2ccc3c(ccc4ccc5nc(-c6ccc(C7CCCCC7)cc6)oc5c43)c2)c2ccccc2-c2ccc3c(c2)oc2ccccc23)cc1 ; [2H]c1c([2H])c([2H])c(-c2nc3c(ccc4ccc5cc(N(c6ccccc6)c6ccccc6-c6ccc7sc8ccccc8c7c6)ccc5c43)s2)c([2H])c1[2H] ; c1ccc(-c2nc3ccc4ccc5cc(N(c6ccccc6)c6ccccc6-c6ccc7c(c6)sc6ccc(C8CCCCC8)cc67)ccc5c4c3s2)cc1 ; c1ccc(-c2nc3ccc4ccc5cc(N(c6ccccc6)c6ccccc6-c6ccc7oc8cccc(-c9ccccc9)c8c7c6)ccc5c4c3o2)cc1]

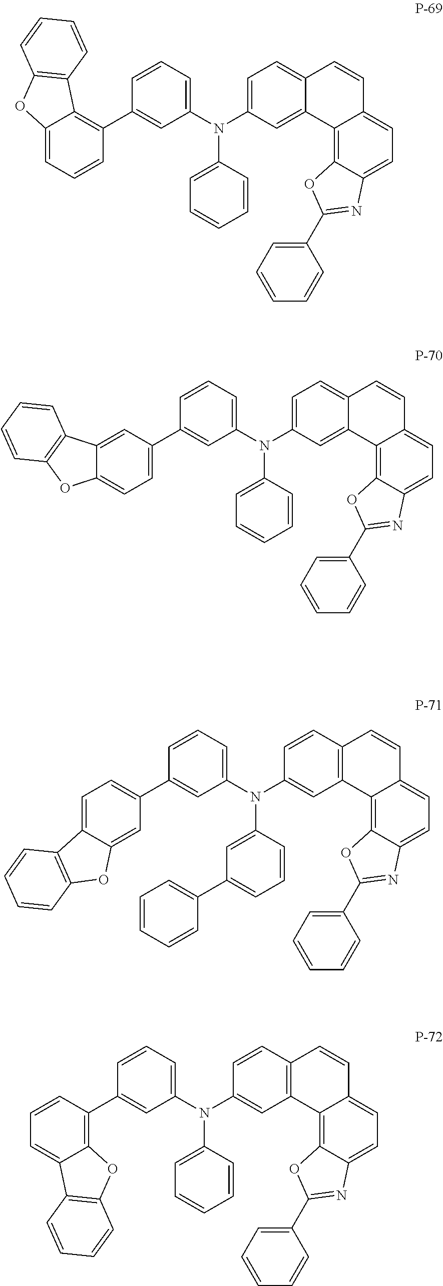 [SMILES: c1ccc(-c2nc3ccc4ccc5ccc(N(c6ccccc6)c6cccc(-c7cccc8oc9ccccc9c78)c6)cc5c4c3o2)cc1 ; c1ccc(-c2nc3ccc4ccc5ccc(N(c6ccccc6)c6cccc(-c7ccc8oc9ccccc9c8c7)c6)cc5c4c3o2)cc1 ; c1ccc(-c2cccc(N(c3cccc(-c4ccc5c(c4)oc4ccccc45)c3)c3ccc4ccc5ccc6nc(-c7ccccc7)oc6c5c4c3)c2)cc1 ; c1ccc(-c2nc3ccc4ccc5ccc(N(c6ccccc6)c6cccc(-c7cccc8c7oc7ccccc78)c6)cc5c4c3o2)cc1]

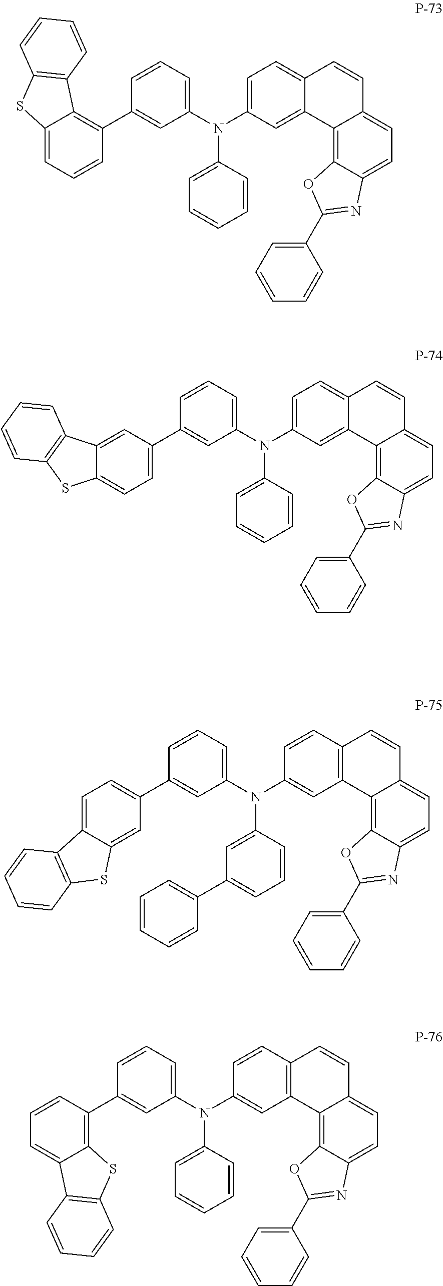 [SMILES: c1ccc(-c2nc3ccc4ccc5ccc(N(c6ccccc6)c6cccc(-c7cccc8sc9ccccc9c78)c6)cc5c4c3o2)cc1 ; c1ccc(-c2nc3ccc4ccc5ccc(N(c6ccccc6)c6cccc(-c7ccc8sc9ccccc9c8c7)c6)cc5c4c3o2)cc1 ; c1ccc(-c2cccc(N(c3cccc(-c4ccc5c(c4)sc4ccccc45)c3)c3ccc4ccc5ccc6nc(-c7ccccc7)oc6c5c4c3)c2)cc1 ; c1ccc(-c2nc3ccc4ccc5ccc(N(c6ccccc6)c6cccc(-c7cccc8c7sc7ccccc78)c6)cc5c4c3o2)cc1]

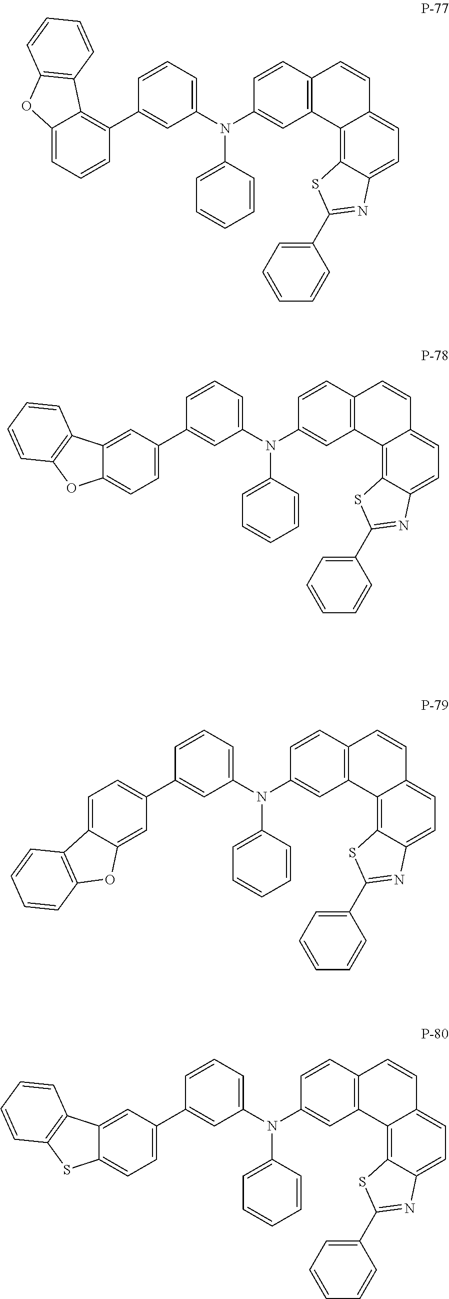 [SMILES: c1ccc(-c2nc3ccc4ccc5ccc(N(c6ccccc6)c6cccc(-c7cccc8oc9ccccc9c78)c6)cc5c4c3s2)cc1 ; c1ccc(-c2nc3ccc4ccc5ccc(N(c6ccccc6)c6cccc(-c7ccc8oc9ccccc9c8c7)c6)cc5c4c3s2)cc1 ; c1ccc(-c2nc3ccc4ccc5ccc(N(c6ccccc6)c6cccc(-c7ccc8c(c7)oc7ccccc78)c6)cc5c4c3s2)cc1 ; c1ccc(-c2nc3ccc4ccc5ccc(N(c6ccccc6)c6cccc(-c7ccc8sc9ccccc9c8c7)c6)cc5c4c3s2)cc1]

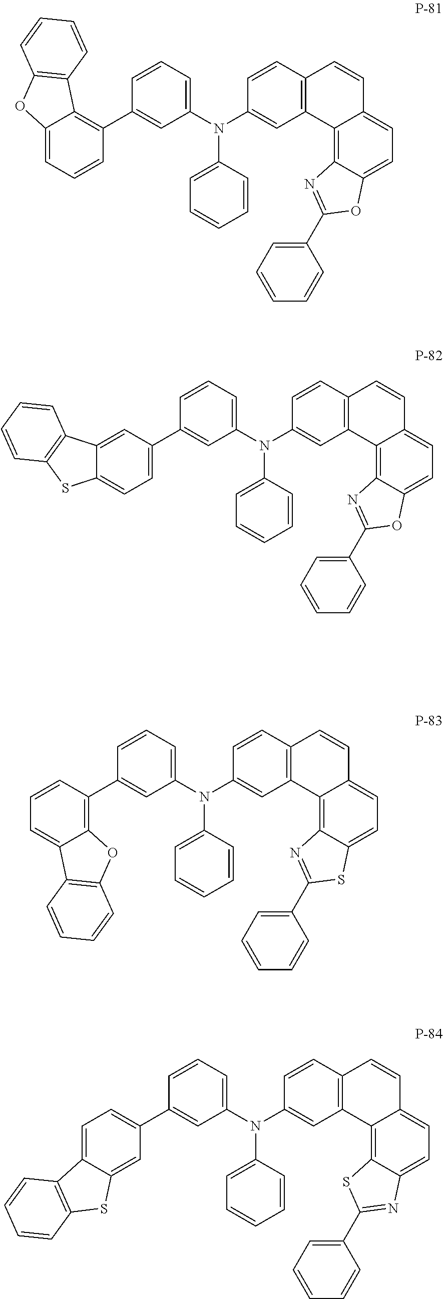 [SMILES: c1ccc(-c2nc3c(ccc4ccc5ccc(N(c6ccccc6)c6cccc(-c7cccc8oc9ccccc9c78)c6)cc5c43)o2)cc1 ; c1ccc(-c2nc3c(ccc4ccc5ccc(N(c6ccccc6)c6cccc(-c7ccc8sc9ccccc9c8c7)c6)cc5c43)o2)cc1 ; c1ccc(-c2nc3c(ccc4ccc5ccc(N(c6ccccc6)c6cccc(-c7cccc8c7oc7ccccc78)c6)cc5c43)s2)cc1 ; c1ccc(-c2nc3ccc4ccc5ccc(N(c6ccccc6)c6cccc(-c7ccc8c(c7)sc7ccccc78)c6)cc5c4c3s2)cc1]

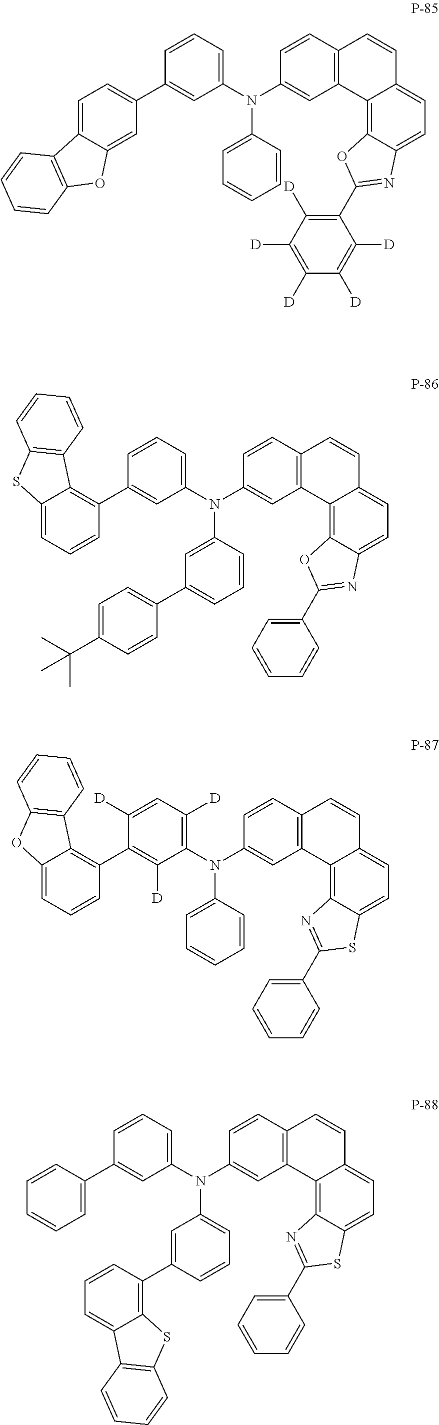 [SMILES: [2H]c1c([2H])c([2H])c(-c2nc3ccc4ccc5ccc(N(c6ccccc6)c6cccc(-c7ccc8c(c7)oc7ccccc78)c6)cc5c4c3o2)c([2H])c1[2H] ; CC(C)(C)c1ccc(-c2cccc(N(c3cccc(-c4cccc5sc6ccccc6c45)c3)c3ccc4ccc5ccc6nc(-c7ccccc7)oc6c5c4c3)c2)cc1 ; [2H]c1cc([2H])c(N(c2ccccc2)c2ccc3ccc4ccc5sc(-c6ccccc6)nc5c4c3c2)c([2H])c1-c1cccc2oc3ccccc3c12 ; c1ccc(-c2cccc(N(c3cccc(-c4cccc5c4sc4ccccc45)c3)c3ccc4ccc5ccc6sc(-c7ccccc7)nc6c5c4c3)c2)cc1]

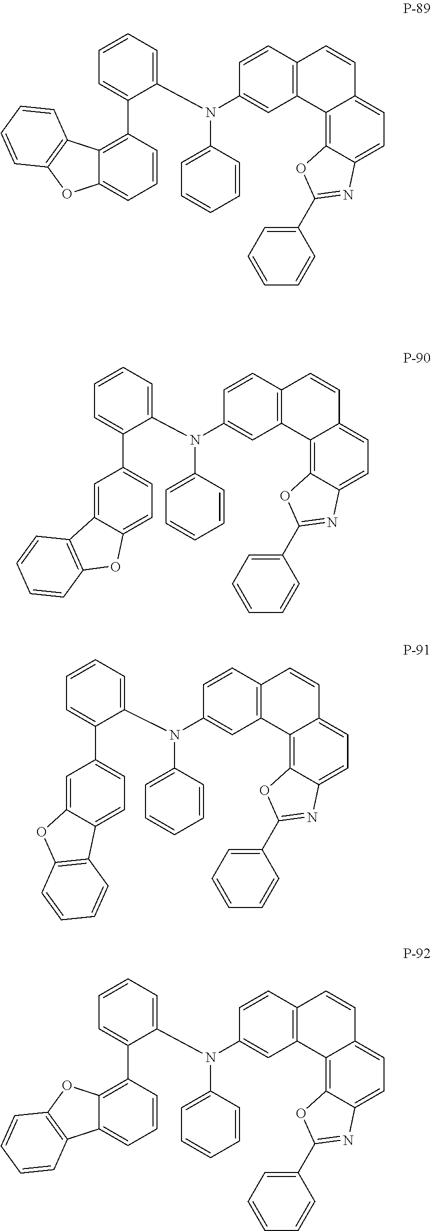 [SMILES: c1ccc(-c2nc3ccc4ccc5ccc(N(c6ccccc6)c6ccccc6-c6cccc7oc8ccccc8c67)cc5c4c3o2)cc1 ; c1ccc(-c2nc3ccc4ccc5ccc(N(c6ccccc6)c6ccccc6-c6ccc7oc8ccccc8c7c6)cc5c4c3o2)cc1 ; c1ccc(-c2nc3ccc4ccc5ccc(N(c6ccccc6)c6ccccc6-c6ccc7c(c6)oc6ccccc67)cc5c4c3o2)cc1 ; c1ccc(-c2nc3ccc4ccc5ccc(N(c6ccccc6)c6ccccc6-c6cccc7c6oc6ccccc67)cc5c4c3o2)cc1]

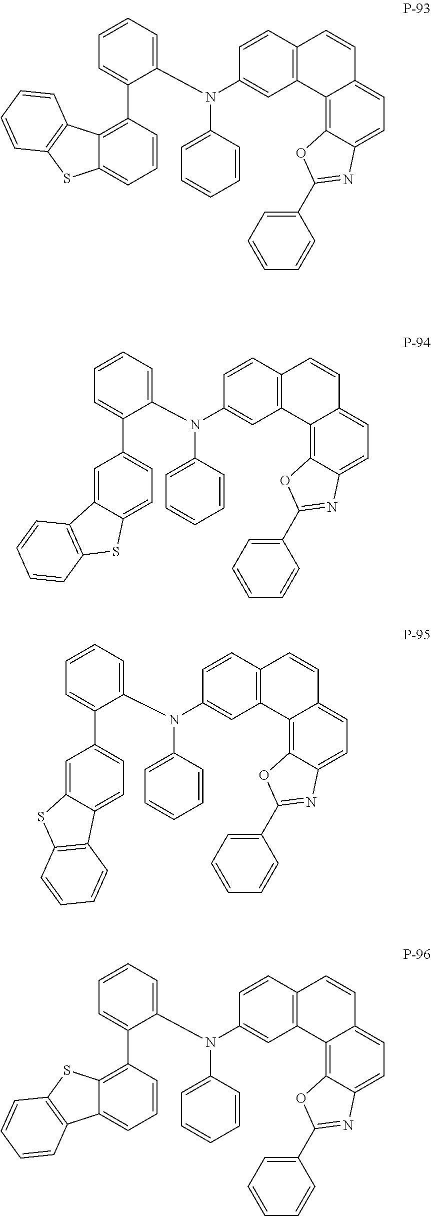 [SMILES: c1ccc(-c2nc3ccc4ccc5ccc(N(c6ccccc6)c6ccccc6-c6cccc7sc8ccccc8c67)cc5c4c3o2)cc1 ; c1ccc(-c2nc3ccc4ccc5ccc(N(c6ccccc6)c6ccccc6-c6ccc7sc8ccccc8c7c6)cc5c4c3o2)cc1 ; c1ccc(-c2nc3ccc4ccc5ccc(N(c6ccccc6)c6ccccc6-c6ccc7c(c6)sc6ccccc67)cc5c4c3o2)cc1 ; c1ccc(-c2nc3ccc4ccc5ccc(N(c6ccccc6)c6ccccc6-c6cccc7c6sc6ccccc67)cc5c4c3o2)cc1]

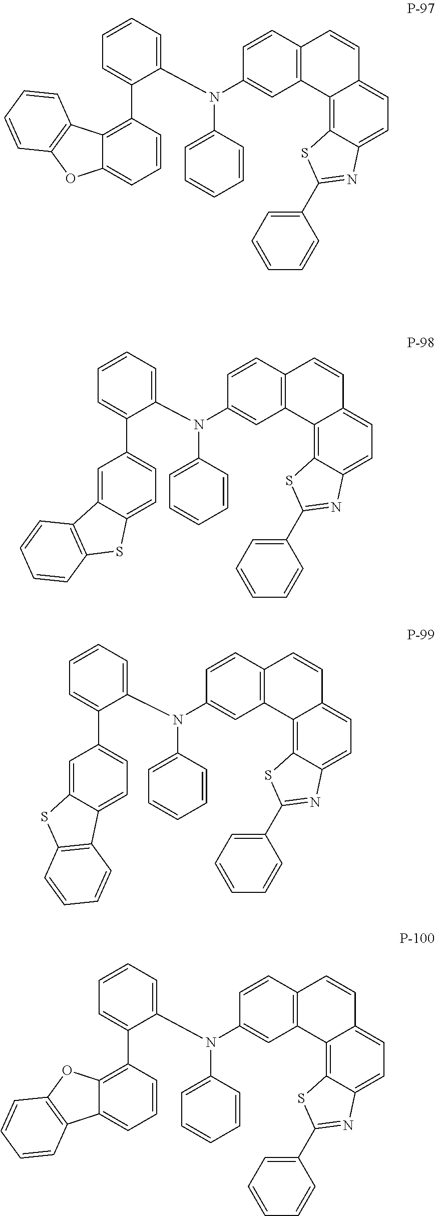 [SMILES: c1ccc(-c2nc3ccc4ccc5ccc(N(c6ccccc6)c6ccccc6-c6cccc7oc8ccccc8c67)cc5c4c3s2)cc1 ; c1ccc(-c2nc3ccc4ccc5ccc(N(c6ccccc6)c6ccccc6-c6ccc7sc8ccccc8c7c6)cc5c4c3s2)cc1 ; c1ccc(-c2nc3ccc4ccc5ccc(N(c6ccccc6)c6ccccc6-c6ccc7c(c6)sc6ccccc67)cc5c4c3s2)cc1 ; c1ccc(-c2nc3ccc4ccc5ccc(N(c6ccccc6)c6ccccc6-c6cccc7c6oc6ccccc67)cc5c4c3s2)cc1]

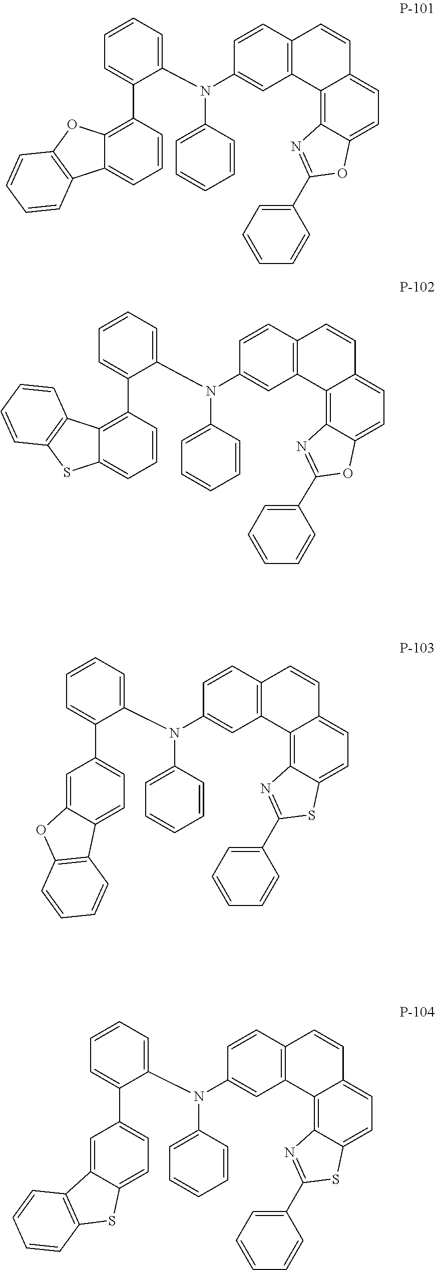 [SMILES: c1ccc(-c2nc3c(ccc4ccc5ccc(N(c6ccccc6)c6ccccc6-c6cccc7c6oc6ccccc67)cc5c43)o2)cc1 ; c1ccc(-c2nc3c(ccc4ccc5ccc(N(c6ccccc6)c6ccccc6-c6cccc7sc8ccccc8c67)cc5c43)o2)cc1 ; c1ccc(-c2nc3c(ccc4ccc5ccc(N(c6ccccc6)c6ccccc6-c6ccc7c(c6)oc6ccccc67)cc5c43)s2)cc1 ; c1ccc(-c2nc3c(ccc4ccc5ccc(N(c6ccccc6)c6ccccc6-c6ccc7sc8ccccc8c7c6)cc5c43)s2)cc1]

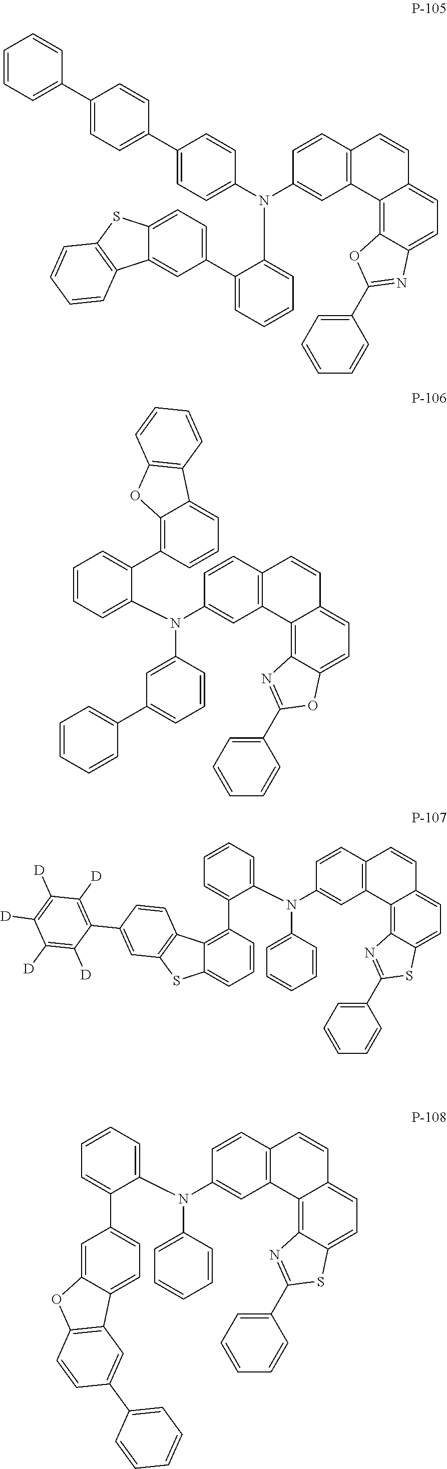 [SMILES: c1ccc(-c2ccc(-c3ccc(N(c4ccc5ccc6ccc7nc(-c8ccccc8)oc7c6c5c4)c4ccccc4-c4ccc5sc6ccccc6c5c4)cc3)cc2)cc1 ; c1ccc(-c2cccc(N(c3ccc4ccc5ccc6oc(-c7ccccc7)nc6c5c4c3)c3ccccc3-c3cccc4c3oc3ccccc34)c2)cc1 ; [2H]c1c([2H])c([2H])c(-c2ccc3c(c2)sc2cccc(-c4ccccc4N(c4ccccc4)c4ccc5ccc6ccc7sc(-c8ccccc8)nc7c6c5c4)c23)c([2H])c1[2H] ; c1ccc(-c2ccc3oc4cc(-c5ccccc5N(c5ccccc5)c5ccc6ccc7ccc8sc(-c9ccccc9)nc8c7c6c5)ccc4c3c2)cc1]

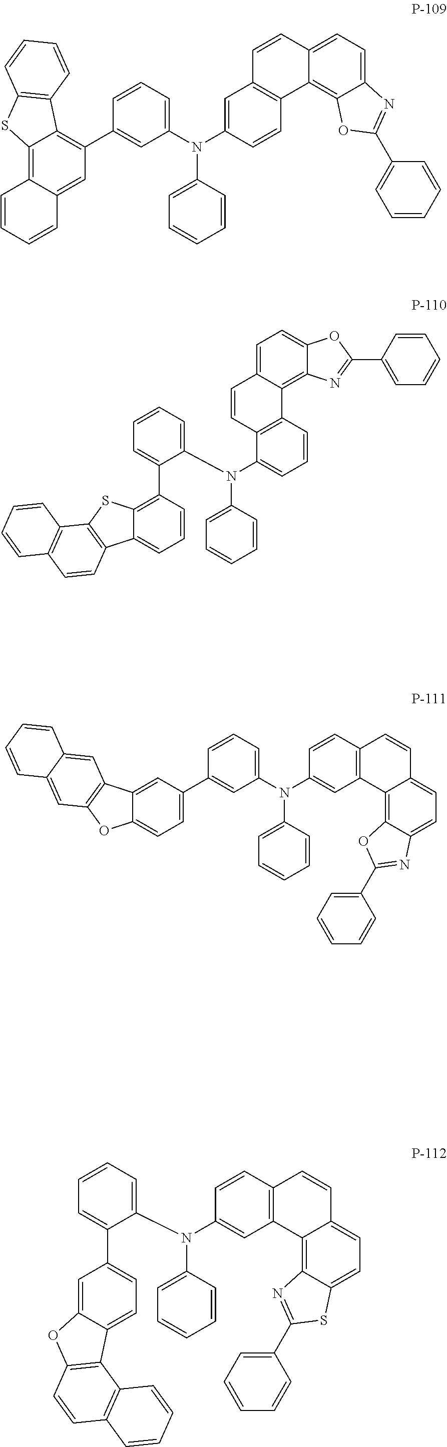 [SMILES: c1ccc(-c2nc3ccc4ccc5cc(N(c6ccccc6)c6cccc(-c7cc8ccccc8c8sc9ccccc9c78)c6)ccc5c4c3o2)cc1 ; c1ccc(-c2nc3c(ccc4ccc5c(N(c6ccccc6)c6ccccc6-c6cccc7c6sc6c8ccccc8ccc76)cccc5c43)o2)cc1 ; c1ccc(-c2nc3ccc4ccc5ccc(N(c6ccccc6)c6cccc(-c7ccc8oc9cc%10ccccc%10cc9c8c7)c6)cc5c4c3o2)cc1 ; c1ccc(-c2nc3c(ccc4ccc5ccc(N(c6ccccc6)c6ccccc6-c6ccc7c(c6)oc6ccc8ccccc8c67)cc5c43)s2)cc1]

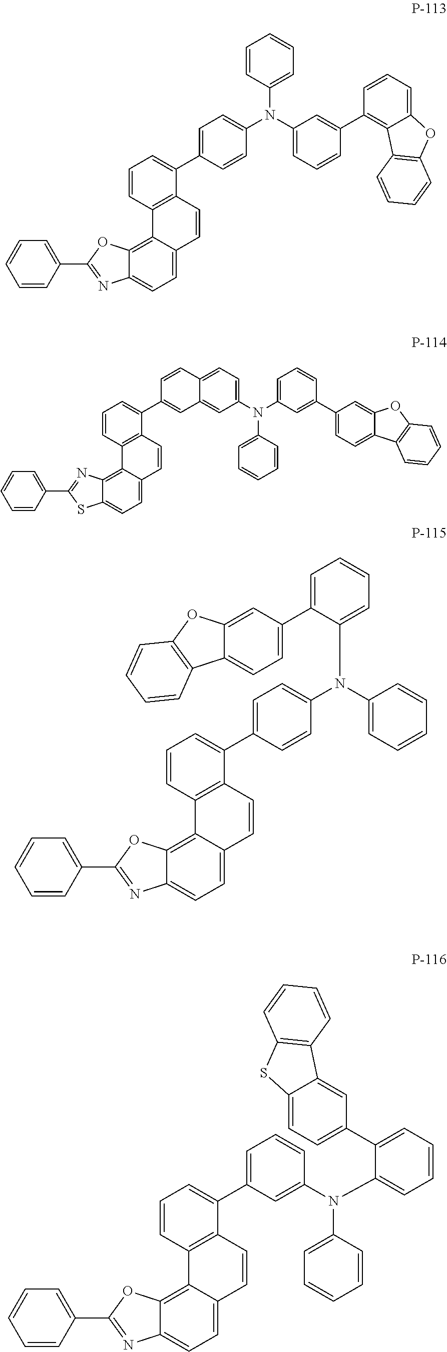 [SMILES: c1ccc(-c2nc3ccc4ccc5c(-c6ccc(N(c7ccccc7)c7cccc(-c8cccc9oc%10ccccc%10c89)c7)cc6)cccc5c4c3o2)cc1 ; c1ccc(-c2nc3c(ccc4ccc5c(-c6ccc7ccc(N(c8ccccc8)c8cccc(-c9ccc%10c(c9)oc9ccccc9%10)c8)cc7c6)cccc5c43)s2)cc1 ; c1ccc(-c2nc3ccc4ccc5c(-c6ccc(N(c7ccccc7)c7ccccc7-c7ccc8c(c7)oc7ccccc78)cc6)cccc5c4c3o2)cc1 ; c1ccc(-c2nc3ccc4ccc5c(-c6cccc(N(c7ccccc7)c7ccccc7-c7ccc8sc9ccccc9c8c7)c6)cccc5c4c3o2)cc1]

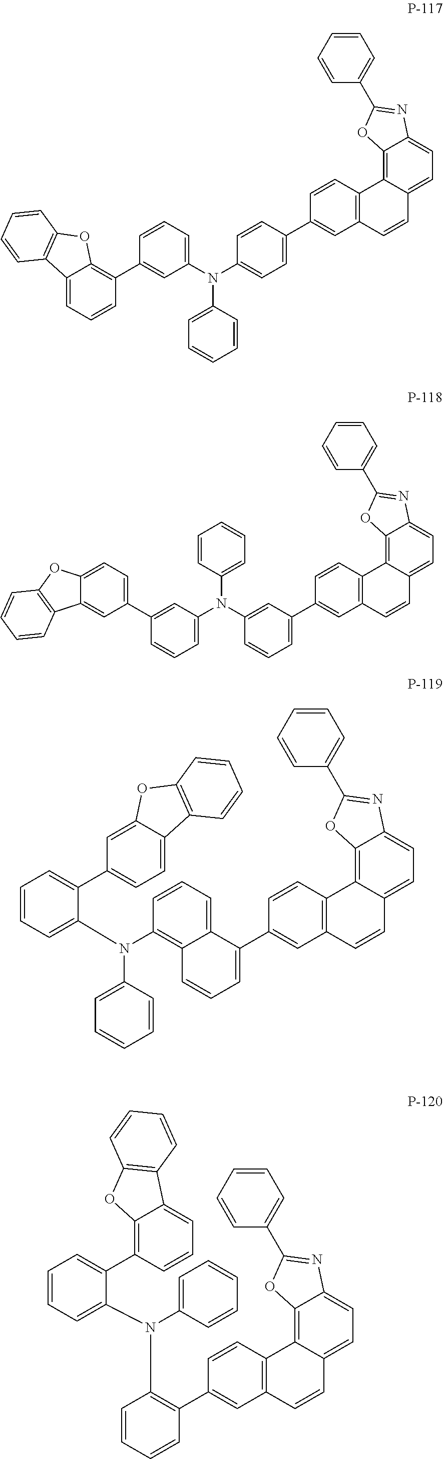 [SMILES: c1ccc(-c2nc3ccc4ccc5cc(-c6ccc(N(c7ccccc7)c7cccc(-c8cccc9c8oc8ccccc89)c7)cc6)ccc5c4c3o2)cc1 ; c1ccc(-c2nc3ccc4ccc5cc(-c6cccc(N(c7ccccc7)c7cccc(-c8ccc9oc%10ccccc%10c9c8)c7)c6)ccc5c4c3o2)cc1 ; c1ccc(-c2nc3ccc4ccc5cc(-c6cccc7c(N(c8ccccc8)c8ccccc8-c8ccc9c(c8)oc8ccccc89)cccc67)ccc5c4c3o2)cc1 ; c1ccc(-c2nc3ccc4ccc5cc(-c6ccccc6N(c6ccccc6)c6ccccc6-c6cccc7c6oc6ccccc67)ccc5c4c3o2)cc1]

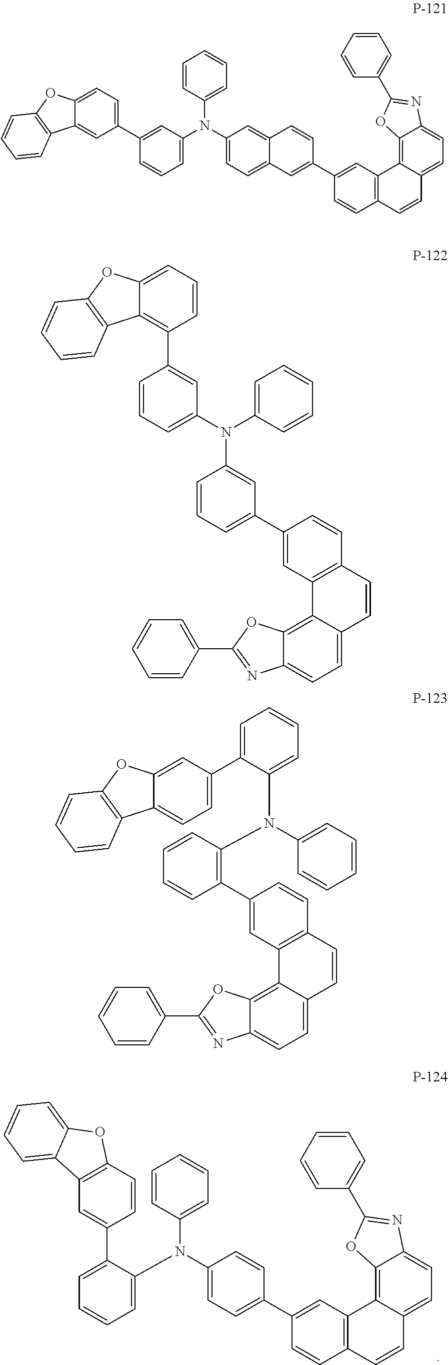

The reorganization energy (RE) value of the compound represented by Formula 1 is 0.10 to 0.19, preferably 0.11 to 0.17.

Hereinafter, reorganization energy is described.

Reorganization energy is the energy lost due to changes in molecular geometry during charge (electron or hole) transfer. It is dependent on molecular geometry and tends to be smaller when the difference between the potential energy surfaces (PES) of the neutral and charged states is smaller. The RE value can be obtained using the following equation.

$${RE}_{hole} \text{:} {λ}^{+} = ( {E}_{NOCE} - {E}_{COCE} ) + ( {E}_{CONE} - {E}_{NONE} )$$

$${RE}_{elec} \text{:} {λ}^{-} = ( {E}_{NOAE} - {E}_{AOAE} ) + ( {E}_{AONE} - {E}_{NONE} )$$

NONE: Neutral geometry of neutral molecular (=NO opt.)
NOAE: Anion geometry of neutral molecular
NOCE: Cation geometry of neutral molecular
AONE: Neutral geometry of Anion molecular
AOAE: Anion geometry of Anion molecular (=AO opt.)
CONE: Neutral geometry of cation molecular
COCE: Cation geometry of cation molecular (=CO opt.)

Reorganization energy and charge mobility have an inverse relationship. When rand T are held constant, the RE value directly influences the mobility of each material.

The relationship between reorganization energy (RE) and mobility can be expressed as the following equation, and it is described based on the charge transfer matrix element.

$$μ = k \frac{{r}^{2}}{2 {k}_{B} T / e}$$

$$k = ( \frac{4 {π}^{2}}{h} ) \frac{{t}^{2}}{\sqrt{4 {πλk}_{B} T}} exp [ - \frac{λ}{4 {k}_{B} T} ]$$

λ: Reorganization energy
μ: mobility
r: dimer displacement
t: intermolecular charge transfer matrix element

As shown in the equation above, charge mobility increases as the RE value decreases.

To calculate the reorganization energy, a simulation tool that can compute the potential energy of a molecule based on its geometry is necessary. For example, Gaussian09 (hereinafter referred to as G09) and the Jaguar (hereinafter referred to as JG) module in Schrödinger Materials Science can be used. Both G09 and JG are quantum mechanical (QM) computational tools used for analyzing molecular properties, and they are capable of optimizing molecular structures and computing the single-point energy for a given molecular structure.

Quantum mechanical (QM) calculations performed on molecular structures require substantial computational resources. For example, such calculations may be executed using two cluster servers, each comprising four node workstations and one master workstation. Each node workstation is capable of performing molecular QM calculations via parallel processing based on symmetric multiprocessing (SMP), utilizing CPUs having 36 or more cores.

Using Gaussian 09 (G09), molecular structures optimized for neutral and charged states are calculated along with their corresponding potential energies (NONE and COCE), which are necessary for determining the reorganization energy. Subsequently, by altering only the charge of each of the two optimized structures, the potential energy of the neutral-state-optimized structure under the charged state (NOCE) and the potential energy of the charged-state-optimized structure under the neutral state (CONE) are calculated. The reorganization energy is then determined based on the following relationship.

$${RE}_{charge} \text{:} λ = ( {E}_{NOCE} - {E}_{COCE} ) + ( {E}_{CONE} - {E}_{NONE} )$$

Since Schrödinger provides a function for automatically performing the aforementioned calculation process, the JG module is capable of sequentially calculating the potential energies of each state and determining the rearrangement energy (RE) value by simply inputting the molecular structure (NO) of the ground state.

According to another embodiment of the present invention, an organic electronic device is provided, which comprises a first electrode, a second electrode, and an organic layer disposed between the first and second electrodes, wherein the organic layer comprises a compound represented by Formula 1.

The organic layer includes a phosphorescent light-emitting layer, which comprises a compound represented by Formula 1 and a compound represented by Formula 2.

According to another embodiment of the present invention, an organic electronic device is provided, which comprises a first electrode, a second electrode, and an organic layer disposed between the first and second electrodes, wherein the organic layer comprises a compound represented by Formula 1.

The organic layer includes a phosphorescent light-emitting layer, which comprises a compound represented by Formula 1 and a compound represented by Formula 2.

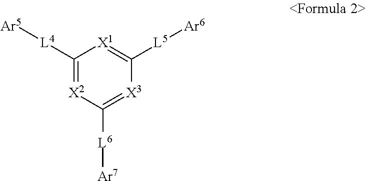

In Formula 2, each symbol may be defined as follows.

X¹ to X³ are independently C(R′) or N, and at least one of X¹ to X³ is N.

L⁴ to L⁶ are each independently selected from the group consisting of a single bond, a C₆-C₆₀ arylene group, a fluorenylene group, a C₃-C₆₀ aliphatic ring group, and a C₂-C₆₀ heterocyclic group containing at least one heteroatom of O, N, S, Si and P.

Ar⁵ to Ar⁷ are each independently selected from the group consisting of a C₆-C₆₀ aryl group, a fluorenyl group, a C₃-C₆₀ aliphatic ring group, and a C₂-C₆₀ heterocyclic group containing at least one heteroatom of O, N, S, Si and P.

R′ is independently selected from the group consisting of hydrogen, deuterium, halogen, a cyano group, a nitro group, a C₆-C₆₀ aryl group, a fluorenyl group, a C₂-C₆₀ heterocyclic group containing at least one heteroatom of O, N, S, Si and P, a C₃-C₆₀ aliphatic ring group, a C₁-C₂₀ alkyl group, a C₂-C₂₀ alkenyl group, a C₂-C₂₀ alkynyl group, a C₁-C₂₀ alkoxyl group, and a C₆-C₆₀ aryloxy group.

When at least one of Ar⁵ to Ar⁷, R′ is an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, or a C₁₈ aryl group, specifically, phenyl, biphenyl, naphthyl, terphenyl, phenanthrene, triphenylene, or the like.

When at least one of L⁴ to L⁶ is an arylene group, the arylene group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, or a C₁₈ arylene group, specifically, phenylene, biphenyl, naphthylene, terphenyl, phenanthrene, triphenylene, or the like.

When at least one of Ar⁵ to Ar⁷, R′, L⁴ to L⁶ is a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, or a C₂₉ heterocyclic group, specifically, pyridine, pyrimidine, pyrazine, pyridazine, triazine, furan, pyrrole, silole, indene, indole, phenyl-indole, benzoindole, phenyl-benzoindole, pyrazinoindol, quinoline, isoquinoline, benzoquinoline, pyridoquinoline, quinazoline, benzoquinazoline, dibenzoquinazoline, phenanthroquinazoline, quinoxaline, benzoquinoxaline, dibenzoquinoxaline, benzofuran, naphthobenzofuran, dibenzofuran, dinaphthofuran, thiophene, benzothiophene, dibenzothiophene, naphthobenzothiophene, dinaphthothiophene, carbazole, phenyl-carbazole, benzocarbazole, phenyl-benzocarbazole, naphthyl-benzocarbazole, dibenzocarbazole, indolocarbazole, benzofuropyridine, benzothienopyridine, benzofuropyridine, benzothienopyrimidine, benzofuropyrimidine, benzothienopyrazine, benzofuropyrazine, benzoimidazole, benzothiazole, benzooxazole, benzosiloe, phenanthroline, dihydro-phenylphenazine, 10-phenyl-10H-phenoxazine, phenoxazine, phenothiazine, dibenzodioxin, benzodibenzodioxin, thianthrene, 9,9-dimethyl-9H-xanthene, 9,9-dimethyl-9H-thioxanthene, dihydrodimethylphenylacridine, spiro[fluorene-9,9′-xanthene] and the like.

When at least one of Ar⁵ to Ar⁷, R′ is a fluorenyl group or at least one of L⁴ to L⁶ are a fluorenylene group, the fluorenyl group or the fluorenylene group may be, for example, 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl)9-phenyl-9H-fluorene, and the like.

The aryl group, the arylene group, the fluorenyl group, the fluorenylene group, the heterocyclic group, the aliphatic ring group, the alkyl group, the alkenyl group, the alkynyl group, the alkoxyl group, and the aryloxyl group may be each substituted with one or more substituents selected from the group consisting of deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group.

When at least one of the aryl group, the arylene group, the fluorenyl group, the fluorenylene group, the heterocyclic group, the aliphatic ring group, the alkyl group, the alkenyl group, the alkynyl group, the alkoxyl group, and the aryloxyl group is substituted with an aryl group, the aryl group may be, for example, a C₆-C₃₀, a C₆-C₂₉, a C₆-C₂₈, a C₆-C₂₇, a C₆-C₂₆, a C₆-C₂₅, a C₆-C₂₄, a C₆-C₂₃, a C₆-C₂₂, a C₆-C₂₁, a C₆-C₂₀, a C₆-C₁₉, a C₆-C₁₈, a C₆-C₁₇, a C₆-C₁₆, a C₆-C₁₅, a C₆-C₁₄, a C₆-C₁₃, a C₆-C₁₂, a C₆-C₁₁, a C₆-C₁₀, a C₆, a C₁₀, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉, or a C₃₀ aryl group.

When at least one of the aryl group, the arylene group, the fluorenyl group, the fluorenylene group, the heterocyclic group, the aliphatic ring group, the alkyl group, the alkenyl group, the alkynyl group, the alkoxyl group, and the aryloxyl group is substituted with a heterocyclic group, the heterocyclic group may be, for example, a C₂-C₃₀, a C₂-C₂₉, a C₂-C₂₈, a C₂-C₂₇, a C₂-C₂₆, a C₂-C₂₅, a C₂-C₂₄, a C₂-C₂₃, a C₂-C₂₂, a C₂-C₂₁, a C₂-C₂₀, a C₂-C₁₉, a C₂-C₁₈, a C₂-C₁₇, a C₂-C₁₆, a C₂-C₁₅, a C₂-C₁₄, a C₂-C₁₃, a C₂-C₁₂, a C₂-C₁₁, a C₂-C₁₀, a C₂-C₉, a C₂-C₈, a C₂-C₇, a C₂-C₆, a C₂-C₅, a C₂-C₄, a C₂-C₃, a C₂, a C₃, a C₄, a C₅, a C₆, a C₇, a C₈, a C₉, a C₁₀, a C₁₁, a C₁₂, a C₁₃, a C₁₄, a C₁₅, a C₁₆, a C₁₇, a C₁₈, a C₁₉, a C₂₀, a C₂₁, a C₂₂, a C₂₃, a C₂₄, a C₂₅, a C₂₆, a C₂₇, a C₂₈, a C₂₉, or a C₃₀ heterocyclic group.

When at least one of the aryl group, the arylene group, the fluorenyl group, the fluorenylene group, the heterocyclic group, the aliphatic ring group, the alkyl group, the alkenyl group, the alkynyl group, the alkoxyl group, and the aryloxyl group is substituted with a fluorenyl group, the fluorenyl group may be 9,9-dimethyl-9H-fluorene, 9,9-diphenyl-9H-fluorene, 9,9′-spirobifluorene, spiro[benzo[b]fluorene-11,9′-fluorene], benzo[b]fluorene, 11,11-diphenyl-11H-benzo[b]fluorene, 9-(naphthalen-2-yl) 9-phenyl-9H-fluorene, and the like.

At least one of Ar⁵ to Ar⁷ may be selected from the group consisting of Formula Ar-1 to Formula Ar-8.

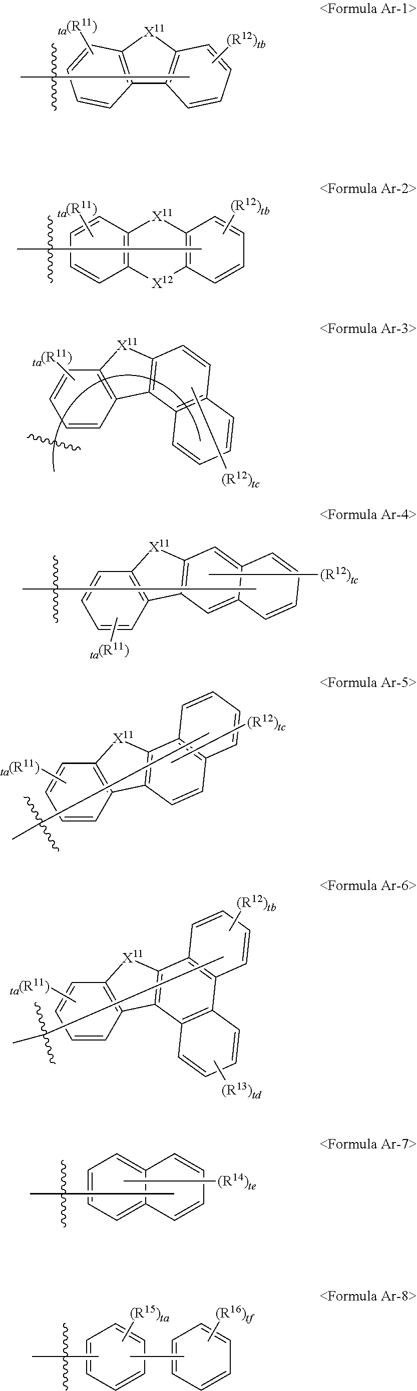

In Formula Ar-1 to Formula Ar-8, each symbol may be defined as follows.

X¹¹ and X¹² are independently N(Ar¹¹), O, S or C(R¹⁷)(R¹⁸).

R¹¹ to R¹⁸ are each independently selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent groups may be bonded to each other to form a ring, and R¹⁷ and R¹⁸ may be bonded to each other to form a ring. A spiro compound is formed when R¹⁷ and R¹⁸ are connected to form a ring structure.

ta, tb, td are each an integer of 0 to 4, tc is an integer of 0 to 6, te is an integer of 0 to 7, tf is an integer of 0 to 5,
Ar¹¹ is selected from the group consisting of a C₆-C₃₀ aryl group, a fluorenyl group, a C₂-C₃₀ heterocyclic group containing at least one heteroatom of O, N, S, Si and P, and a C₃-C₃₀ aliphatic ring group.

Formula 2 may be represented by one of the following Formula 2-1 to Formula 2-6.

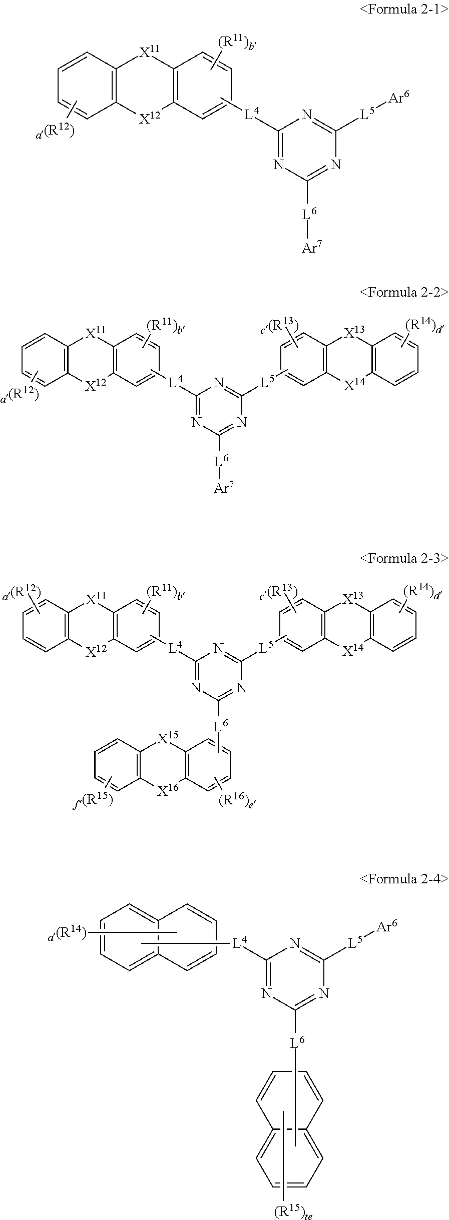

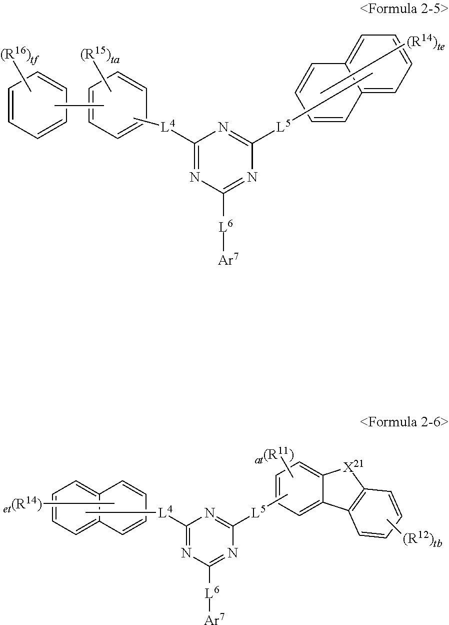

In Formula 2-1 to Formula 2-6, L⁴ to L⁶, Ar⁶, Ar⁷ are the same as defined for Formula 2.

X¹¹, X¹³, X¹⁵ and X²¹ are independently NAr¹¹, O, S or C(R¹⁷)(R¹⁸), and X¹², X¹⁴ and X¹⁶ are independently a single bond, N(Ar¹²), O, S or C(R²¹)(R²²).

Ar¹¹ and Ar¹² are each independently selected from the group consisting of a C₆-C₃₀ aryl group, a fluorenyl group, a C₂-C₃₀ heterocyclic group containing at least one heteroatom of O, N, S, Si and P, and a C₃-C₃₀ aliphatic ring group.

R¹¹ to R¹⁸, R²¹, R²² are each independently selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent groups may be bonded to each other to form a ring, R¹⁷ and R¹⁸ may be bonded to each other to form a ring, and R²¹ and R²² may be bonded to each other to form a ring. A spiro compound is formed when R¹⁷ and R¹⁸, or R²¹ and R²² are connected to form a ring structure.

a′, d′ and f are each an integer of 0 to 4, b′, c′ and e′ are each an integer of 0 to 3, ta, tb and td are each an integer of 0 to 4, te is an integer of 0 to 7, tf is an integer of 0 to 5.

In Formula 2, at least one of L⁴ to L⁶ is selected from the group consisting of the following Formula b-1 to b-13.

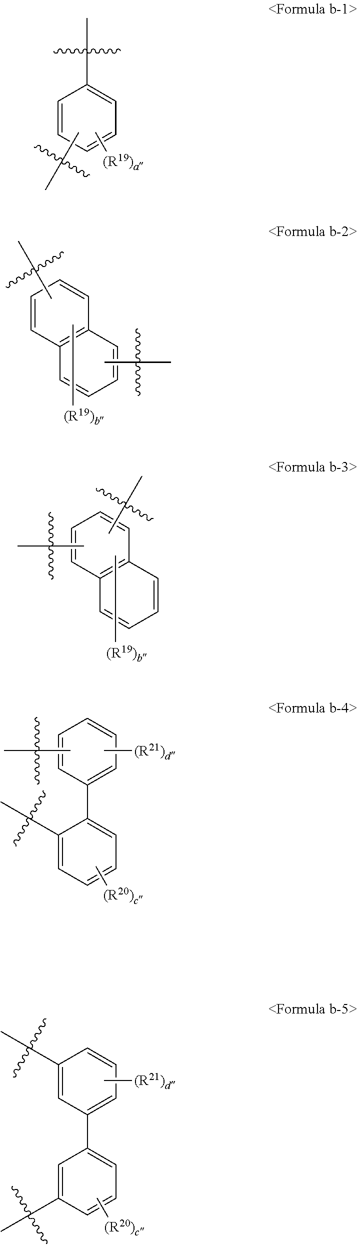

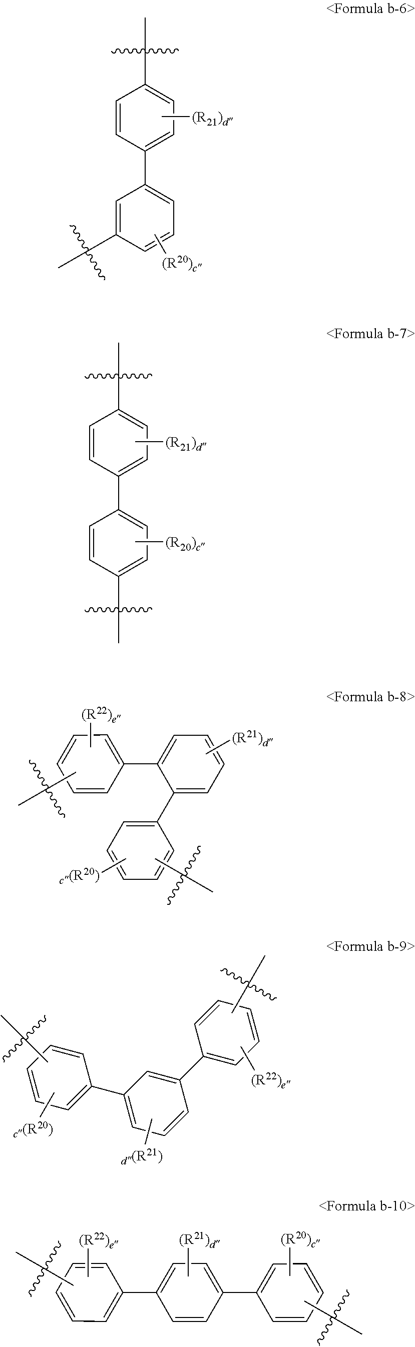

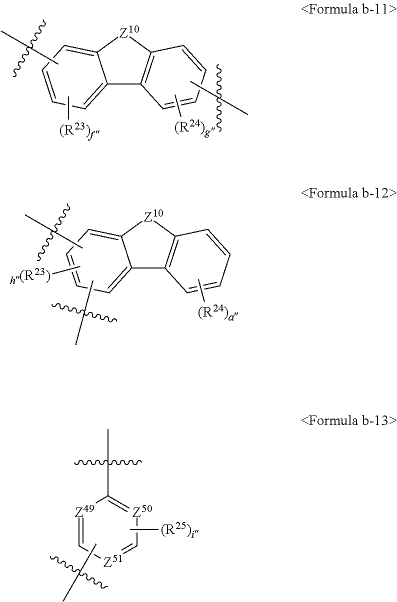

In Formula b-1 to Formula b-13, each symbol may be defined as follows.

Z¹⁰ is S, O, C(R₁)(R₂) or N(R₃).

Z⁴⁹ to Z⁵¹ are independently C(R₄) or N, and at least one of Z⁴⁹ to Z⁵¹ is N.

R¹⁹ to R²⁴, R₁ to R₄ are each independently selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent groups may be bonded to each other to form a ring, and R₁ and R₂ may be bonded to each other to form a ring. A spiro compound is formed when R₁ and R₂ are connected to form a ring structure.

a″, c″, d″ and e″ are each an integer of 0 to 4, b″ is an integer of 0 to 6, f″ and g″ are each an integer of 0 to 3, h″ is an integer of 0 to 2, i″ is an integer of 0 to 3.

Specifically, the compound represented by Formula 2 may be one of the following compounds, but there is no limitation thereto.

[chemical structure]

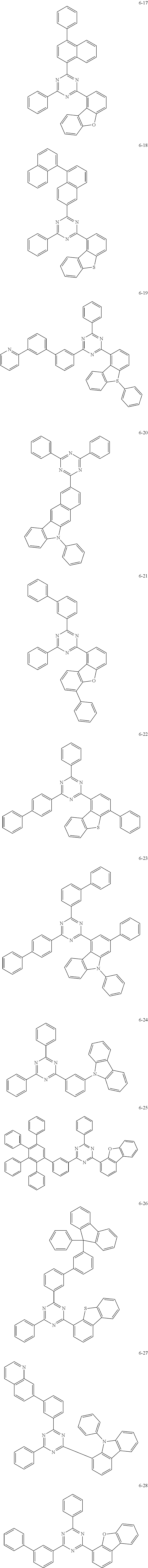

[chemical structure]

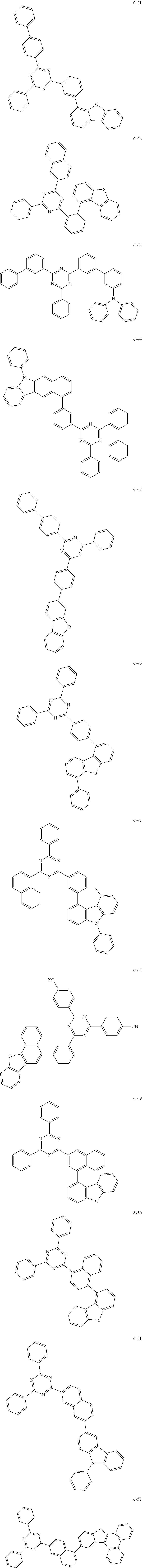

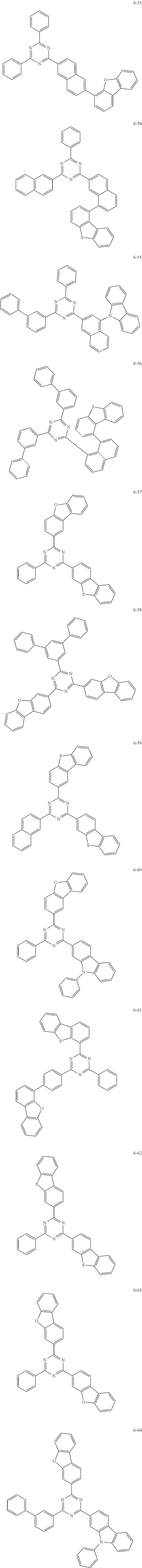

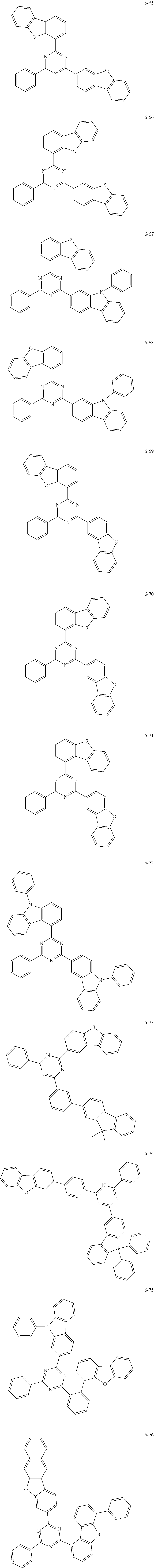

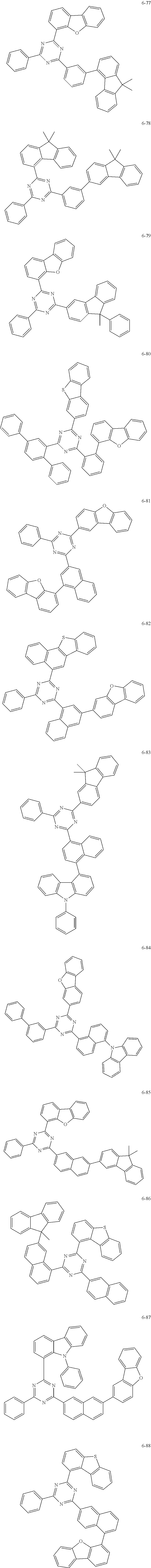

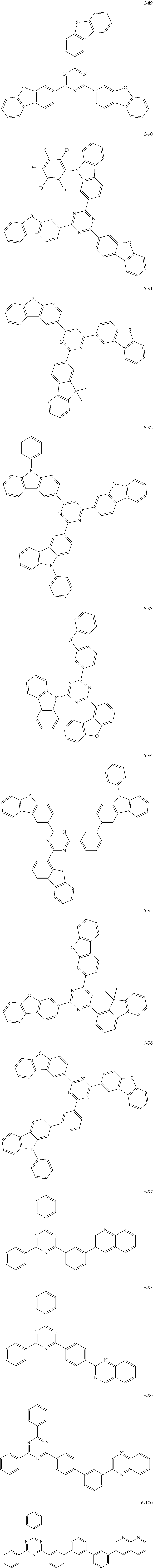

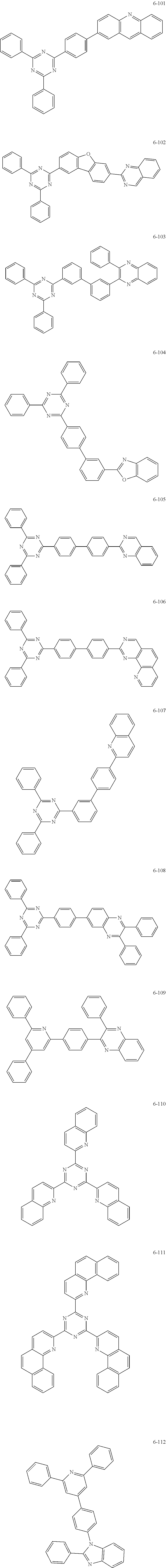

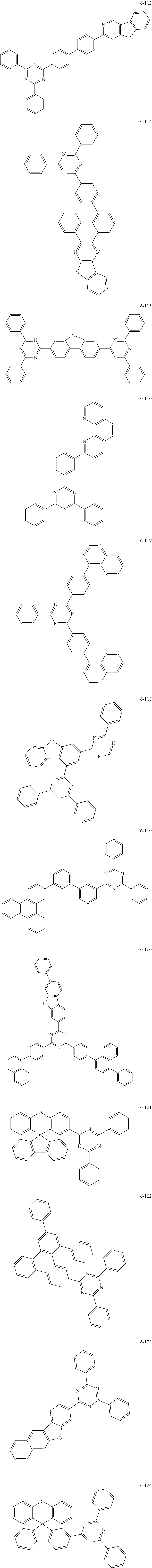

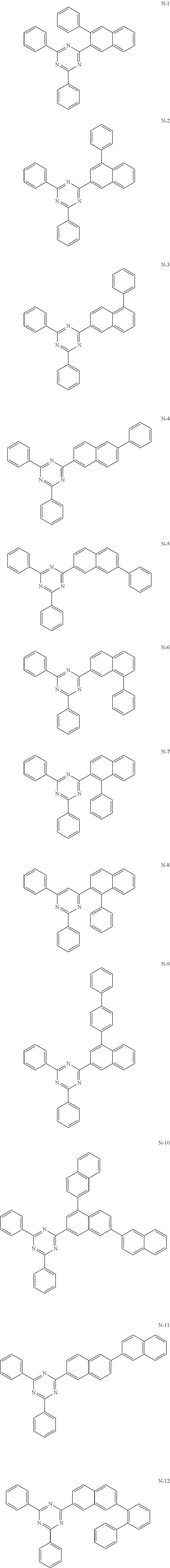

[chemical structure]

[chemical structure]

[chemical structure]

[chemical structure]

[chemical structure]

[chemical structure]

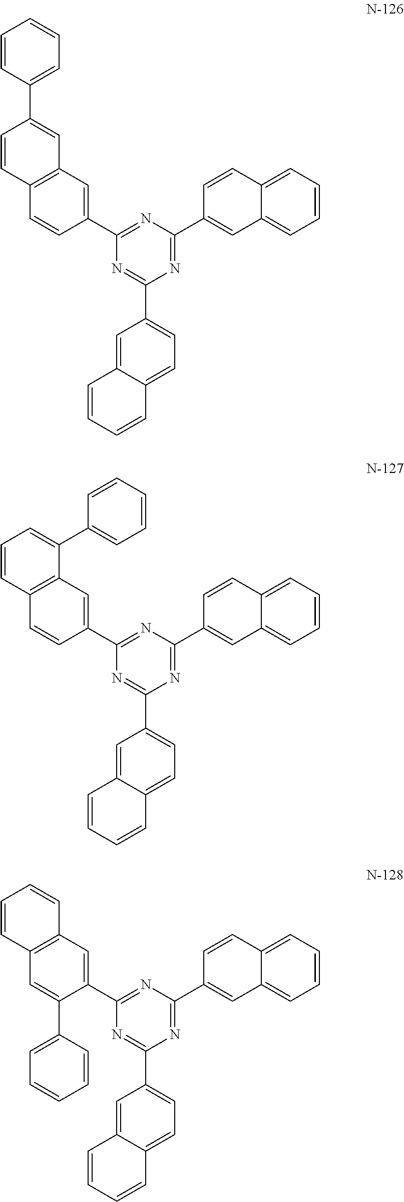

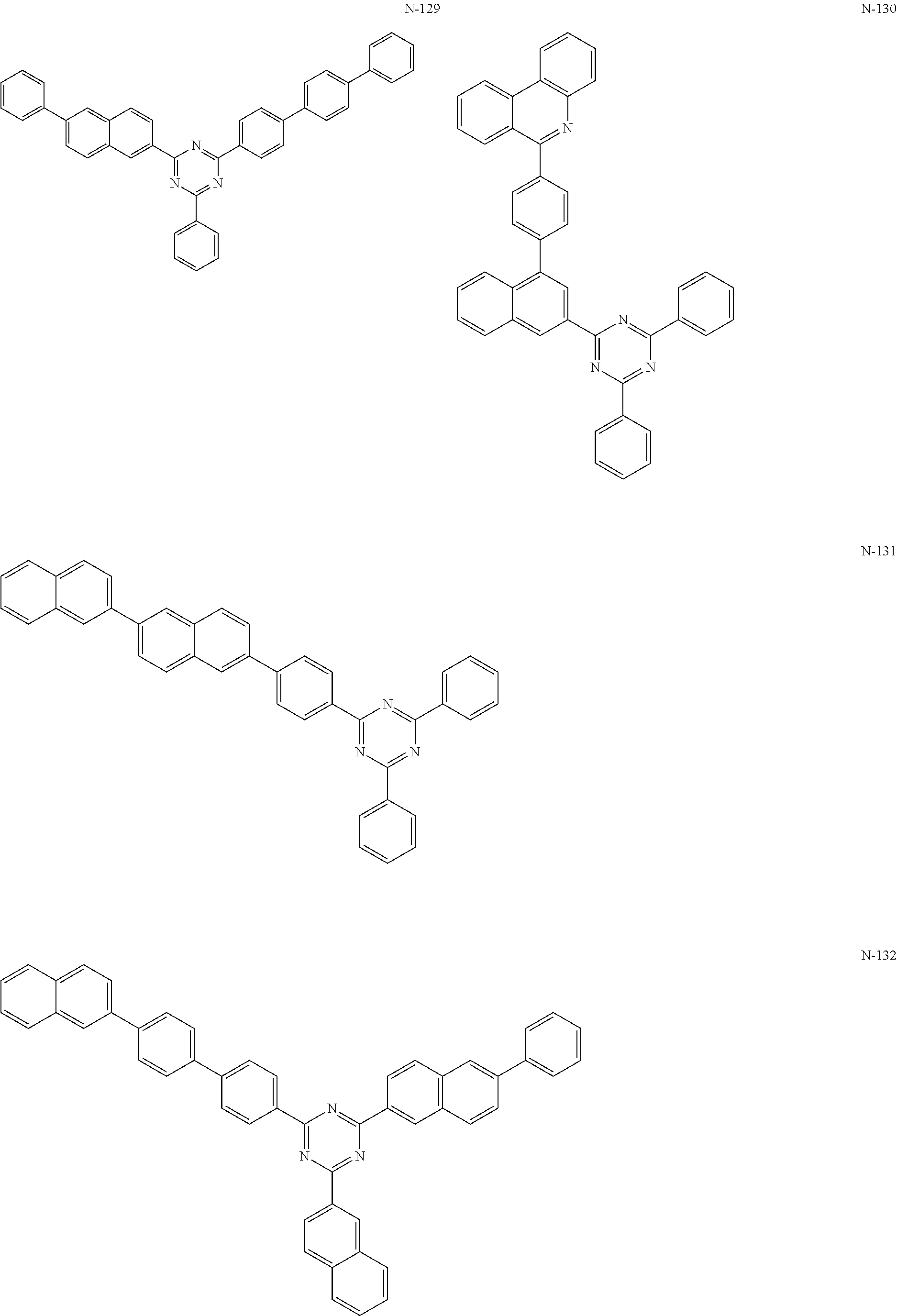 [SMILES: c1ccc(-c2ccc(-c3ccc(-c4nc(-c5ccccc5)nc(-c5ccc6cc(-c7ccccc7)ccc6c5)n4)cc3)cc2)cc1 ; c1ccc(-c2nc(-c3ccccc3)nc(-c3cc(-c4ccc(-c5nc6ccccc6c6ccccc56)cc4)c4ccccc4c3)n2)cc1 ; c1ccc(-c2nc(-c3ccccc3)nc(-c3ccc(-c4ccc5cc(-c6ccc7ccccc7c6)ccc5c4)cc3)n2)cc1 ; c1ccc(-c2ccc3cc(-c4nc(-c5ccc(-c6ccc(-c7ccc8ccccc8c7)cc6)cc5)nc(-c5ccc6ccccc6c5)n4)ccc3c2)cc1]

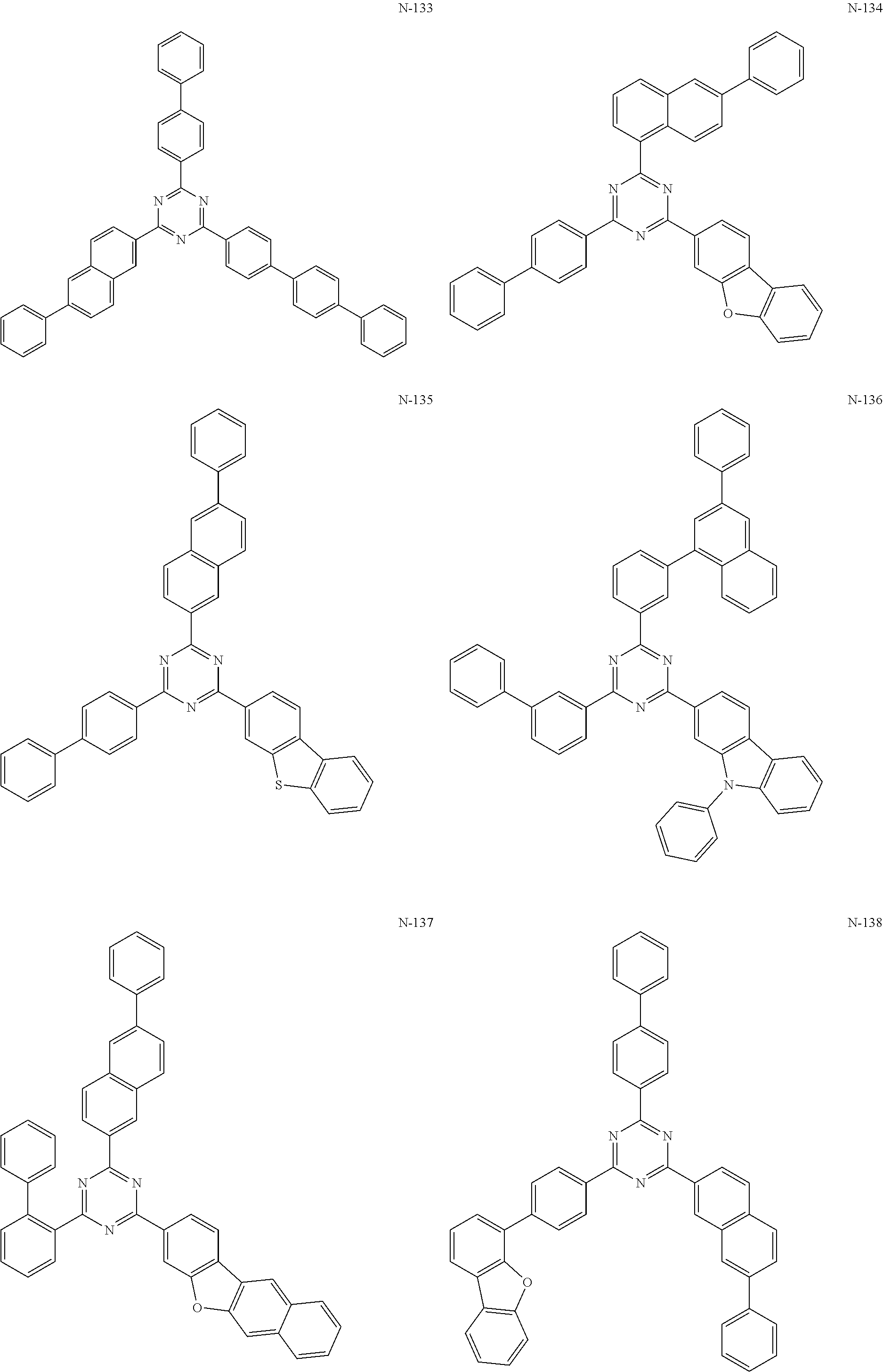

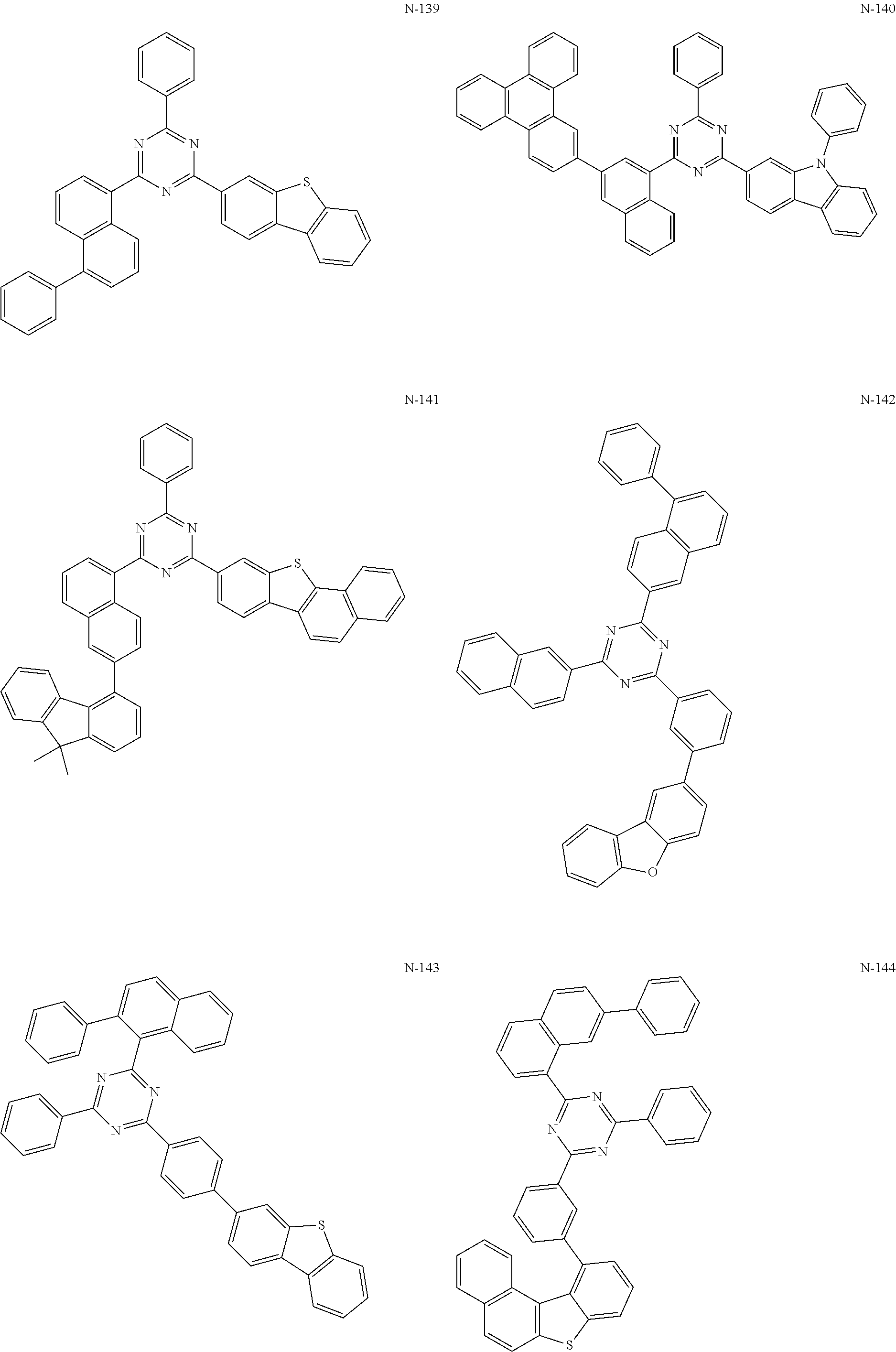

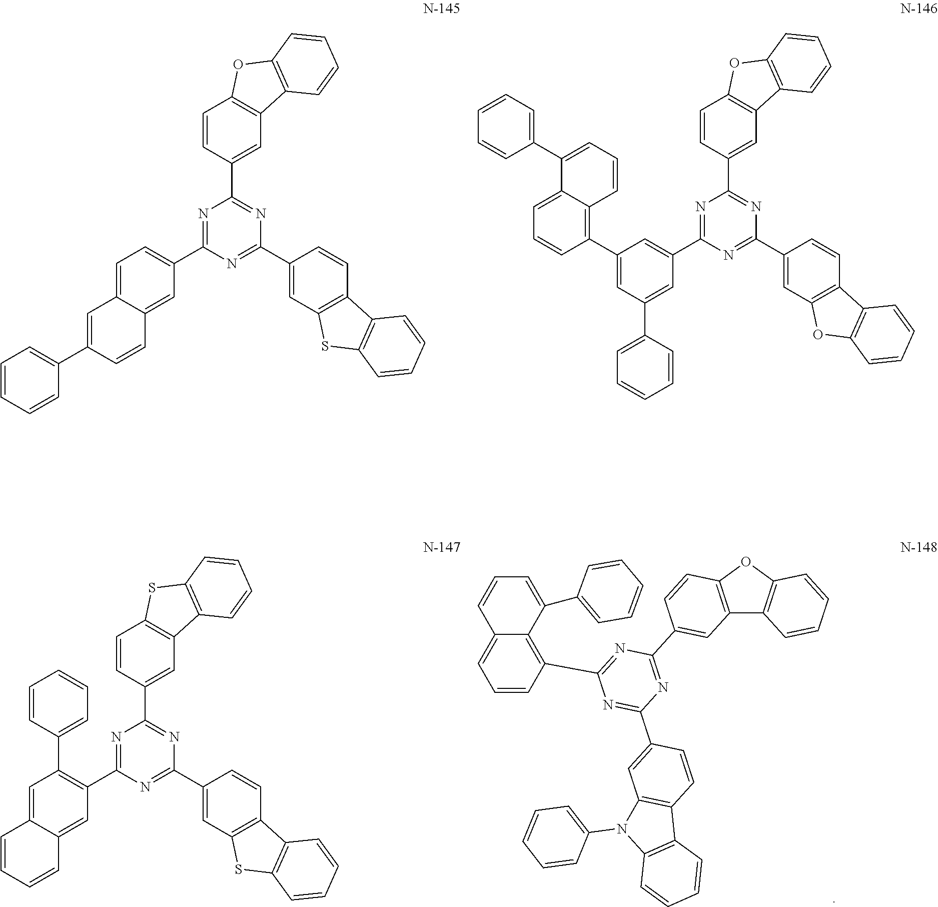 [SMILES: c1ccc(-c2ccc3cc(-c4nc(-c5ccc6c(c5)sc5ccccc56)nc(-c5ccc6oc7ccccc7c6c5)n4)ccc3c2)cc1 ; c1ccc(-c2cc(-c3nc(-c4ccc5c(c4)oc4ccccc45)nc(-c4ccc5oc6ccccc6c5c4)n3)cc(-c3cccc4c(-c5ccccc5)cccc34)c2)cc1 ; c1ccc(-c2cc3ccccc3cc2-c2nc(-c3ccc4c(c3)sc3ccccc34)nc(-c3ccc4sc5ccccc5c4c3)n2)cc1 ; c1ccc(-c2cccc3cccc(-c4nc(-c5ccc6oc7ccccc7c6c5)nc(-c5ccc6c7ccccc7n(-c7ccccc7)c6c5)n4)c23)cc1]

In another aspect, the present invention provides an electronic apparatus comprising a display device comprising an organic electronic device, and a control unit configured to drive the display device, wherein the organic electronic device comprises a compound represented by Formula 1.

In another aspect, the present invention provides a compound represented by Formula 1, which is obtained by recovering and purifying a material of an organic layer from a deposition apparatus after deposition of the organic layer in a process for manufacturing an organic electronic device. The compound obtained through the recovery and purification process has a purity of 99.9% or higher.

In another aspect, the present invention provides a method for recovering a compound represented by Formula 1, the method comprising: depositing an organic layer material comprising the compound represented by Formula 1; recovering the organic layer material that is attached to a deposition apparatus; and purifying the recovered organic layer material to obtain the compound represented by Formula 1 having a purity of 99.9% or higher.

The purification step may comprise: a recrystallization step in which the recovered material of an organic layer is recrystallized using a recrystallization solvent; an adsorption and separation step using an adsorbent; and a sublimation and purification step.

The recrystallization step may comprise a preliminary purification process in which a compound represented by Formula 1 having a purity of 98% is obtained using a recrystallization solvent.

As the recrystallization solvent, a polar solvent having a polarity index (PI) of 5.5 to 7.2 is preferably used, or a mixed solvent comprising a polar solvent having a polarity index of 5.5 to 7.2 and a non-polar solvent having a polarity index of 2.0 to 4.7 may be used.

When a mixed solvent of a polar solvent and a non-polar solvent is used as the recrystallization solvent, the non-polar solvent may be used in an amount of 15% (v/v) or less relative to the volume of the polar solvent.

As the recrystallization solvent, a single solvent of methylpyrrolidone (N-methylpyrrolidone; NMP) is preferably used. Alternatively, a mixed polar solvent in which methylpyrrolidone is mixed with at least one solvent selected from the group consisting of 1,3-dimethyl-2-imidazolidinone, 2-pyrrolidone, N,N-dimethylformamide, dimethylacetamide, and dimethyl sulfoxide may be used. In addition, a single or mixed non-polar solvent selected from the group consisting of toluene, dichloromethane (DCM), dichloroethane (DCE), tetrahydrofuran (THF), chloroform, ethyl acetate, and butanone, or a mixture of the polar solvent and the non-polar solvent may also be used.

The preliminary purification process may comprise dissolving the unpurified organic electroluminescent material, which has been recovered from a deposition apparatus, in a polar solvent at a temperature of 90° C. to 120° C., and cooling the solution to a temperature of 0° C. to 5° C. to precipitate crystals.

The preliminary purification process may comprise dissolving the unpurified organic electroluminescent material, recovered from a deposition apparatus, in a polar solvent at a temperature of 90° C. to 120° C.; cooling the solution to a temperature of 35° C. to 40° C.; adding a non-polar solvent to the cooled solution; and further cooling the resulting mixture to a temperature of 0° C. to 5° C. to precipitate crystals.

The preliminary purification process may comprise dissolving the unpurified organic electroluminescent material, recovered from a deposition apparatus, in a non-polar solvent, and concentrating the solution to remove the non-polar solvent while precipitating crystals.

The preliminary purification process may comprise a step of recrystallization using a polar solvent, followed by a subsequent recrystallization step using a non-polar solvent.

In the adsorption separation step using an adsorbent, the adsorbent may be selected from the group consisting of activated carbon, silica gel, alumina, and other materials known for use in adsorption.

Hereinafter, the present invention will be described in further detail with reference to specific examples regarding the synthesis of compounds represented by Formula 1 and Formula 2 and the fabrication of an organic electronic device. However, the present invention is not limited to the following examples.

### [Synthesis Example 1] Compound of Formula 1

The compound (final product) represented by Formula 1 according to the present invention may be synthesized by reacting Sub 1 with Sub 2 as illustrated in Reaction Scheme 1. However, the present invention is not limited thereto.

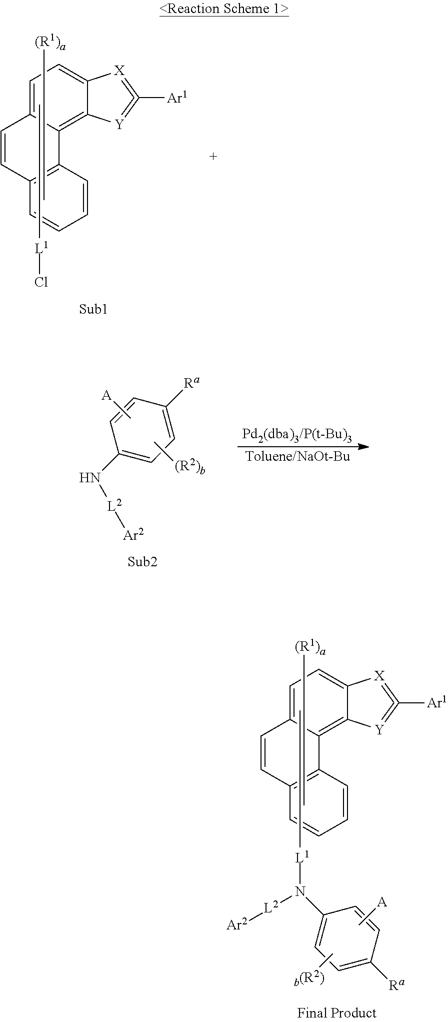

### Synthesis Example of Sub1

Sub1 of Reaction Scheme 1 was synthesized as disclosed in Korean Registered Patent No. 10-2112786 (published on May 13, 2020), and the compounds belonging to Sub1 may be, but not limited to, the following compounds. Table 1 shows the FD-MS (Field Desorption-Mass Spectrometry) values of the following compounds.

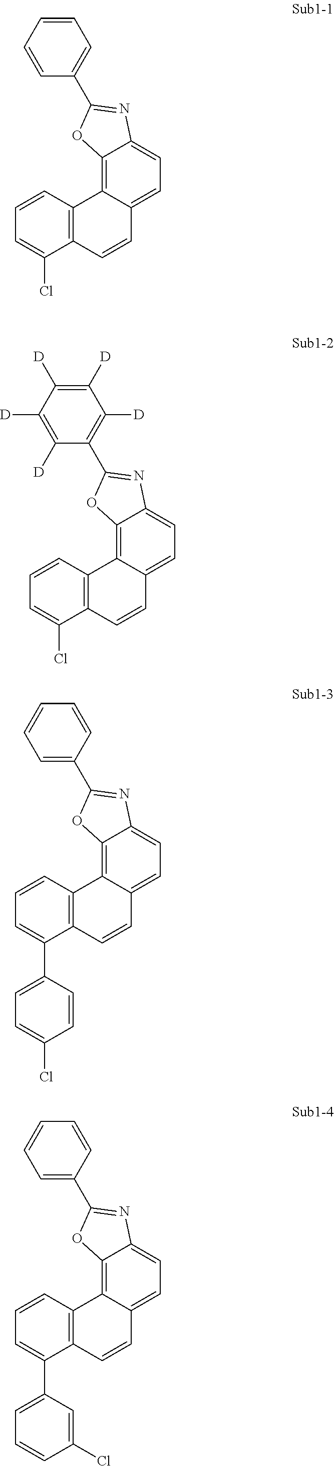

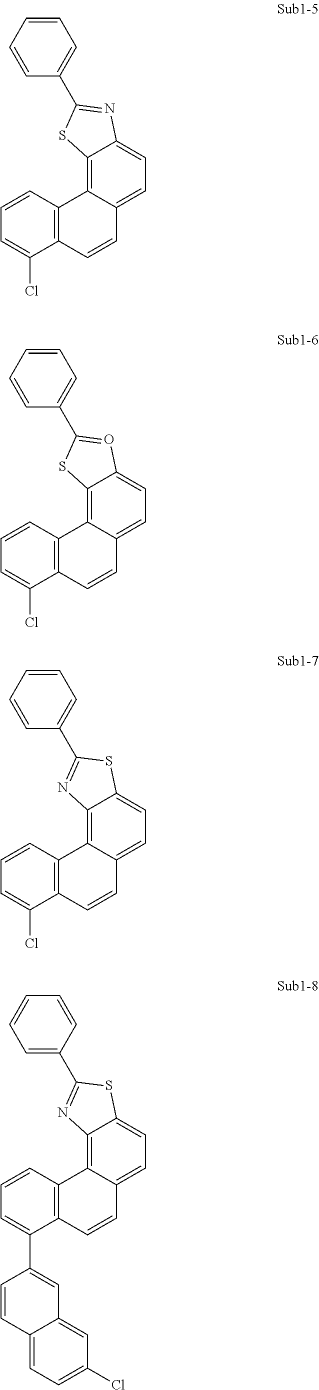

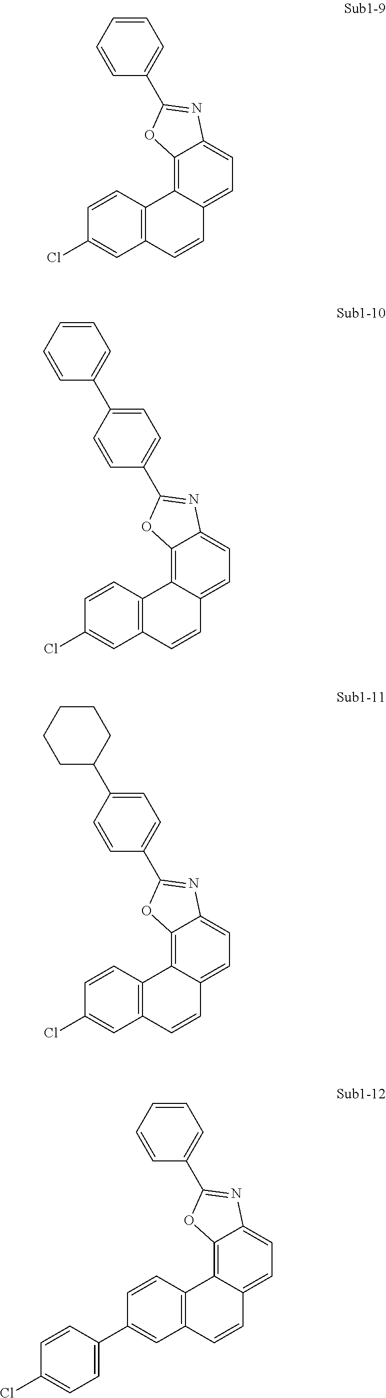 [SMILES: Clc1ccc2c(ccc3ccc4nc(-c5ccccc5)oc4c32)c1 ; Clc1ccc2c(ccc3ccc4nc(-c5ccc(-c6ccccc6)cc5)oc4c32)c1 ; Clc1ccc2c(ccc3ccc4nc(-c5ccc(C6CCCCC6)cc5)oc4c32)c1 ; Clc1ccc(-c2ccc3c(ccc4ccc5nc(-c6ccccc6)oc5c43)c2)cc1]

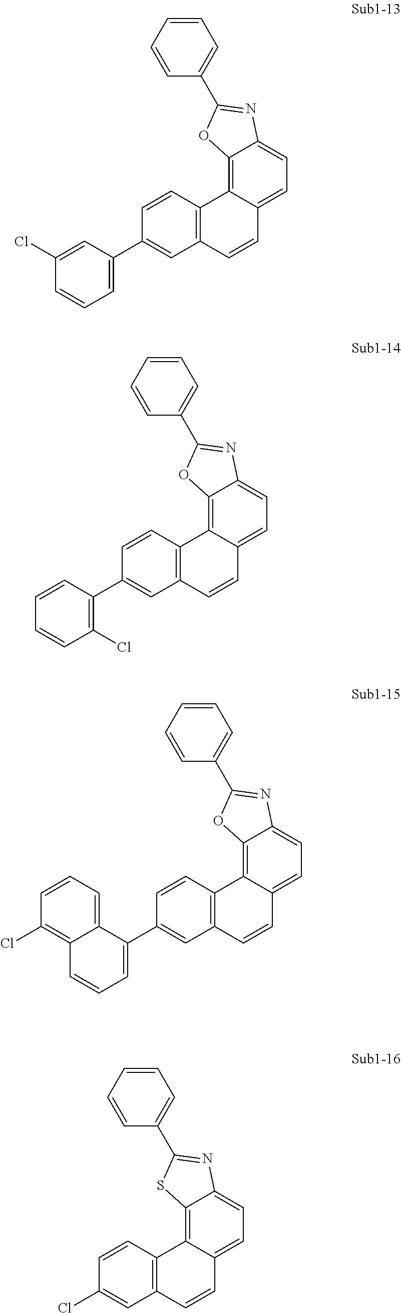 [SMILES: Clc1cccc(-c2ccc3c(ccc4ccc5nc(-c6ccccc6)oc5c43)c2)c1 ; Clc1ccccc1-c1ccc2c(ccc3ccc4nc(-c5ccccc5)oc4c32)c1 ; Clc1cccc2c(-c3ccc4c(ccc5ccc6nc(-c7ccccc7)oc6c54)c3)cccc12 ; Clc1ccc2c(ccc3ccc4nc(-c5ccccc5)sc4c32)c1]

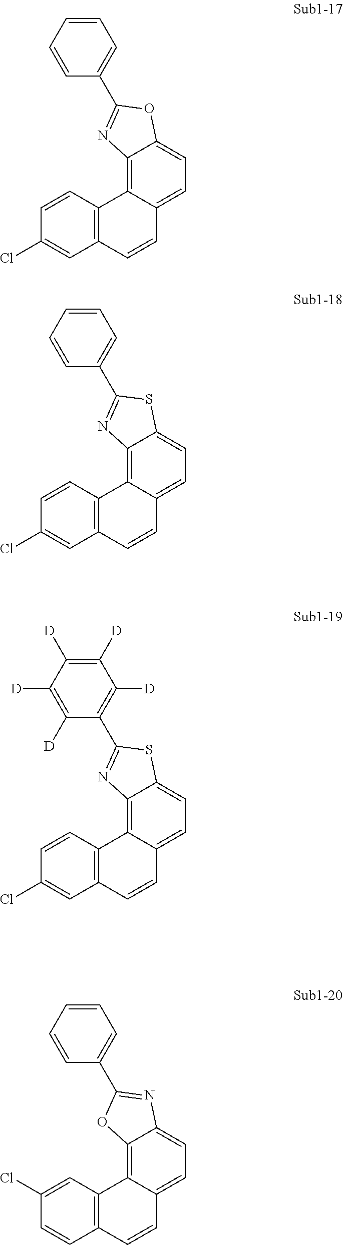 [SMILES: Clc1ccc2c(ccc3ccc4oc(-c5ccccc5)nc4c32)c1 ; Clc1ccc2c(ccc3ccc4sc(-c5ccccc5)nc4c32)c1 ; [2H]c1c([2H])c([2H])c(-c2nc3c(ccc4ccc5cc(Cl)ccc5c43)s2)c([2H])c1[2H] ; Clc1ccc2ccc3ccc4nc(-c5ccccc5)oc4c3c2c1]

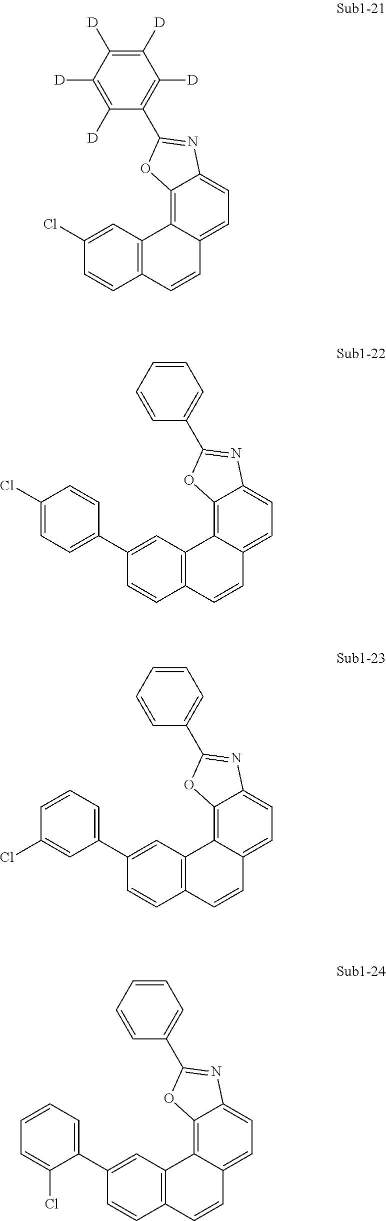 [SMILES: [2H]c1c([2H])c([2H])c(-c2nc3ccc4ccc5ccc(Cl)cc5c4c3o2)c([2H])c1[2H] ; Clc1ccc(-c2ccc3ccc4ccc5nc(-c6ccccc6)oc5c4c3c2)cc1 ; Clc1cccc(-c2ccc3ccc4ccc5nc(-c6ccccc6)oc5c4c3c2)c1 ; Clc1ccccc1-c1ccc2ccc3ccc4nc(-c5ccccc5)oc4c3c2c1]

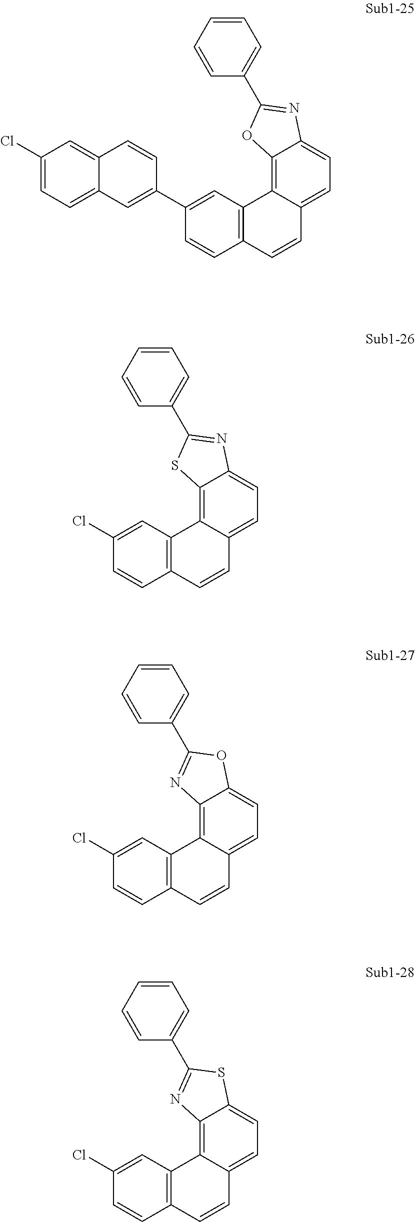 [SMILES: Clc1ccc2cc(-c3ccc4ccc5ccc6nc(-c7ccccc7)oc6c5c4c3)ccc2c1 ; Clc1ccc2ccc3ccc4nc(-c5ccccc5)sc4c3c2c1 ; Clc1ccc2ccc3ccc4oc(-c5ccccc5)nc4c3c2c1 ; Clc1ccc2ccc3ccc4sc(-c5ccccc5)nc4c3c2c1]

TABLE 1

| Compound | FD-MS | Compound | FD-MS |
|---|---|---|---|
| Sub1-1 | m/z = 329.06(C₂₁H₁₂CINO = 329.78) | Sub1-2 | m/z = 334.09(C₂₁H₇D₅CINO = 334.81) |
| Sub1-3 | m/z = 405.09(C₂₇H₁₆CINO = 405.88) | Sub1-4 | m/z = 405.09(C₂₇H₁₆CINO = 405.88) |
| Sub1-5 | m/z = 345.04(C₂₁H₁₂CINS = 345.84) | Sub1-6 | m/z = 329.06(C₂₁H₁₂CINO = 329.78) |
| Sub1-7 | m/z = 345.04(C₂₁H₁₂CINS = 345.84) | Sub1-8 | m/z = 471.08(C₃₁H₁₈CINS = 472) |
| Sub1-9 | m/z = 329.06(C₂₁H₁₂CINO = 329.78) | Sub1-10 | m/z = 405.09(C₂₇H₁₆CINO = 405.88) |
| Sub1-11 | m/z = 411.14(C₂₇H₂₂CINO = 411.93) | Sub1-12 | m/z = 405.09(C₂₇H₁₆CINO = 405.88) |
| Sub1-13 | m/z = 405.09(C₂₇H₁₆CINO = 405.88) | Sub1-14 | m/z = 405.09(C₂₇H₁₆CINO = 405.88) |
| Sub1-15 | m/z = 455.11(C₃₁H₁₈CINO = 455.94) | Sub1-16 | m/z = 345.04(C₂₁H₁₂CINS = 345.84) |
| Sub1-17 | m/z = 329.06(C₂₁H₁₂CINO = 329.78) | Sub1-18 | m/z = 345.04(C₂₁H₁₂CINS = 345.84) |
| Sub1-19 | m/z = 350.07(C₂₁H₇D₅CINS = 350.87) | Sub1-20 | m/z = 329.06(C₂₁H₁₂CINO = 329.78) |
| Sub1-21 | m/z = 334.09(C₂₁H₇D₅CINO = 334.81) | Sub1-22 | m/z = 405.09(C₂₇H₁₆CINO = 405.88) |
| Sub1-23 | m/z = 405.09(C₂₇H₁₆CINO = 405.88) | Sub1-24 | m/z = 405.09(C₂₇H₁₆CINO = 405.88) |
| Sub1-25 | m/z = 455.11(C₃₁H₁₈CINO = 455.94) | Sub1-26 | m/z = 345.04(C₂₁H₁₂CINS = 345.84) |
| Sub1-27 | m/z = 329.06(C₂₁H₁₂CINO = 329.78) | Sub1-28 | m/z = 345.04(C₂₁H₁₂CINS = 345.84) |

### II. Synthesis Example of Sub2

Sub2 in Reaction Scheme 1 may be synthesized via the reaction pathway illustrated in Reaction Scheme 2, but is not limited thereto.

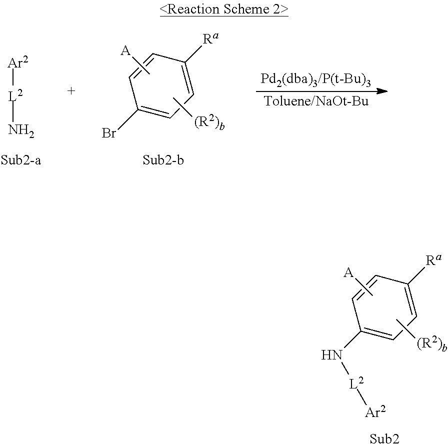

### Synthesis Example of Sub2-1

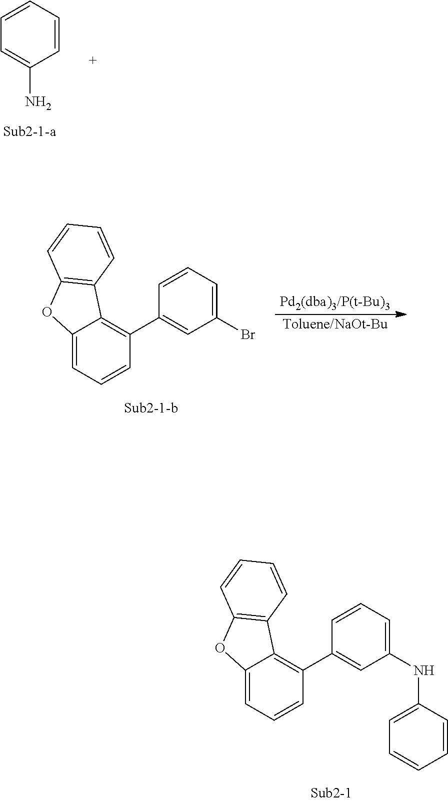 [SMILES: Nc1ccccc1 ; Brc1cccc(-c2cccc3oc4ccccc4c23)c1 ; "Toluene/NaOt-Bu" ; c1ccc(Nc2cccc(-c3cccc4oc5ccccc5c34)c2)cc1]

Sub2-1-a (10.0 g, 107 mmol) was dissolved in toluene (540 mL), followed by the addition of Sub2-1-b (34.7 g, 107 mmol), Pd₂(dba)₃ (2.95 g, 3.22 mmol), P(t-Bu)₃ (1.30 g, 6.44 mmol), and NaOt-Bu (20.6 g, 215 mmol). The reaction was carried out at 80° C. When the reaction was completed, the reaction products were extracted with CH₂Cl₂ and water, and the organic layer was dried over MgSO₄ and concentrated. Then, the concentrate was purified by silica gel column chromatography and recrystallized to afford 25.9 g of the product (yield: 72%).

### 2. Synthesis Example of Sub2-4

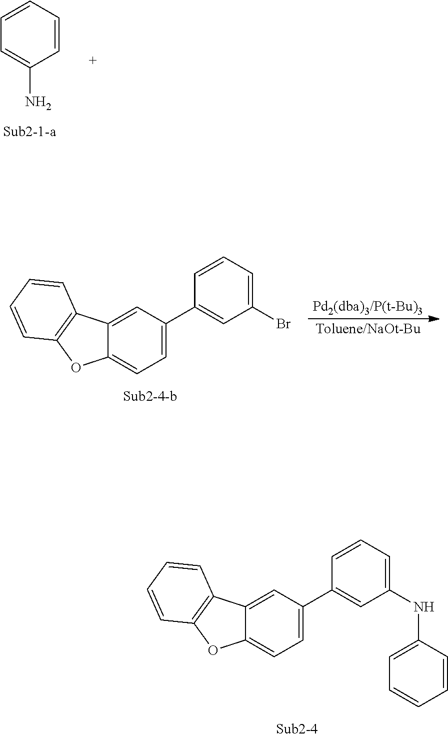 [SMILES: Nc1ccccc1 ; Brc1cccc(-c2ccc3oc4ccccc4c3c2)c1 ; "Toluene/NaOt-Bu" ; c1ccc(Nc2cccc(-c3ccc4oc5ccccc5c4c3)c2)cc1]

Sub2-1-a (10.0 g, 107 mmol) was dissolved in toluene (540 mL), followed by the addition of Sub2-4-b (34.7 g, 107 mmol), Pd₂(dba)₃ (2.95 g, 3.22 mmol), P(t-Bu)₃ (1.30 g, 6.44 mmol), and NaOt-Bu (20.6 g, 215 mmol). The reaction was carried out using the same method as described in the synthesis example of Sub2-1, affording the product (27.4 g, yield: 76%).

### 3. Synthesis Example of Sub2-16

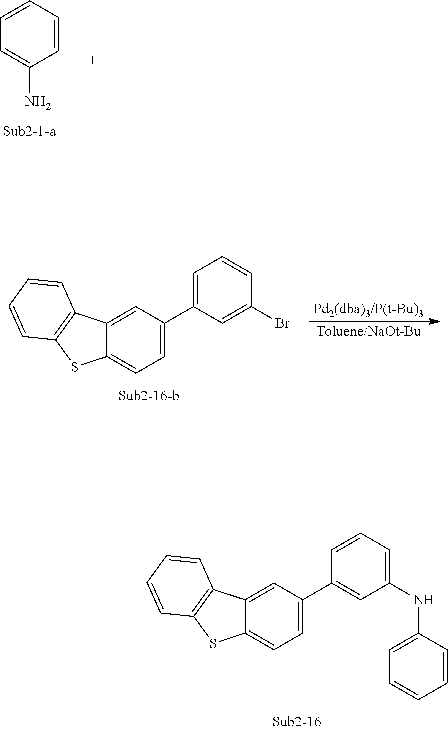 [SMILES: Nc1ccccc1 ; Brc1cccc(-c2ccc3sc4ccccc4c3c2)c1 ; "Toluene/NaOt-Bu" ; c1ccc(Nc2cccc(-c3ccc4sc5ccccc5c4c3)c2)cc1]

Sub2-1-a (5.0 g, 53.7 mmol) was dissolved in toluene (270 mL), followed by the addition of Sub2-16-b (18.2 g, 53.7 mmol), Pd₂(dba)₃ (1.47 g, 1.61 mmol), P(t-Bu)₃ (0.65 g, 3.22 mmol), and NaOt-Bu (10.3 g, 107 mmol). The reaction was carried out using the same method as described in the synthesis example of Sub2-1, affording the product 14.0 g (yield: 74%).

### 4. Synthesis Example of Sub2-19

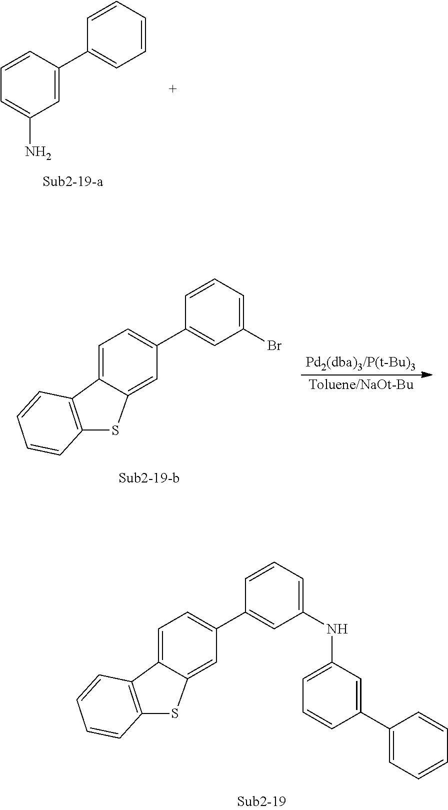 [SMILES: Nc1cccc(-c2ccccc2)c1 ; Brc1cccc(-c2ccc3c(c2)sc2ccccc23)c1 ; "Toluene/NaOt-Bu" ; c1ccc(-c2cccc(Nc3cccc(-c4ccc5c(c4)sc4ccccc45)c3)c2)cc1]

Sub2-19-a (5.0 g, 29.5 mmol) was dissolved in toluene (150 mL), followed by the addition of Sub2-19-b (10.0 g, 29.5 mmol), Pd₂(dba)₃ (0.81 g, 0.89 mmol), P(t-Bu)₃ (0.36 g, 1.77 mmol), and NaOt-Bu (5.7 g, 59.1 mmol). The reaction was carried out using the same method as described in the synthesis example of Sub2-1, affording the product 9.9 g (yield: 78%).

### 5. Synthesis Example of Sub2-24

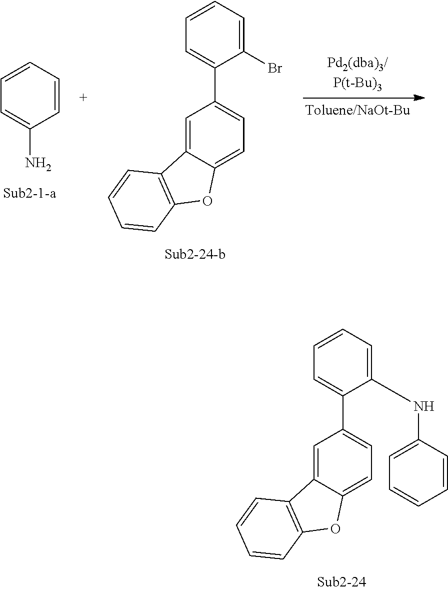 [SMILES: Nc1ccccc1 ; Brc1ccccc1-c1ccc2oc3ccccc3c2c1 ; c1ccc(Nc2ccccc2-c2ccc3oc4ccccc4c3c2)cc1]

Sub2-1-a (5.0 g, 53.7 mmol) was dissolved in toluene (270 mL), followed by the addition of Sub2-24-b (17.4 g, 53.7 mmol), Pd₂(dba)₃ (1.47 g, 1.61 mmol), P(t-Bu)₃ (0.65 g, 3.22 mmol), and NaOt-Bu (10.3 g, 107 mmol). The reaction was carried out using the same method as described in the synthesis example of Sub2-1, affording the product 12.2 g (yield: 68%).

### 6. Synthesis Example of Sub2-30

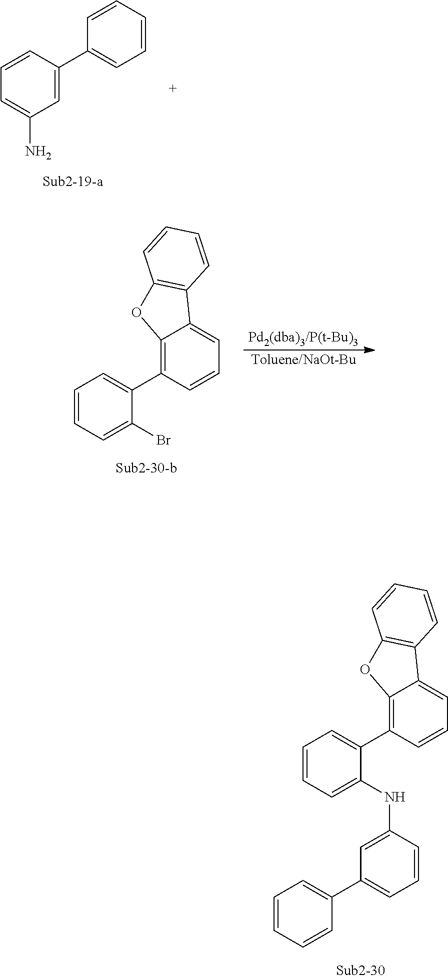 [SMILES: Nc1cccc(-c2ccccc2)c1 ; Brc1ccccc1-c1cccc2c1oc1ccccc12 ; "Toluene/NaOt-Bu" ; c1ccc(-c2cccc(Nc3ccccc3-c3cccc4c3oc3ccccc34)c2)cc1]

Sub2-19-a (5.0 g, 29.5 mmol) was dissolved in toluene (150 mL), followed by the addition of Sub2-30-b (9.5 g, 29.5 mmol), Pd₂(dba)₃ (0.81 g, 0.89 mmol), P(t-Bu)₃ (0.36 g, 1.77 mmol), and NaOt-Bu (5.7 g, 59.1 mmol). The reaction was carried out using the same method as described in the synthesis example of Sub2-1, affording the product 8.0 g (yield: 66%).

### 7. Synthesis Example of Sub2-39

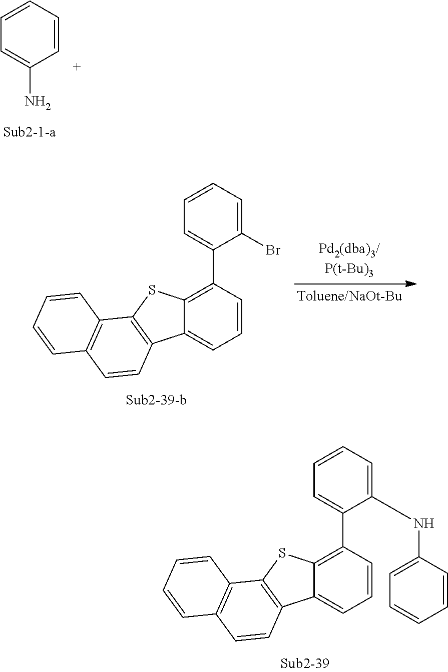 [SMILES: Nc1ccccc1 ; Brc1ccccc1-c1cccc2c1sc1c3ccccc3ccc21 ; c1ccc(Nc2ccccc2-c2cccc3c2sc2c4ccccc4ccc32)cc1]

Sub2-1-a (5.0 g, 53.7 mmol) was dissolved in toluene (270 mL), followed by the addition of Sub2-39-b (20.9 g, 53.7 mmol), Pd₂(dba)₃ (1.47 g, 1.61 mmol), P(t-Bu)₃ (0.65 g, 3.22 mmol), and NaOt-Bu (10.3 g, 107 mmol). The reaction was carried out using the same method as described in the synthesis example of Sub2-1, affording the product 14.9 g (yield: 69%).

### 8. Synthesis Example of Sub2-41

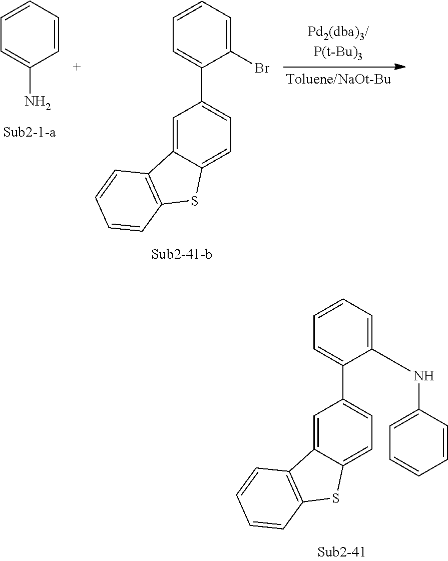 [SMILES: Nc1ccccc1 ; Brc1ccccc1-c1ccc2sc3ccccc3c2c1 ; c1ccc(Nc2ccccc2-c2ccc3sc4ccccc4c3c2)cc1]

Sub2-1-a (5.0 g, 53.7 mmol) was dissolved in toluene (150 mL), followed by the addition of Sub2-41-b (18.2 g, 53.7 mmol), Pd₂(dba)₃ (1.47 g, 1.61 mmol), P(t-Bu)₃ (0.65 g, 3.22 mmol), and NaOt-Bu (10.3 g, 107 mmol). The reaction was carried out using the same method as described in the synthesis example of Sub2-1, affording the product 12.6 g (yield: 67%).

Compounds belonging to Sub 2 may be, but not limited to, the following compounds, and Table 2 shows FD-MS values of the following compounds.

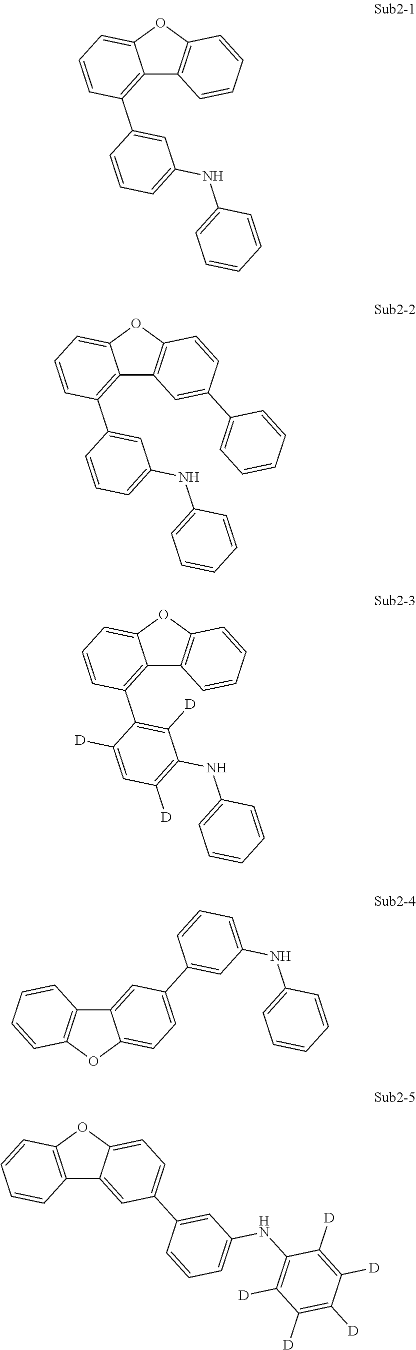 [SMILES: c1ccc(Nc2cccc(-c3cccc4oc5ccccc5c34)c2)cc1 ; c1ccc(Nc2cccc(-c3cccc4oc5ccc(-c6ccccc6)cc5c34)c2)cc1 ; [2H]c1cc([2H])c(-c2cccc3oc4ccccc4c23)c([2H])c1Nc1ccccc1 ; c1ccc(Nc2cccc(-c3ccc4oc5ccccc5c4c3)c2)cc1 ; [2H]c1c([2H])c([2H])c(Nc2cccc(-c3ccc4oc5ccccc5c4c3)c2)c([2H])c1[2H]]

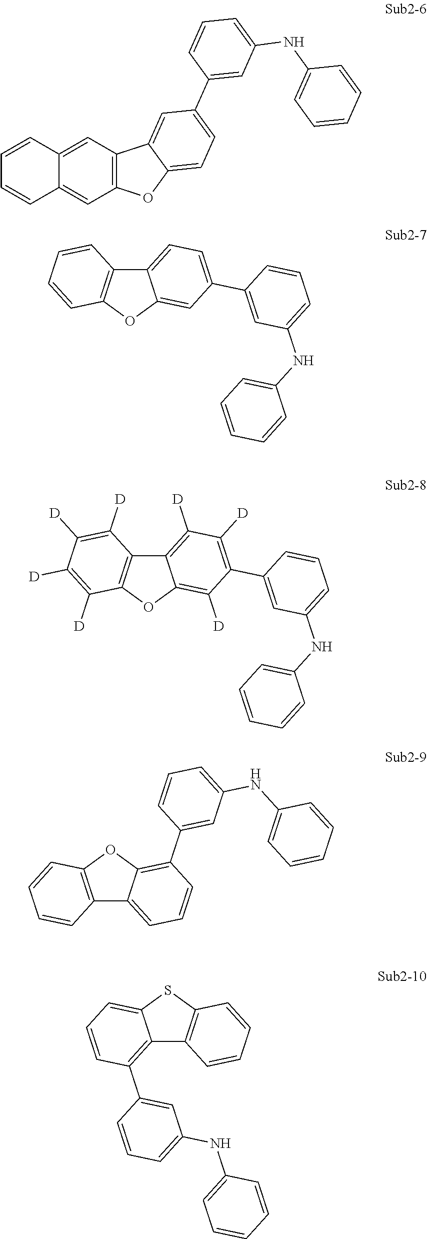

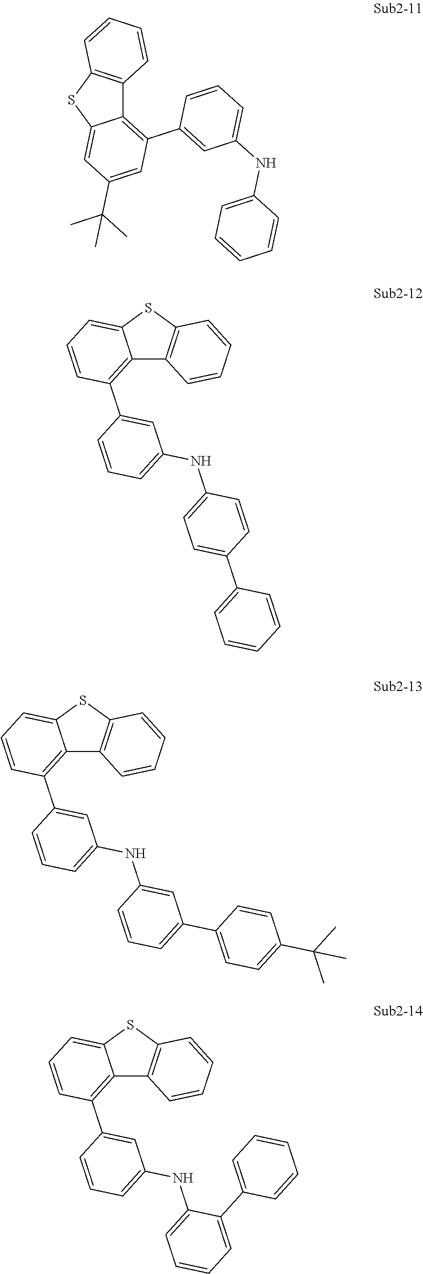 [SMILES: CC(C)(C)c1cc(-c2cccc(Nc3ccccc3)c2)c2c(c1)sc1ccccc12 ; c1ccc(-c2ccc(Nc3cccc(-c4cccc5sc6ccccc6c45)c3)cc2)cc1 ; CC(C)(C)c1ccc(-c2cccc(Nc3cccc(-c4cccc5sc6ccccc6c45)c3)c2)cc1 ; c1ccc(-c2ccccc2Nc2cccc(-c3cccc4sc5ccccc5c34)c2)cc1]

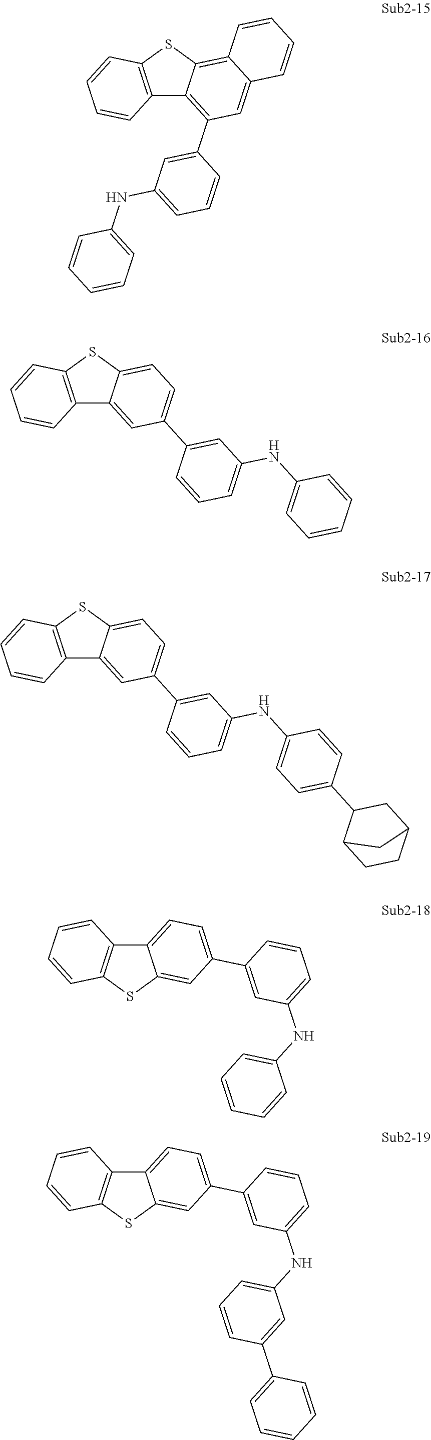 [SMILES: c1ccc(Nc2cccc(-c3cc4ccccc4c4sc5ccccc5c34)c2)cc1 ; c1ccc(Nc2cccc(-c3ccc4sc5ccccc5c4c3)c2)cc1 ; c1cc(Nc2ccc(C3CC4CCC3C4)cc2)cc(-c2ccc3sc4ccccc4c3c2)c1 ; c1ccc(Nc2cccc(-c3ccc4c(c3)sc3ccccc34)c2)cc1 ; c1ccc(-c2cccc(Nc3cccc(-c4ccc5c(c4)sc4ccccc45)c3)c2)cc1]

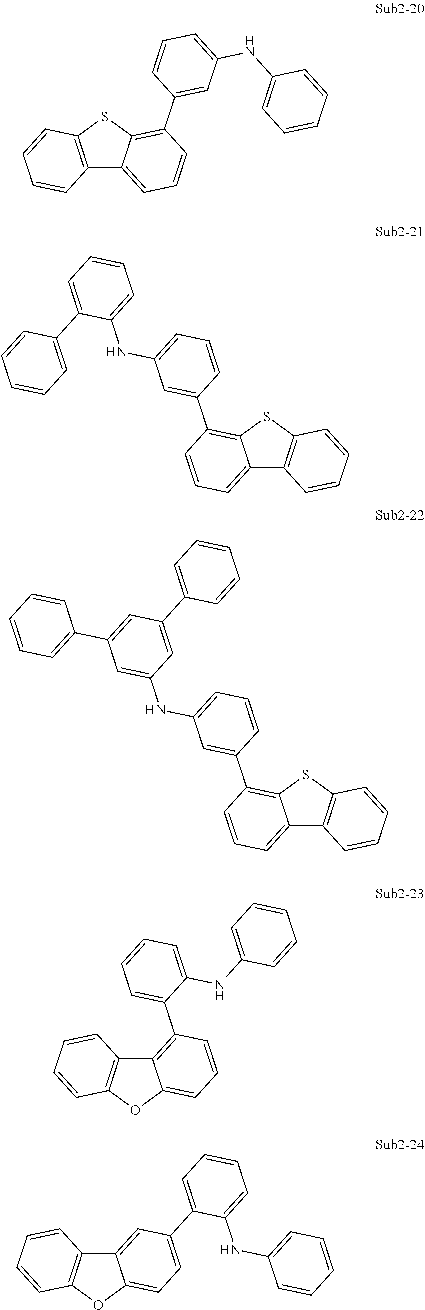 [SMILES: c1ccc(Nc2cccc(-c3cccc4c3sc3ccccc34)c2)cc1 ; c1ccc(-c2ccccc2Nc2cccc(-c3cccc4c3sc3ccccc34)c2)cc1 ; c1ccc(-c2cc(Nc3cccc(-c4cccc5c4sc4ccccc45)c3)cc(-c3ccccc3)c2)cc1 ; c1ccc(Nc2ccccc2-c2cccc3oc4ccccc4c23)cc1 ; c1ccc(Nc2ccccc2-c2ccc3oc4ccccc4c3c2)cc1]

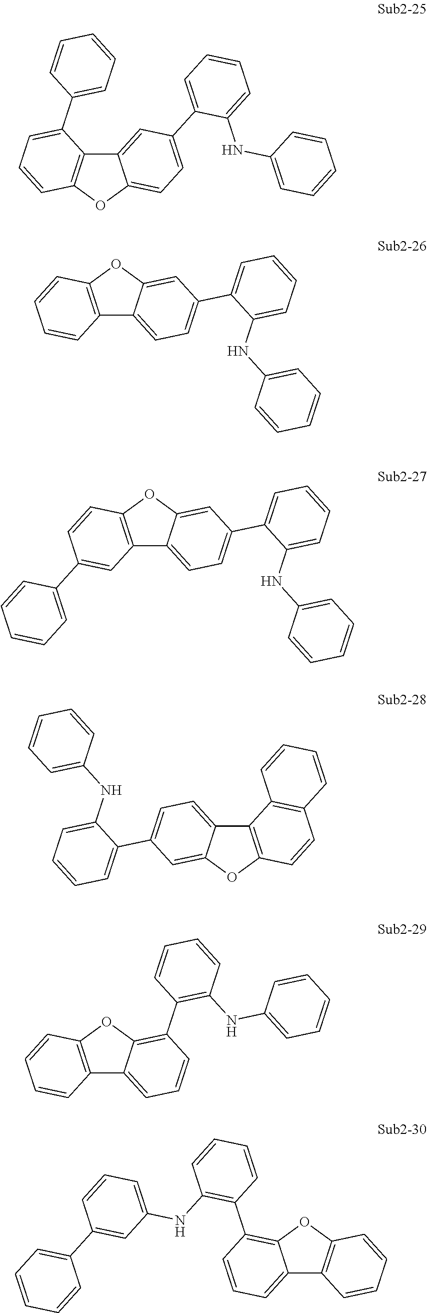 [SMILES: c1ccc(Nc2ccccc2-c2ccc3oc4cccc(-c5ccccc5)c4c3c2)cc1 ; c1ccc(Nc2ccccc2-c2ccc3c(c2)oc2ccccc23)cc1 ; c1ccc(Nc2ccccc2-c2ccc3c(c2)oc2ccc(-c4ccccc4)cc23)cc1 ; c1ccc(Nc2ccccc2-c2ccc3c(c2)oc2ccc4ccccc4c23)cc1 ; c1ccc(Nc2ccccc2-c2cccc3c2oc2ccccc23)cc1 ; c1ccc(-c2cccc(Nc3ccccc3-c3cccc4c3oc3ccccc34)c2)cc1]

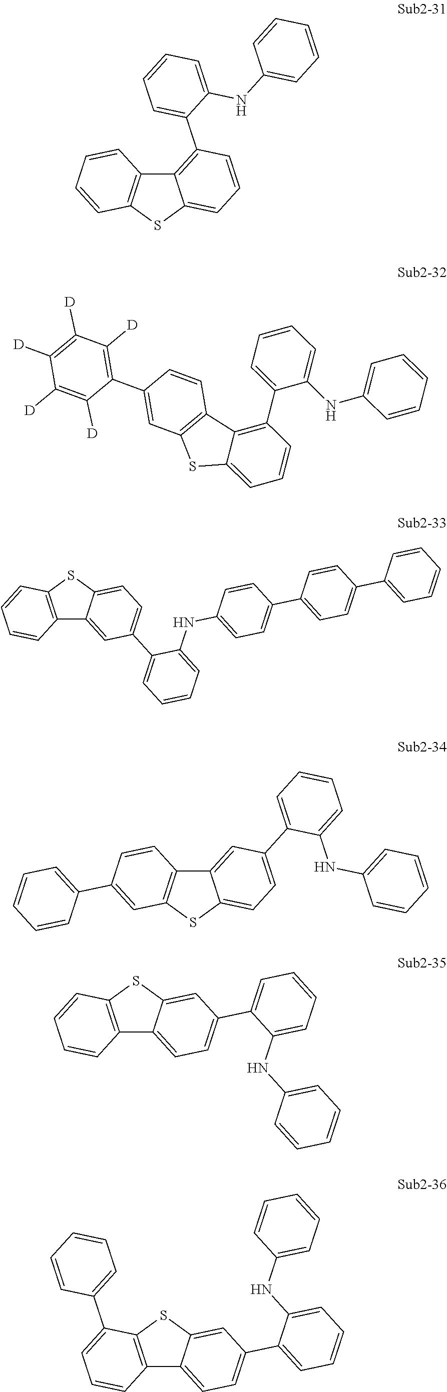

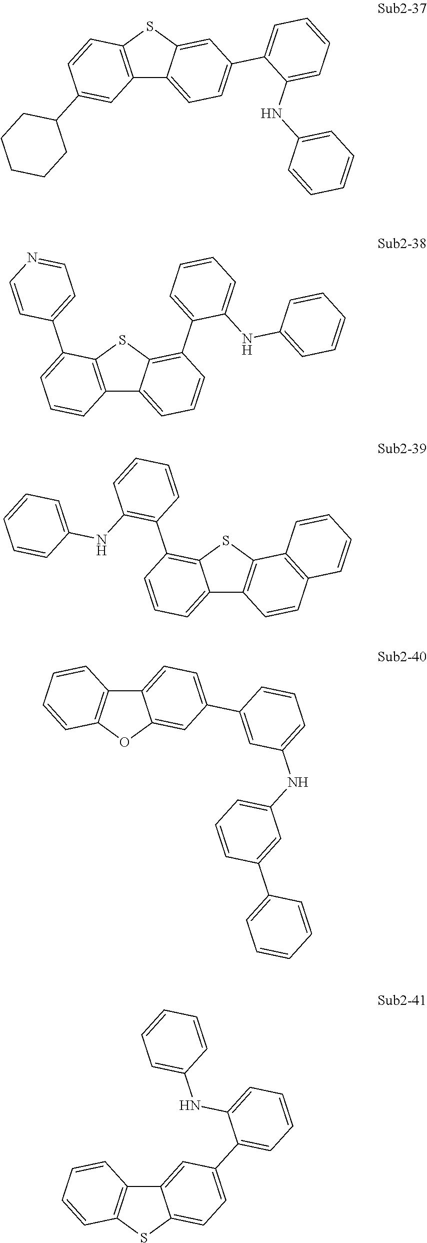 [SMILES: c1ccc(Nc2ccccc2-c2ccc3c(c2)sc2ccc(C4CCCCC4)cc23)cc1 ; c1ccc(Nc2ccccc2-c2cccc3c2sc2c(-c4ccncc4)cccc23)cc1 ; c1ccc(Nc2ccccc2-c2cccc3c2sc2c4ccccc4ccc32)cc1 ; c1ccc(-c2cccc(Nc3cccc(-c4ccc5c(c4)oc4ccccc45)c3)c2)cc1 ; c1ccc(Nc2ccccc2-c2ccc3sc4ccccc4c3c2)cc1]

TABLE 2

| Compound | FD-MS | Compound | FD-MS |
|---|---|---|---|
| Sub2-1 | m/z = 335.13(C₂₄H₁₇NO = 335.41) | Sub2-2 | m/z = 411.16(C₃₀H₂₁NO = 411.5) |
| Sub2-3 | m/z = 338.15(C₂₄H₁₄D₃NO = 338.42) | Sub2-4 | m/z = 335.13(C₂₄H₁₇NO = 335.41) |
| Sub2-5 | m/z = 340.16(C₂₄H₁₂D₅NO = 340.44) | Sub2-6 | m/z = 385.15(C₂₈H₁₉NO = 385.47) |
| Sub2-7 | m/z = 335.13(C₂₄H₁₇NO = 335.41) | Sub2-8 | m/z = 342.17(C₂₄H₁₀D₇NO = 342.45) |
| Sub2-9 | m/z = 335.13(C₂₄H₁₇NO = 335.41) | Sub2-10 | m/z = 351.11(C₂₄H₁₇NS = 351.47) |
| Sub2-11 | m/z = 407.17(C₂₈H₂₅NS = 407.58) | Sub2-12 | m/z = 427.14(C₃₀H₂₁NS = 427.57) |
| Sub2-13 | m/z = 483.2(C₃₄H₂₉NS = 483.67) | Sub2-14 | m/z = 427.14(C₃₀H₂₁NS = 427.57) |
| Sub2-15 | m/z = 401.12(C₂₈H₁₉NS = 401.53) | Sub2-16 | m/z = 351.11(C₂₄H₁₇NS = 351.47) |
| Sub2-17 | m/z = 445.19(C₃₁H₂₇NS = 445.62) | Sub2-18 | m/z = 351.11(C₂₄H₁₇NS = 351.47) |
| Sub2-19 | m/z = 427.14(C₃₀H₂₁NS = 427.57) | Sub2-20 | m/z = 351.11(C₂₄H₁₇NS = 351.47) |
| Sub2-21 | m/z = 427.14(C₃₀H₂₁NS = 427.57) | Sub2-22 | m/z = 503.17(C₃₆H₂₅NS = 503.66) |
| Sub2-23 | m/z = 335.13(C₂₄H₁₇NO = 335.41) | Sub2-24 | m/z = 335.13(C₂₄H₁₇NO = 335.41) |
| Sub2-25 | m/z = 411.16(C₃₀H₂₁NO = 411.5) | Sub2-26 | m/z = 335.13(C₂₄H₁₇NO = 335.41) |
| Sub2-27 | m/z = 411.16(C₃₀H₂₁NO = 411.5) | Sub2-28 | m/z = 385.15(C₂₈H₁₉NO = 385.47) |
| Sub2-29 | m/z = 335.13(C₂₄H₁₇NO = 335.41) | Sub2-30 | m/z = 411.16(C₃₀H₂₁NO = 411.5) |
| Sub2-31 | m/z = 351.11(C₂₄H₁₇NS = 351.47) | Sub2-32 | m/z = 432.17(C₃₀H₁₆D₅NS = 432.6) |
| Sub2-33 | m/z = 503.17(C₃₆H₂₅NS = 503.66) | Sub2-34 | m/z = 427.14(C₃₀H₂₁NS = 427.57) |
| Sub2-35 | m/z = 351.11(C₂₄H₁₇NS = 351.47) | Sub2-36 | m/z = 427.14(C₃₀H₂₁NS = 427.57) |
| Sub2-37 | m/z = 433.19(C₃₀H₂₇NS = 433.61) | Sub2-38 | m/z = 428.13(C₂₉H₂₀N₂S = 428.55) |
| Sub2-39 | m/z = 401.12(C₂₈H₁₉NS = 401.53) | Sub2-40 | m/z = 411.16(C₃₀H₂₁NO = 411.50) |
| Sub2-41 | m/z = 351.11(C₂₄H₁₇NS = 351.47) | | |

### III. Synthesis Example of Final Products

### Synthesis Example of P-1

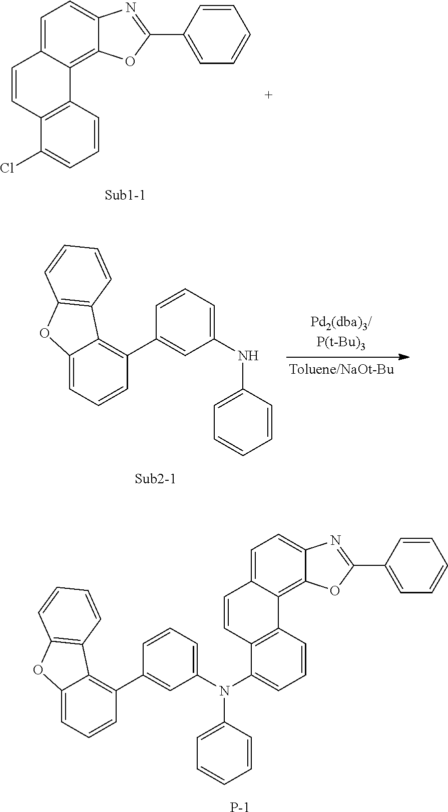

Sub1-1 (5.0 g, 15.2 mmol) was dissolved in toluene (76 mL), followed by the addition of Sub2-1 (5.1 g, 15.2 mmol) Pd₂(dba)₃ (0.42 g, 0.45 mmol), P(t-Bu)₃ (0.18 g, 0.91 mmol), and NaOt-Bu (2.9 g, 30.3 mmol). The reaction was carried out at 80° C. When the reaction was completed, the reaction products were extracted with CH₂Cl₂ and water, and the organic layer was dried over MgSO₄ and concentrated. Then, the concentrate was purified by silica gel column chromatography and recrystallized to afford 7.4 g of the product (yield: 78%).

### 2. Synthesis Example of P-19

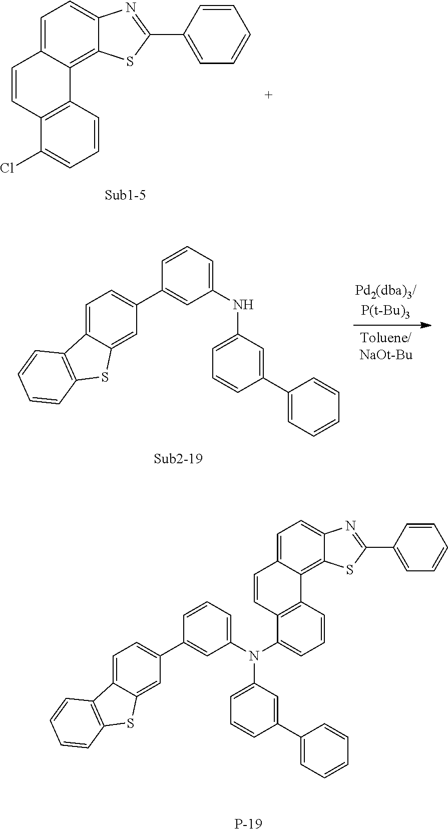

Sub1-5 (5.0 g, 14.5 mmol) was dissolved in toluene (72 mL), followed by the addition of Sub2-19 (6.2 g, 14.5 mmol), Pd₂(dba)₃ (0.40 g, 0.43 mmol), P(t-Bu)₃ (0.18 g, 0.87 mmol), and NaOt-Bu (2.8 g, 28.9 mmol). The reaction was carried out using the same method as described in the synthesis example of P-1, affording 8.0 g of the product (yield: 75%).

### 3. Synthesis Example of P-31

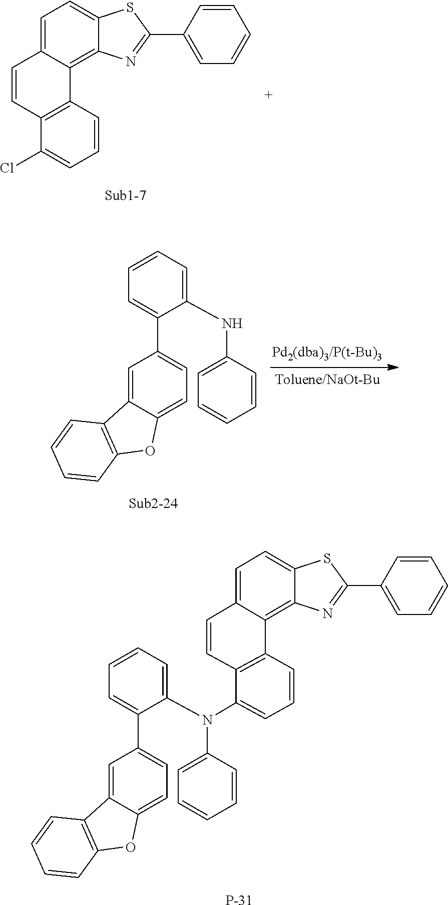

Sub1-7 (5.0 g, 14.5 mmol) was dissolved in toluene (72 mL), followed by the addition of Sub2-24 (4.8 g, 14.5 mmol), Pd₂(dba)₃ (0.40 g, 0.43 mmol), P(t-Bu)₃ (0.18 g, 0.87 mmol), and NaOt-Bu (2.8 g, 28.9 mmol). The reaction was carried out using the same method as described in the synthesis example of P-1, affording 6.7 g of the product (yield: 72%).

### 4. Synthesis Example of P-38

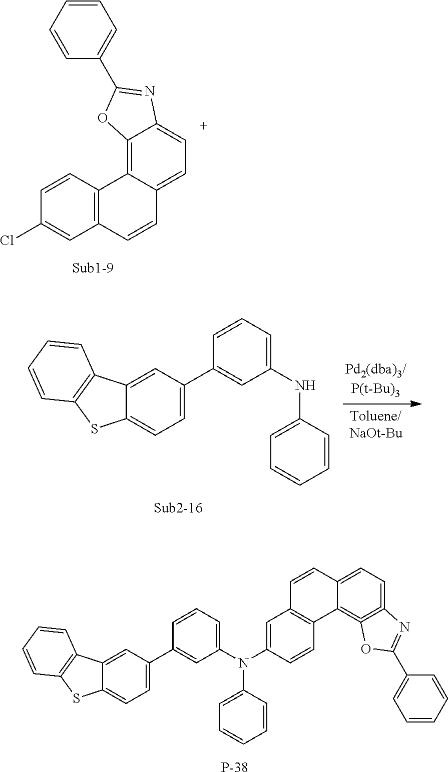

Sub1-9 (5.0 g, 14.5 mmol) was dissolved in toluene (72 mL), followed by the addition of Sub2-16 (4.8 g, 14.5 mmol), Pd₂(dba)₃ (0.40 g, 0.43 mmol), P(t-Bu)₃ (0.18 g, 0.87 mmol), and NaOt-Bu (2.8 g, 28.9 mmol). The reaction was carried out using the same method as described in the synthesis example of P-1, affording 7.2 g of the product (yield: 77%).

### 5. Synthesis Example of P-45

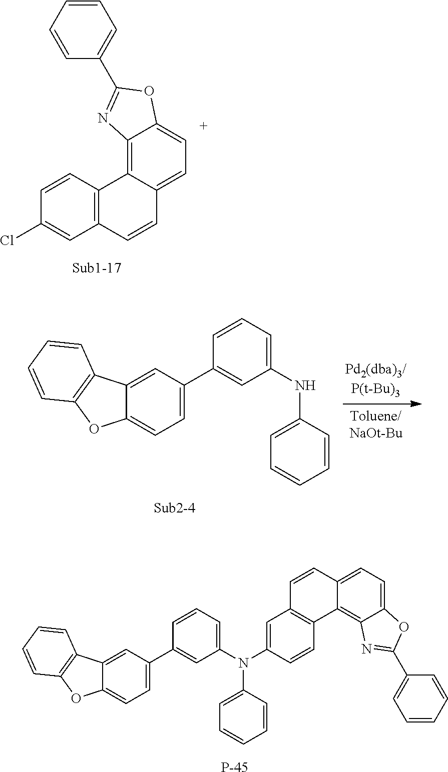

Sub1-17 (5.0 g, 15.2 mmol) was dissolved in toluene (76 mL), followed by the addition of Sub2-4 (5.1 g, 15.2 mmol), Pd₂(dba)₃ (0.42 g, 0.45 mmol), P(t-Bu)₃ (0.18 g, 0.91 mmol), and NaOt-Bu (2.9 g, 30.3 mmol). The reaction was carried out using the same method as described in the synthesis example of P-1, affording 7.5 g of the product (yield: 79%).

### 6. Synthesis Example of P-66

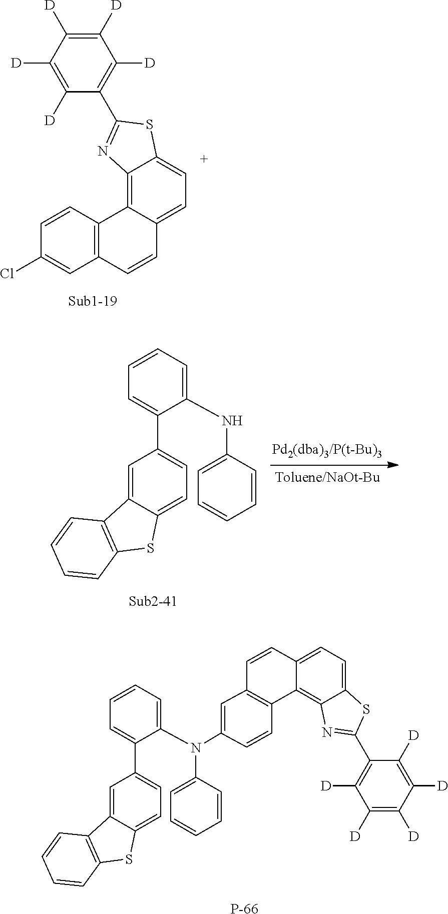

Sub1-19 (5.0 g, 14.3 mmol) was dissolved in toluene (71 mL), followed by the addition of Sub2-41 (5.0 g, 14.3 mmol), Pd₂(dba)₃ (0.39 g, 0.43 mmol), P(t-Bu)₃ (0.17 g, 0.86 mmol), and NaOt-Bu (2.7 g, 28.5 mmol). The reaction was carried out using the same method as described in the synthesis example of P-1, affording 6.9 g of the product (yield: 73%).

### 7. Synthesis Example of P-70

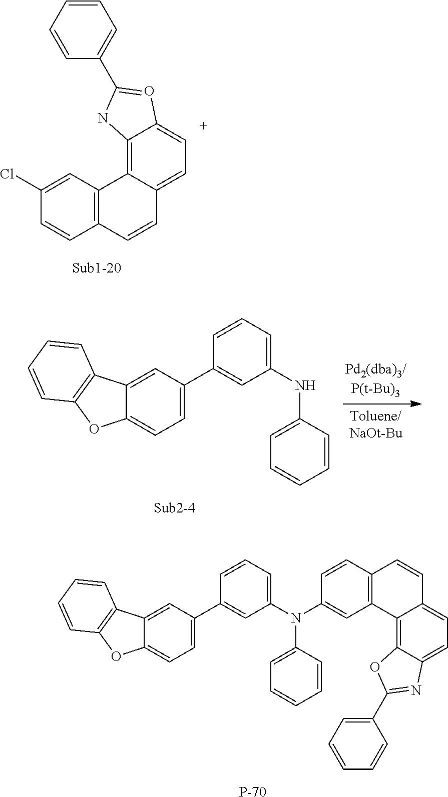

Sub1-20 (5.0 g, 15.2 mmol) was dissolved in toluene (76 mL), followed by the addition of Sub2-4 (5.1 g, 15.2 mmol), Pd₂(dba)₃ (0.42 g, 0.45 mmol), P(t-Bu)₃ (0.18 g, 0.91 mmol), and NaOt-Bu (2.9 g, 30.3 mmol). The reaction was carried out using the same method as described in the synthesis example of P-1, affording 7.2 g of the product (yield: 76%).

### 8. Synthesis Example of P-106

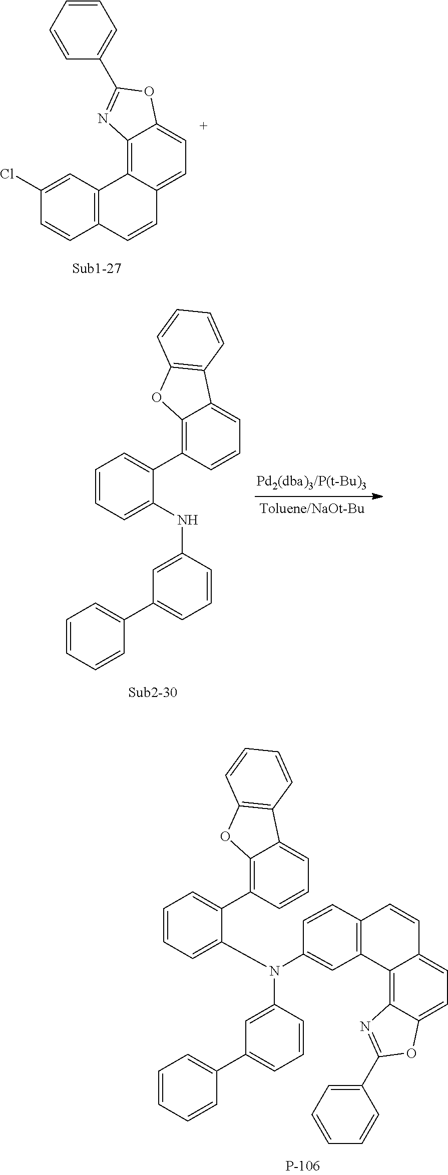 [SMILES: Clc1ccc2ccc3ccc4oc(-c5ccccc5)nc4c3c2c1 ; c1ccc(-c2cccc(Nc3ccccc3-c3cccc4c3oc3ccccc34)c2)cc1 ; c1ccc(-c2cccc(N(c3ccc4ccc5ccc6oc(-c7ccccc7)nc6c5c4c3)c3ccccc3-c3cccc4c3oc3ccccc34)c2)cc1]

Sub1-27 (5.0 g, 15.2 mmol) was dissolved in toluene (76 mL), followed by the addition of Sub2-30 (6.2 g, 15.2 mmol), Pd₂(dba)₃ (0.42 g, 0.45 mmol), P(t-Bu)₃ (0.18 g, 0.91 mmol), and NaOt-Bu (2.9 g, 30.3 mmol). The reaction was carried out using the same method as described in the synthesis example of P-1, affording 7.5 g of the product (yield: 70%).

### 9. Synthesis Example of P-110

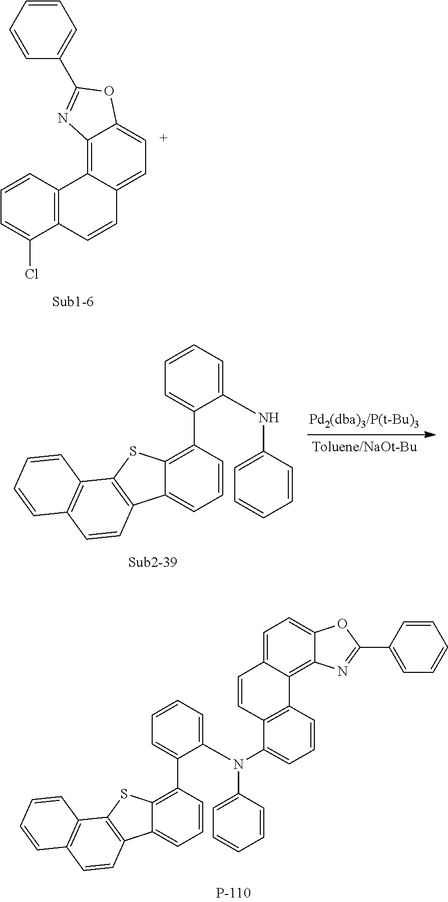 [SMILES: Clc1cccc2c1ccc1ccc3oc(-c4ccccc4)nc3c12 ; c1ccc(Nc2ccccc2-c2cccc3c2sc2c4ccccc4ccc32)cc1 ; c1ccc(-c2nc3c(ccc4ccc5c(N(c6ccccc6)c6ccccc6-c6cccc7c6sc6c8ccccc8ccc76)cccc5c43)o2)cc1]

Sub1-6 (5.0 g, 15.2 mmol) was dissolved in toluene (76 mL), followed by the addition of Sub2-39 (6.1 g, 15.2 mmol), Pd₂(dba)₃ (0.42 g, 0.45 mmol), P(t-Bu)₃ (0.18 g, 0.91 mmol), and NaOt-Bu (2.9 g, 30.3 mmol). The reaction was carried out using the same method as described in the synthesis example of P-1, affording 7.2 g of the product (yield: 68%).

### 10. Synthesis Example of P-122

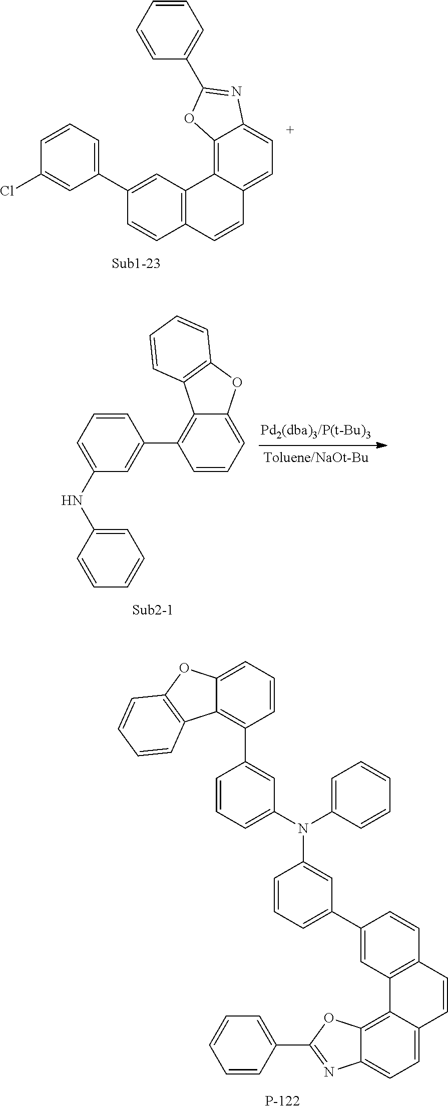 [SMILES: Clc1cccc(-c2ccc3ccc4ccc5nc(-c6ccccc6)oc5c4c3c2)c1 ; c1ccc(Nc2cccc(-c3cccc4oc5ccccc5c34)c2)cc1 ; c1ccc(-c2nc3ccc4ccc5ccc(-c6cccc(N(c7ccccc7)c7cccc(-c8cccc9oc%10ccccc%10c89)c7)c6)cc5c4c3o2)cc1]

Sub1-23 (5.0 g, 12.3 mmol) was dissolved in toluene (62 mL), followed by the addition of Sub2-1 (4.1 g, 12.3 mmol), Pd₂(dba)₃ (0.34 g, 0.37 mmol), P(t-Bu)₃ (0.15 g, 0.74 mmol), and NaOt-Bu (2.4 g, 24.6 mmol). The reaction was carried out using the same method as described in the synthesis example of P-1, affording 6.5 g of the product (yield: 75%).

The FD-MS values of the compounds P-1 to P-124 of the present invention manufactured according to the above synthetic examples are as shown in Table 3 below.

TABLE 3

| Compound | FD-MS | Compound | FD-MS |
|---|---|---|---|
| P-1 | m/z = 628.22(C₄₅H₂₈N₂O₂ = 628.73) | P-2 | m/z = 628.22(C₄₅H₂₈N₂O₂ = 628.73) |
| P-3 | m/z = 628.22(C₄₅H₂₈N₂O₂ = 628.73) | P-4 | m/z = 628.22(C₄₅H₂₈N₂O₂ = 628.73) |
| P-5 | m/z = 704.25(C₅₁H₃₂N₂O₂ = 704.83) | P-6 | m/z = 649.22(C₄₅H₂₃D₅N₂OS = 649.82) |
| P-7 | m/z = 633.25(C₄₅H₂₃D₅N₂O₂ = 633.76) | P-8 | m/z = 644.19(C₄₅H₂₈N₂OS = 644.79) |
| P-9 | m/z = 660.17(C₄₅H₂₈N₂S₂ = 660.85) | P-10 | m/z = 644.19(C₄₅H₂₈N₂OS = 644.79) |
| P-11 | m/z = 644.19(C₄₅H₂₈N₂OS = 644.79) | P-12 | m/z = 660.17(C₄₅H₂₈N₂S₂ = 660.85) |
| P-13 | m/z = 628.22(C₄₅H₂₈N₂O₂ = 628.73) | P-14 | m/z = 644.19(C₄₅H₂₈N₂OS = 644.79) |
| P-15 | m/z = 644.19(C₄₅H₂₈N₂OS = 644.79) | P-16 | m/z = 660.17(C₄₅H₂₈N₂S₂ = 660.85) |
| P-17 | m/z = 716.23(C₄₉H₃₆N₂S₂ = 716.96) | P-18 | m/z = 738.27(C₅₂H₃₈N₂OS = 738.95) |
| P-19 | m/z = 736.2(C₅₁H₃₂N₂S₂ = 736.95) | P-20 | m/z = 720.22(C₅₁H₃₂N₂OS = 720.89) |
| P-21 | m/z = 628.22(C₄₅H₂₈N₂O₂ = 628.73) | P-22 | m/z = 704.25(C₅₁H₃₂N₂O₂ = 704.83) |
| P-23 | m/z = 633.25(C₄₅H₂₃D₅N₂O₂ = 633.76) | P-24 | m/z = 644.19(C₄₅H₂₈N₂OS = 644.79) |
| P-25 | m/z = 644.19(C₄₅H₂₈N₂OS = 644.79) | P-26 | m/z = 736.2(C₅₁H₃₂N₂S₂ = 736.95) |
| P-27 | m/z = 720.22(C₅₁H₃₂N₂OS = 720.89) | P-28 | m/z = 737.2(C₅₀H₃₁N₃S₂ = 737.94) |
| P-29 | m/z = 644.19(C₄₅H₂₈N₂OS = 644.79) | P-30 | m/z = 628.22(C₄₅H₂₈N₂O₂ = 628.73) |
| P-31 | m/z = 644.19(C₄₅H₂₈N₂OS = 644.79) | P-32 | m/z = 660.17(C₄₅H₂₈N₂S₂ = 660.85) |
| P-33 | m/z = 628.22(C₄₅H₂₈N₂O₂ = 628.73) | P-34 | m/z = 628.22(C₄₅H₂₈N₂O₂ = 628.73) |
| P-35 | m/z = 628.22(C₄₅H₂₈N₂O₂ = 628.73) | P-36 | m/z = 628.22(C₄₅H₂₈N₂O₂ = 628.73) |
| P-37 | m/z = 644.19(C₄₅H₂₈N₂OS = 644.79) | P-38 | m/z = 644.19(C₄₅H₂₈N₂OS = 644.79) |
| P-39 | m/z = 644.19(C₄₅H₂₈N₂OS = 644.79) | P-40 | m/z = 644.19(C₄₅H₂₈N₂OS = 644.79) |
| P-41 | m/z = 660.17(C₄₅H₂₈N₂S₂ = 660.85) | P-42 | m/z = 644.19(C₄₅H₂₈N₂OS = 644.79) |
| P-43 | m/z = 644.19(C₄₅H₂₈N₂OS = 644.79) | P-44 | m/z = 660.17(C₄₅H₂₈N₂S₂ = 660.85) |
| P-45 | m/z = 628.22(C₄₅H₂₈N₂O₂ = 628.73) | P-46 | m/z = 644.19(C₄₅H₂₈N₂OS = 644.79) |
| P-47 | m/z = 644.19(C₄₅H₂₈N₂OS = 644.79) | P-48 | m/z = 660.17(C₄₅H₂₈N₂S₂ = 660.85) |
| P-49 | m/z = 704.25(C₅₁H₃₂N₂O₂ = 704.83) | P-50 | m/z = 720.22(C₅₁H₃₂N₂OS = 720.89) |
| P-51 | m/z = 635.26(C₄₅H₂₁D₇N₂O₂ = 635.77) | P-52 | m/z = 720.22(C₅₁H₃₂N₂OS = 720.89) |
| P-53 | m/z = 628.22(C₄₅H₂₈N₂O₂ = 628.73) | P-54 | m/z = 628.22(C₄₅H₂₈N₂O₂ = 628.73) |
| P-55 | m/z = 628.22(C₄₅H₂₈N₂O₂ = 628.73) | P-56 | m/z = 628.22(C₄₅H₂₈N₂O₂ = 628.73) |
| P-57 | m/z = 644.19(C₄₅H₂₈N₂OS = 644.79) | P-58 | m/z = 644.19(C₄₅H₂₈N₂OS = 644.79) |
| P-59 | m/z = 644.19(C₄₅H₂₈N₂OS = 644.79) | P-60 | m/z = 644.19(C₄₅H₂₈N₂OS = 644.79) |
| P-61 | m/z = 628.22(C₄₅H₂₈N₂O₂ = 628.73) | P-62 | m/z = 644.19(C₄₅H₂₈N₂OS = 644.79) |
| P-63 | m/z = 644.19(C₄₅H₂₈N₂OS = 644.79) | P-64 | m/z = 660.17(C₄₅H₂₈N₂S₂ = 660.85) |
| P-65 | m/z = 710.29(C₅₁H₃₈N₂O₂ = 710.88) | P-66 | m/z = 665.2(C₄₅H₂₃D₅N₂S₂ = 665.88) |
| P-67 | m/z = 742.25(C₅₁H₃₈N₂S₂ = 743) | P-68 | m/z = 704.25(C₅₁H₃₂N₂O₂ = 704.83) |
| P-69 | m/z = 628.22(C₄₅H₂₈N₂O₂ = 628.73) | P-70 | m/z = 628.22(C₄₅H₂₈N₂O₂ = 628.73) |
| P-71 | m/z = 704.25(C₅₁H₃₂N₂O₂ = 704.83) | P-72 | m/z = 628.22(C₄₅H₂₈N₂O₂ = 628.73) |
| P-73 | m/z = 644.19(C₄₅H₂₈N₂OS = 644.79) | P-74 | m/z = 644.19(C₄₅H₂₈N₂OS = 644.79) |
| P-75 | m/z = 644.19(C₄₅H₂₈N₂OS = 644.79) | P-76 | m/z = 644.19(C₄₅H₂₈N₂OS = 644.79) |
| P-77 | m/z = 644.19(C₄₅H₂₈N₂OS = 644.79) | P-78 | m/z = 644.19(C₄₅H₂₈N₂OS = 644.79) |
| P-79 | m/z = 644.19(C₄₅H₂₈N₂OS = 644.79) | P-80 | m/z = 660.17(C₄₅H₂₈N₂S₂ = 660.85) |
| P-81 | m/z = 628.22(C₄₅H₂₈N₂O₂ = 628.73) | P-82 | m/z = 644.19(C₄₅H₂₈N₂OS = 644.79) |
| P-83 | m/z = 644.19(C₄₅H₂₈N₂OS = 644.79) | P-84 | m/z = 660.17(C₄₅H₂₈N₂S₂ = 660.85) |
| P-85 | m/z = 633.25(C₄₅H₂₃D₅N₂O₂ = 633.76) | P-86 | m/z = 776.29(C₅₅H₄₀N₂OS = 777) |
| P-87 | m/z = 647.21(C₄₅H₂₅D₃N₂OS = 647.81) | P-88 | m/z = 812.23(C₅₇H₃₆N₂S₂ = 813.05) |
| P-89 | m/z = 628.22(C₄₅H₂₈N₂O₂ = 628.73) | P-90 | m/z = 628.22(C₄₅H₂₈N₂O₂ = 628.73) |
| P-91 | m/z = 628.22(C₄₅H₂₈N₂O₂ = 628.73) | P-92 | m/z = 628.22(C₄₅H₂₈N₂O₂ = 628.73) |
| P-93 | m/z = 644.19(C₄₅H₂₈N₂OS = 644.79) | P-94 | m/z = 644.19(C₄₅H₂₈N₂OS = 644.79) |
| P-95 | m/z = 644.19(C₄₅H₂₈N₂OS = 644.79) | P-96 | m/z = 644.19(C₄₅H₂₈N₂OS = 644.79) |
| P-97 | m/z = 644.19(C₄₅H₂₈N₂OS = 644.79) | P-98 | m/z = 660.17(C₄₅H₂₈N₂S₂ = 660.85) |
| P-99 | m/z = 660.17(C₄₅H₂₈N₂S₂ = 660.85) | P-100 | m/z = 644.19(C₄₅H₂₈N₂OS = 644.79) |
| P-101 | m/z = 628.22(C₄₅H₂₈N₂O₂ = 628.73) | P-102 | m/z = 644.19(C₄₅H₂₈N₂OS = 644.79) |
| P-103 | m/z = 644.19(C₄₅H₂₈N₂OS = 644.79) | P-104 | m/z = 660.17(C₄₅H₂₈N₂S₂ = 660.85) |
| P-105 | m/z = 796.25(C₅₇H₃₆N₂OS = 796.99) | P-106 | m/z = 704.25(C₅₁H₃₂N₂O₂ = 704.83) |
| P-107 | m/z = 741.23(C₅₁H₂₇D₅N₂S₂ = 741.98) | P-108 | m/z = 720.22(C₅₁H₃₂N₂OS = 720.89) |
| P-109 | m/z = 694.21(C₄₉H₃₀N₂OS = 694.85) | P-110 | m/z = 694.21(C₄₉H₃₀N₂OS = 694.85) |
| P-111 | m/z = 678.23(C₄₉H₃₀N₂O₂ = 678.79) | P-112 | m/z = 694.21(C₄₉H₃₀N₂OS = 694.85) |
| P-113 | m/z = 704.25(C₅₁H₃₂N₂O₂ = 704.83) | P-114 | m/z = 770.24(C₅₅H₃₄N₂OS = 770.95) |
| P-115 | m/z = 704.25(C₅₁H₃₂N₂O₂ = 704.83) | P-116 | m/z = 720.22(C₅₁H₃₂N₂OS = 720.89) |
| P-117 | m/z = 704.25(C₅₁H₃₂N₂O₂ = 704.83) | P-118 | m/z = 704.25(C₅₁H₃₂N₂O₂ = 704.83) |
| P-119 | m/z = 754.26(C₅₅H₃₄N₂O₂ = 754.89) | P-120 | m/z = 704.25(C₅₁H₃₂N₂O₂ = 704.83) |
| P-121 | m/z = 754.26(C₅₅H₃₄N₂O₂ = 754.89) | P-122 | m/z = 704.25(C₅₁H₃₂N₂O₂ = 704.83) |
| P-123 | m/z = 704.25(C₅₁H₃₂N₂O₂ = 704.83) | P-124 | m/z = 704.25(C₅₁H₃₂N₂O₂ = 704.83) |

### [Synthesis Example of 2] Formula 2

### 1. Synthesis Example of N-19

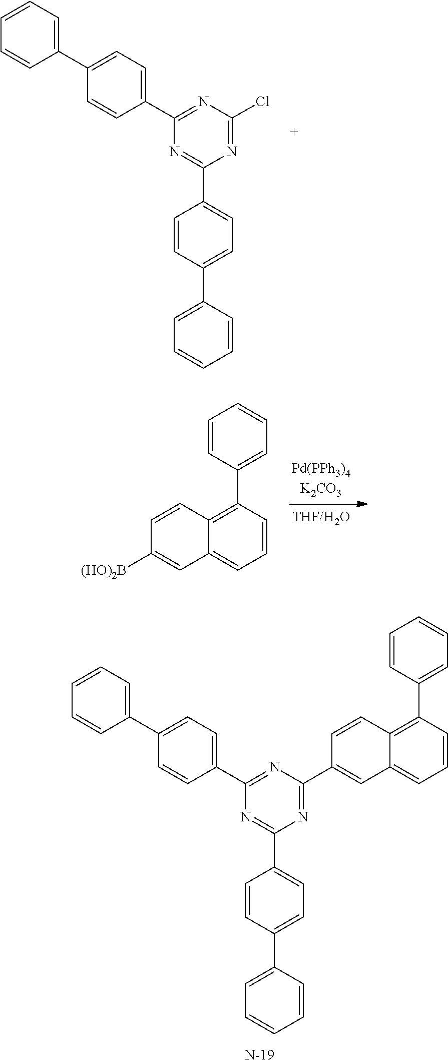

2,4-di([1,1′-biphenyl]-4-yl)-6-chloro-1,3,5-triazine (5.0 g, 11.9 mmol), (5-phenylnaphthalen-2-yl) boronic acid (3.0 g, 11.9 mmol) was dissolved in THF (60 ml), followed by the addition of Pd(PPh₃)₄ (0.83 g, 0.71 mmol), K₂CO₃ (4.9 g, 35.7 mmol) and water (30 ml), and the mixture was stirred under reflux. When the reaction was completed, the reaction products were extracted with ether and water. The organic layer was concentrated, dried over MgSO₄, and concentrated again. The final residue was purified by silica gel column chromatography and recrystallized to afford 5.5 g of the product (yield: 78%).

### 2. Synthesis Example of N-33

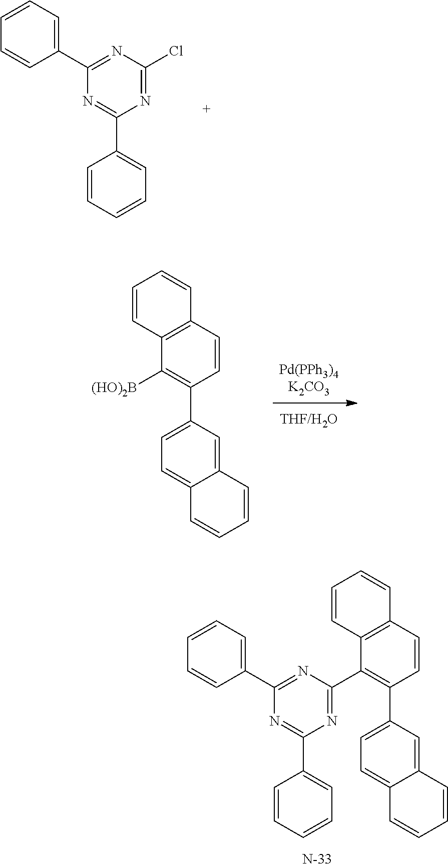

2-chloro-4,6-diphenyl-1,3,5-triazine (5.0 g, 18.7 mmol) was dissolved in THF (60 ml), followed by the addition of [2,2′-binaphthalen]-1-ylboronic acid (5.6 g, 18.7 mmol), Pd(PPh₃)₄ (1.29 g, 1.12 mmol) and K₂CO₃ (7.7 g, 56.0 mmol), and the mixture was stirred under reflux. When the reaction was completed, the reaction products were extracted with ether and water. The organic layer was concentrated, dried over MgSO₄, and concentrated again. The final residue was purified by silica gel column chromatography and recrystallized to afford 6.4 g of the product (yield: 71%).

The FD-MS values of the compounds 6-1 to 6-124, and the compounds N-1 to N-148 of the present invention manufactured according to the above synthetic examples are as shown in Table 4 below.

TABLE 4

| Compound | FD-MS | Compound | FD-MS |
|---|---|---|---|
| 6-1 | m/z = 399.14(C₂₇H₁₇N₃O = 399.45) | 6-2 | m/z = 415.11(C₂₇H₁₇N₃S = 415.51) |
| 6-3 | m/z = 474.18(C₃₃H₂₂N₄ = 474.57) | 6-4 | m/z = 449.15(C₃₁H₁₉N₃O = 449.51) |
| 6-5 | m/z = 449.15(C₃₁H₁₉N₃O = 449.51) | 6-6 | m/z = 515.15(C₃₅H₂₁N₃S = 515.63) |
| 6-7 | m/z = 600.23(C₄₃H₂₈N₄ = 600.73) | 6-8 | m/z = 499.17(C₃₅H₂₁N₃O = 499.57) |
| 6-9 | m/z = 551.20(C₃₉H₂₅N₃O = 551.65) | 6-10 | m/z = 567.18(C₃₉H₂₅N₃S = 567.71) |
| 6-11 | m/z = 702.28(C₅₁H₃₄N₄ = 702.86) | 6-12 | m/z = 657.22(C₄₆H₃₁N₃S = 657.84) |
| 6-13 | m/z = 551.20(C₃₉H₂₅N₃O = 551.65) | 6-14 | m/z = 541.16(C₃₇H₂₃N₃S = 541.67) |
| 6-15 | m/z = 700.26(C₅₁H₃₂N₄ = 700.85) | 6-16 | m/z = 703.21(C₅₀H₂₉N₃S = 703.86) |
| 6-17 | m/z = 525.18(C₃₇H₂₃N₃O = 525.61) | 6-18 | m/z = 591.18(C₄₁H₂₅N₃S = 591.73) |
| 6-19 | m/z = 627.24(C₄₄H₂₉N₅ = 627.75) | 6-20 | m/z = 524.20(C₃₇H₂₄N₄ = 524.63) |
| 6-21 | m/z = 551.20(C₃₉H₂₅N₃O = 551.65) | 6-22 | m/z = 567.18(C₃₉H₂₅N₃S = 567.71) |
| 6-23 | m/z = 702.28(C₅₁H₃₄N₄ = 702.86) | 6-24 | m/z = 474.18(C₃₃H₂₂N₄ = 474.57) |
| 6-25 | m/z = 779.29(C₅₇H₃₇N₃O = 779.94) | 6-26 | m/z = 731.24(C₅₂H₃₃N₃S = 731.92) |
| 6-27 | m/z = 601.23(C₄₂H₂₇N₅ = 601.71) | 6-28 | m/z = 475.17(C₃₃H₂₁N₃O = 475.55) |
| 6-29 | m/z = 641.21(C₄₅H₂₇N₃O₂ = 641.73) | 6-30 | m/z = 746.21(C₅₁H₃₀N₄OS = 746.89) |
| 6-31 | m/z = 716.26(C₅₁H₃₂N₄O = 716.84) | 6-32 | m/z = 681.19(C₄₇H₂₇N₃OS = 681.81) |
| 6-33 | m/z = 475.17(C₃₃H₂₁N₃O = 475.55) | 6-34 | m/z = 491.15(C₃₃H₂₁N₃S = 491.61) |
| 6-35 | m/z = 550.22(C₃₉H₂₆N₄ = 550.67) | 6-36 | m/z = 525.18(C₃₇H₂₃N₃O = 525.61) |
| 6-37 | m/z = 475.17(C₃₃H₂₁N₃O = 475.55) | 6-38 | m/z = 491.15(C₃₃H₂₁N₃S = 491.61) |
| 6-39 | m/z = 704.27(C₄₉H₃₂N₆ = 704.84) | 6-40 | m/z = 541.16(C₃₇H₂₃N₃S = 541.67) |
| 6-41 | m/z = 551.20(C₃₉H₂₅N₃O = 551.65) | 6-42 | m/z = 541.16(C₃₇H₂₃N₃S = 541.67) |
| 6-43 | m/z = 626.25(C₄₅H₃₀N₄ = 626.76) | 6-44 | m/z = 676.26(C₄₉H₃₂N₄ = 676.82) |
| 6-45 | m/z = 551.2(C₃₉H₂₅N₃O = 551.65) | 6-46 | m/z = 567.18(C₃₉H₂₅N₃S = 567.71) |
| 6-47 | m/z = 614.25(C₄₄H₃₀N₄ = 614.75) | 6-48 | m/z = 575.17(C₃₉H₂₁N₅O = 575.63) |
| 6-49 | m/z = 525.18(C₃₇H₂₃N₃O = 525.61) | 6-50 | m/z = 541.16(C₃₇H₂₃N₃S = 541.67) |
| 6-51 | m/z = 600.23(C₄₃H₂₈N₄ = 600.73) | 6-52 | m/z = 625.22(C₄₅H₂₇N₃O = 625.73) |
| 6-53 | m/z = 525.18(C₃₇H₂₃N₃O = 525.61) | 6-54 | m/z = 591.18(C₄₁H₂₅N₃S = 591.73) |
| 6-55 | m/z = 600.23(C₄₃H₂₈N₄ = 600.73) | 6-56 | m/z = 693.22(C₄₉H₃₁N₃S = 693.87) |
| 6-57 | m/z = 505.12(C₃₃H₁₉N₃OS = 505.60) | 6-58 | m/z = 641.21(C₄₅H₂₇N₃O₂ = 641.73) |
| 6-59 | m/z = 571.12(C₃₇H₂₁N₃S₂ = 571.72) | 6-60 | m/z = 564.20(C₃₉H₂₄N₄O = 564.65) |
| 6-61 | m/z = 581.16(C₃₉H₂₃N₃OS = 581.69) | 6-62 | m/z = 521.10(C₃₃H₁₉N₃S₂ = 521.66) |
| 6-63 | m/z = 489.15(C₃₃H₁₉N₃O₂ = 489.53) | 6-64 | m/z = 640.23(C₄₅H₂₈N₄O = 640.75) |
| 6-65 | m/z = 489.15(C₃₃H₁₉N₃O₂ = 489.53) | 6-66 | m/z = 505.12(C₃₃H₁₉N₃OS = 505.60) |
| 6-67 | m/z = 580.17(C₃₉H₂₄N₄S = 580.71) | 6-68 | m/z = 564.20(C₃₉H₂₄N₄O = 564.65) |
| 6-69 | m/z = 489.15(C₃₃H₁₉N₃O₂ = 489.53) | 6-70 | m/z = 505.12(C₃₃H₁₉N₃OS = 505.60) |
| 6-71 | m/z = 505.12(C₃₃H₁₉N₃OS = 505.60) | 6-72 | m/z = 639.24(C₄₅H₂₉N₅ = 639.76) |
| 6-73 | m/z = 607.21(C₄₂H₂₉N₃S = 607.78) | 6-74 | m/z = 715.26(C₅₂H₃N₃O = 715.86) |
| 6-75 | m/z = 640.23(C₄₅H₂₈N₄O = 640.75) | 6-76 | m/z = 707.20(C₄₉H₂₉N₃OS = 707.85) |
| 6-77 | m/z = 591.23(C₄₂H₂₉N₃O = 591.71) | 6-78 | m/z = 617.28(C₄₅H₃₅N₃ = 617.80) |
| 6-79 | m/z = 653.25(C₄₇H₃₁N₃O = 653.79) | 6-80 | m/z = 733.22(C₅₁H₃₁N₃OS = 733.89) |
| 6-81 | m/z = 615.19(C₄₃H₂₅N₃O₂ = 615.69) | 6-82 | m/z = 681.19(C₄₇H₂₇N₃OS = 681.81) |
| 6-83 | m/z = 716.29(C₅₂H₃₆N₄ = 716.89) | 6-84 | m/z = 690.24(C₄₉H₃₀N₄O = 690.81) |
| 6-85 | m/z = 641.25(C₄₆H₃₁N₃O = 641.77) | 6-86 | m/z = 693.22(C₄₉H₃₁N₃S = 693.87) |
| 6-87 | m/z = 690.24(C₄₉H₃₀N₄O = 690.81) | 6-88 | m/z = 631.17(C₄₃H₂₅N₃OS = 631.75) |
| 6-89 | m/z = 595.14(C₃₉H₂₁N₃O₂S = 595.68) | 6-90 | m/z = 659.24(C₄₅H₂₁D₅N₄O₂ = 659.76) |
| 6-91 | m/z = 637.16(C₄₂H₂₇N₃S₂ = 637.82) | 6-92 | m/z = 729.25(C₅₁H₃₁N₅O = 729.84) |
| 6-93 | m/z = 578.17(C₃₉H₂₂N₄O₂ = 578.63) | 6-94 | m/z = 746.21(C₅₁H₃₀N₄OS = 746.89) |
| 6-95 | m/z = 681.24(C₄₈H₃₁N₃O₂ = 681.80) | 6-96 | m/z = 762.19(C₅₁H₃₀N₄S₂ = 762.95) |
| 6-97 | m/z = 436.17(C₃₀H₂₀N₄ = 436.52) | 6-98 | m/z = 437.16(C₂₉H₁₉N₅ = 437.51) |
| 6-99 | m/z = 513.20(C₃₅H₂₃N₅ = 513.60) | 6-100 | m/z = 589.23(C₄₁H₂₇N₅ = 589.70) |
| 6-101 | m/z = 486.18(C₃₄H₂₂N₄ = 486.58) | 6-102 | m/z = 527.17(C₃₅H₂₁N₅O = 527.59) |
| 6-103 | m/z = 589.23(C₄₁H₂₇N₅ = 589.70) | 6-104 | m/z = 502.18(C₃₄H₂₂N₄O = 502.58) |
| 6-105 | m/z = 511.20(C₃₇H₂₅N₃ = 511.63) | 6-106 | m/z = 563.21(C₃₉H₂₅N₅ = 563.66) |
| 6-107 | m/z = 511.20(C₃₇H₂₅N₃ = 511.63) | 6-108 | m/z = 589.23(C₄₁H₂₇N₅ = 589.70) |
| 6-109 | m/z = 513.20(C₃₅H₂₃N₅ = 513.60) | 6-110 | m/z = 462.16(C₃₀H₁₈N₆ = 462.52) |
| 6-111 | m/z = 612.21(C₄₂H₂₄N₆ = 612.70) | 6-112 | m/z = 499.20(C₃₆H₂₅N₃ = 499.62) |
| 6-113 | m/z = 569.17(C₃₇H₂₃N₅S = 569.69) | 6-114 | m/z = 629.22(C₄₃H₂₇N₅O = 629.72) |
| 6-115 | m/z = 629.22(C₄₃H₂₇N₅O = 629.72) | 6-116 | m/z = 563.21(C₃₉H₂₅N₅ = 563.66) |
| 6-117 | m/z = 565.2(C₃₇H₂₃N₇ = 565.64) | 6-118 | m/z = 630.22(C₄₂H₂₆N₆O = 630.71) |
| 6-119 | m/z = 611.24(C₄₅H₂₉N₃ = 611.75) | 6-120 | m/z = 803.29(C₅₉H₃₇N₃O = 803.97) |
| 6-121 | m/z = 563.20(C₄₀H₂₅N₃O = 563.66) | 6-122 | m/z = 549.22(C₄₀H₂₇N₃ = 549.68) |
| 6-123 | m/z = 449.15(C₃₁H₁₉N₃O = 449.51) | 6-124 | m/z = 579.18(C₄₀H₂₅N₃S = 579.72) |
| N-1 | m/z = 435.17(C₃₁H₂₁N₃ = 435.53) | N-2 | m/z = 435.17(C₃₁H₂₁N₃ = 435.53) |
| N-3 | m/z = 435.17(C₃₁H₂₁N₃ = 435.53) | N-4 | m/z = 435.17(C₃₁H₂₁N₃ = 435.53) |
| N-5 | m/z = 435.17(C₃₁H₂₁N₃ = 435.53) | N-6 | m/z = 435.17(C₃₁H₂₁N₃ = 435.53) |
| N-7 | m/z = 435.17(C₃₁H₂₁N₃ = 435.53) | N-8 | m/z = 434.18(C₃₂H₂₂N₂ = 434.54) |
| N-9 | m/z = 511.20(C₃₇H₂₅N₃ = 511.63) | N-10 | m/z = 611.24(C₄₅H₂₉N₃ = 611.75) |
| N-11 | m/z = 485.19(C₃₅H₂₃N₃ = 485.59) | N-12 | m/z = 511.2(C₃₇H₂₅N₃ = 511.63) |
| N-13 | m/z = 511.20(C₃₇H₂₅N₃ = 511.63) | N-14 | m/z = 485.19(C₃₅H₂₃N₃ = 485.59) |
| N-15 | m/z = 434.18(C₃₂H₂₂N₂ = 434.54) | N-16 | m/z = 434.18(C₃₂H₂₂N₂ = 434.54) |
| N-17 | m/z = 511.20(C₃₇H₂₅N₃ = 511.63) | N-18 | m/z = 561.22(C₄₁H₂₇N₃ = 561.69) |
| N-19 | m/z = 587.24(C₄₃H₂₉N₃ = 587.73) | N-20 | m/z = 511.20(C₃₇H₂₅N₃ = 511.63) |
| N-21 | m/z = 511.20(C₃₇H₂₅N₃ = 511.63) | N-22 | m/z = 511.20(C₃₇H₂₅N₃ = 511.63) |
| N-23 | m/z = 511.20(C₃₇H₂₅N₃ = 511.63) | N-24 | m/z = 587.24(C₄₃H₂₉N₃ = 587.73) |
| N-25 | m/z = 435.17(C₃₁H₂₁N₃ = 435.53) | N-26 | m/z = 435.17(C₃₁H₂₁N₃ = 435.53) |
| N-27 | m/z = 435.17(C₃₁H₂₁N₃ = 435.53) | N-28 | m/z = 435.17(C₃₁H₂₁N₃ = 435.53) |
| N-29 | m/z = 435.17(C₃₁H₂₁N₃ = 435.53) | N-30 | m/z = 435.17(C₃₁H₂₁N₃ = 435.53) |
| N-31 | m/z = 435.17(C₃₁H₂₁N₃ = 435.53) | N-32 | m/z = 434.18(C₃₂H₂₂N₂ = 434.54) |
| N-33 | m/z = 485.19(C₃₅H₂₃N₃ = 485.59) | N-34 | m/z = 511.2(C₃₇H₂₅N₃ = 511.63) |
| N-35 | m/z = 511.20(C₃₇H₂₅N₃ = 511.63) | N-36 | m/z = 511.20(C₃₇H₂₅N₃ = 511.63) |
| N-37 | m/z = 485.19(C₃₅H₂₃N₃ = 485.59) | N-38 | m/z = 511.20(C₃₇H₂₅N₃ = 511.63) |
| N-39 | m/z = 485.19(C₃₅H₂₃N₃ = 485.59) | N-40 | m/z = 611.24(C₄₅H₂₉N₃ = 611.75) |
| N-41 | m/z = 511.20(C₃₇H₂₅N₃ = 511.63) | N-42 | m/z = 511.20(C₃₇H₂₅N₃ = 511.63) |
| N-43 | m/z = 587.24(C₄₃H₂₉N₃ = 587.73) | N-44 | m/z = 587.24(C₄₃H₂₉N₃ = 587.73) |
| N-45 | m/z = 587.24(C₄₃H₂₉N₃ = 587.73) | N-46 | m/z = 561.22(C₄₁H₂₇N₃ = 561.69) |
| N-47 | m/z = 511.20(C₃₇H₂₅N₃ = 511.63) | N-48 | m/z = 587.24(C₄₃H₂₉N₃ = 587.73) |
| N-49 | m/z = 485.19(C₃₅H₂₃N₃ = 485.59) | N-50 | m/z = 485.19(C₃₅H₂₃N₃ = 485.59) |
| N-51 | m/z = 485.19(C₃₅H₂₃N₃ = 485.59) | N-52 | m/z = 485.19(C₃₅H₂₃N₃ = 485.59) |
| N-53 | m/z = 485.19(C₃₅H₂₃N₃ = 485.59) | N-54 | m/z = 535.20(C₃₉H₂₅N₃ = 535.65) |
| N-55 | m/z = 485.19(C₃₅H₂₃N₃ = 485.59) | N-56 | m/z = 485.19(C₃₅H₂₃N₃ = 485.59) |
| N-57 | m/z = 561.22(C₄₁H₂₇N₃ = 561.69) | N-58 | m/z = 561.22(C₄₁H₂₇N₃ = 561.69) |
| N-59 | m/z = 561.22(C₄₁H₂₇N₃ = 561.69) | N-60 | m/z = 637.25(C₄₇H₃₁N₃ = 637.79) |
| N-61 | m/z = 561.22(C₄₁H₂₇N₃ = 561.69) | N-62 | m/z = 561.22(C₄₁H₂₇N₃ = 561.69) |
| N-63 | m/z = 637.25(C₄₇H₃₁N₃ = 637.79) | N-64 | m/z = 637.25(C₄₇H₃₁N₃ = 637.79) |
| N-65 | m/z = 637.25(C₄₇H₃₁N₃ = 637.79) | N-66 | m/z = 485.19(C₃₅H₂₃N₃ = 485.59) |
| N-67 | m/z = 485.19(C₃₅H₂₃N₃ = 485.59) | N-68 | m/z = 611.24(C₄₅H₂₉N₃ = 611.75) |
| N-69 | m/z = 485.19(C₃₅H₂₃N₃ = 485.59) | N-70 | m/z = 485.19(C₃₅H₂₃N₃ = 485.59) |
| N-71 | m/z = 611.24(C₄₅H₂₉N₃ = 611.75) | N-72 | m/z = 485.19(C₃₅H₂₃N₃ = 485.59) |
| N-73 | m/z = 485.19(C₃₅H₂₃N₃ = 485.59) | N-74 | m/z = 561.22(C₄₁H₂₇N₃ = 561.69) |
| N-75 | m/z = 485.19(C₃₅H₂₃N₃ = 485.59) | N-76 | m/z = 485.19(C₃₅H₂₃N₃ = 485.59) |
| N-77 | m/z = 611.24(C₄₅H₂₉N₃ = 611.75) | N-78 | m/z = 611.24(C₄₅H₂₉N₃ = 611.75) |
| N-79 | m/z = 485.19(C₃₅H₂₃N₃ = 485.59) | N-80 | m/z = 485.19(C₃₅H₂₃N₃ = 485.59) |
| N-81 | m/z = 485.19(C₃₅H₂₃N₃ = 485.59) | N-82 | m/z = 485.19(C₃₅H₂₃N₃ = 485.59) |
| N-83 | m/z = 535.20(C₃₉H₂₅N₃ = 535.65) | N-84 | m/z = 535.2(C₃₉H₂₅N₃ = 535.65) |
| N-85 | m/z = 585.22(C₄₃H₂₇N₃ = 585.71) | N-86 | m/z = 535.2(C₃₉H₂₅N₃ = 535.65) |
| N-87 | m/z = 585.22(C₄₃H₂₇N₃ = 585.71) | N-88 | m/z = 585.22(C₄₃H₂₇N₃ = 585.71) |
| N-89 | m/z = 611.24(C₄₅H₂₉N₃ = 611.75) | N-90 | m/z = 611.24(C₄₅H₂₉N₃ = 611.75) |
| N-91 | m/z = 585.22(C₄₃H₂₇N₃ = 585.71) | N-92 | m/z = 611.24(C₄₅H₂₉N₃ = 611.75) |
| N-93 | m/z = 687.27(C₅₁H₃₃N₃ = 687.85) | N-94 | m/z = 611.24(C₄₅H₂₉N₃ = 611.75) |
| N-95 | m/z = 511.20(C₃₇H₂₅N₃ = 511.63) | N-96 | m/z = 611.24(C₄₅H₂₉N₃ = 611.75) |
| N-97 | m/z = 561.22(C₄₁H₂₇N₃ = 561.69) | N-98 | m/z = 587.24(C₄₃H₂₉N₃ = 587.73) |
| N-99 | m/z = 663.27(C₄₉H₃₃N₃ = 663.82) | N-100 | m/z = 713.28(C₅₃H₃₅N₃ = 713.88) |
| N-101 | m/z = 575.20(C₄₁H₂₅N₃O = 575.67) | N-102 | m/z = 601.22(C₄₃H₂₇N₃O = 601.71) |
| N-103 | m/z = 700.26(C₅₁H₃₂N₄ = 700.85) | N-104 | m/z = 701.25(C₅₁H₃₁N₃O = 701.83) |
| N-105 | m/z = 667.21(C₄₇H₂₉N₃S = 667.83) | N-106 | m/z = 541.16(C₃₇H₂₃N₃S = 541.67) |
| N-107 | m/z = 612.23(C₄₄H₂₈N₄ = 612.74) | N-108 | m/z = 562.22(C₄₀H₂₆N₄ = 562.68) |
| N-109 | m/z = 689.26(C₄₉H₃₁N₅ = 689.82) | N-110 | m/z = 639.24(C₄₅H₂₉N₅ = 639.76) |
| N-111 | m/z = 701.25(C₅₁H₃₁N₃O = 701.83) | N-112 | m/z = 631.17(C₄₃H₂₅N₃OS = 631.75) |
| N-113 | m/z = 625.22(C₄₅H₂₇N₃O = 625.73) | N-114 | m/z = 591.18(C₄₁H₂₅N₃S = 591.73) |
| N-115 | m/z = 687.27(C₅₁H₃₃N₃ = 687.85) | N-116 | m/z = 701.25(C₅₁H₃₁N₃O = 701.83) |
| N-117 | m/z = 619.30(C₄₅H₃₇N₃ = 619.81) | N-118 | m/z = 601.25(C₄₄H₃₁N₃ = 601.75) |
| N-119 | m/z = 667.23(C₄₇H₂₉N₃O₂ = 667.77) | N-120 | m/z = 540.24(C₃₉H₂₀D₅N₃ = 540.68) |
| N-121 | m/z = 521.17(C₃₅H₂₁F₂N₃ = 521.57) | N-122 | m/z = 510.18(C₃₆H₂₂N₄ = 510.60) |
| N-123 | m/z = 652.23(C₄₆H₂₈N₄O = 652.76) | N-124 | m/z = 527.24(C₃₈H₂₉N₃ = 527.67) |
| N-125 | m/z = 535.20(C₃₉H₂₅N₃ = 535.65) | N-126 | m/z = 535.20(C₃₉H₂₅N₃ = 535.65) |
| N-127 | m/z = 535.20(C₃₉H₂₅N₃ = 535.65) | N-128 | m/z = 535.20(C₃₉H₂₅N₃ = 535.65) |
| N-129 | m/z = 587.24(C₄₃H₂₉N₃ = 587.73) | N-130 | m/z = 612.23(C₄₄H₂₈N₄ = 612.74) |
| N-131 | m/z = 561.22(C₄₁H₂₇N₃ = 561.69) | N-132 | m/z = 687.27(C₅₁H₃₃N₃ = 687.85) |
| N-133 | m/z = 663.27(C₄₉H₃₃N₃ = 663.82) | N-134 | m/z = 601.22(C₄₃H₂₇N₃O = 601.71) |
| N-135 | m/z = 617.19(C₄₃H₂₇N₃S = 617.77) | N-136 | m/z = 752.29(C₅₅H₃₆N₄ = 752.92) |
| N-137 | m/z = 651.23(C₄₇H₂₉N₃O = 651.77) | N-138 | m/z = 677.25(C₄₉H₃₁N₃O = 677.81) |
| N-139 | m/z = 541.16(C₃₇H₂₃N₃S = 541.67) | N-140 | m/z = 750.28(C₅₅H₃₄N₄ = 750.91) |
| N-141 | m/z = 707.24(C₅₀H₃₃N₃S = 707.90) | N-142 | m/z = 651.23(C₄₇H₂₉N₃O = 651.77) |
| N-143 | m/z = 617.19(C₄₃H₂₇N₃S = 617.77) | N-144 | m/z = 667.21(C₄₇H₂₉N₃S = 667.83) |
| N-145 | m/z = 631.17(C₄₃H₂₅N₃OS = 631.75) | N-146 | m/z = 767.26(C₅₅H₃₃N₃O₂ = 767.89) |
| N-147 | m/z = 647.15(C₄₃H₂₅N₃S₂ = 647.81) | N-148 | m/z = 690.24(C₄₉H₃₀N₄O = 690.81) |

### Manufacturing and Evaluation of Organic Electronic Device

### [Test Example 1] Red Organic Electroluminescent Device (Phosphorescent Host)

A hole-injecting layer having a thickness of 60 nm was formed by vacuum-depositing N1-(naphthalen-2-yl)-N4,N4-bis(4-(naphthalen-2-yl(phenyl)amino)phenyl)-N1-phenylbenzene-1,4-diamine (hereinafter referred to as “2-TNATA”) on an ITO layer (anode). Subsequently, a hole-transporting layer was formed by vacuum-depositing 4,4-bis[N-(1-naphthyl)-N-phenylamino]biphenyl (hereinafter referred to as “NPD”) to a thickness of 50 nm on the hole-injecting layer.

Next, a light-emitting auxiliary layer was formed by vacuum-depositing tris(4-(9H-carbazol-9-yl)phenyl)amine (hereinafter referred to as “TCTA”) to a thickness of 10 nm on the hole-transporting layer.

Thereafter, a light-emitting layer having a thickness of 30 nm was formed on the light-emitting auxiliary layer using a host-dopant system, wherein the host was a mixture of compound P-1 represented by Formula 1 and compound N-19 represented by Formula 2 of the present invention in a weight ratio of 5:5, and the dopant was bis(1-phenylisoquinolyl)iridium (III) acetylacetonate (hereinafter referred to as “(piq)₂Ir(acac)”). The dopant was introduced such that the weight ratio of the host to the dopant was 95:5.

Subsequently, a hole blocking layer having a thickness of 10 nm was formed by vacuum-depositing (1,1′-biphenyl-4-olato)bis(2-methyl-8-quinolinolato)aluminum (hereinafter abbreviated as “BAlq”) on the light-emitting layer. An electron transporting layer was then formed by vacuum-depositing bis(10-hydroxybenzo[h]quinolinato) beryllium (hereinafter abbreviated as “BeBq₂”) to a thickness of 45 nm on the hole blocking layer. Thereafter, an electron-injecting layer having a thickness of 0.2 nm was formed by depositing LiF on the electron transporting layer, followed by deposition of Al to form a cathode having a thickness of 150 nm.

### [Test Example 2] to [Test Example 26]

Except that a mixture of the compound represented by Formula 1 of the present invention as listed in Table 6 and compound N-19 was used as the host material of the light-emitting layer instead of the mixture of compound P-1 and compound N-19, the organic electroluminescent device was fabricated in the same manner as in Example 1.

### [Comparative Example 1] and [Comparative Example 2]

Except that a mixture of Comparative Compound A or Comparative Compound B and Compound N-1 was used as the host material of the light-emitting layer instead of the mixture of Compound P-1 and Compound N-19 of the present invention, the organic electroluminescent device was fabricated in the same manner as in Example 1.

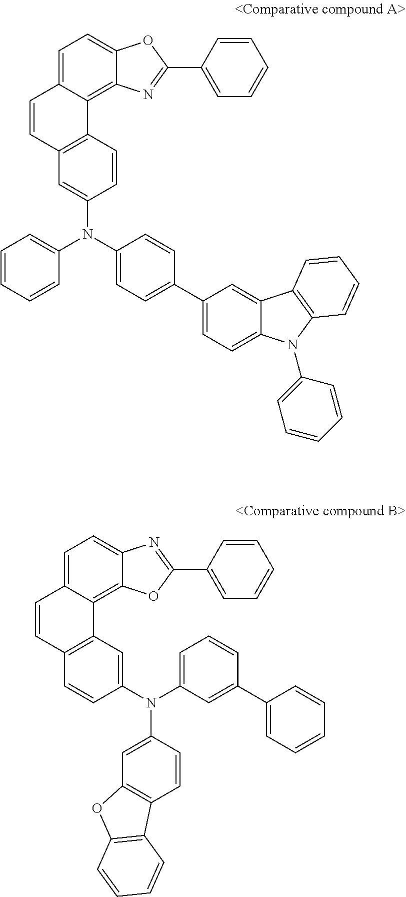 [SMILES: c1ccc(-c2nc3c(ccc4ccc5cc(N(c6ccccc6)c6ccc(-c7ccc8c(c7)c7ccccc7n8-c7ccccc7)cc6)ccc5c43)o2)cc1 ; c1ccc(-c2cccc(N(c3ccc4c(c3)oc3ccccc34)c3ccc4ccc5ccc6nc(-c7ccccc7)oc6c5c4c3)c2)cc1]

The electroluminescent (EL) characteristics of the organic electroluminescent devices fabricated according to the Examples and Comparative Examples of the present invention were measured by applying a forward DC voltage using a PR-650 photometer from Photo Research. The T95 lifetime was measured at a standard luminance of 2500 cd/m² using a lifetime measurement system manufactured by Mc Science. The measurement results are shown in Table 5 below.

TABLE 5

| | | | Current | | | | |
|---|---|---|---|---|---|---|---|
| | | Voltage | Density | Efficiency | Lifetime | CIE | |
| | Compound | (V) | (mA/cm²) | (cd/A) | T(95) | x | y |
| comp. Ex1 | Comp. | 5.5 | 8.5 | 29.3 | 113.2 | 0.644 | 0.346 |
| | compdA | | | | | | |
| comp. Ex2 | Comp. | 5.5 | 8.3 | 30.1 | 114.8 | 0.646 | 0.339 |
| | compdB | | | | | | |
| Test Ex. 1 | Com. (P-1) | 5.3 | 7.7 | 32.5 | 132.6 | 0.661 | 0.324 |
| Test Ex. 2 | Com. (P-3) | 5.3 | 7.6 | 32.9 | 125.6 | 0.660 | 0.330 |
| Test Ex. 3 | Com. (P-4) | 5.4 | 7.9 | 31.8 | 122.8 | 0.657 | 0.342 |
| Test Ex. 4 | Com. (P-6) | 5.2 | 7.8 | 32.1 | 130.4 | 0.658 | 0.321 |
| Test Ex. 5 | Com. (P-9) | 5.2 | 7.9 | 31.6 | 128.5 | 0.657 | 0.344 |
| Test Ex. 6 | Com. (P-13) | 5.3 | 7.7 | 32.3 | 122.7 | 0.658 | 0.346 |
| Test Ex. 7 | Com. (P-19) | 5.1 | 7.8 | 31.9 | 121.8 | 0.663 | 0.341 |
| Test Ex. 8 | Com. (P-21) | 5.4 | 7.5 | 33.1 | 130.5 | 0.663 | 0.329 |
| Test Ex. 9 | Com. (P-23) | 5.4 | 7.4 | 33.8 | 127.8 | 0.655 | 0.329 |
| Test Ex. 10 | Com. (P-31) | 5.3 | 7.9 | 31.7 | 120.4 | 0.661 | 0.349 |
| Test Ex. 11 | Com. (P-33) | 5.2 | 7.5 | 33.2 | 136.7 | 0.662 | 0.322 |
| Test Ex. 12 | Com. (P-34) | 5.1 | 7.6 | 32.8 | 130.0 | 0.660 | 0.336 |
| Test Ex. 13 | Com. (P-35) | 5.2 | 7.4 | 33.6 | 129.2 | 0.659 | 0.349 |
| Test Ex. 14 | Com. (P-38) | 5.0 | 7.7 | 32.3 | 127.6 | 0.662 | 0.327 |
| Test Ex. 15 | Com. (P-42) | 5.1 | 7.7 | 32.6 | 129.1 | 0.659 | 0.340 |
| Test Ex. 16 | Com. (P-45) | 5.2 | 7.8 | 32.2 | 127.3 | 0.664 | 0.341 |
| Test Ex. 17 | Com. (P-54) | 5.2 | 7.5 | 33.5 | 127.9 | 0.665 | 0.342 |
| Test Ex. 18 | Com. (P-66) | 5.1 | 7.8 | 32.1 | 126.6 | 0.660 | 0.331 |
| Test Ex. 19 | Com. (P-70) | 5.1 | 7.5 | 33.3 | 131.4 | 0.661 | 0.330 |
| Test Ex. 20 | Com. (P-71) | 5.1 | 7.4 | 33.8 | 130.1 | 0.660 | 0.343 |
| Test Ex. 21 | Com. (P-74) | 5.0 | 7.5 | 33.2 | 127.7 | 0.658 | 0.325 |
| Test Ex. 22 | Com. (P-85) | 5.2 | 7.3 | 34.2 | 135.7 | 0.663 | 0.320 |
| Test Ex. 23 | Com. (P-91) | 5.2 | 7.2 | 34.5 | 128.0 | 0.663 | 0.341 |
| Test Ex. 24 | Com. (P-106) | 5.3 | 7.5 | 33.4 | 125.2 | 0.656 | 0.322 |
| Test Ex. 25 | Com. (P-111) | 5.1 | 7.6 | 33.0 | 135.2 | 0.663 | 0.331 |
| Test Ex. 26 | Com. (P-122) | 5.2 | 7.4 | 33.9 | 140.2 | 0.662 | 0.322 |

As shown in Table 5, when the compound of the present invention is used as a host material for the light-emitting layer, it was confirmed that the driving voltage is lowered and both efficiency and lifetime are significantly improved compared to the case where Comparative Compound A or Comparative Compound B is used. Comparative Compound A differs from the compound of the present invention in that the substituent corresponding to Formula A of the present invention is a carbazolyl group, whereas the compound of the present invention contains a dibenzofuran or dibenzothiophene moiety. Due to this structural difference, it can be seen that even compounds having similar core structures may exhibit differences in device characteristics depending on the type of substituents, considering that the device characteristics were improved when the compound of the present invention was used as a host compared to when Comparative Compound A was used.

Table 6 below shows the reorganization energy (RE) values for Comparative Compound A and Compound P-45 of the present invention, wherein the REₕₒₗₑ values were calculated according to the aforementioned equation.

TABLE 6

| | Reorganization |
|---|---|
| Compound | Energy (RE) |
| Comp. compd A | 0.20 |
| P-45 | 0.12 |

As shown in Table 6, Compound P-45 of the present invention exhibits a lower RE value compared to Comparative Compound A. This indicates that the reorganization energy (RE) can vary depending on the type of substituent on the amine group. A lower reorganization energy (RE) value results in enhanced hole mobility, thereby enabling faster operation of the Hole Only Device (HOD). Accordingly, it appears to lower the driving voltage, while improving both efficiency and lifetime of a device, when the compound of the present invention is used as a host material.

Particularly, when a mixture of two or more compounds is used as the host material in the light-emitting layer, the driving voltage, efficiency, and lifetime are determined by the ease of hole and electron injection into the dopant. A well-balanced injection of holes and electrons (i.e., proper charge balance) results in a significant improvement in both efficiency and lifetime of a device.

Accordingly, it can be concluded that even within the same molecular backbone, the RE value varies depending on the type and position of substituents, which results in differences in device characteristics. This is supported by the observation that a specific combination of substituents on the amine moiety can positively influence overall hole mobility, thereby improving charge balance between holes and electrons—as well as enhancing energy balance and stability—ultimately leading to improved overall device performance.

Comparative Compound B differs from the compounds of the present invention in that the dibenzofuran is directly substituted on the nitrogen of the amine group, whereas in the compounds of the present invention, dibenzofuran or dibenzothiophene is substituted via a phenylene linker.

In view of the fact that the presence or absence of a linker affects the device characteristics, it is presumed that the physical properties of these compounds differ. Accordingly, the bond dissociation energy (BDE) of Comparative Compound B and Compound P-71 of the present invention was measured.

Table 7 below presents the bond dissociation energy (BDE) values of the weakest bonds in Comparative Compound B and Compound P-71 of the present invention, calculated using molecular simulations performed with Gaussian 09 Rev. C.01 and the Schrödinger Materials Science Suite 4.1.161.

The BDE values listed in Table 7 were calculated in the oxidized state, in which an electron is removed from the molecule, resulting in a positive charge being introduced into the tertiary amine. In other words, measuring the BDE in the oxidized state allows for the evaluation of hole stability. It is considered that the higher the BDE, the greater the stability with respect to holes.

TABLE 7

| Structure | Comp. | |
|---|---|---|
| Name | compd B | P-71 |
| BDE (eV) | 40.4 | 43.2 |

As shown in Table 7, it can be confirmed that the BDE value of Compound P-71 of the present invention is higher than that of Comparative Compound B. In organic electronic device, a lower crystallinity of the thin film may result in an amorphous state. Owing to its isotropic and homogeneous characteristics, the amorphous state can reduce grain boundaries, thereby enhancing the mobility of both charges and holes. Nevertheless, the quantum mechanical bond dissociation energy (BDE) of a molecule in the amorphous solid state can vary depending on its molecular structure due to intermolecular interactions in the solid phase. The higher the BDE value, the more stable the compound is intrinsically.

Accordingly, when Compound P-71 of the present invention is used as the host in the organic electronic device, rather than Comparative Compound B, the stability of holes transferring from a hole-transporting layer to the a light-emitting layer is significantly enhanced, which is expected to improve the lifetime of a device.

In addition, the x value of the CIE (color index) of the compound of the present invention was slightly increased compared to that of the comparative compound, indicating that the use of the compound of the present invention as a host may affect the color of a device. That is, when phenanthrooxazole and phenyl-dibenzofuran are used as substituents on the amine group, as in the present invention, it appears that the synergistic effect of these groups contributes to the enhanced performance.

The foregoing description is merely illustrative of the present invention and various modifications may be made by those of ordinary skill in the art without departing from the essential characteristics of the invention. The scope of protection of the present invention should be interpreted based on the following claims, and all technical equivalents thereof should be construed as falling within the scope of the invention.

1. A compound of Formula 1: 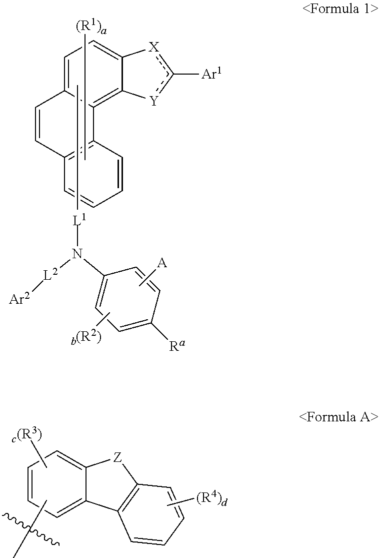
wherein,
X and Y are each independently N, O or S, and one of X and Y is N and the other is O or S,
Ar¹ and Ar² are each independently selected from the group consisting of a C₆-C₆₀ aryl group, a fluorenyl group, a C₃-C₆₀ aliphatic ring group, and a C₂-C₃₀ heterocyclic group containing at least one heteroatom of O, N, S, Si and P,
A is Formula A,
L¹ and L² are each independently selected from the group consisting of a single bond, a C₆-C₆₀ arylene group, a fluorenylene group, a C₃-C₆₀ aliphatic ring group, and a C₂-C₆₀ heterocyclic group containing at least one heteroatom of O, N, S, Si and P,
Z is O or S,
R¹ to R⁴ and R^{a} are each independently selected from the group consisting of hydrogen, deuterium, halogen, a cyano group, a nitro group, a C₆-C₆₀ aryl group, a fluorenyl group, a C₂-C₆₀ heterocyclic group containing at least one heteroatom of O, N, S, Si and P, a C₃-C₆₀ aliphatic ring group, a C₁-C₂₀ alkyl group, a C₂-C₂₀ alkenyl group, a C₂-C₂₀ alkynyl group, a C₁-C₂₀ alkoxyl group, and a C₆-C₆₀ aryloxy group, and adjacent R³ or adjacent R⁴ may be bonded to each other to form a ring,
a is an integer of 0 to 7, b and c are each an integer of 0 to 3, d is an integer of 0 to 4, and
the aryl group, the arylene group, the fluorenyl group, the fluorenylene group, the heterocyclic group, the aliphatic ring group, the alkyl group, the alkenyl group, the alkynyl group, the alkoxyl group, the aryloxyl group, and the ring formed by adjacent R³s or adjacent R⁴s may be each substituted with one or more substituents selected from the group consisting of deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group.

2. The compound of claim 1, wherein Formula 1 is represented by one of Formula 1-1 or Formula 1-2: 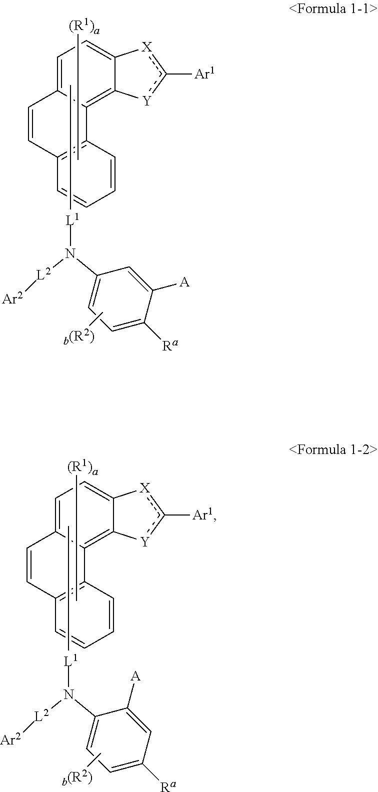
in Formula 1-1 and Formula 1-2, X, Y, R¹, R², R^{a}, L¹, L², Ar¹, Ar², A, a, b are the same as defined in claim 1.

3. The compound of claim 1, wherein Formula 1 is represented by Formula 1-3: 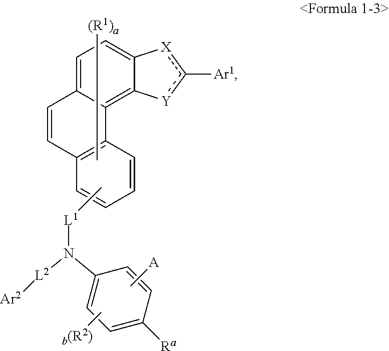
in Formula 1-3, X, Y, R¹, R², R^{a}, L¹, L², Ar¹, Ar, A, a, b are the same as defined in claim 1.

4. The compound of claim 1, wherein Formula A is represented by one of Formula A-1 to Formula A-4: 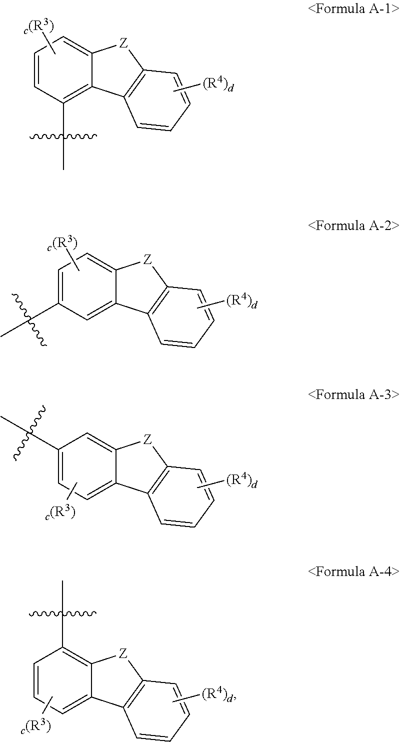
in Formula A-1 to Formula A-4, Z, R³, R⁴, c, d are the same as defined in claim 1.

5. The compound of claim 1, wherein L¹ or L² is selected from the group consisting of Formula L-1 to Formula L-3: 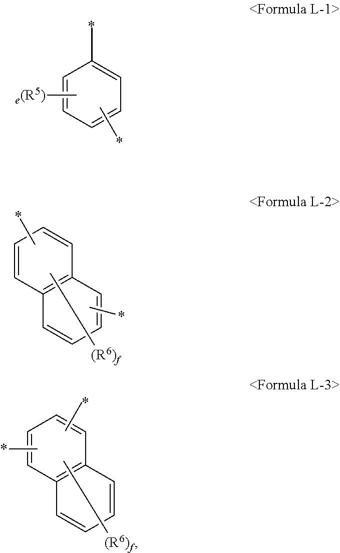
in Formula L-1 to Formula L-3,
R⁵ and R⁶ are selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent groups may be bonded to each other to form a ring, and
e is an integer of 0 to 4, and f is an integer of 0 to 6.

6. The compound of claim 1, wherein the compound represented by Formula 1 is one of the following compounds:

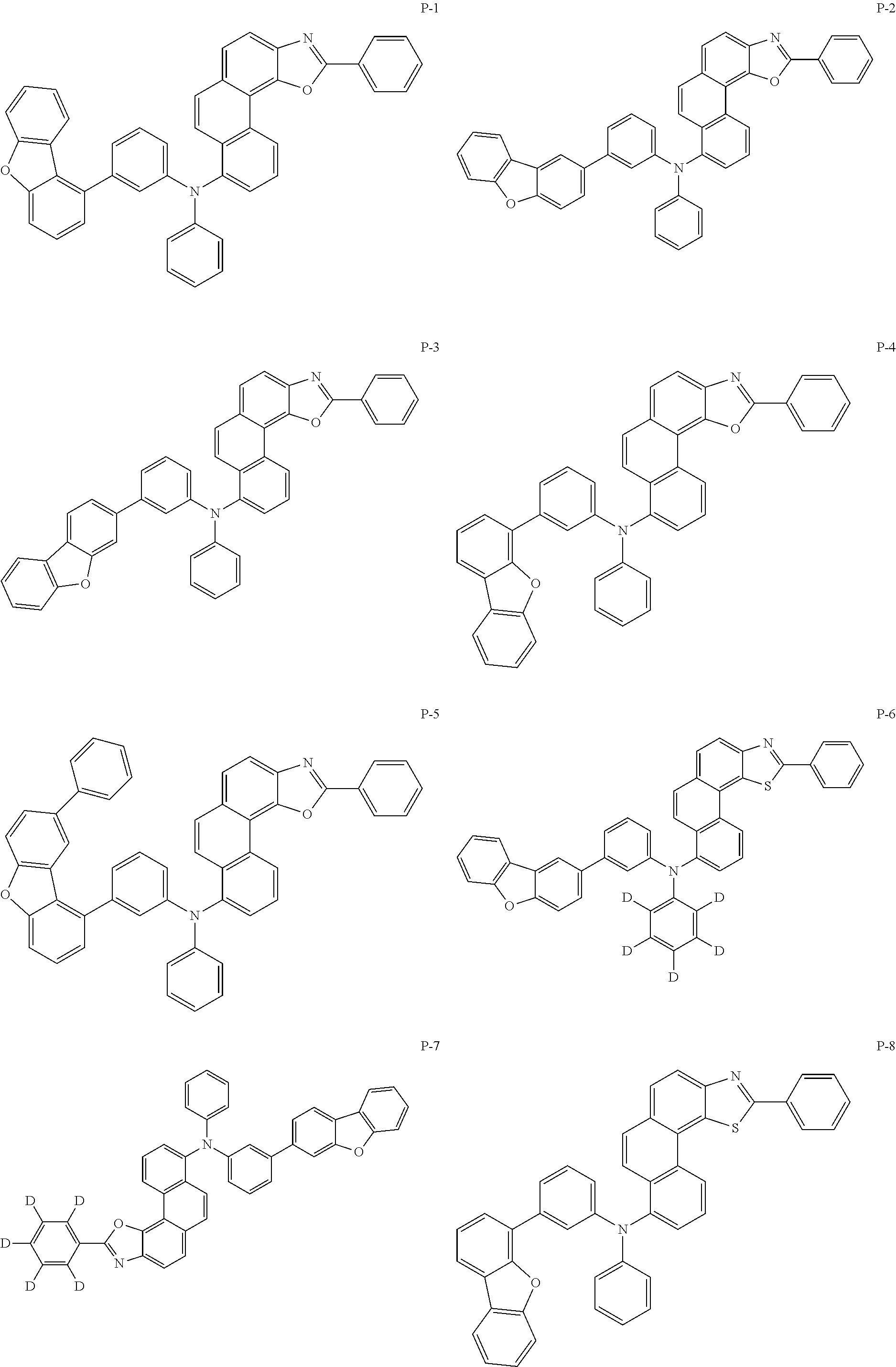

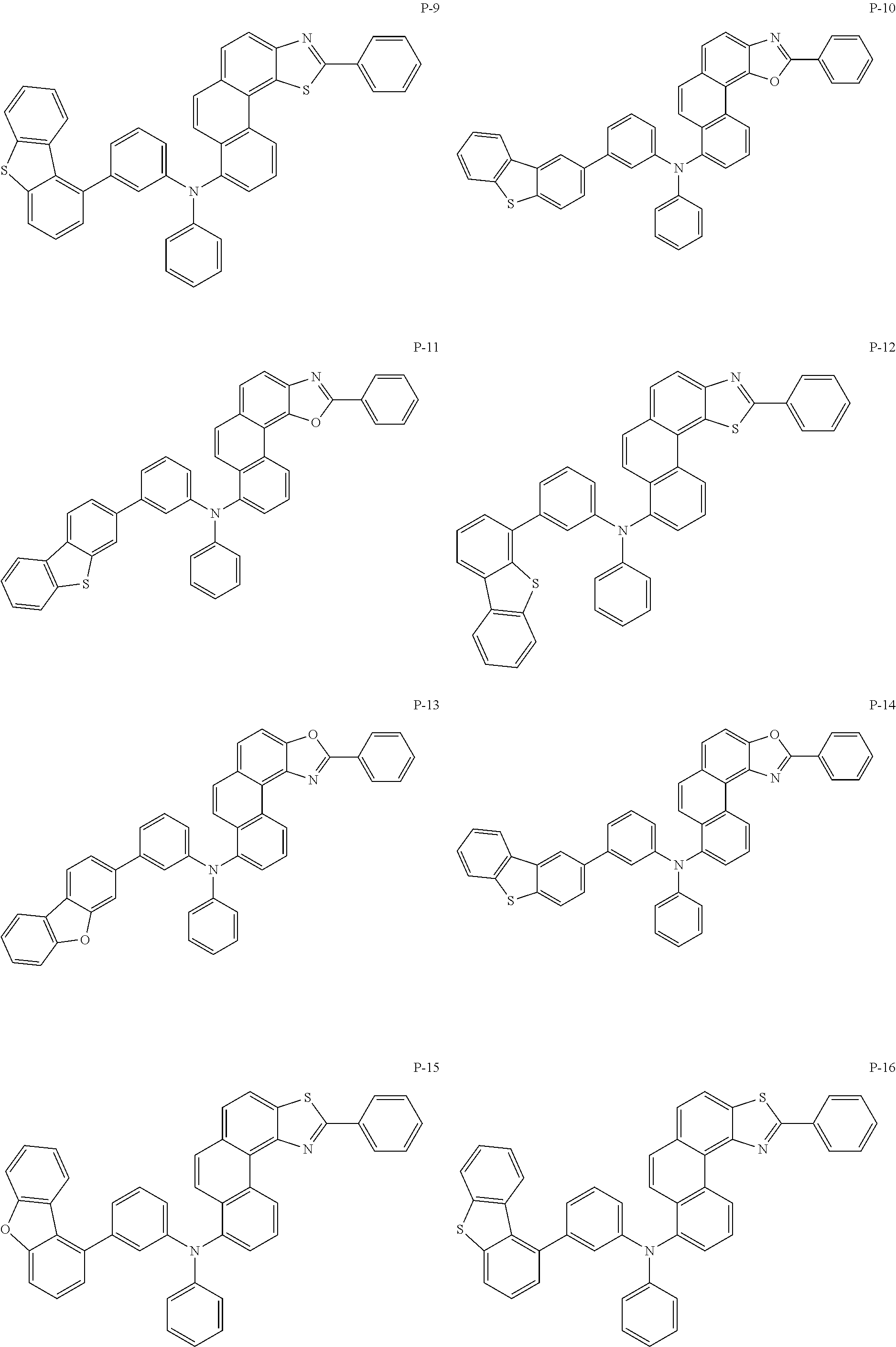

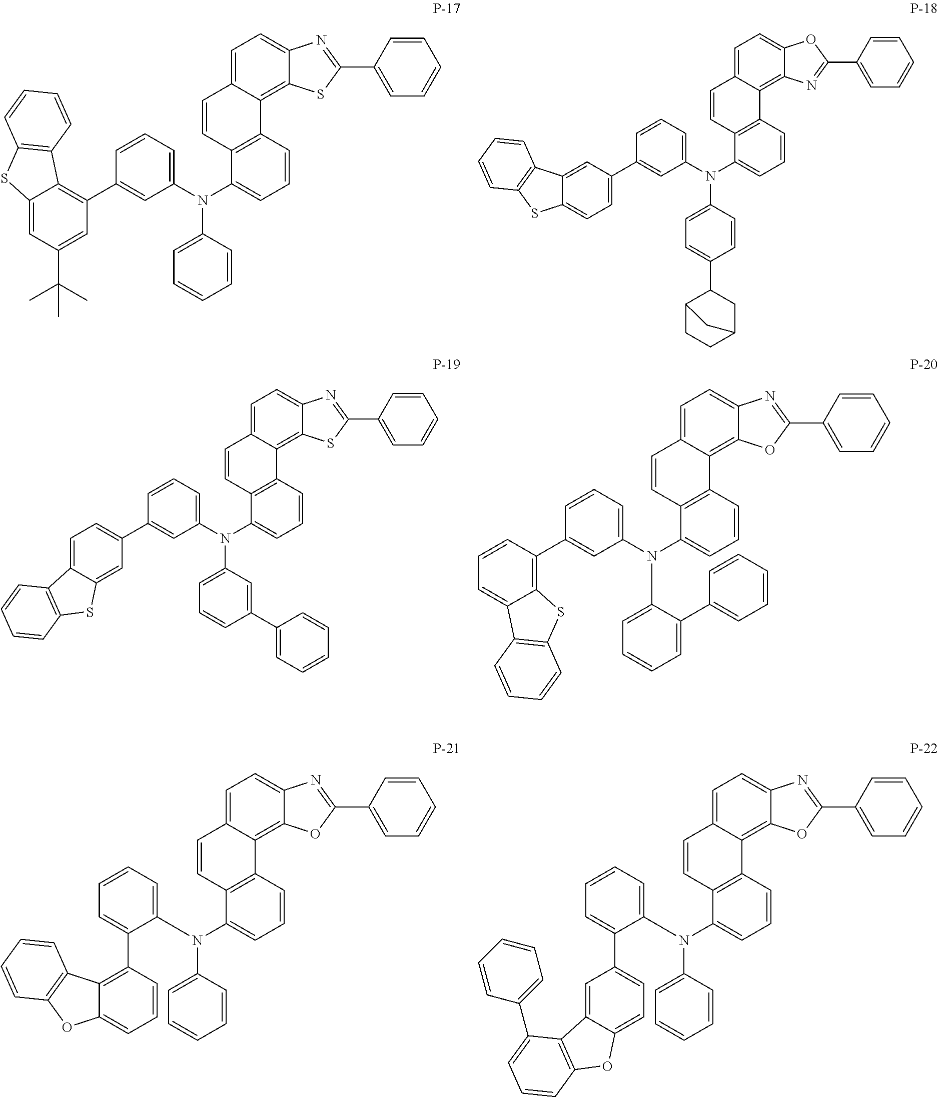

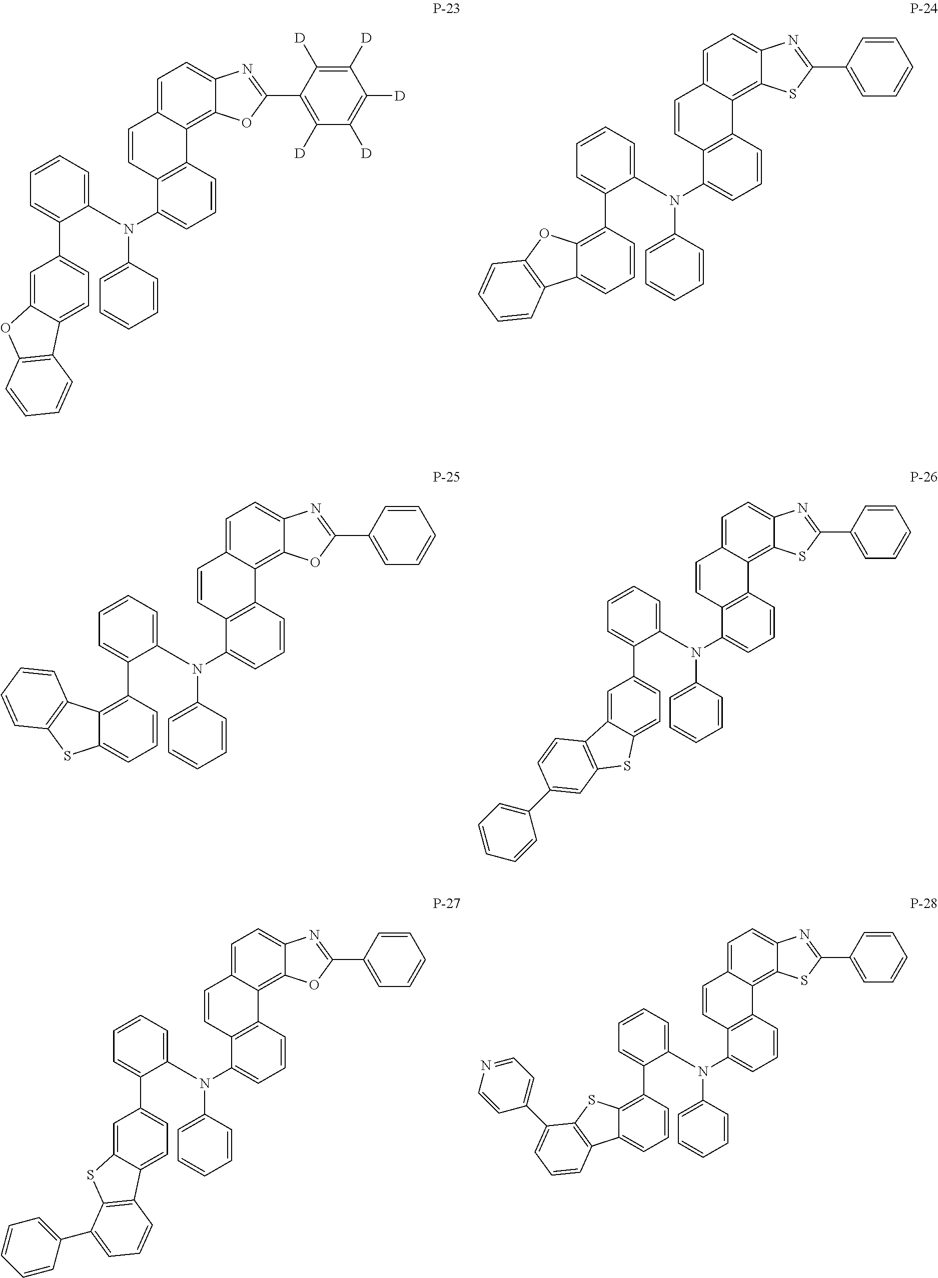

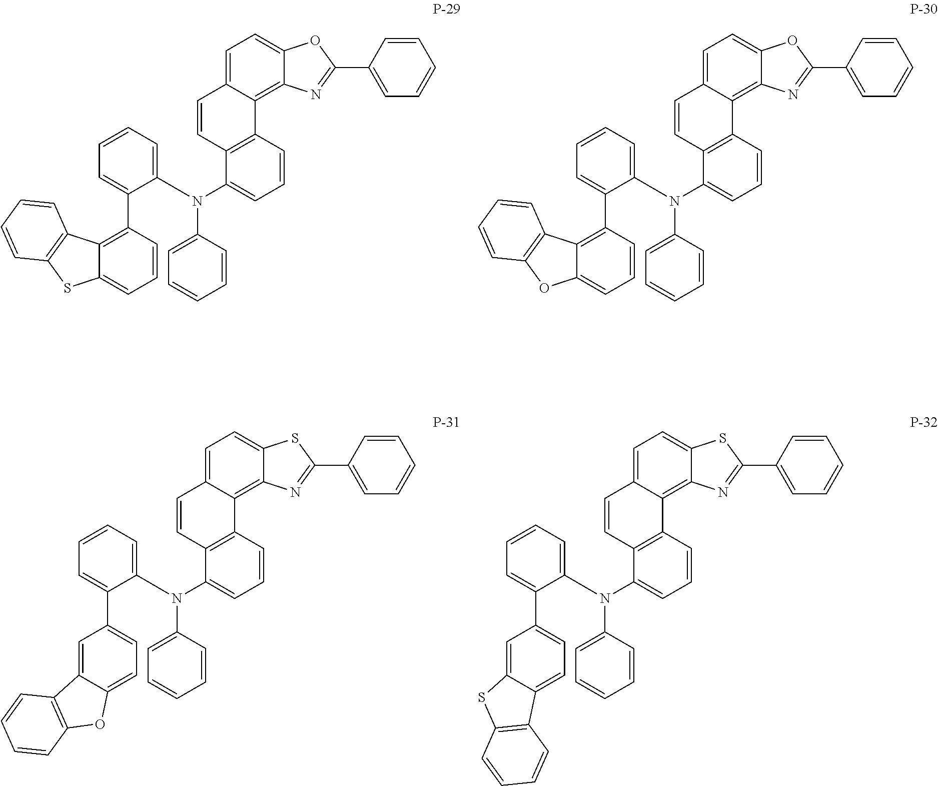 [SMILES: c1ccc(-c2nc3c(ccc4ccc5c(N(c6ccccc6)c6ccccc6-c6cccc7sc8ccccc8c67)cccc5c43)o2)cc1 ; c1ccc(-c2nc3c(ccc4ccc5c(N(c6ccccc6)c6ccccc6-c6cccc7oc8ccccc8c67)cccc5c43)o2)cc1 ; c1ccc(-c2nc3c(ccc4ccc5c(N(c6ccccc6)c6ccccc6-c6ccc7oc8ccccc8c7c6)cccc5c43)s2)cc1 ; c1ccc(-c2nc3c(ccc4ccc5c(N(c6ccccc6)c6ccccc6-c6ccc7c(c6)sc6ccccc67)cccc5c43)s2)cc1]

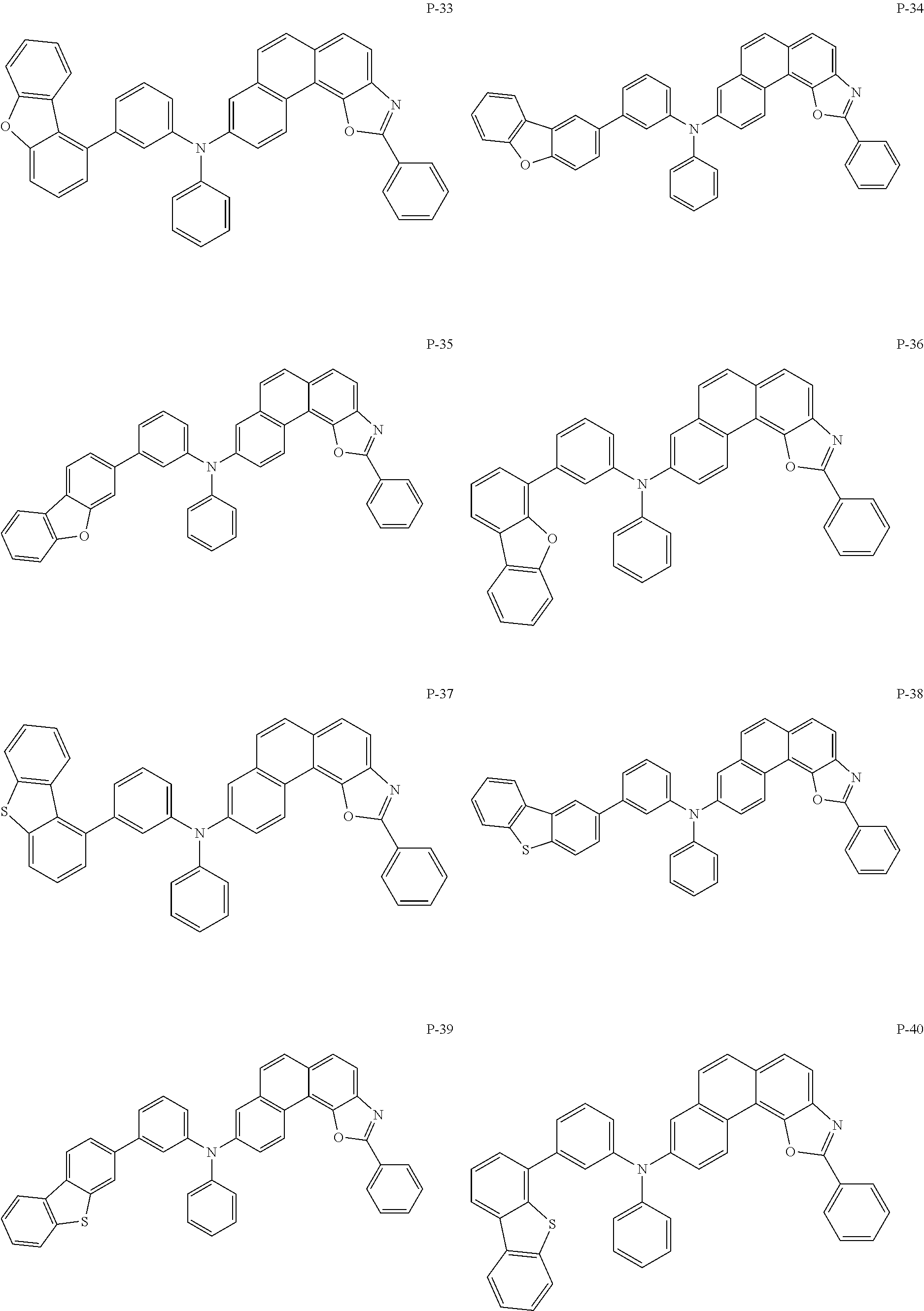

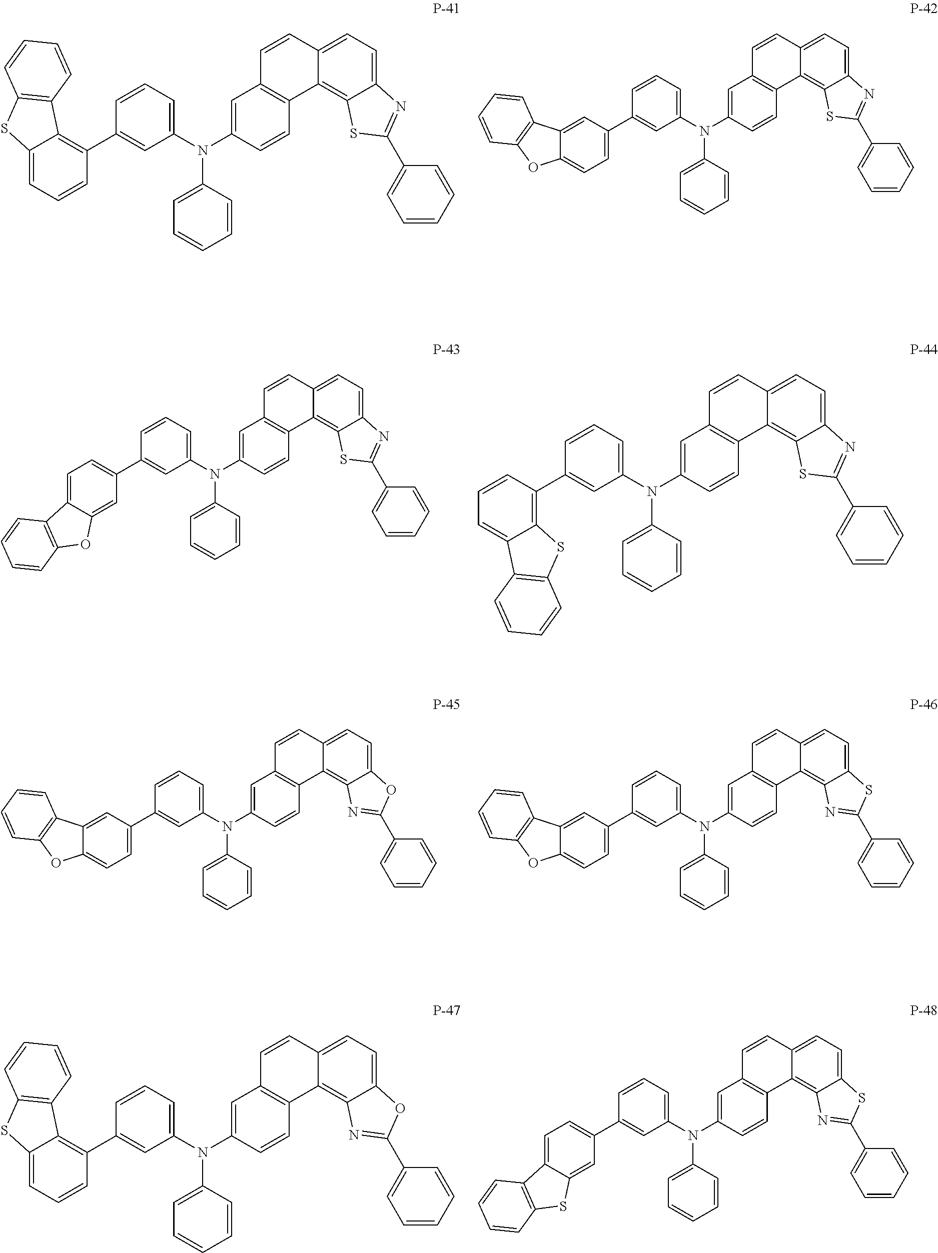

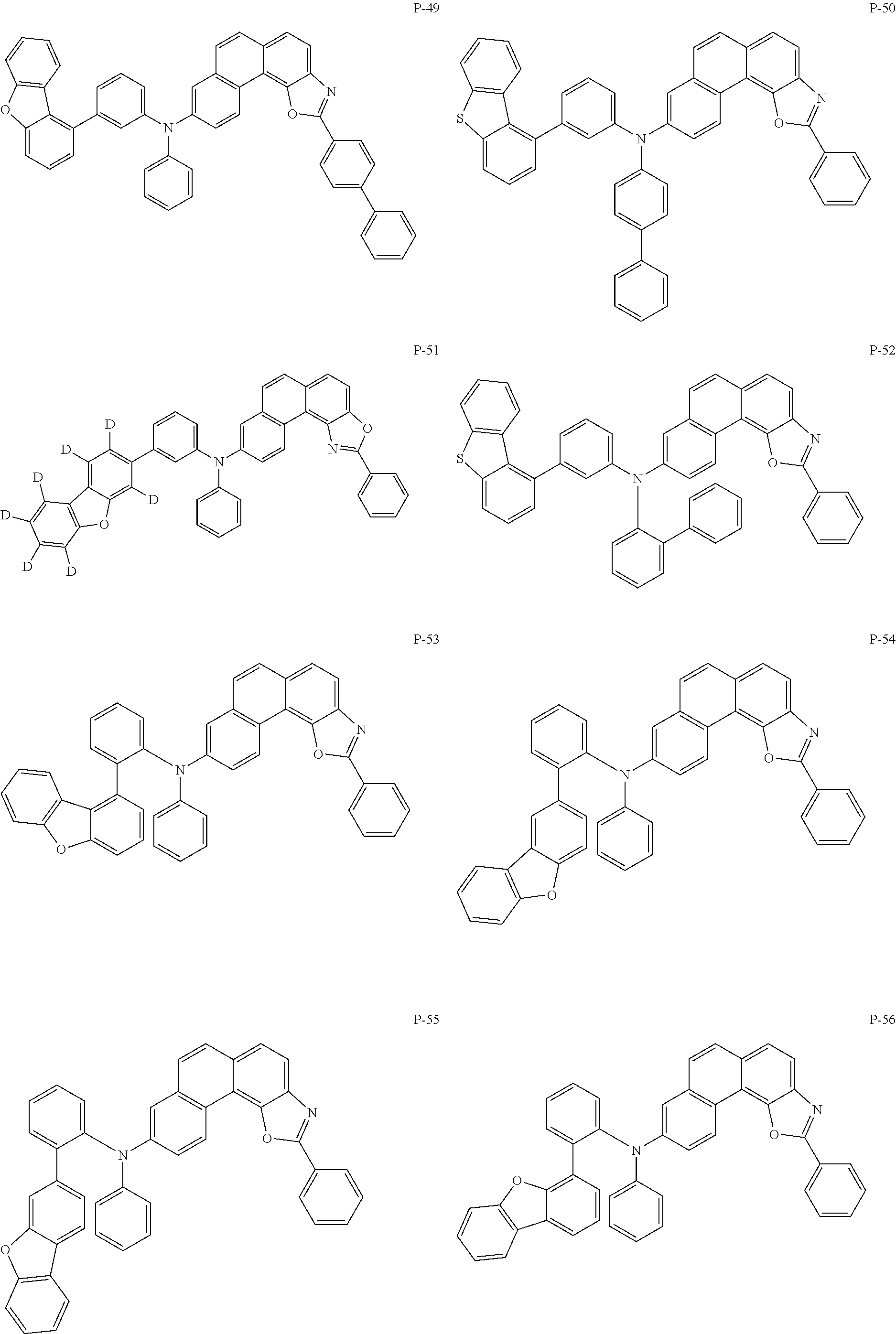

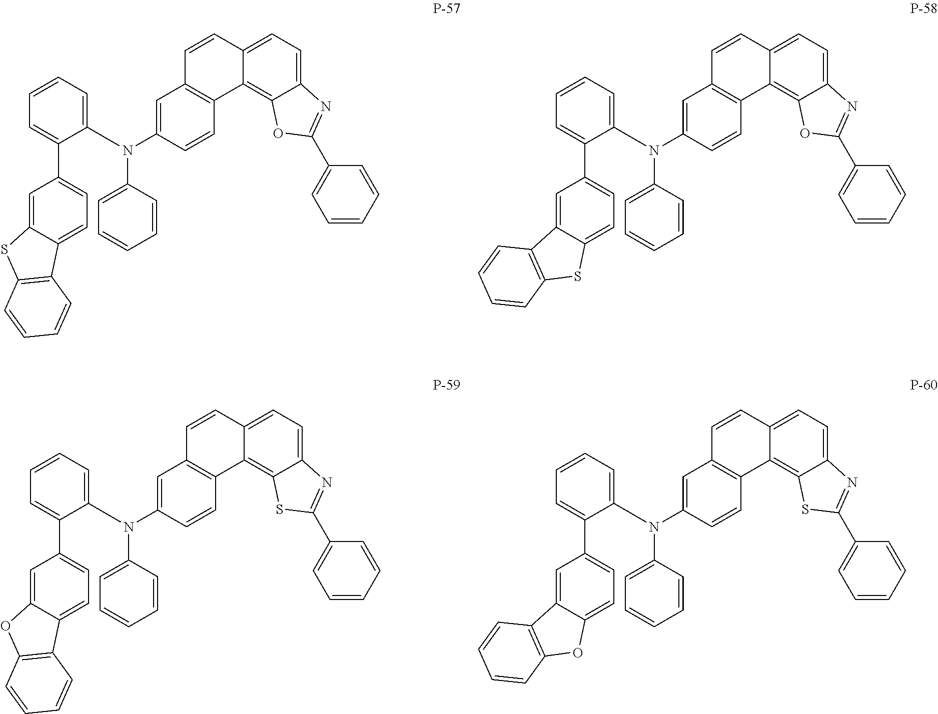 [SMILES: c1ccc(-c2nc3ccc4ccc5cc(N(c6ccccc6)c6ccccc6-c6ccc7c(c6)sc6ccccc67)ccc5c4c3o2)cc1 ; c1ccc(-c2nc3ccc4ccc5cc(N(c6ccccc6)c6ccccc6-c6ccc7sc8ccccc8c7c6)ccc5c4c3o2)cc1 ; c1ccc(-c2nc3ccc4ccc5cc(N(c6ccccc6)c6ccccc6-c6ccc7c(c6)oc6ccccc67)ccc5c4c3s2)cc1 ; c1ccc(-c2nc3ccc4ccc5cc(N(c6ccccc6)c6ccccc6-c6ccc7oc8ccccc8c7c6)ccc5c4c3s2)cc1]

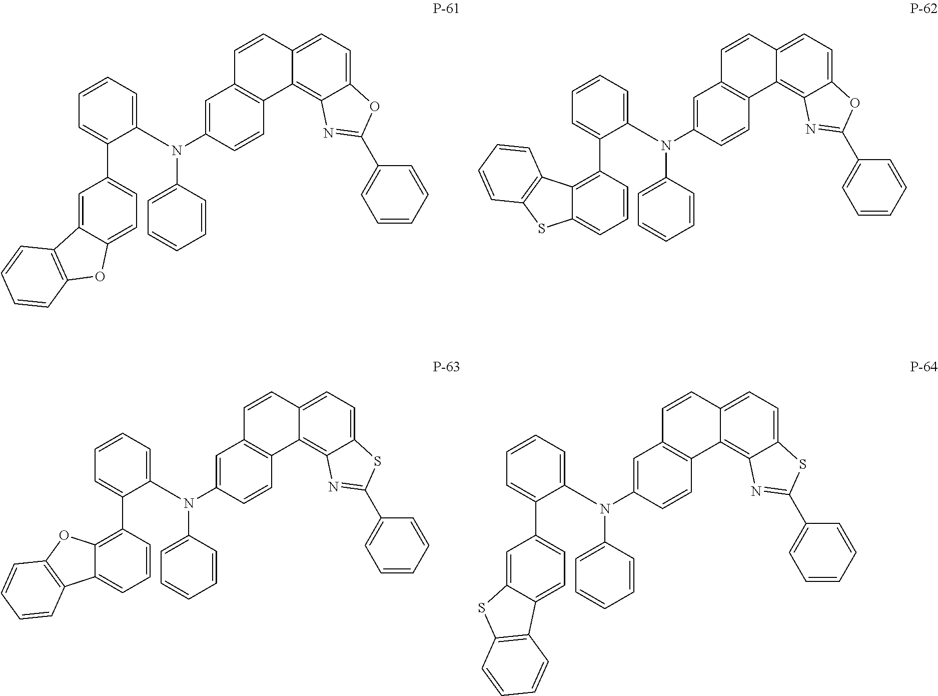 [SMILES: c1ccc(-c2nc3c(ccc4ccc5cc(N(c6ccccc6)c6ccccc6-c6ccc7oc8ccccc8c7c6)ccc5c43)o2)cc1 ; c1ccc(-c2nc3c(ccc4ccc5cc(N(c6ccccc6)c6ccccc6-c6cccc7sc8ccccc8c67)ccc5c43)o2)cc1 ; c1ccc(-c2nc3c(ccc4ccc5cc(N(c6ccccc6)c6ccccc6-c6cccc7c6oc6ccccc67)ccc5c43)s2)cc1 ; c1ccc(-c2nc3c(ccc4ccc5cc(N(c6ccccc6)c6ccccc6-c6ccc7c(c6)sc6ccccc67)ccc5c43)s2)cc1]

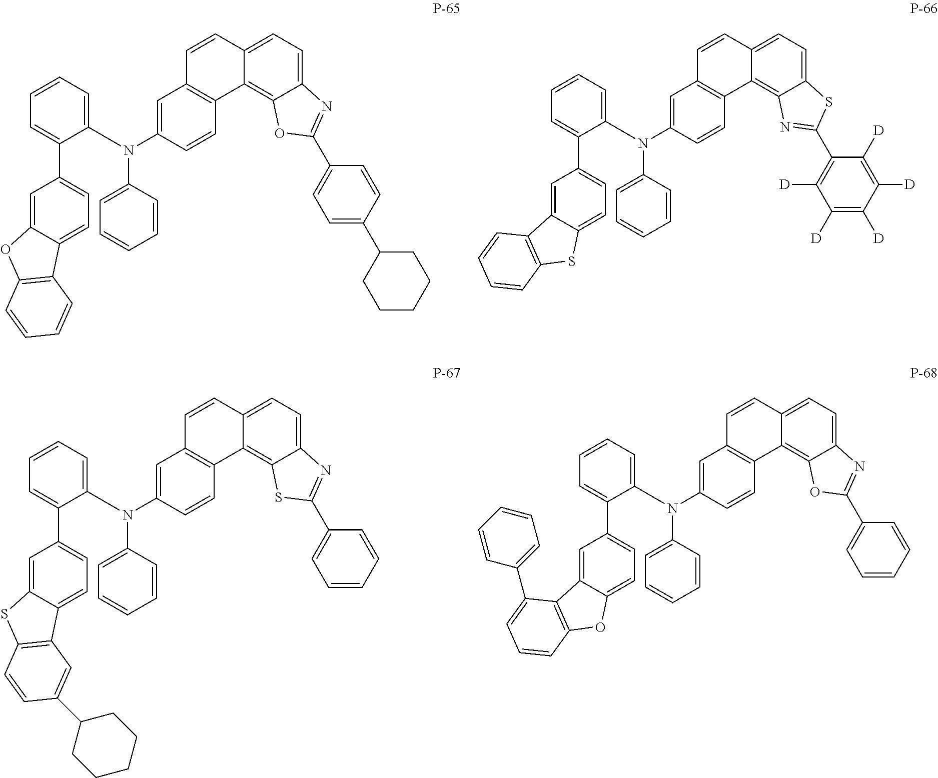 [SMILES: c1ccc(N(c2ccc3c(ccc4ccc5nc(-c6ccc(C7CCCCC7)cc6)oc5c43)c2)c2ccccc2-c2ccc3c(c2)oc2ccccc23)cc1 ; [2H]c1c([2H])c([2H])c(-c2nc3c(ccc4ccc5cc(N(c6ccccc6)c6ccccc6-c6ccc7sc8ccccc8c7c6)ccc5c43)s2)c([2H])c1[2H] ; c1ccc(-c2nc3ccc4ccc5cc(N(c6ccccc6)c6ccccc6-c6ccc7c(c6)sc6ccc(C8CCCCC8)cc67)ccc5c4c3s2)cc1 ; c1ccc(-c2nc3ccc4ccc5cc(N(c6ccccc6)c6ccccc6-c6ccc7oc8cccc(-c9ccccc9)c8c7c6)ccc5c4c3o2)cc1]

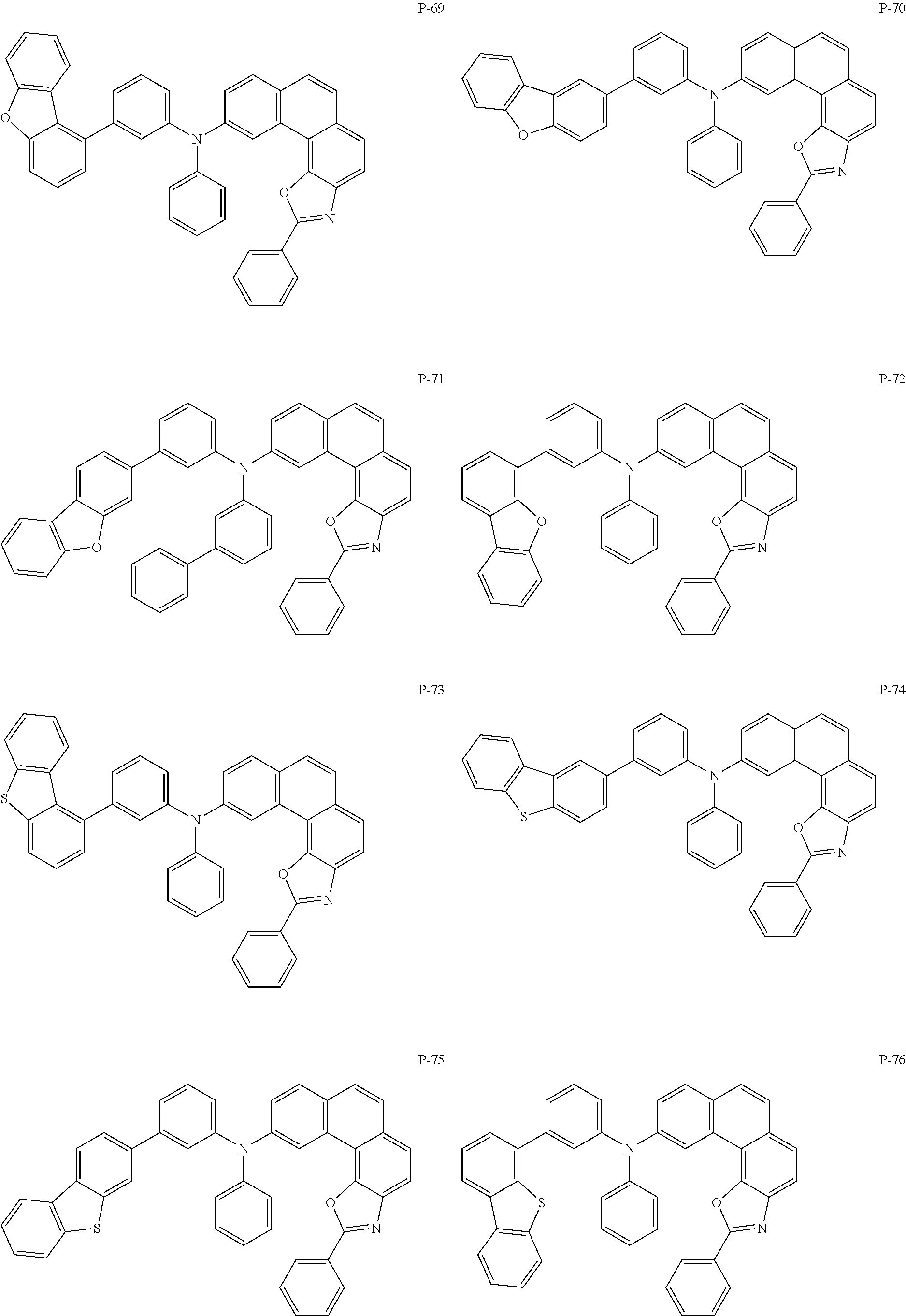

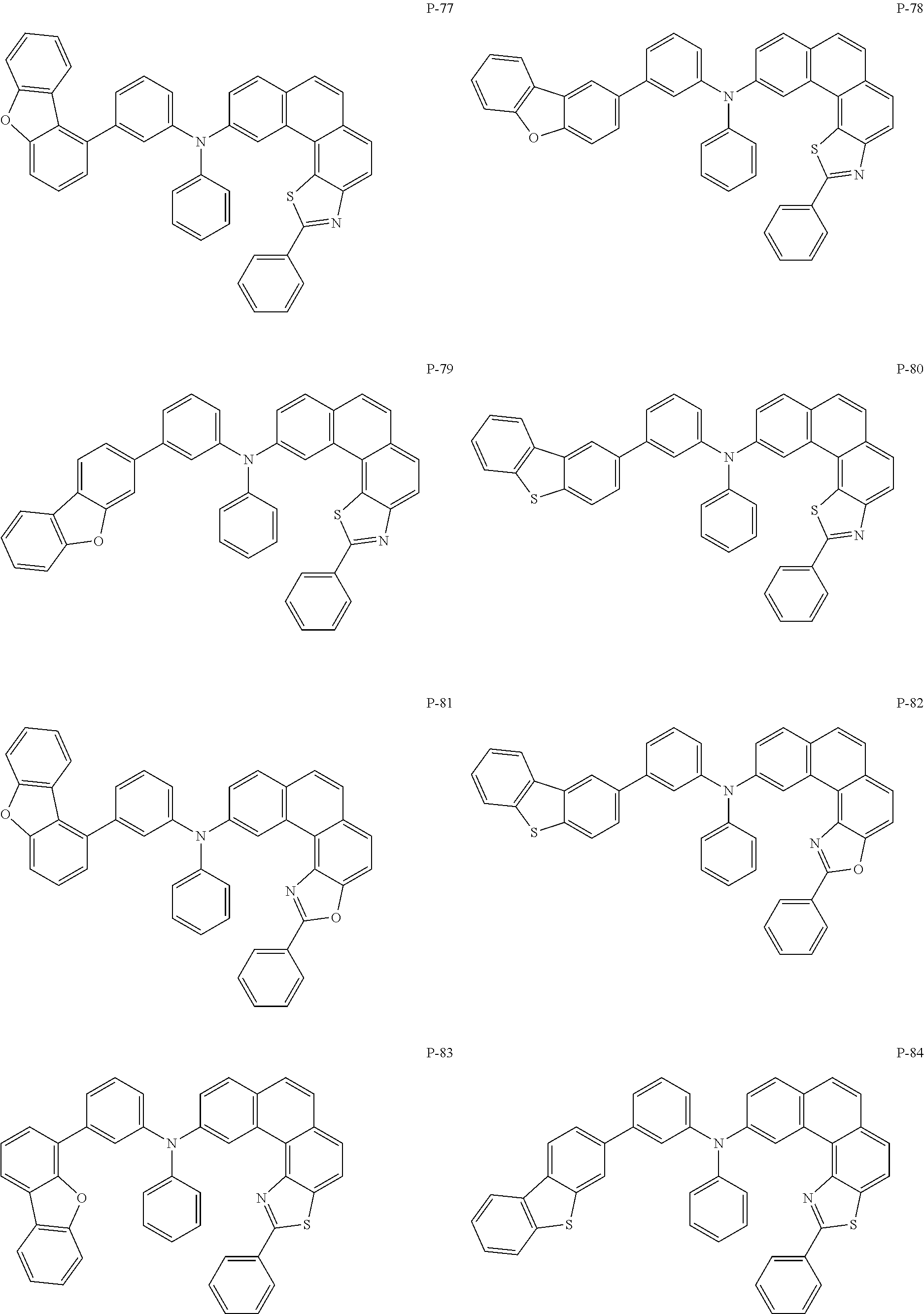

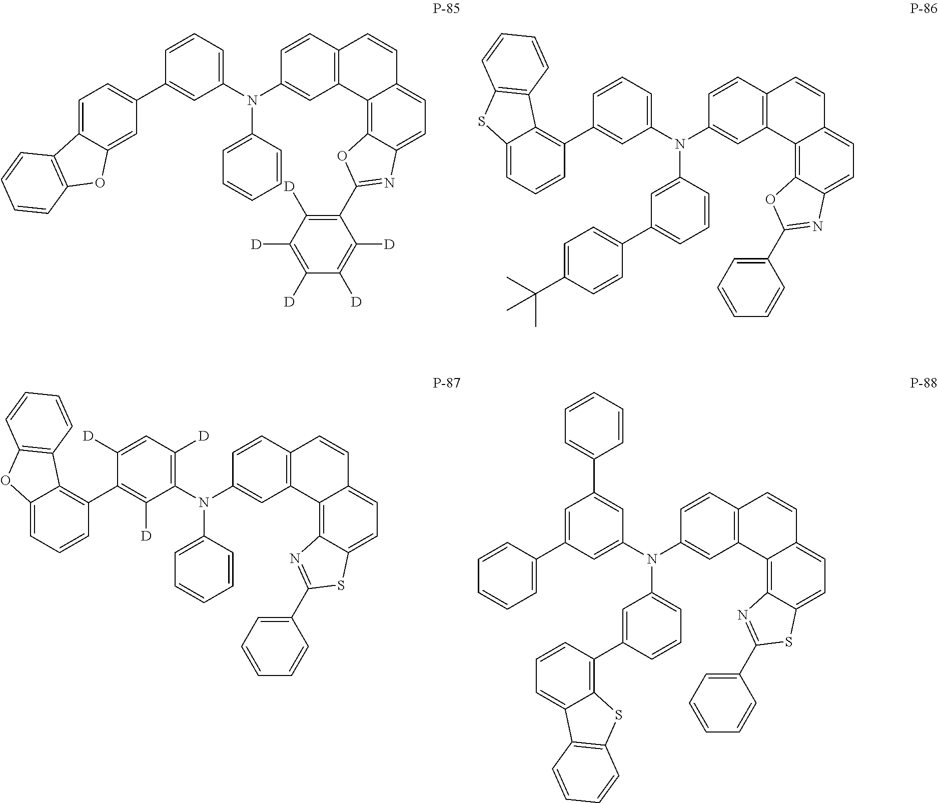 [SMILES: [2H]c1c([2H])c([2H])c(-c2nc3ccc4ccc5ccc(N(c6ccccc6)c6cccc(-c7ccc8c(c7)oc7ccccc78)c6)cc5c4c3o2)c([2H])c1[2H] ; CC(C)(C)c1ccc(-c2cccc(N(c3cccc(-c4cccc5sc6ccccc6c45)c3)c3ccc4ccc5ccc6nc(-c7ccccc7)oc6c5c4c3)c2)cc1 ; [2H]c1cc([2H])c(N(c2ccccc2)c2ccc3ccc4ccc5sc(-c6ccccc6)nc5c4c3c2)c([2H])c1-c1cccc2oc3ccccc3c12 ; c1ccc(-c2cc(-c3ccccc3)cc(N(c3cccc(-c4cccc5c4sc4ccccc45)c3)c3ccc4ccc5ccc6sc(-c7ccccc7)nc6c5c4c3)c2)cc1]

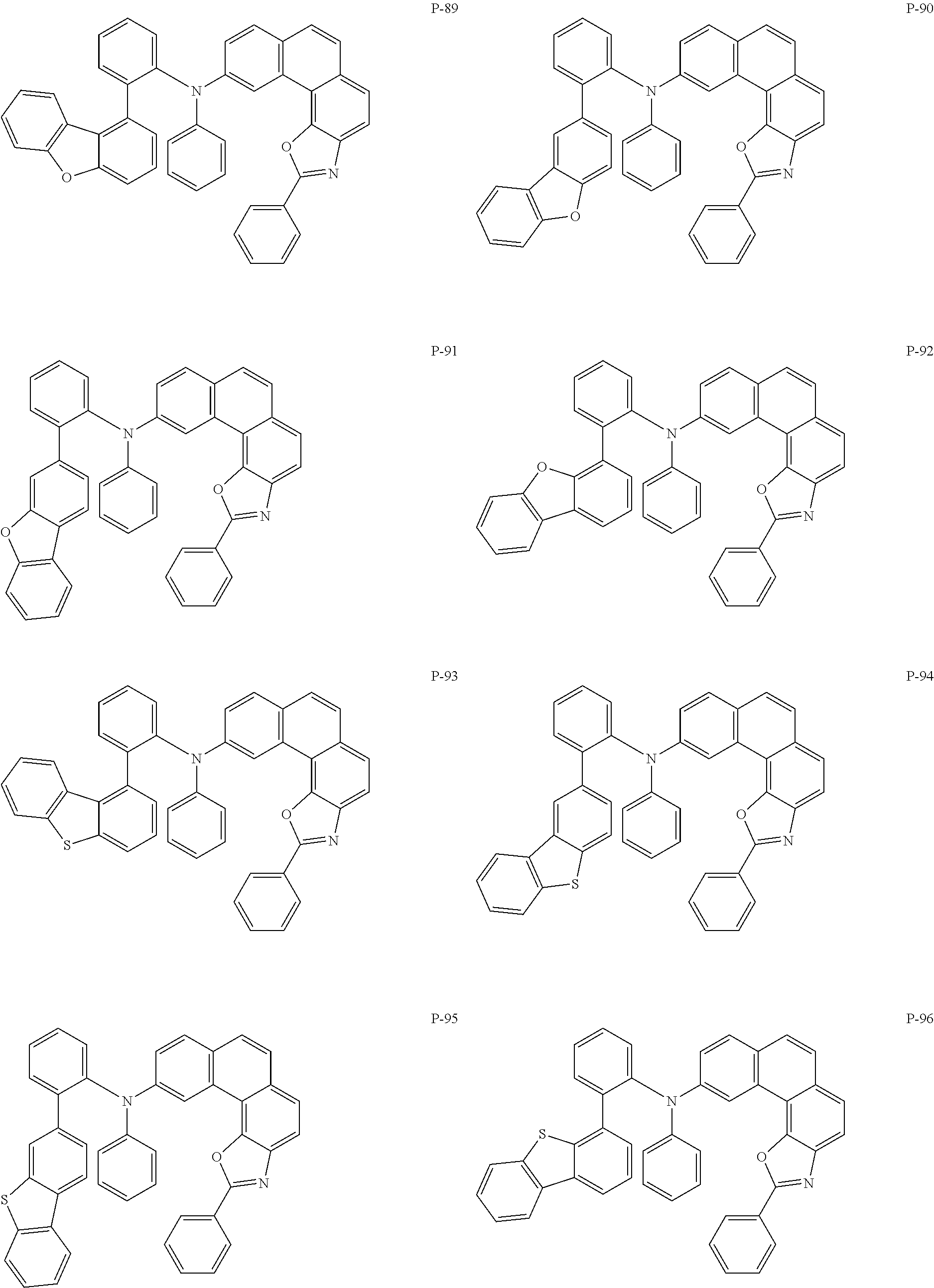

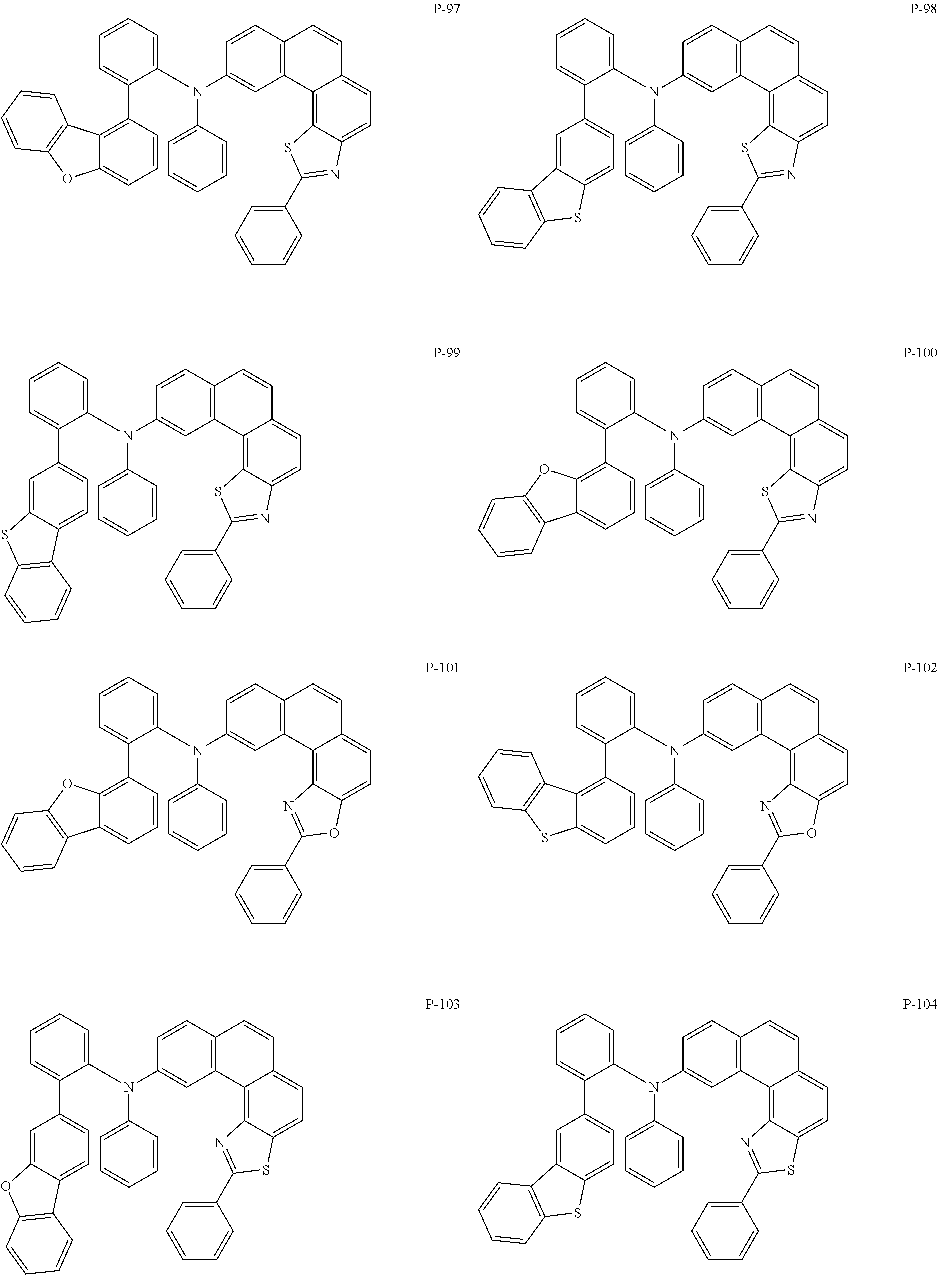

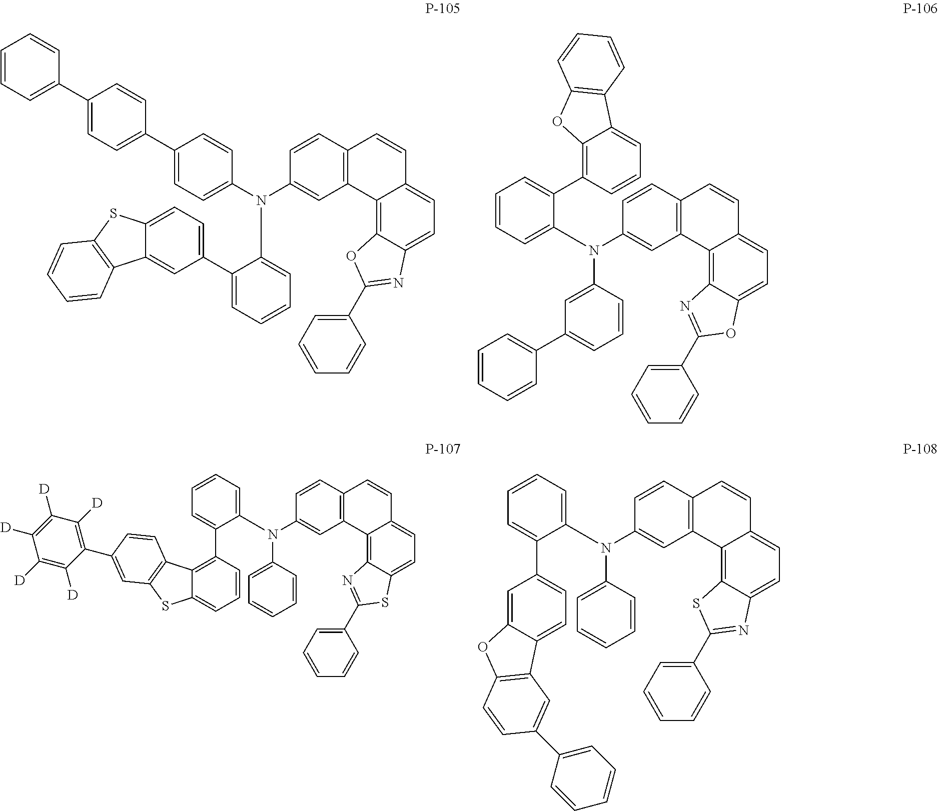 [SMILES: c1ccc(-c2ccc(-c3ccc(N(c4ccc5ccc6ccc7nc(-c8ccccc8)oc7c6c5c4)c4ccccc4-c4ccc5sc6ccccc6c5c4)cc3)cc2)cc1 ; c1ccc(-c2cccc(N(c3ccc4ccc5ccc6oc(-c7ccccc7)nc6c5c4c3)c3ccccc3-c3cccc4c3oc3ccccc34)c2)cc1 ; [2H]c1c([2H])c([2H])c(-c2ccc3c(c2)sc2cccc(-c4ccccc4N(c4ccccc4)c4ccc5ccc6ccc7sc(-c8ccccc8)nc7c6c5c4)c23)c([2H])c1[2H] ; c1ccc(-c2ccc3oc4cc(-c5ccccc5N(c5ccccc5)c5ccc6ccc7ccc8nc(-c9ccccc9)sc8c7c6c5)ccc4c3c2)cc1]

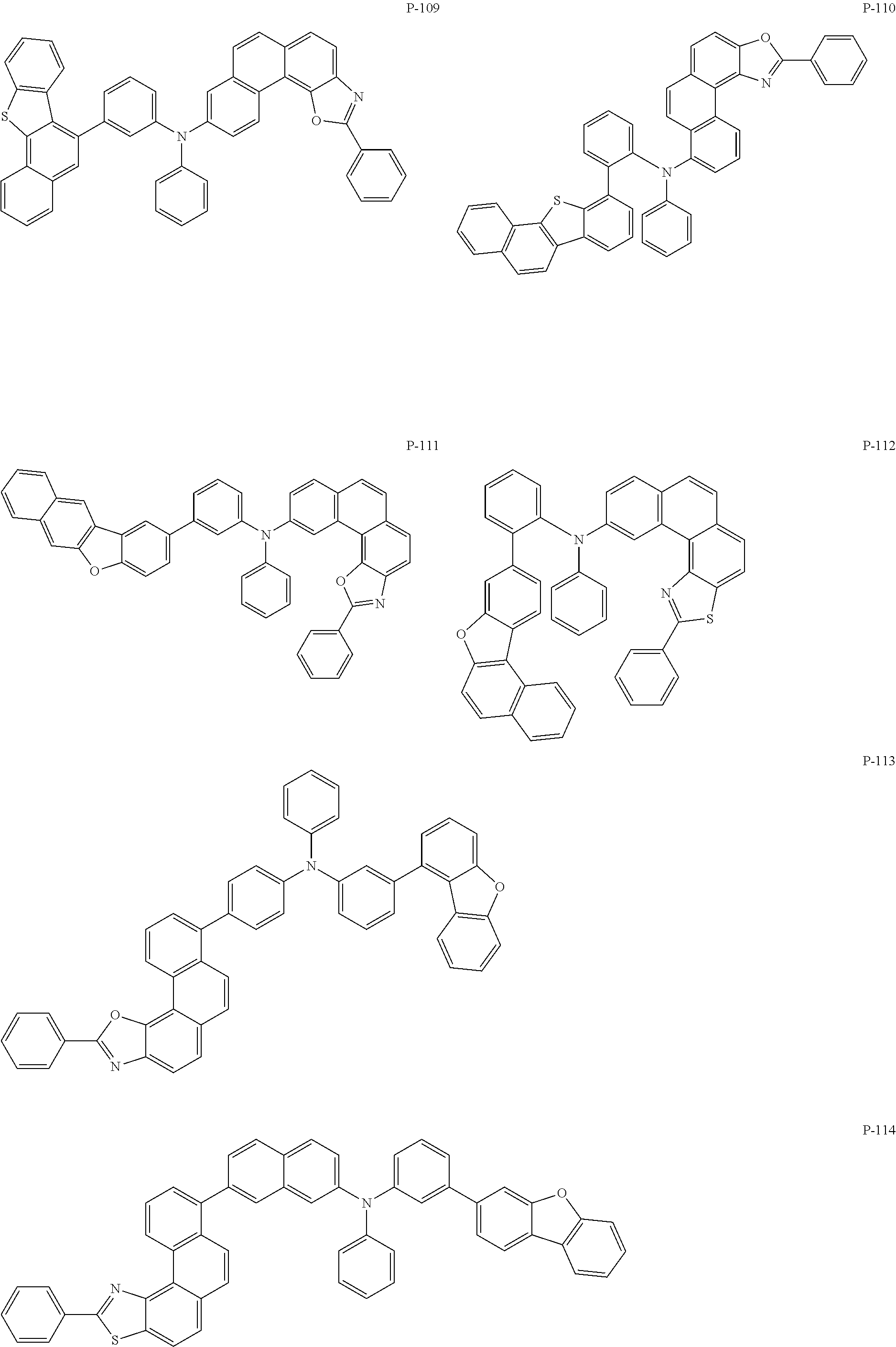

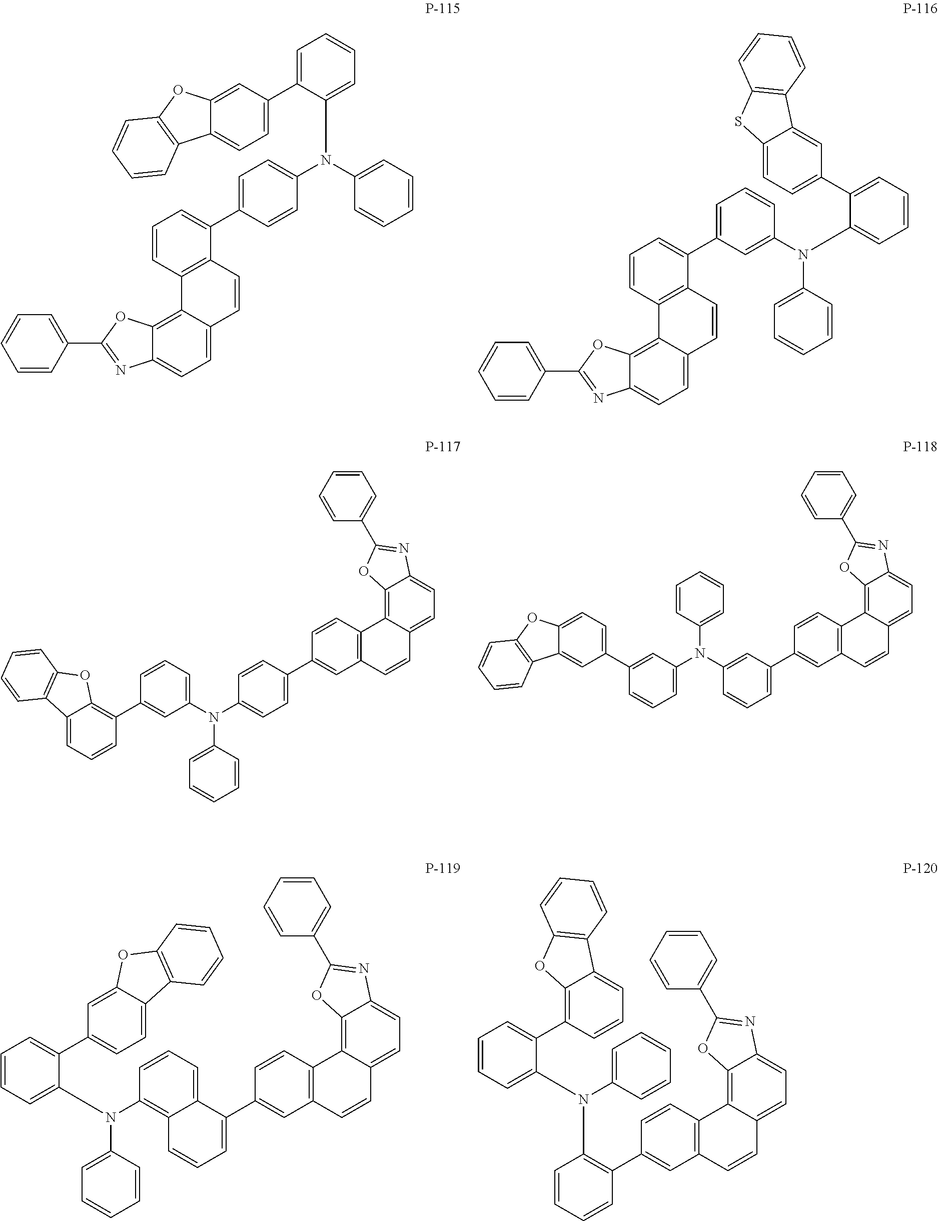

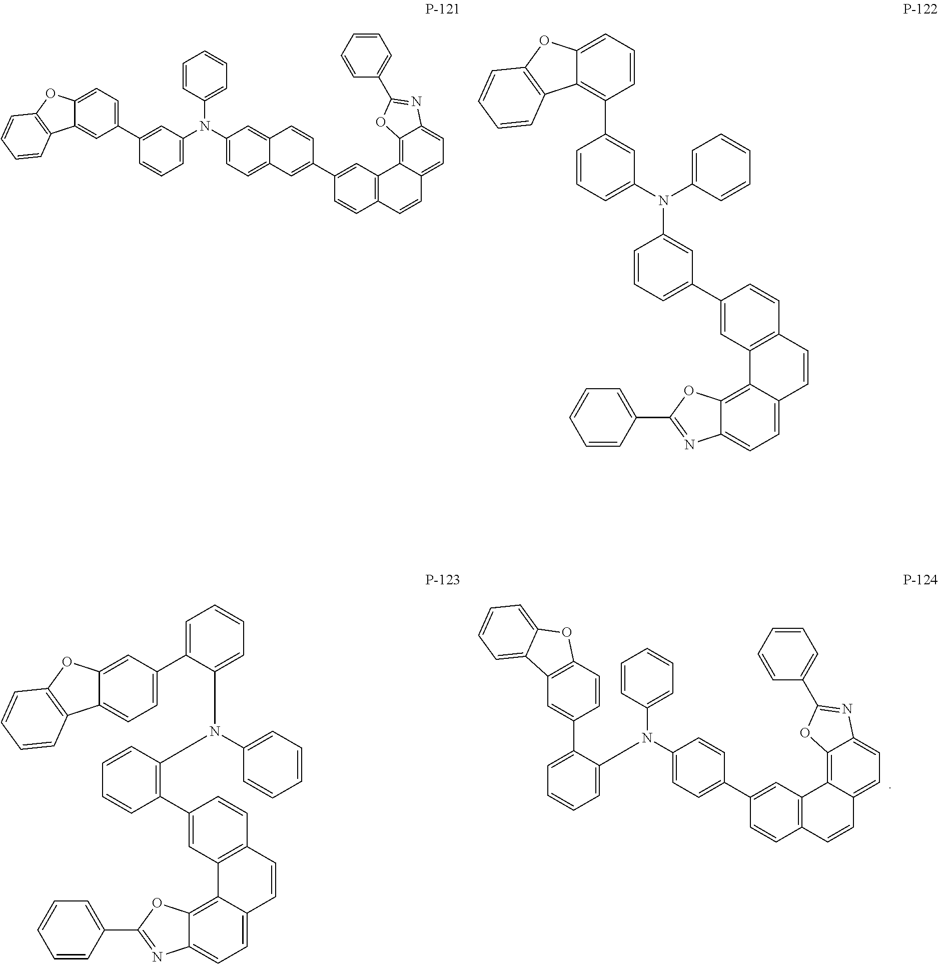 [SMILES: c1ccc(-c2nc3ccc4ccc5ccc(-c6ccc7cc(N(c8ccccc8)c8cccc(-c9ccc%10oc%11ccccc%11c%10c9)c8)ccc7c6)cc5c4c3o2)cc1 ; c1ccc(-c2nc3ccc4ccc5ccc(-c6cccc(N(c7ccccc7)c7cccc(-c8cccc9oc%10ccccc%10c89)c7)c6)cc5c4c3o2)cc1 ; c1ccc(-c2nc3ccc4ccc5ccc(-c6ccccc6N(c6ccccc6)c6ccccc6-c6ccc7c(c6)oc6ccccc67)cc5c4c3o2)cc1 ; c1ccc(-c2nc3ccc4ccc5ccc(-c6ccc(N(c7ccccc7)c7ccccc7-c7ccc8oc9ccccc9c8c7)cc6)cc5c4c3o2)cc1]

7. The compound of claim 1, wherein a reorganization energy value of the compound represented by Formula 1 is 0.10 to 0.19.

8. An organic electronic device comprising a first electrode, a second electrode, and an organic layer disposed between the first electrode and the second electrode, wherein the organic layer comprises the compound of Formula 1 of claim 1.

9. The organic electronic device of claim 8, wherein the organic layer comprises a phosphorescent light-emitting layer, and the phosphorescent light-emitting layer comprises the compound of claim 1 and a compound represented by Formula 2. 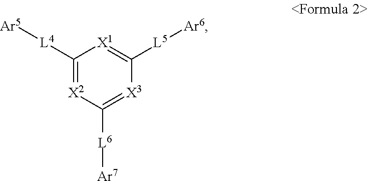
in Formula 2,
X¹ to X³ are independently C(R′) or N, and at least one of X¹ to X³ is N,
L⁴ to L⁶ are each independently selected from the group consisting of a single bond, a C₆-C₆₀ arylene group, a fluorenylene group, a C₃-C₆₀ aliphatic ring group, and a C₂-C₆₀ heterocyclic group containing at least one heteroatom of O, N, S, Si and P,
Ar⁵ to Ar⁷ are each independently selected from the group consisting of a C₆-C₆₀ aryl group, a fluorenyl group, a C₃-C₆₀ aliphatic ring group, and a C₂-C₆₀ heterocyclic group containing at least one heteroatom of O, N, S, Si and P,
R′ is independently selected from the group consisting of hydrogen, deuterium, halogen, a cyano group, a nitro group, a C₆-C₆₀ aryl group, a fluorenyl group, a C₂-C₆₀ heterocyclic group containing at least one heteroatom of O, N, S, Si and P, a C₃-C₆₀ aliphatic ring group, a C₁-C₂₀ alkyl group, a C₂-C₂₀ alkenyl group, a C₂-C₂₀ alkynyl group, a C₁-C₂₀ alkoxyl group, and a C₆-C₆₀ aryloxy group, and
the aryl group, the arylene group, the fluorenyl group, the fluorenylene group, the heterocyclic group, the aliphatic ring group, the alkyl group, the alkenyl group, the alkynyl group, the alkoxyl group, the aryloxyl group may be each substituted with one or more substituents selected from the group consisting of deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group.

10. The organic electronic device of claim 9, wherein at least one of Ar⁵ to Ar⁷ is selected from the group consisting of Formula Ar-1 to Formula Ar-8: 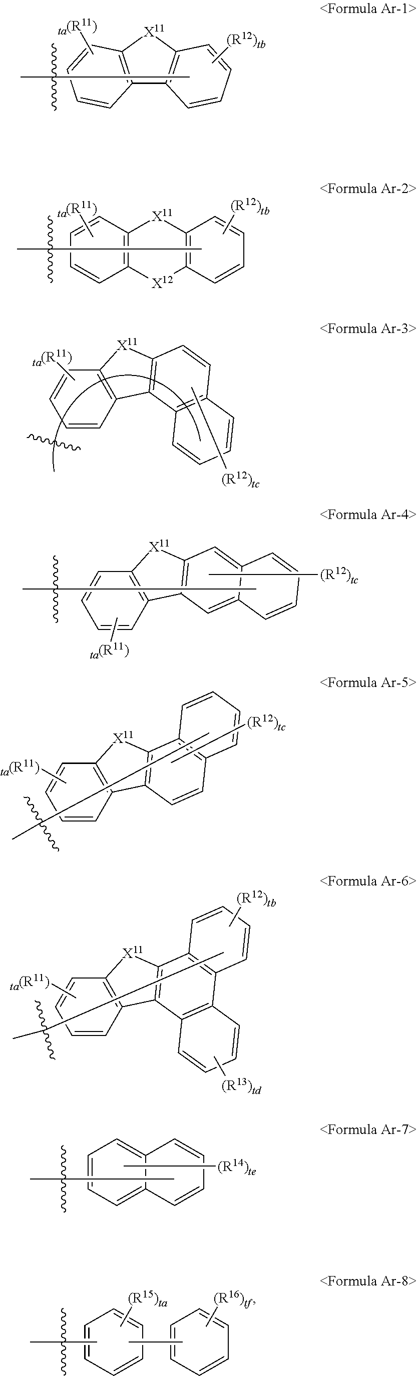
in Formula Ar-1 to Formula Ar-8,
X¹¹ and X¹² are independently N(Ar¹¹), O, S or C(R¹⁷)(R¹⁸),
R¹¹ to R¹⁸ are each independently selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent groups may be bonded to each other to form a ring, and R¹⁷ and R¹⁸ may be bonded to each other to form a ring,
ta, tb, td are each an integer of 0 to 4, tc is an integer of 0 to 6, te is an integer of 0 to 7, tf is an integer of 0 to 5, and
Ar¹¹ is selected from the group consisting of a C₆-C₃₀ aryl group, a fluorenyl group, a C₂-C₃₀ heterocyclic group containing at least one heteroatom of O, N, S, Si and P, and a C₃-C₃₀ aliphatic ring group.

11. The organic electronic device of claim 9, wherein Formula 2 is represented by one of Formula 2-1 to Formula 2-6: 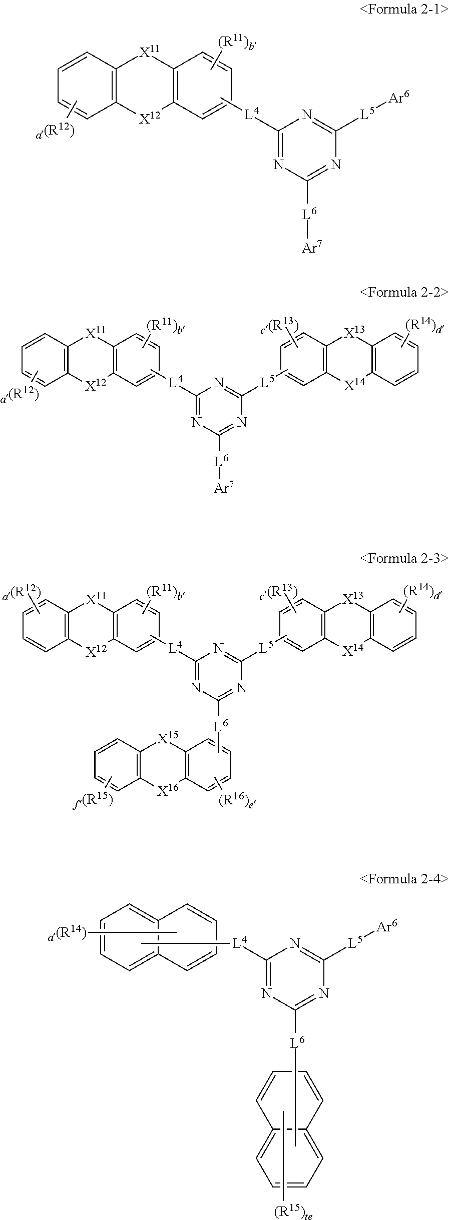 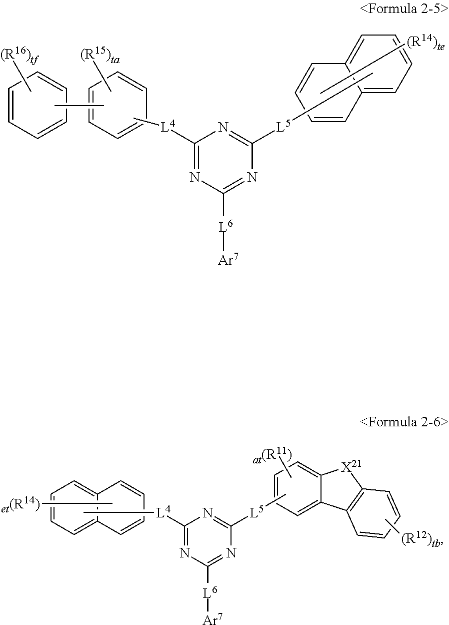
in Formula 2-1 to Formula 2-6, L⁴ to L⁶, Ar⁶, Ar⁷ are the same as defined in claim 10,
X¹¹, X¹³, X¹⁵ and X²¹ are independently NAr¹¹, O, S or C(R¹⁷)(R¹⁸),
X¹², X¹⁴ and X¹⁶ are independently a single bond, N(Ar¹²), O, S or C(R²¹)(R²²),
Ar¹¹ and Ar¹² are each independently selected from the group consisting of a C₆-C₃₀ aryl group, a fluorenyl group, a C₂-C₃₀ heterocyclic group containing at least one heteroatom of O, N, S, Si and P, and a C₃-C₃₀ aliphatic ring group,
R¹¹ to R¹⁸, R²¹, R²² are each independently selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent groups may be bonded to each other to form a ring, R¹⁷ and R¹⁸ may be bonded to each other to form a ring, and R²¹ and R²² may be bonded to each other to form a ring, and
a′, d′ and f′ are each an integer of 0 to 4, b′, c′ and e′ are each an integer of 0 to 3, ta, tb and td are each an integer of 0 to 4, te is an integer of 0 to 7, and tf is an integer of 0 to 5.

12. The organic electronic device of claim 9, wherein at least one of L⁴ to L⁶ is selected from the group consisting of the following Formula b-1 to b-13: 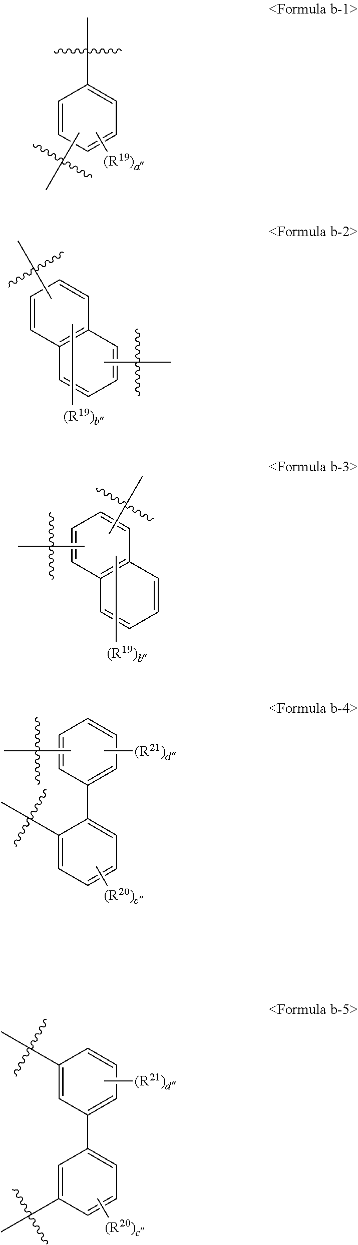 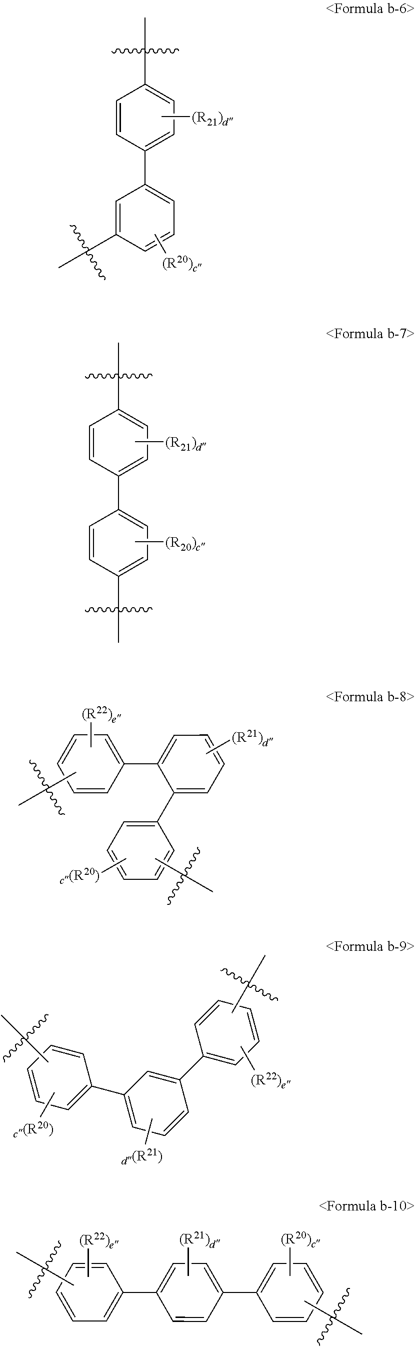 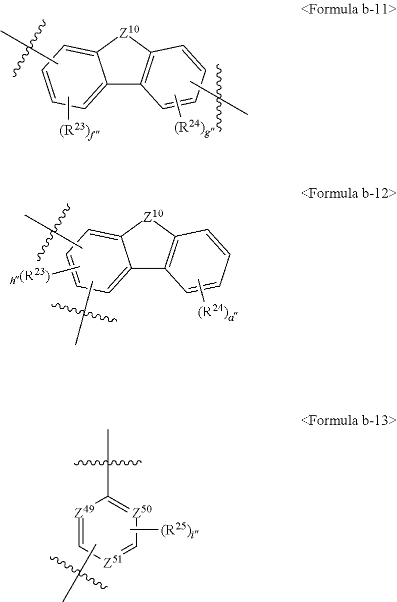
in Formula b-1 to Formula b-13,
Z¹⁰ is S, O, C(R₁)(R₂) or N(R₃),
Z⁴⁹ to Z⁵¹ are independently C(R₄) or N, and at least one of Z⁴⁹ to Z⁵¹ is N,
R¹⁹ to R²⁴, R₁ to R₄ are each independently selected from the group consisting of hydrogen, deuterium, halogen, a silane group unsubstituted or substituted with a C₁-C₂₀ alkyl group or a C₆-C₂₀ aryl group, a phosphine oxide substituted or unsubstituted with a C₁-C₂₀ alkyl group or a C₆-C₂₀ aryl group, siloxane group, a cyano group, a nitro group, C₁-C₂₀ alkylthio group, C₁-C₂₀ alkoxy group, C₆-C₃₀ aryloxy group, C₆-C₃₀ arylthio group, a C₁-C₂₀ alkyl group, a C₂-C₂₀ alkenyl group, a C₂-C₂₀ alkynyl group, a C₆-C₃₀ aryl group, a fluorenyl group, a C₂-C₃₀ heterocyclic group comprising at least one heteroatom selected from the group consisting of O, N, S, Si and P, and a C₃-C₃₀ aliphatic ring group, and adjacent groups may be bonded to each other to form a ring, and R₁ and R₂ may be bonded to each other to form a ring, and
a″, c″, d″ and e″ are each an integer of 0 to 4, b″ is an integer of 0 to 6, f″ and g″ are each an integer of 0 to 3, h″ is an integer of 0 to 2, and i″ is an integer of 0 to 3.

13. The organic electronic device of claim 9, wherein the compound represented by Formula 2 is one of the following compounds:

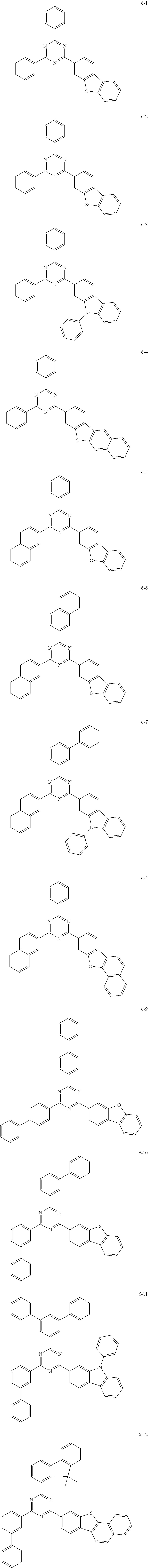

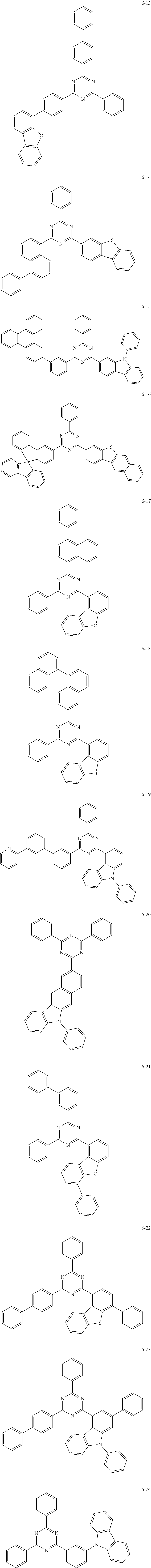

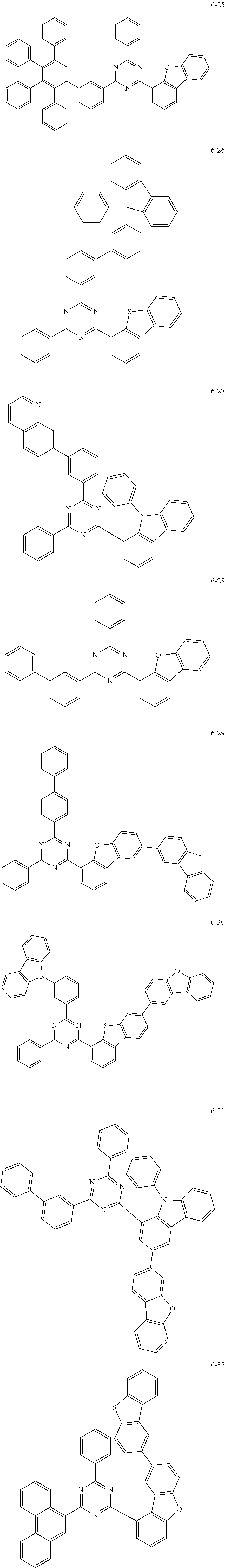

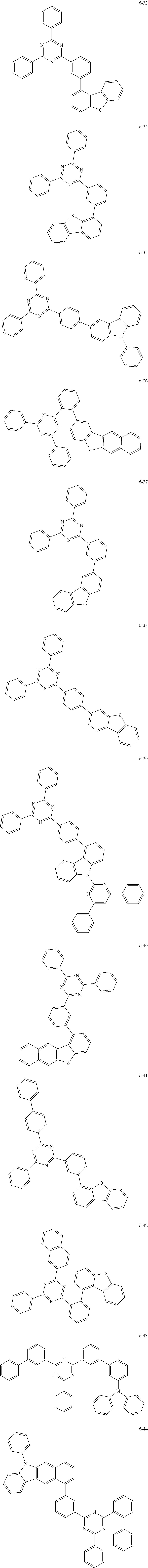

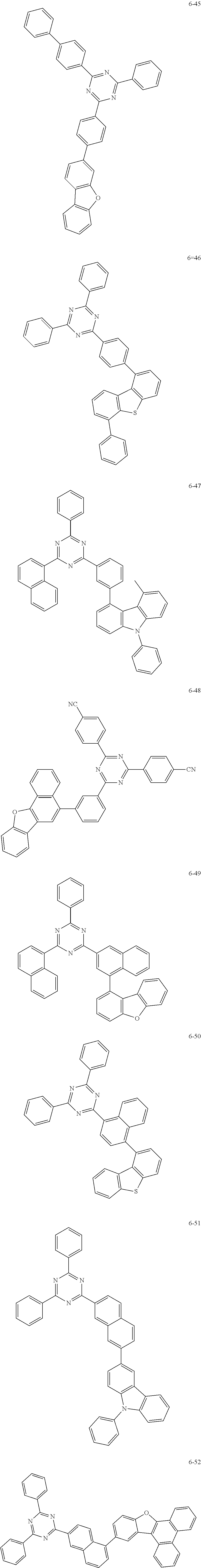

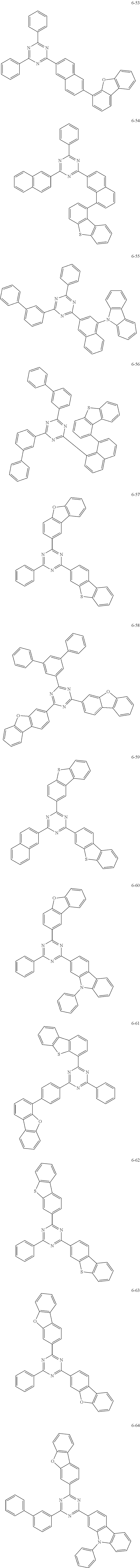

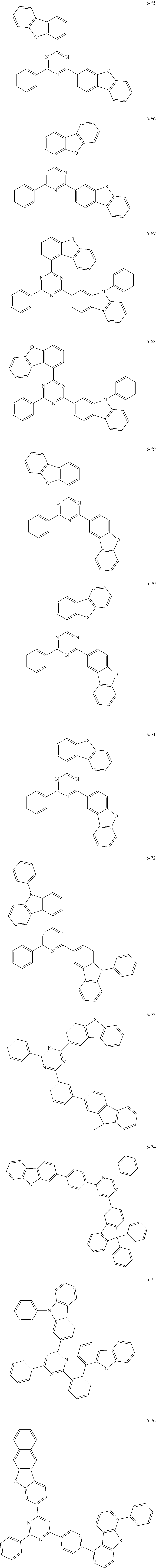

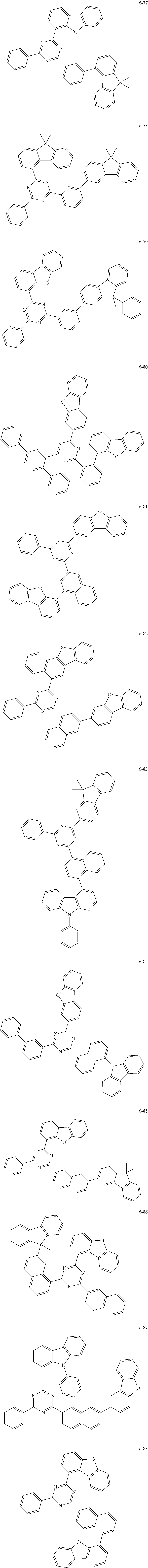

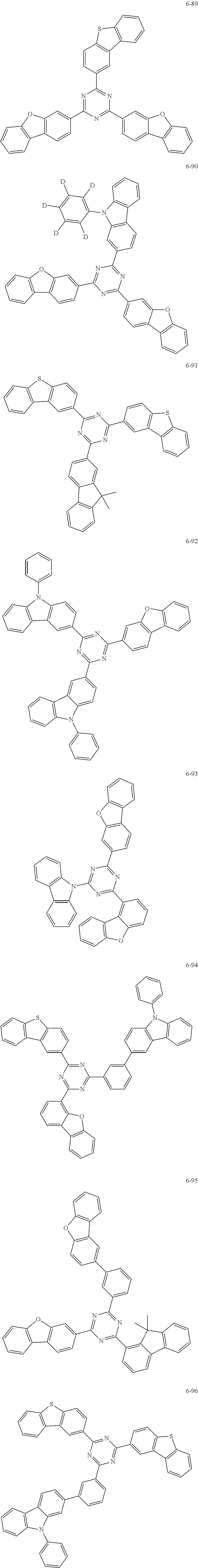

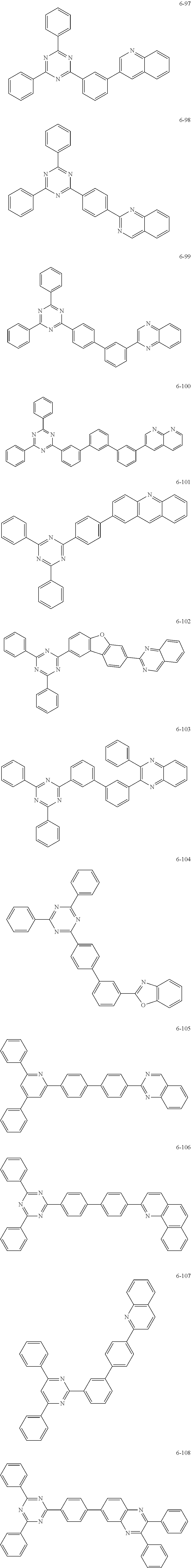

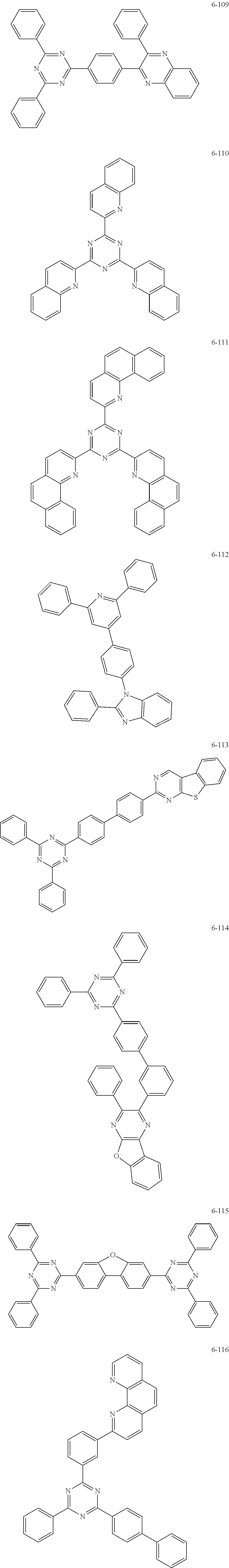

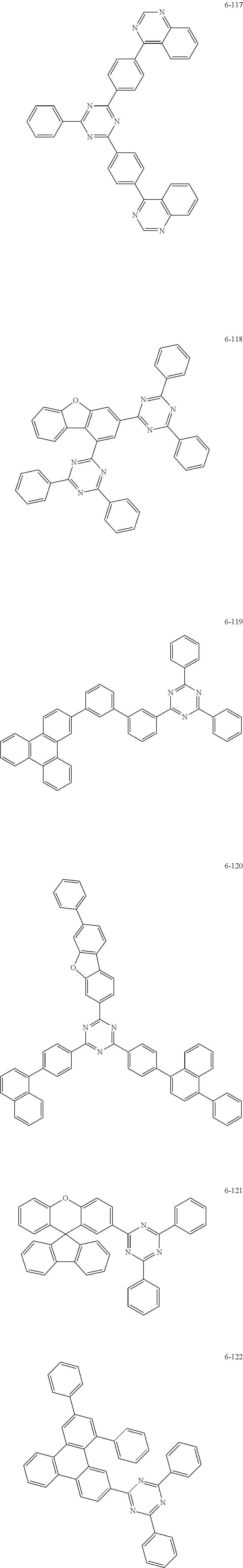

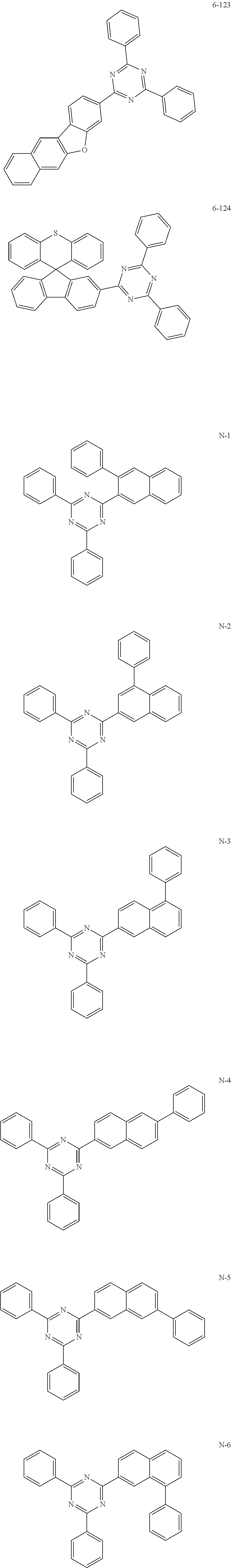

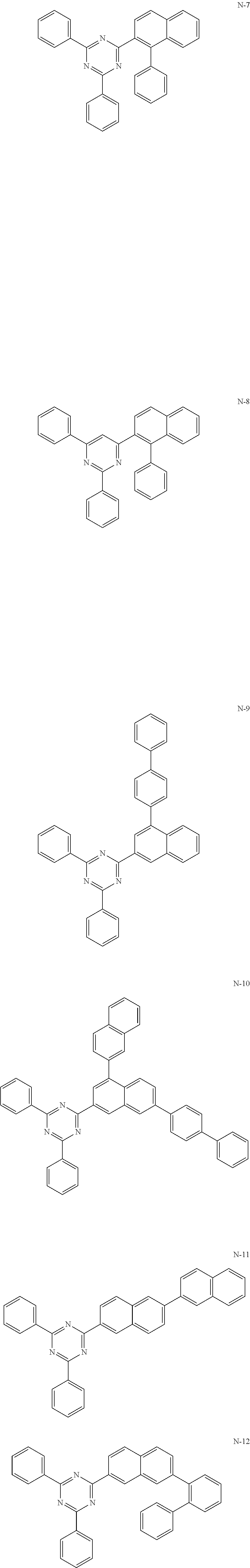 [SMILES: c1ccc(-c2nc(-c3ccccc3)nc(-c3ccc4ccccc4c3-c3ccccc3)n2)cc1 ; c1ccc(-c2cc(-c3ccc4ccccc4c3-c3ccccc3)nc(-c3ccccc3)n2)cc1 ; c1ccc(-c2ccc(-c3cc(-c4nc(-c5ccccc5)nc(-c5ccccc5)n4)cc4ccccc34)cc2)cc1 ; c1ccc(-c2ccc(-c3ccc4c(-c5ccc6ccccc6c5)cc(-c5nc(-c6ccccc6)nc(-c6ccccc6)n5)cc4c3)cc2)cc1 ; c1ccc(-c2nc(-c3ccccc3)nc(-c3ccc4cc(-c5ccc6ccccc6c5)ccc4c3)n2)cc1 ; c1ccc(-c2nc(-c3ccccc3)nc(-c3ccc4ccc(-c5ccccc5-c5ccccc5)cc4c3)n2)cc1]

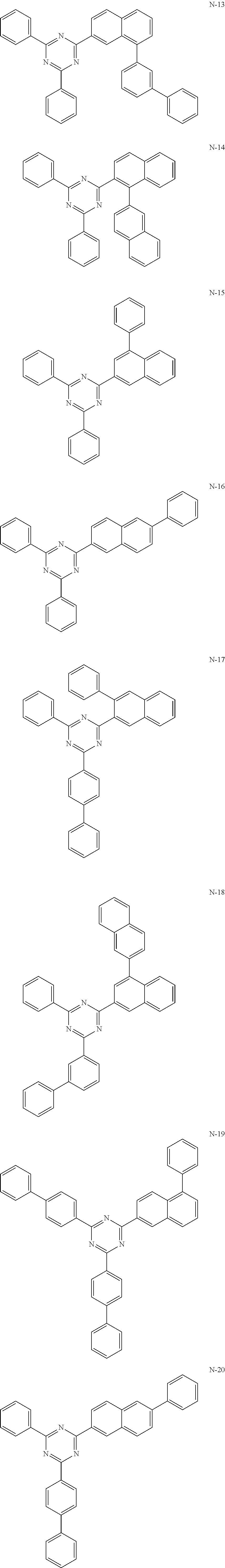

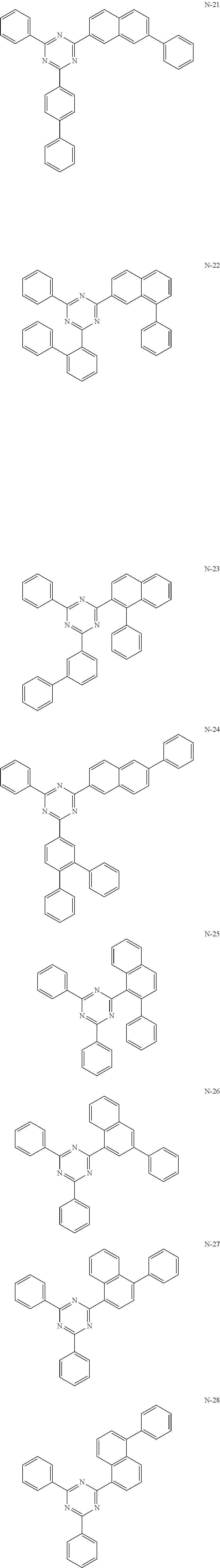

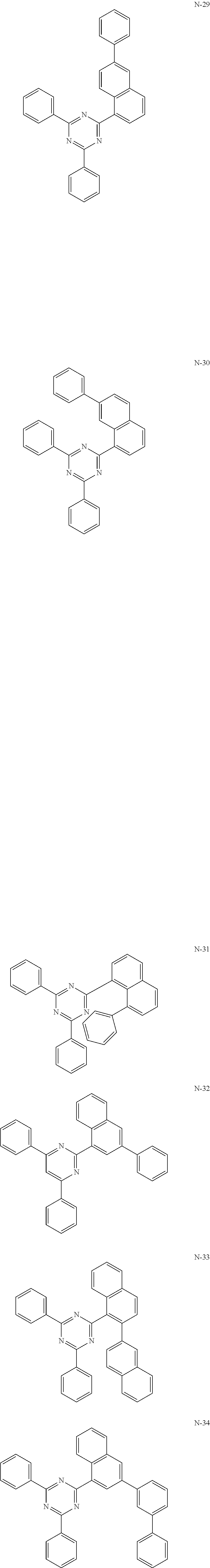 [SMILES: c1ccc(-c2ccc3c(-c4nc(-c5ccccc5)nc(-c5ccccc5)n4)cccc3c2)cc1 ; c1ccc(-c2ccc3cccc(-c4nc(-c5ccccc5)nc(-c5ccccc5)n4)c3c2)cc1 ; c1ccc(-c2nc(-c3ccccc3)nc(-c3cccc4cccc(-c5ccccc5)c34)n2)cc1 ; c1ccc(-c2cc(-c3nc(-c4ccccc4)cc(-c4ccccc4)n3)c3ccccc3c2)cc1 ; c1ccc(-c2nc(-c3ccccc3)nc(-c3c(-c4ccc5ccccc5c4)ccc4ccccc34)n2)cc1 ; c1ccc(-c2cccc(-c3cc(-c4nc(-c5ccccc5)nc(-c5ccccc5)n4)c4ccccc4c3)c2)cc1]

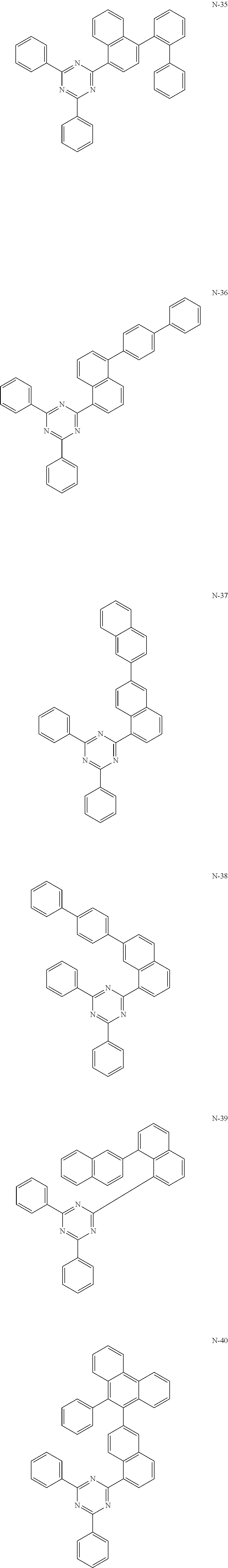 [SMILES: c1ccc(-c2nc(-c3ccccc3)nc(-c3ccc(-c4ccccc4-c4ccccc4)c4ccccc34)n2)cc1 ; c1ccc(-c2ccc(-c3cccc4c(-c5nc(-c6ccccc6)nc(-c6ccccc6)n5)cccc34)cc2)cc1 ; c1ccc(-c2nc(-c3ccccc3)nc(-c3cccc4cc(-c5ccc6ccccc6c5)ccc34)n2)cc1 ; c1ccc(-c2ccc(-c3ccc4cccc(-c5nc(-c6ccccc6)nc(-c6ccccc6)n5)c4c3)cc2)cc1 ; c1ccc(-c2nc(-c3ccccc3)nc(-c3cccc4cccc(-c5ccc6ccccc6c5)c34)n2)cc1 ; c1ccc(-c2nc(-c3ccccc3)nc(-c3cccc4cc(-c5c(-c6ccccc6)c6ccccc6c6ccccc56)ccc34)n2)cc1]

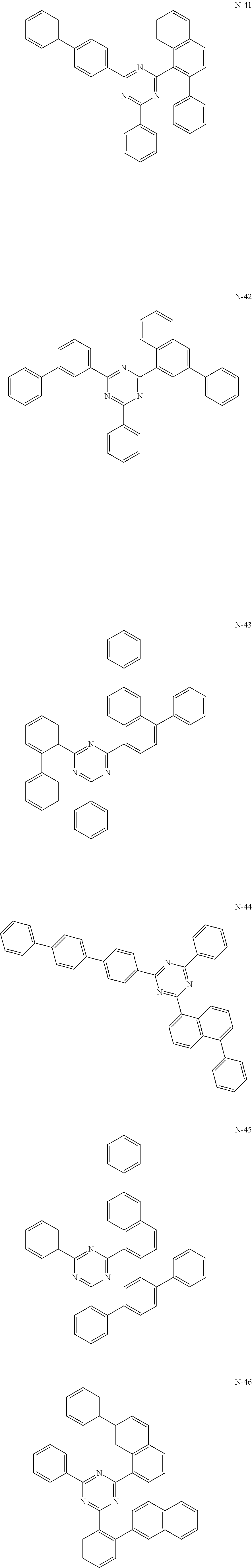 [SMILES: c1ccc(-c2ccc(-c3nc(-c4ccccc4)nc(-c4c(-c5ccccc5)ccc5ccccc45)n3)cc2)cc1 ; c1ccc(-c2cccc(-c3nc(-c4ccccc4)nc(-c4cc(-c5ccccc5)cc5ccccc45)n3)c2)cc1 ; c1ccc(-c2ccc3c(-c4nc(-c5ccccc5)nc(-c5ccccc5-c5ccccc5)n4)ccc(-c4ccccc4)c3c2)cc1 ; c1ccc(-c2ccc(-c3ccc(-c4nc(-c5ccccc5)nc(-c5cccc6c(-c7ccccc7)cccc56)n4)cc3)cc2)cc1 ; c1ccc(-c2ccc(-c3ccccc3-c3nc(-c4ccccc4)nc(-c4cccc5cc(-c6ccccc6)ccc45)n3)cc2)cc1 ; c1ccc(-c2ccc3cccc(-c4nc(-c5ccccc5)nc(-c5ccccc5-c5ccc6ccccc6c5)n4)c3c2)cc1]

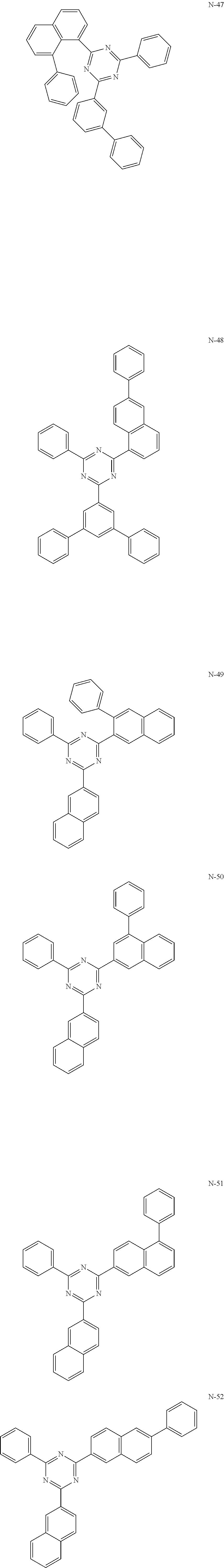 [SMILES: c1ccc(-c2cccc(-c3nc(-c4ccccc4)nc(-c4cccc5cccc(-c6ccccc6)c45)n3)c2)cc1 ; c1ccc(-c2cc(-c3ccccc3)cc(-c3nc(-c4ccccc4)nc(-c4cccc5cc(-c6ccccc6)ccc45)n3)c2)cc1 ; c1ccc(-c2nc(-c3ccc4ccccc4c3)nc(-c3cc4ccccc4cc3-c3ccccc3)n2)cc1 ; c1ccc(-c2nc(-c3ccc4ccccc4c3)nc(-c3cc(-c4ccccc4)c4ccccc4c3)n2)cc1 ; c1ccc(-c2nc(-c3ccc4ccccc4c3)nc(-c3ccc4c(-c5ccccc5)cccc4c3)n2)cc1 ; c1ccc(-c2ccc3cc(-c4nc(-c5ccccc5)nc(-c5ccc6ccccc6c5)n4)ccc3c2)cc1]

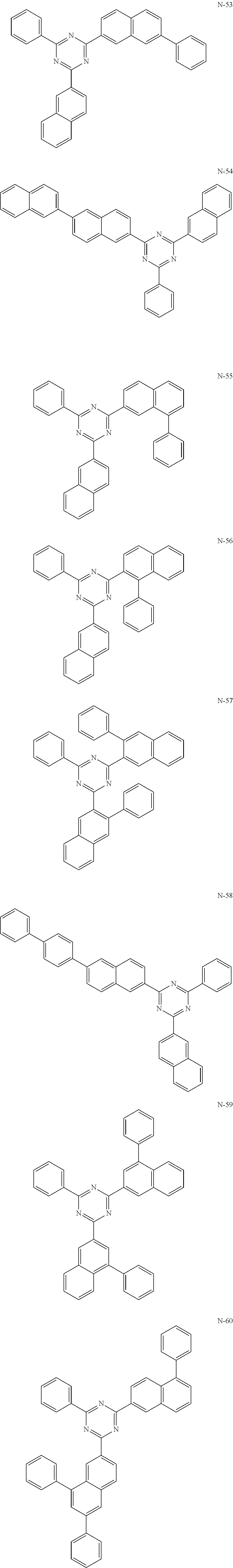

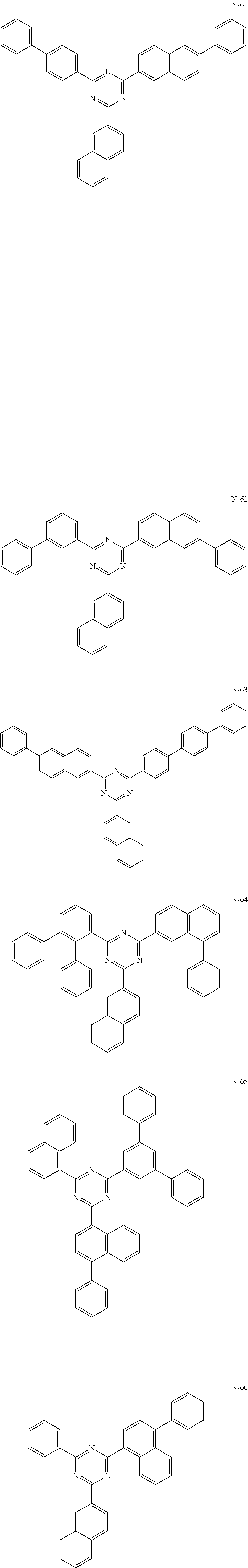 [SMILES: c1ccc(-c2ccc(-c3nc(-c4ccc5ccccc5c4)nc(-c4ccc5cc(-c6ccccc6)ccc5c4)n3)cc2)cc1 ; c1ccc(-c2cccc(-c3nc(-c4ccc5ccccc5c4)nc(-c4ccc5ccc(-c6ccccc6)cc5c4)n3)c2)cc1 ; c1ccc(-c2ccc(-c3ccc(-c4nc(-c5ccc6ccccc6c5)nc(-c5ccc6cc(-c7ccccc7)ccc6c5)n4)cc3)cc2)cc1 ; c1ccc(-c2cccc(-c3nc(-c4ccc5ccccc5c4)nc(-c4ccc5cccc(-c6ccccc6)c5c4)n3)c2-c2ccccc2)cc1 ; c1ccc(-c2cc(-c3ccccc3)cc(-c3nc(-c4cccc5ccccc45)nc(-c4ccc(-c5ccccc5)c5ccccc45)n3)c2)cc1 ; c1ccc(-c2nc(-c3ccc4ccccc4c3)nc(-c3ccc(-c4ccccc4)c4ccccc34)n2)cc1]

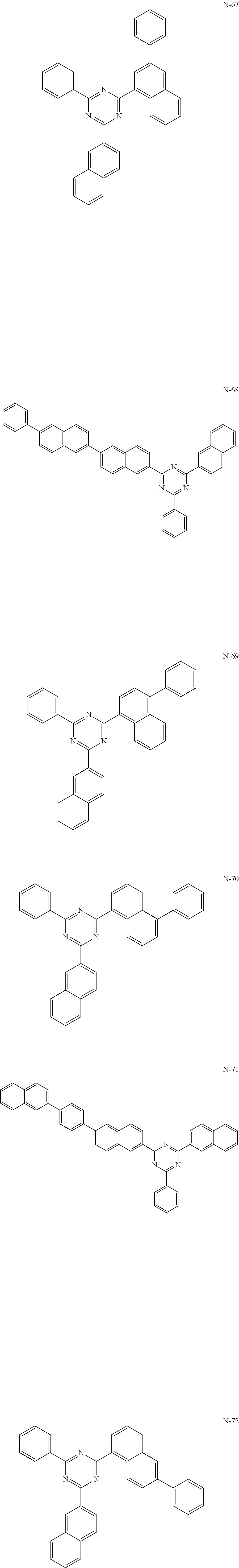 [SMILES: c1ccc(-c2cc(-c3nc(-c4ccccc4)nc(-c4ccc5ccccc5c4)n3)c3ccccc3c2)cc1 ; c1ccc(-c2ccc3cc(-c4ccc5cc(-c6nc(-c7ccccc7)nc(-c7ccc8ccccc8c7)n6)ccc5c4)ccc3c2)cc1 ; c1ccc(-c2nc(-c3ccc4ccccc4c3)nc(-c3ccc(-c4ccccc4)c4ccccc34)n2)cc1 ; c1ccc(-c2nc(-c3ccc4ccccc4c3)nc(-c3cccc4c(-c5ccccc5)cccc34)n2)cc1 ; c1ccc(-c2nc(-c3ccc4ccccc4c3)nc(-c3ccc4cc(-c5ccc(-c6ccc7ccccc7c6)cc5)ccc4c3)n2)cc1 ; c1ccc(-c2ccc3c(-c4nc(-c5ccccc5)nc(-c5ccc6ccccc6c5)n4)cccc3c2)cc1]

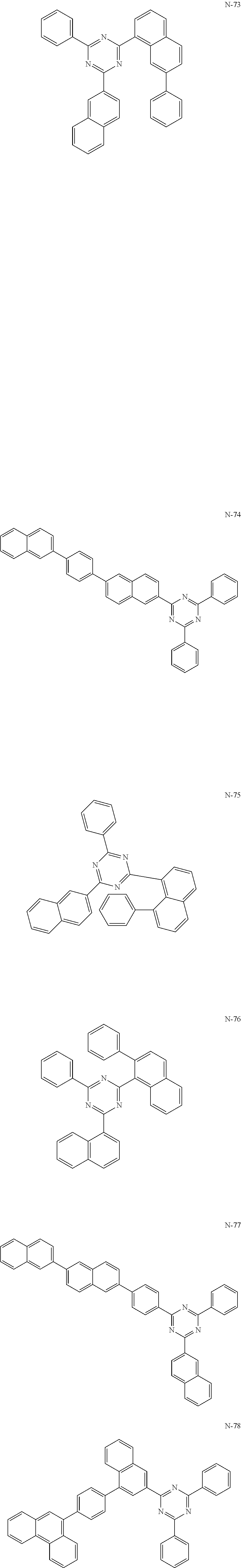 [SMILES: c1ccc(-c2ccc3cccc(-c4nc(-c5ccccc5)nc(-c5ccc6ccccc6c5)n4)c3c2)cc1 ; c1ccc(-c2nc(-c3ccccc3)nc(-c3ccc4cc(-c5ccc(-c6ccc7ccccc7c6)cc5)ccc4c3)n2)cc1 ; c1ccc(-c2nc(-c3ccc4ccccc4c3)nc(-c3cccc4cccc(-c5ccccc5)c34)n2)cc1 ; c1ccc(-c2nc(-c3cccc4ccccc34)nc(-c3c(-c4ccccc4)ccc4ccccc34)n2)cc1 ; c1ccc(-c2nc(-c3ccc(-c4ccc5cc(-c6ccc7ccccc7c6)ccc5c4)cc3)nc(-c3ccc4ccccc4c3)n2)cc1 ; c1ccc(-c2nc(-c3ccccc3)nc(-c3cc(-c4ccc(-c5cc6ccccc6c6ccccc56)cc4)c4ccccc4c3)n2)cc1]

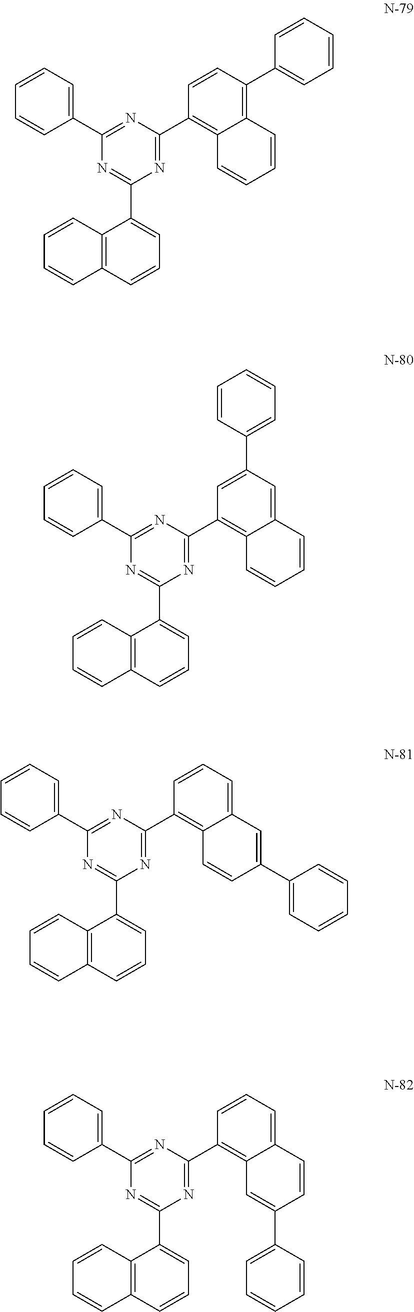 [SMILES: c1ccc(-c2nc(-c3cccc4ccccc34)nc(-c3ccc(-c4ccccc4)c4ccccc34)n2)cc1 ; c1ccc(-c2cc(-c3nc(-c4ccccc4)nc(-c4cccc5ccccc45)n3)c3ccccc3c2)cc1 ; c1ccc(-c2ccc3c(-c4nc(-c5ccccc5)nc(-c5cccc6ccccc56)n4)cccc3c2)cc1 ; c1ccc(-c2ccc3cccc(-c4nc(-c5ccccc5)nc(-c5cccc6ccccc56)n4)c3c2)cc1]

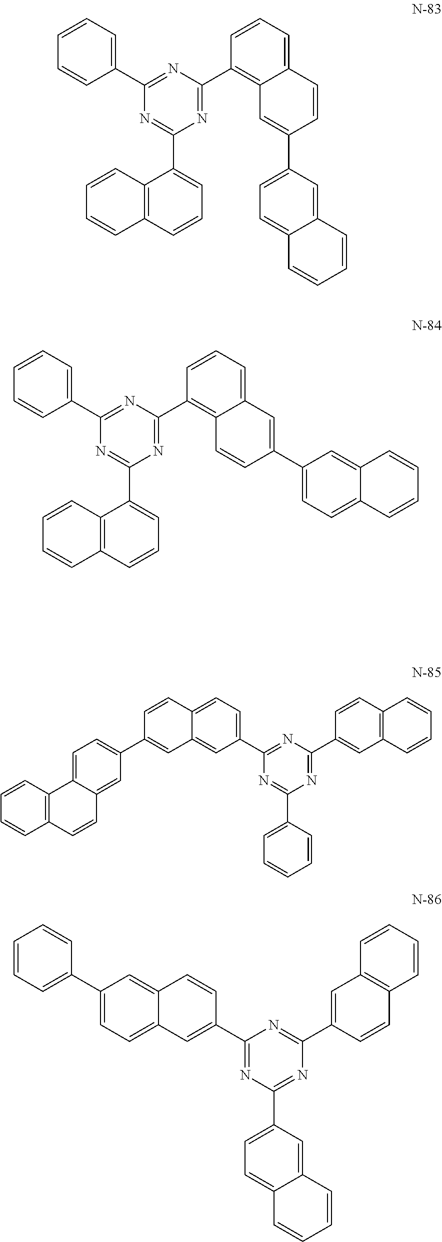 [SMILES: c1ccc(-c2nc(-c3cccc4ccccc34)nc(-c3cccc4ccc(-c5ccc6ccccc6c5)cc34)n2)cc1 ; c1ccc(-c2nc(-c3cccc4ccccc34)nc(-c3cccc4cc(-c5ccc6ccccc6c5)ccc34)n2)cc1 ; c1ccc(-c2nc(-c3ccc4ccccc4c3)nc(-c3ccc4ccc(-c5ccc6c(ccc7ccccc76)c5)cc4c3)n2)cc1 ; c1ccc(-c2ccc3cc(-c4nc(-c5ccc6ccccc6c5)nc(-c5ccc6ccccc6c5)n4)ccc3c2)cc1]

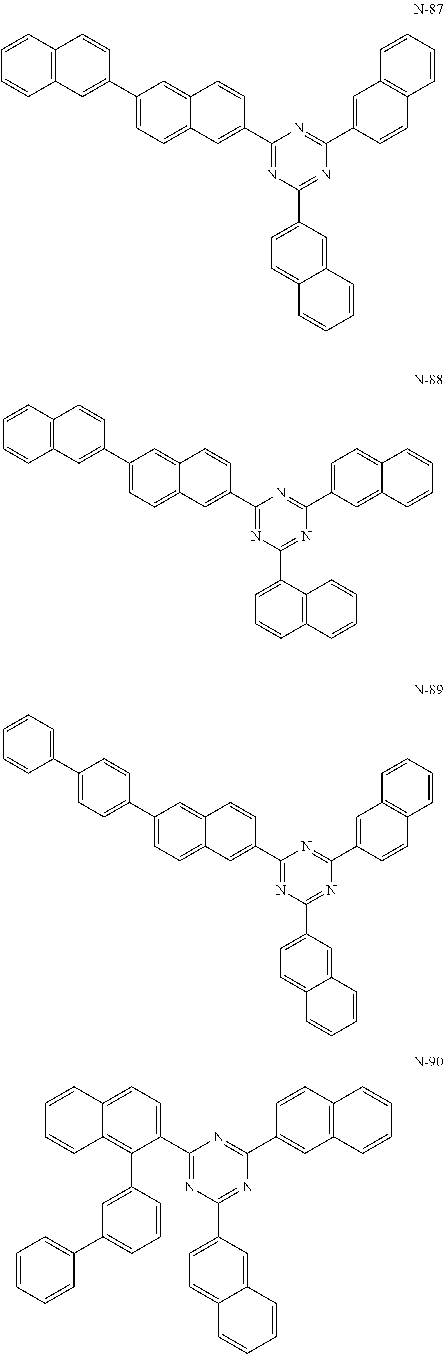 [SMILES: c1ccc2cc(-c3ccc4cc(-c5nc(-c6ccc7ccccc7c6)nc(-c6ccc7ccccc7c6)n5)ccc4c3)ccc2c1 ; c1ccc2cc(-c3ccc4cc(-c5nc(-c6ccc7ccccc7c6)nc(-c6cccc7ccccc67)n5)ccc4c3)ccc2c1 ; c1ccc(-c2ccc(-c3ccc4cc(-c5nc(-c6ccc7ccccc7c6)nc(-c6ccc7ccccc7c6)n5)ccc4c3)cc2)cc1 ; c1ccc(-c2cccc(-c3c(-c4nc(-c5ccc6ccccc6c5)nc(-c5ccc6ccccc6c5)n4)ccc4ccccc34)c2)cc1]

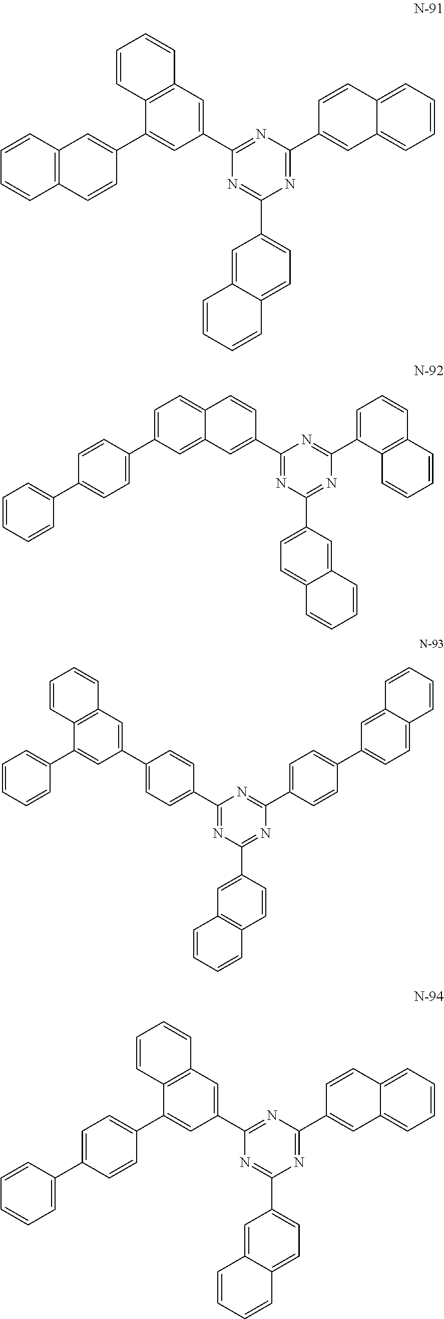 [SMILES: c1ccc2cc(-c3nc(-c4ccc5ccccc5c4)nc(-c4cc(-c5ccc6ccccc6c5)c5ccccc5c4)n3)ccc2c1 ; c1ccc(-c2ccc(-c3ccc4ccc(-c5nc(-c6ccc7ccccc7c6)nc(-c6cccc7ccccc67)n5)cc4c3)cc2)cc1 ; c1ccc(-c2cc(-c3ccc(-c4nc(-c5ccc(-c6ccc7ccccc7c6)cc5)nc(-c5ccc6ccccc6c5)n4)cc3)cc3ccccc23)cc1 ; c1ccc(-c2ccc(-c3cc(-c4nc(-c5ccc6ccccc6c5)nc(-c5ccc6ccccc6c5)n4)cc4ccccc34)cc2)cc1]

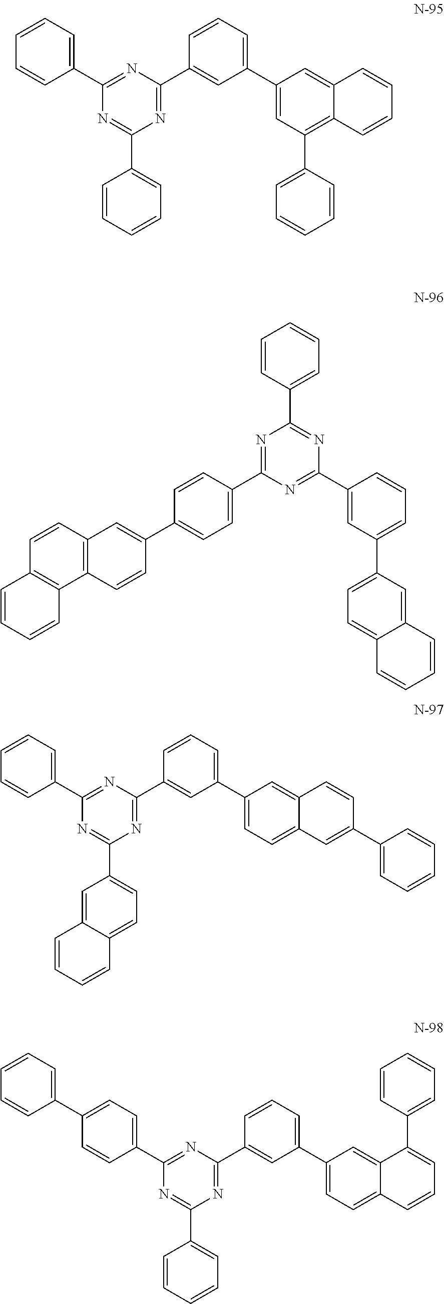 [SMILES: c1ccc(-c2nc(-c3ccccc3)nc(-c3cccc(-c4cc(-c5ccccc5)c5ccccc5c4)c3)n2)cc1 ; c1ccc(-c2nc(-c3ccc(-c4ccc5c(ccc6ccccc65)c4)cc3)nc(-c3cccc(-c4ccc5ccccc5c4)c3)n2)cc1 ; c1ccc(-c2ccc3cc(-c4cccc(-c5nc(-c6ccccc6)nc(-c6ccc7ccccc7c6)n5)c4)ccc3c2)cc1 ; c1ccc(-c2ccc(-c3nc(-c4ccccc4)nc(-c4cccc(-c5ccc6cccc(-c7ccccc7)c6c5)c4)n3)cc2)cc1]

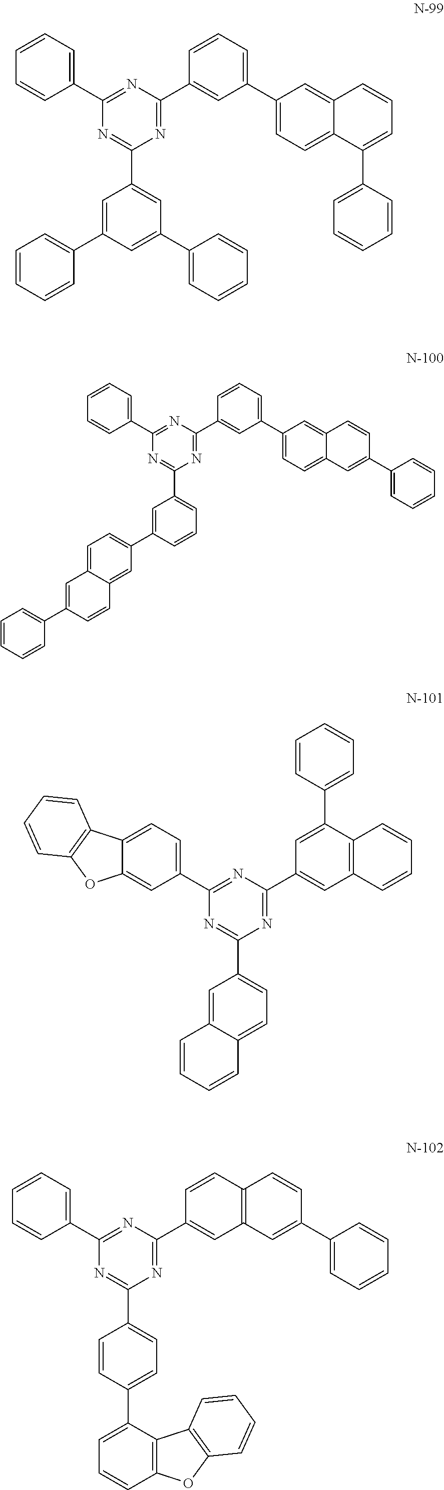 [SMILES: c1ccc(-c2cc(-c3ccccc3)cc(-c3nc(-c4ccccc4)nc(-c4cccc(-c5ccc6c(-c7ccccc7)cccc6c5)c4)n3)c2)cc1 ; c1ccc(-c2ccc3cc(-c4cccc(-c5nc(-c6ccccc6)nc(-c6cccc(-c7ccc8cc(-c9ccccc9)ccc8c7)c6)n5)c4)ccc3c2)cc1 ; c1ccc(-c2cc(-c3nc(-c4ccc5ccccc5c4)nc(-c4ccc5c(c4)oc4ccccc45)n3)cc3ccccc23)cc1 ; c1ccc(-c2ccc3ccc(-c4nc(-c5ccccc5)nc(-c5ccc(-c6cccc7oc8ccccc8c67)cc5)n4)cc3c2)cc1]

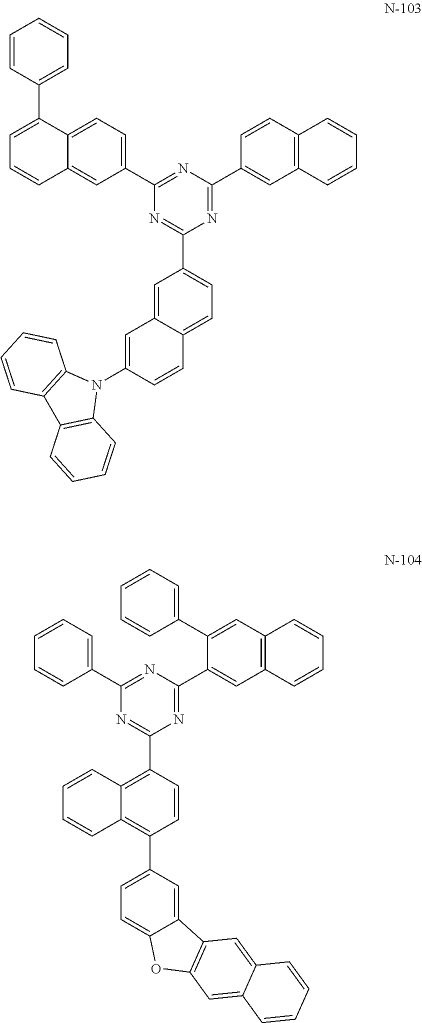 [SMILES: c1ccc(-c2cccc3cc(-c4nc(-c5ccc6ccccc6c5)nc(-c5ccc6ccc(-n7c8ccccc8c8ccccc87)cc6c5)n4)ccc23)cc1 ; c1ccc(-c2nc(-c3cc4ccccc4cc3-c3ccccc3)nc(-c3ccc(-c4ccc5oc6cc7ccccc7cc6c5c4)c4ccccc34)n2)cc1]

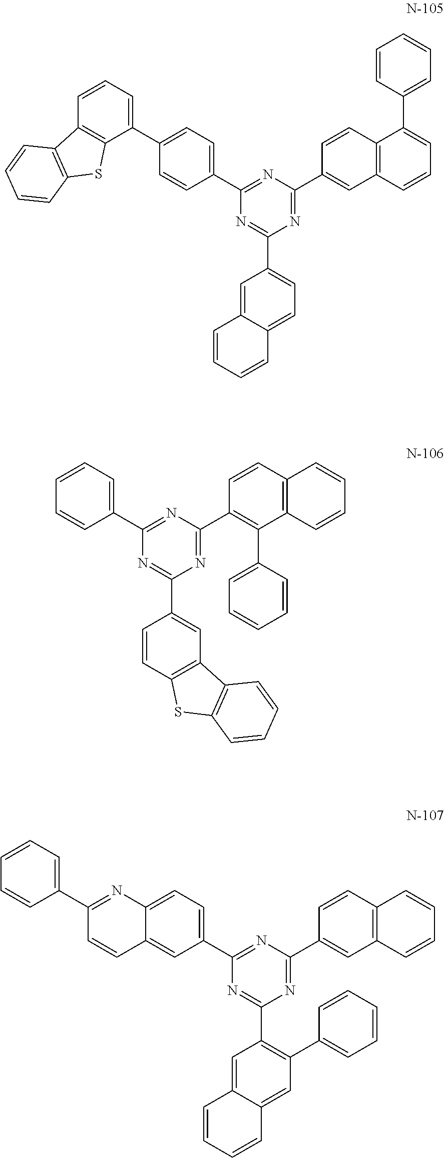 [SMILES: c1ccc(-c2cccc3cc(-c4nc(-c5ccc(-c6cccc7c6sc6ccccc67)cc5)nc(-c5ccc6ccccc6c5)n4)ccc23)cc1 ; c1ccc(-c2nc(-c3ccc4sc5ccccc5c4c3)nc(-c3ccc4ccccc4c3-c3ccccc3)n2)cc1 ; c1ccc(-c2ccc3cc(-c4nc(-c5ccc6ccccc6c5)nc(-c5cc6ccccc6cc5-c5ccccc5)n4)ccc3n2)cc1]

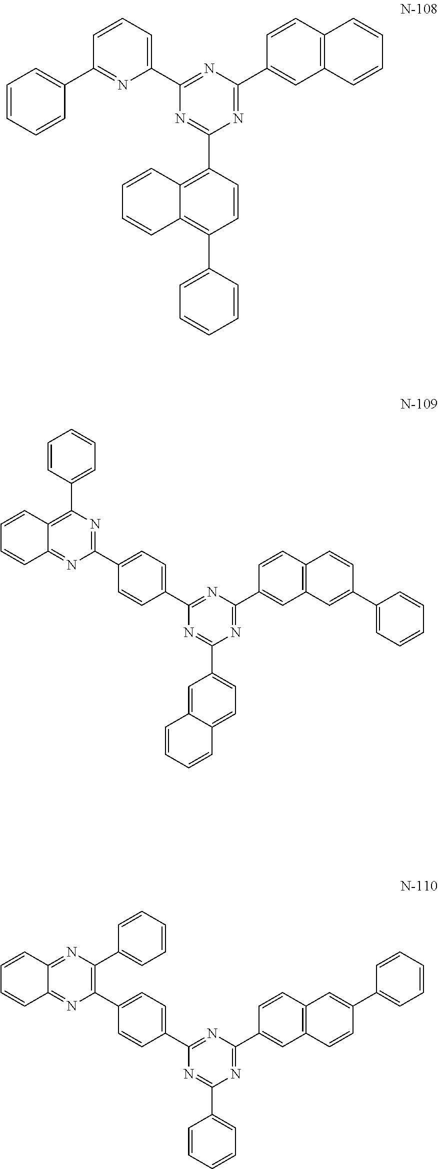 [SMILES: c1ccc(-c2cccc(-c3nc(-c4ccc5ccccc5c4)nc(-c4ccc(-c5ccccc5)c5ccccc45)n3)n2)cc1 ; c1ccc(-c2ccc3ccc(-c4nc(-c5ccc(-c6nc(-c7ccccc7)c7ccccc7n6)cc5)nc(-c5ccc6ccccc6c5)n4)cc3c2)cc1 ; c1ccc(-c2ccc3cc(-c4nc(-c5ccccc5)nc(-c5ccc(-c6nc7ccccc7nc6-c6ccccc6)cc5)n4)ccc3c2)cc1]

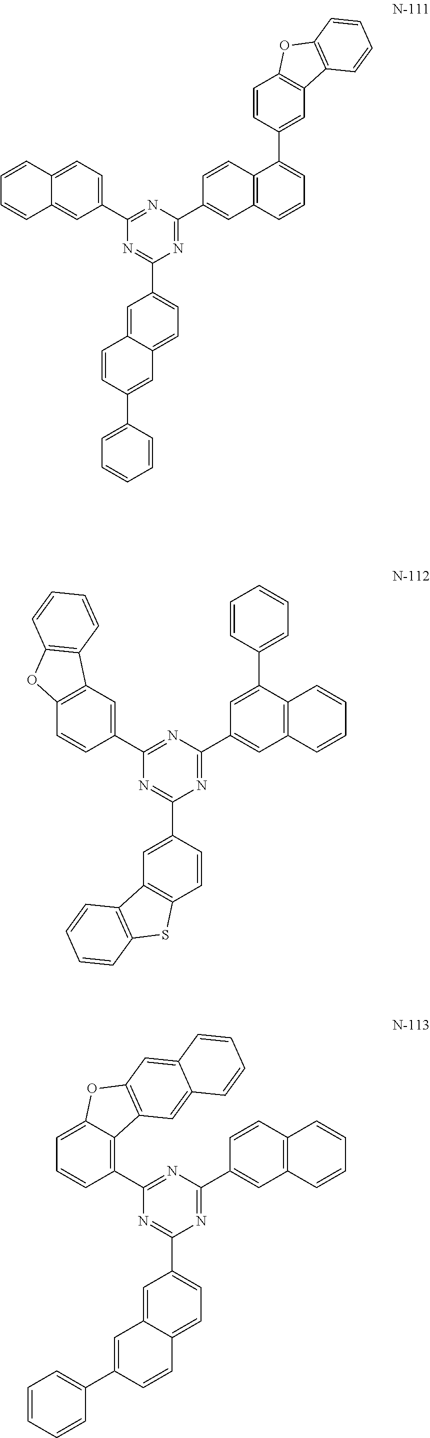 [SMILES: c1ccc(-c2ccc3cc(-c4nc(-c5ccc6ccccc6c5)nc(-c5ccc6c(-c7ccc8oc9ccccc9c8c7)cccc6c5)n4)ccc3c2)cc1 ; c1ccc(-c2cc(-c3nc(-c4ccc5oc6ccccc6c5c4)nc(-c4ccc5sc6ccccc6c5c4)n3)cc3ccccc23)cc1 ; c1ccc(-c2ccc3ccc(-c4nc(-c5ccc6ccccc6c5)nc(-c5cccc6oc7cc8ccccc8cc7c56)n4)cc3c2)cc1]

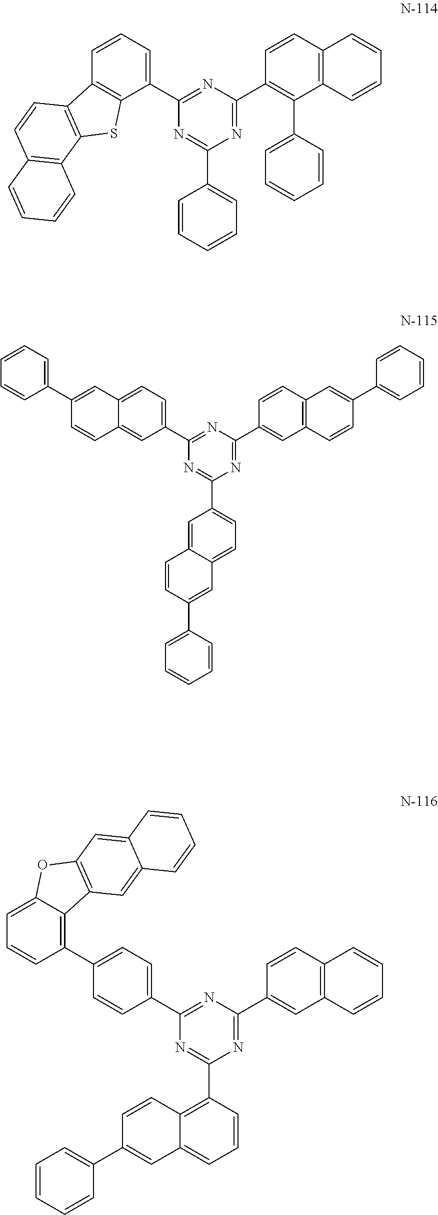 [SMILES: c1ccc(-c2nc(-c3ccc4ccccc4c3-c3ccccc3)nc(-c3cccc4c3sc3c5ccccc5ccc43)n2)cc1 ; c1ccc(-c2ccc3cc(-c4nc(-c5ccc6cc(-c7ccccc7)ccc6c5)nc(-c5ccc6cc(-c7ccccc7)ccc6c5)n4)ccc3c2)cc1 ; c1ccc(-c2ccc3c(-c4nc(-c5ccc(-c6cccc7oc8cc9ccccc9cc8c67)cc5)nc(-c5ccc6ccccc6c5)n4)cccc3c2)cc1]

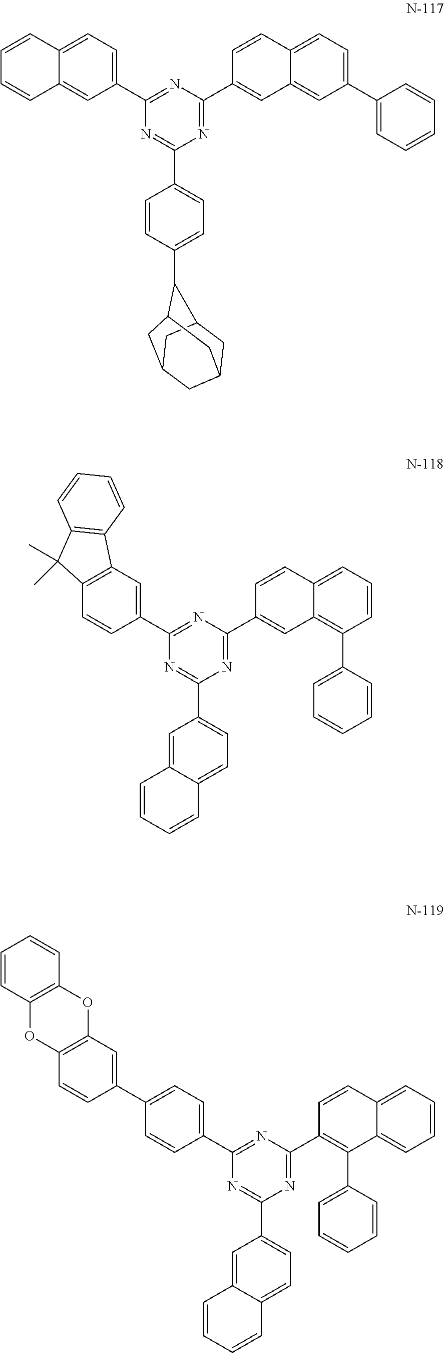 [SMILES: c1ccc(-c2ccc3ccc(-c4nc(-c5ccc(C6C7CC8CC(C7)CC6C8)cc5)nc(-c5ccc6ccccc6c5)n4)cc3c2)cc1 ; CC1(C)c2ccccc2-c2cc(-c3nc(-c4ccc5ccccc5c4)nc(-c4ccc5cccc(-c6ccccc6)c5c4)n3)ccc21 ; c1ccc(-c2c(-c3nc(-c4ccc(-c5ccc6c(c5)Oc5ccccc5O6)cc4)nc(-c4ccc5ccccc5c4)n3)ccc3ccccc23)cc1]

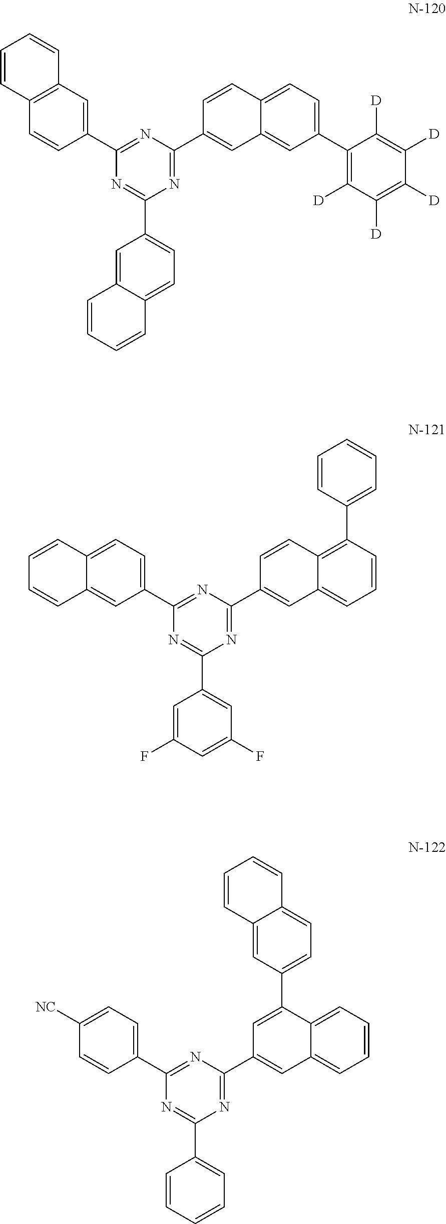 [SMILES: [2H]c1c([2H])c([2H])c(-c2ccc3ccc(-c4nc(-c5ccc6ccccc6c5)nc(-c5ccc6ccccc6c5)n4)cc3c2)c([2H])c1[2H] ; Fc1cc(F)cc(-c2nc(-c3ccc4ccccc4c3)nc(-c3ccc4c(-c5ccccc5)cccc4c3)n2)c1 ; N#Cc1ccc(-c2nc(-c3ccccc3)nc(-c3cc(-c4ccc5ccccc5c4)c4ccccc4c3)n2)cc1]

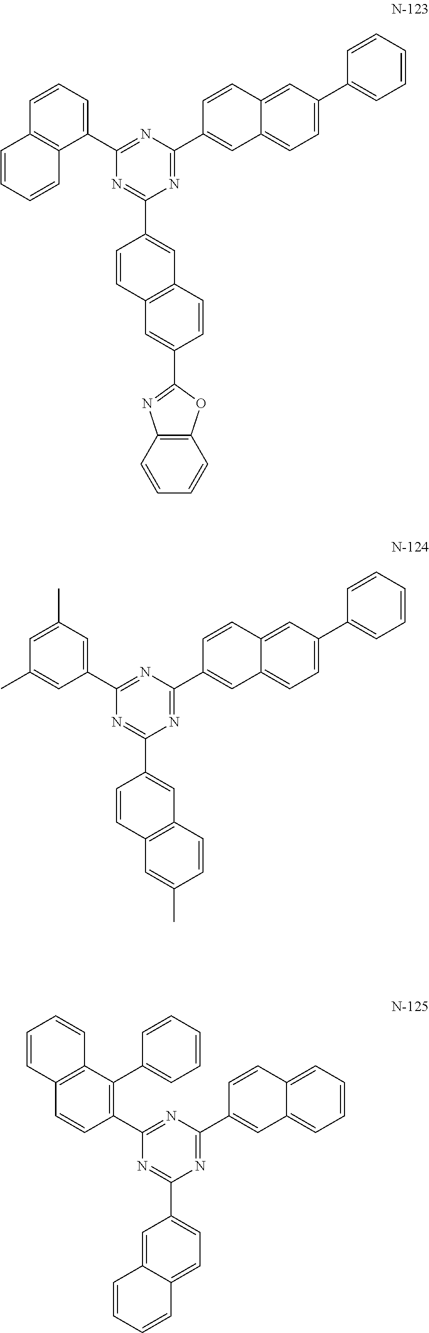 [SMILES: c1ccc(-c2ccc3cc(-c4nc(-c5ccc6cc(-c7nc8ccccc8o7)ccc6c5)nc(-c5cccc6ccccc56)n4)ccc3c2)cc1 ; Cc1cc(C)cc(-c2nc(-c3ccc4cc(C)ccc4c3)nc(-c3ccc4cc(-c5ccccc5)ccc4c3)n2)c1 ; c1ccc(-c2c(-c3nc(-c4ccc5ccccc5c4)nc(-c4ccc5ccccc5c4)n3)ccc3ccccc23)cc1]

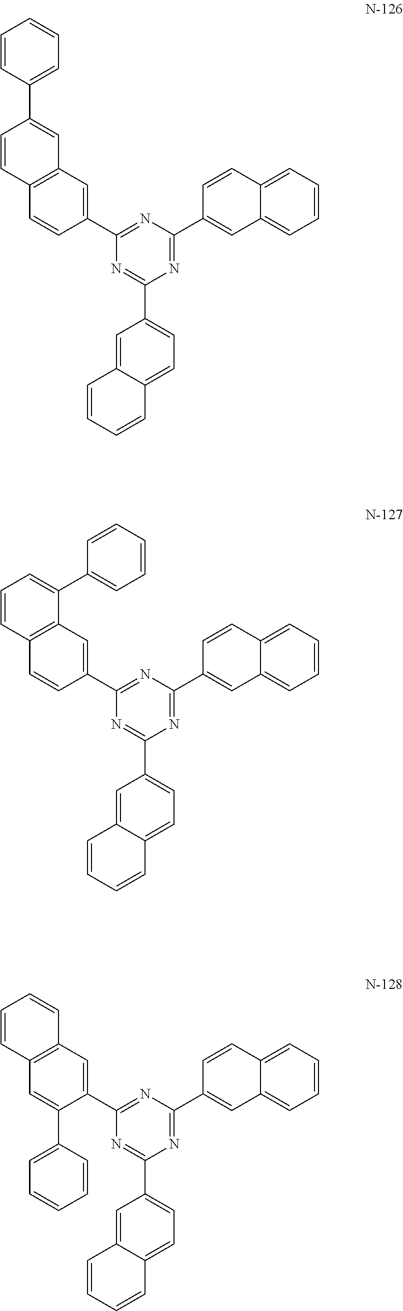 [SMILES: c1ccc(-c2ccc3ccc(-c4nc(-c5ccc6ccccc6c5)nc(-c5ccc6ccccc6c5)n4)cc3c2)cc1 ; c1ccc(-c2cccc3ccc(-c4nc(-c5ccc6ccccc6c5)nc(-c5ccc6ccccc6c5)n4)cc23)cc1 ; c1ccc(-c2cc3ccccc3cc2-c2nc(-c3ccc4ccccc4c3)nc(-c3ccc4ccccc4c3)n2)cc1]

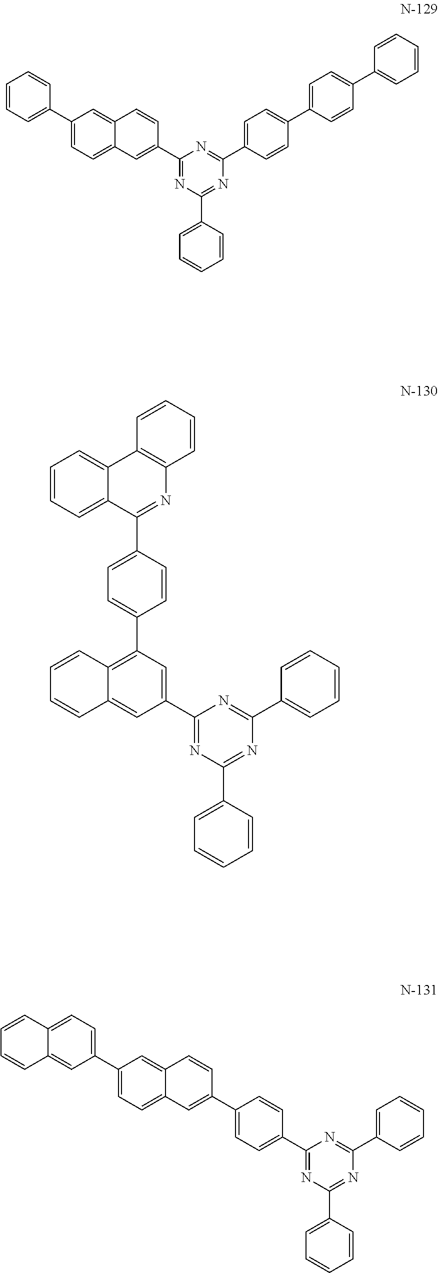 [SMILES: c1ccc(-c2ccc(-c3ccc(-c4nc(-c5ccccc5)nc(-c5ccc6cc(-c7ccccc7)ccc6c5)n4)cc3)cc2)cc1 ; c1ccc(-c2nc(-c3ccccc3)nc(-c3cc(-c4ccc(-c5nc6ccccc6c6ccccc56)cc4)c4ccccc4c3)n2)cc1 ; c1ccc(-c2nc(-c3ccccc3)nc(-c3ccc(-c4ccc5cc(-c6ccc7ccccc7c6)ccc5c4)cc3)n2)cc1]

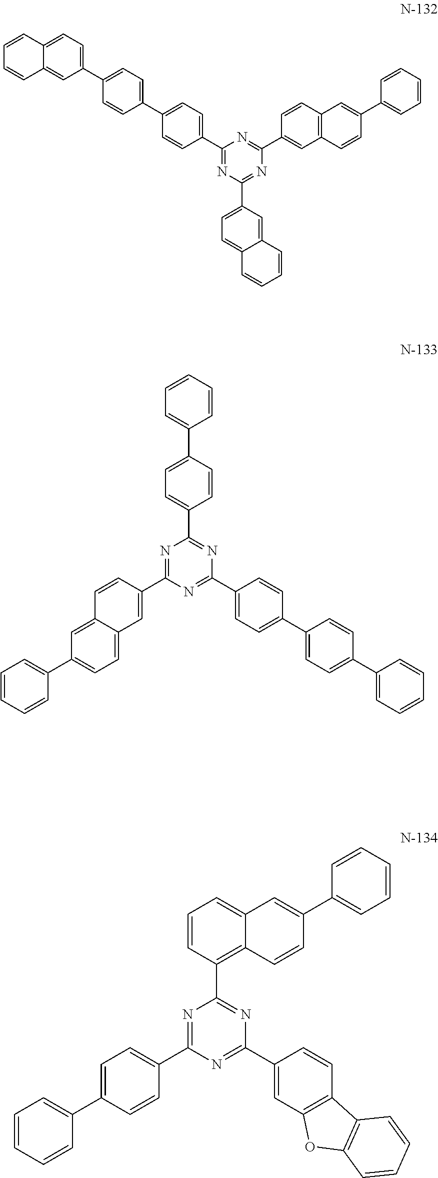 [SMILES: c1ccc(-c2ccc3cc(-c4nc(-c5ccc(-c6ccc(-c7ccc8ccccc8c7)cc6)cc5)nc(-c5ccc6ccccc6c5)n4)ccc3c2)cc1 ; c1ccc(-c2ccc(-c3ccc(-c4nc(-c5ccc(-c6ccccc6)cc5)nc(-c5ccc6cc(-c7ccccc7)ccc6c5)n4)cc3)cc2)cc1 ; c1ccc(-c2ccc(-c3nc(-c4ccc5c(c4)oc4ccccc45)nc(-c4cccc5cc(-c6ccccc6)ccc45)n3)cc2)cc1]

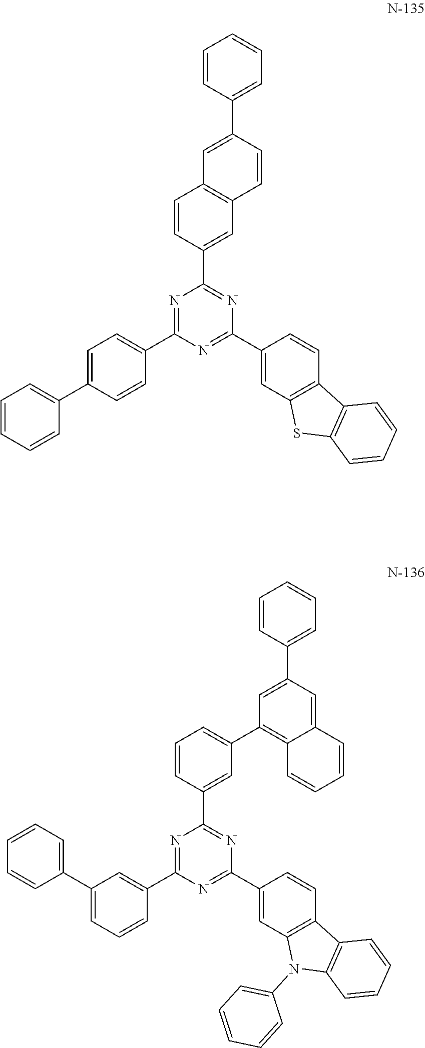 [SMILES: c1ccc(-c2ccc(-c3nc(-c4ccc5cc(-c6ccccc6)ccc5c4)nc(-c4ccc5c(c4)sc4ccccc45)n3)cc2)cc1 ; c1ccc(-c2cccc(-c3nc(-c4cccc(-c5cc(-c6ccccc6)cc6ccccc56)c4)nc(-c4ccc5c6ccccc6n(-c6ccccc6)c5c4)n3)c2)cc1]

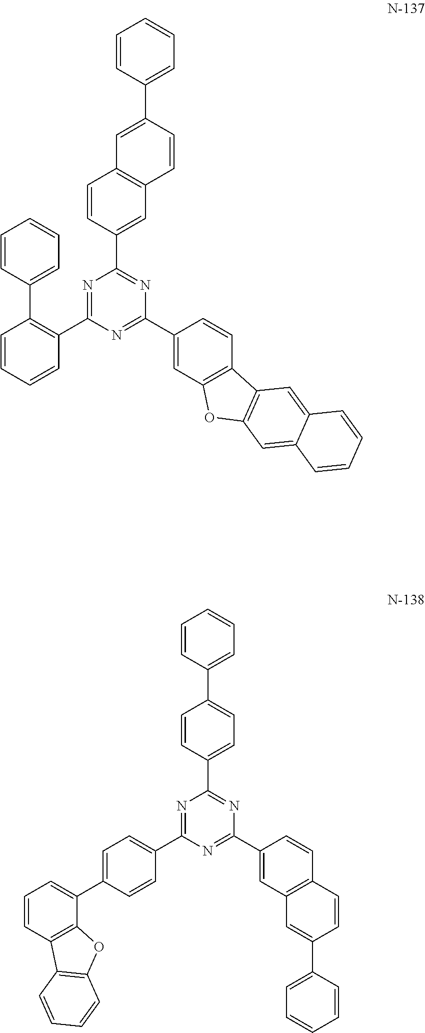 [SMILES: c1ccc(-c2ccc3cc(-c4nc(-c5ccc6c(c5)oc5cc7ccccc7cc56)nc(-c5ccccc5-c5ccccc5)n4)ccc3c2)cc1 ; c1ccc(-c2ccc(-c3nc(-c4ccc(-c5cccc6c5oc5ccccc56)cc4)nc(-c4ccc5ccc(-c6ccccc6)cc5c4)n3)cc2)cc1]

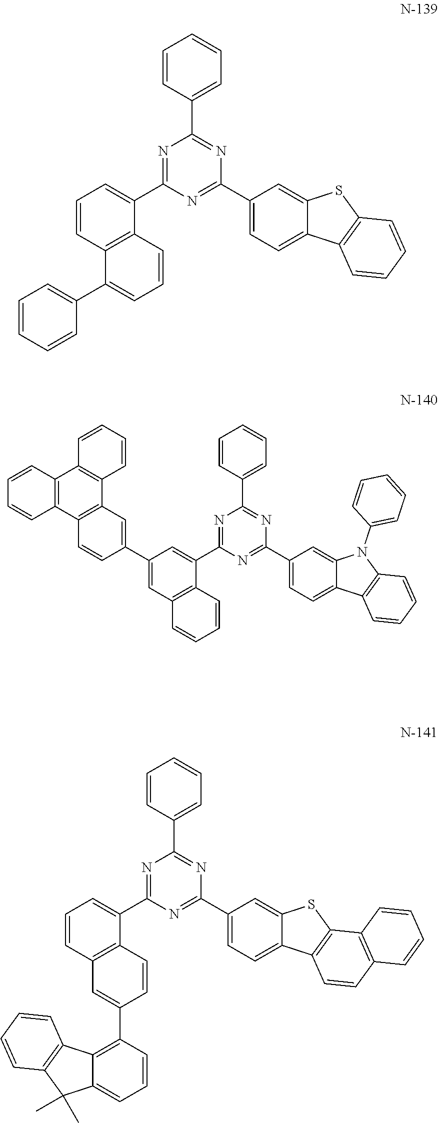 [SMILES: c1ccc(-c2nc(-c3ccc4c(c3)sc3ccccc34)nc(-c3cccc4c(-c5ccccc5)cccc34)n2)cc1 ; c1ccc(-c2nc(-c3ccc4c5ccccc5n(-c5ccccc5)c4c3)nc(-c3cc(-c4ccc5c6ccccc6c6ccccc6c5c4)cc4ccccc34)n2)cc1 ; CC1(C)c2ccccc2-c2c(-c3ccc4c(-c5nc(-c6ccccc6)nc(-c6ccc7c(c6)sc6c8ccccc8ccc76)n5)cccc4c3)cccc21]

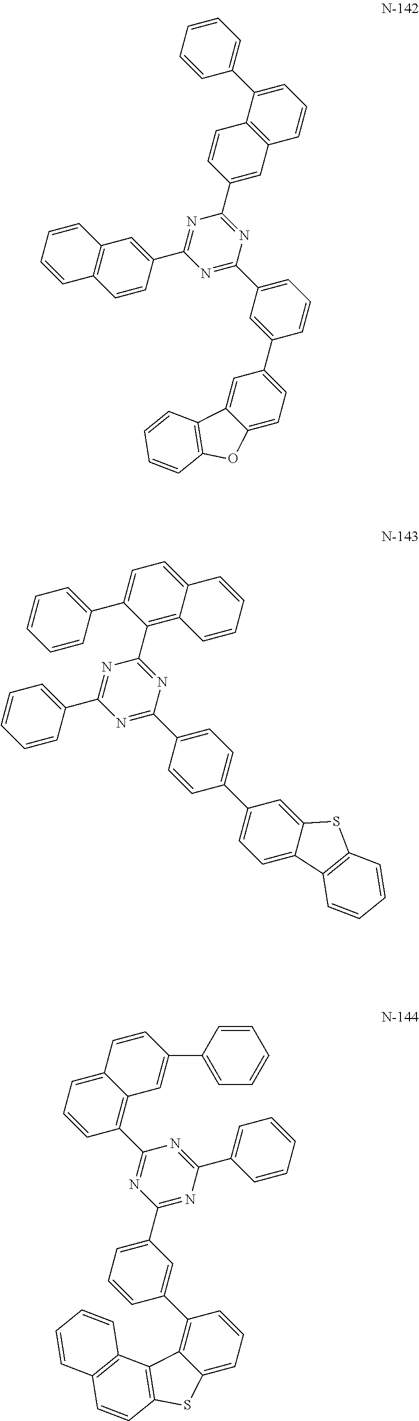 [SMILES: c1ccc(-c2cccc3cc(-c4nc(-c5cccc(-c6ccc7oc8ccccc8c7c6)c5)nc(-c5ccc6ccccc6c5)n4)ccc23)cc1 ; c1ccc(-c2nc(-c3ccc(-c4ccc5c(c4)sc4ccccc45)cc3)nc(-c3c(-c4ccccc4)ccc4ccccc34)n2)cc1 ; c1ccc(-c2ccc3cccc(-c4nc(-c5ccccc5)nc(-c5cccc(-c6cccc7sc8ccc9ccccc9c8c67)c5)n4)c3c2)cc1]

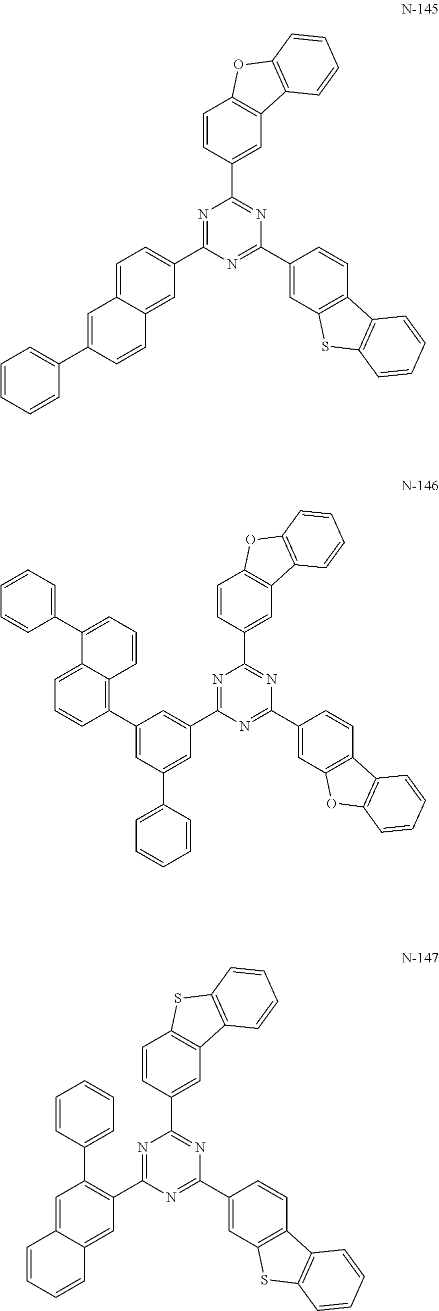 [SMILES: c1ccc(-c2ccc3cc(-c4nc(-c5ccc6c(c5)sc5ccccc56)nc(-c5ccc6oc7ccccc7c6c5)n4)ccc3c2)cc1 ; c1ccc(-c2cc(-c3nc(-c4ccc5c(c4)oc4ccccc45)nc(-c4ccc5oc6ccccc6c5c4)n3)cc(-c3cccc4c(-c5ccccc5)cccc34)c2)cc1 ; c1ccc(-c2cc3ccccc3cc2-c2nc(-c3ccc4c(c3)sc3ccccc34)nc(-c3ccc4sc5ccccc5c4c3)n2)cc1]

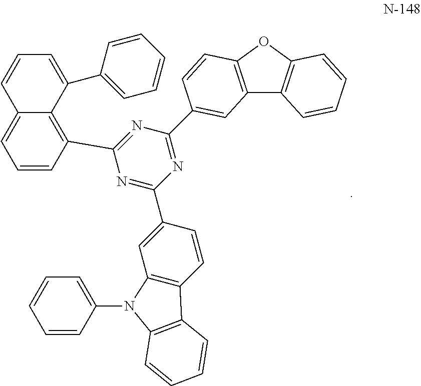 [SMILES: c1ccc(-c2cccc3cccc(-c4nc(-c5ccc6oc7ccccc7c6c5)nc(-c5ccc6c7ccccc7n(-c7ccccc7)c6c5)n4)c23)cc1]

14. The organic electronic device of claim 8, wherein the organic layer comprises two or more stacks, and the two or more stacks each comprise a hole-transporting layer, a light-emitting layer and an electron-transporting layer formed sequentially on the first electrode.

15. The organic electronic device of claim 14, wherein the two or more stacks further comprise a light-emitting auxiliary layer formed between the hole-transporting layer and the light-emitting layer.

16. The organic electronic device of claim 15, wherein the organic layer further comprises a charge generation layer between the two or more stacks.

17. The organic electronic device of claim 8, wherein the organic electronic device further comprises a layer for improving luminous efficiency, and the layer for improving luminous efficiency is formed on one side of either the first electrode or the second, and the one side not facing the organic layer.

18. An electronic apparatus comprising a display device and a control unit configured to drive the display device, wherein the display device comprises the organic electronic device of claim 8.

19. The electronic device of claim 18, wherein the organic electronic device is selected from the group consisting of an organic electroluminescent device, an organic solar cell, an organic photo conductor, an organic transistor, a device for monochromatic illumination and a quantum dot display device.

20. A compound obtained by recovering and purifying the material of an organic layer from the deposition equipment after depositing the organic layer in the manufacturing process of an organic electronic device, wherein the compound is a compound of Formula 1 of claim 1.

21. The compound of claim 20, wherein a purity of the compound is at least 99.9%.

22. A method for recovering a compound represented by Formula 1 comprising:
a step of depositing material of an organic layer comprising a compound represented by Formula 1 of claim 1;
a step of recovering the material of organic layer attached to the deposition equipment; and
a step of purifying the recovered material of an organic layer to obtain a compound represented by Formula 1 having a purity of 99.9% or higher.

23. The method for recovering a compound represented by Formula 1 of claim 22, wherein the step of purifying the recovered material of an organic layer comprises a step of recrystallizing the recovered material of an organic layer using a recrystallization solvent, a step of adsorbing and separating using an adsorbent, and a step of sublimation and purification.
